(19) United States
(12) Patent Application Publication
(10) Pub. No.: US 2005/0272051 A1
(43) Pub. Date: Dec. 8, 2005

(54) Methods of preventing or treating recurrence of myocardial infarction

(75) Inventors: Anna Helgadottir, Hamravik (IS); Mark Gurney, East Grand Rapids, MI (US); Hakon Hakonarson, Reykjavik (IS); Jeffrey R. Gulcher, Lake Barrington, IL (US)

Correspondence Address: David A. Gass, Esq.;Marshall, Gerstein & Borun LLP, 6300 Sears Tower, 233 South Wacker Drive, Chicago, IL, 60606-6357, US

(73) Assignee: deCODE genetics ehf., Reykjavik (IS)

(21) Appl. No.: 10/944,272

(22) Filed: Sep. 17, 2004

Related U.S. Application Data
(60) Provisional application No. 60/503,587, filed on Sep. 17, 2003.

(51) Int. Cl. C12Q 1/68

(52) U.S. Cl. 435/6

(57) ABSTRACT

Linkage of myocardial infarction (MI) with a locus on chromosome 12q23 is disclosed. In particular, the LTA4H gene within this locus is shown by association analysis to be a susceptibility gene for MI. Methods for preventing and/or treating the recurrence of MI, in particular are described.

### RELATED APPLICATION

This application claims the benefit of U.S. Provisional Application No. 60/503,587, filed on Sep. 17, 2003. The entire teachings of the above application are incorporated herein by reference.

### BACKGROUND OF THE INVENTION

Myocardial Infarction (MI) is one of the most common diagnoses in hospitalized patients in industrialized countries. Myocardial Infarction generally occurs when there is an abrupt decrease in coronary blood flow following a thrombotic occlusion of a coronary artery previously narrowed by atherosclerosis. Infarction occurs when a coronary artery thrombus develops rapidly at a site of vascular injury, which is produced or facilitated by factors such as cigarette smoking, hypertension and lipid accumulation. In most cases, infarction occurs when an atherosclerotic plaque fissures, ruptures or ulcerates and when conditions favor thrombogenesis. In rare cases, infarction may be due to coronary artery occlusion caused by coronary emboli, congenital abnormalities, coronary spasm, and a wide variety of systemic, particularly inflammatory diseases.

Although classical risk factors such as smoking, hyperlipidemia, hypertension, and diabetes are associated with many cases of coronary heart disease (CHD) and MI, many patients do not have involvement of these risk factors. In fact, many patients who exhibit one or more of these risk factors do not develop MI. Family history has long been recognized as one of the major risk factors. Although some of the familial clustering of MI reflects the genetic contribution to the other conventional risk factors, a large number of studies have suggested that there are significant genetic susceptibility factors, beyond those of the known risk factors (Friedlander Y, et al., Br Heart J. 1985; 53:382-7, Shea S. et al., J. Am. Coll. Cardiol. 1984; 4:793-801, and Hopkins P. N., et al., Am. J. Cardiol. 1988; 62:703-7). Major genetic susceptibility factors have not yet been published. Currently anti-coagulants (e.g., aspirin) or cholesterol lowering drugs (e.g., statins) are used to prevent or treat the recurrence of myocardial infarction.

### SUMMARY OF THE INVENTION

As described herein, a gene on chromosome 12q23 has been identified as playing a major role in myocardial infarction (MI). The gene comprises nucleic acid that encodes leukotriene A4 hydrolase, herein after referred to as LTA4H.

The invention pertains to methods of treatment (prophylactic and/or therapeutic) for certain diseases and conditions (e.g., MI, ACS, atherosclerosis) associated with LTA4H or with other members of the leukotriene pathway (e.g., biosynthetic enzymes, such as 5-lipoxygenase activating protein (FLAP) and arachidonate 5-lipoxygenase (5-LO); catabolic enzymes, such as leukotriene B4 12-hydroxydehydrogenase (LTB4DH) and leukotriene B4 omega hydroxylase; receptors, modulators and/or binding agents of the enzymes; and receptors for leukotriene B4 (LTB4), including leukotriene B4 receptor 1 (BLT1), and leukotriene B4 receptor 2 (BLT2)). The methods include the following: methods of treatment for myocardial infarction or susceptibility to myocardial infarction; for acute coronary syndrome (ACS), e.g., unstable angina, non-ST-elevation myocardial infarction (NSTEMI) or ST-elevation myocardial infarction (STEMI); for decreasing risk of a second myocardial infarction; for atherosclerosis, such as for patients requiring treatment (e.g., angioplasty, stents, coronary artery bypass graft) to restore blood flow in arteries (e.g., coronary arteries); and/or for decreasing leukotriene synthesis (e.g., for preventing or treating recurrence of myocardial infarction).

In the methods of the invention, a leukotriene synthesis inhibitor is administered to an individual in a therapeutically effective amount. The leukotriene synthesis inhibitor can be an agent that inhibits or antagonizes a member of the leukotriene synthesis pathway (e.g., LTA4H, FLAP, or 5-LO). For example, the leukotriene synthesis inhibitor can be an agent that inhibits or antagonizes LTA4H polypeptide activity (e.g., an LTA4H inhibitor) and/or LTA4H nucleic acid expression, as described herein. In another embodiment, the leukotriene synthesis inhibitor is an agent that inhibits or antagonizes polypeptide activity and/or nucleic acid expression of another member of the leukotriene biosynthetic pathway (e.g., FLAP, 5-LO) or an LTB4 receptor (e.g., BLT1 and/or BLT2). In preferred embodiments, the agent alters activity and/or nucleic acid expression of LTA4H. Preferred agents include those set forth in the Agent Table and in the Additional LTA4H Agent List herein. In another embodiment, preferred agents can be: ethyl-1-[2-[4-(phenylmethyl)phenoxy]ethyl]-4-piperidine-carboxylate, otherwise known as SC-56938; [4-[5-(3-Phenyl-propyl)thiophen-2-yl]butoxy]acetic acid, otherwise known as RP64966; (R)-S-[[4-(dimethylamino)phenyl]methyl]-N-(3-mercapto-2methyl-1-oxopropyl-L-cycteine, otherwise known as SA6541; optically pure enantiomers, salts, chemical derivatives, and analogues. In another embodiment, the agent alters metabolism or activity of a leukotriene (e.g., LTB4), such as leukotriene antagonists or antibodies to leukotrienes, as well as agents which alter activity of a leukotriene receptor (e.g., BLT1 and/or BLT2).

In certain embodiments of the invention, the individual is an individual who has at least one risk factor, such as an at-risk haplotype for myocardial infarction; an at-risk haplotype in the LTA4H gene; a polymorphism in a LTA4H nucleic acid; an at-risk polymorphism in the FLAP gene, an at-risk polymorphism in the 5-LO gene promoter, diabetes; hypertension; hypercholesterolemia; elevated lp(a); obesity; a past or current smoker; an elevated inflammatory marker (e.g., a marker such as C-reactive protein (CRP), serum amyloid A, myeloperoxidase (MPO), N-tyrosine, di-tyrosine, lipoprotein phospholipase A2 (Lp-PLA2), fibrinogen, a leukotriene, a leukotriene metabolite, interleukin-6, tissue necrosis factor-alpha, a soluble vascular cell adhesion molecule (sVCAM), a soluble intervascular adhesion molecule (sICAM), E-selectin, matrix metalloprotease type-1, matrix metalloprotease type-2, matrix metalloprotease type-3, and matrix metalloprotease type-9); increased total cholesterol, LDL cholesterol and/or decreased HDL cholesterol; increased leukotriene synthesis; and/or at least one previous myocardial infarction, ACS, stable angina, atherosclerosis, history of peripheral arterial occlusive disease, previous or acute stroke or transient ischemic attack, and past or acute treatment for restoration of coronary artery blood flow (e.g., angioplasty, stenting, coronary artery bypass graft).

The invention pertains to use of leukotriene synthesis inhibitors for the manufacture of a medicament for the prevention and/or treatment of MI, ACS, and/or atherosclerosis, as described herein, as well as for the manufacture of a medicament for the reduction of leukotriene synthesis.

### BRIEF DESCRIPTION OF THE DRAWINGS

FIG. 1 shows the results of the first step of the linkage analysis: multipoint non-parametric LOD scores for a framework marker map on chromosome 12. A LOD score suggestive of linkage of 1.95 was found at marker D12S2081.

FIG. 2 shows the results of the second step of the linkage analysis: multipoint non-parametric LOD scores for the families after adding 20 fine mapping markers to the candidate region. The inclusion of additional microsatellite markers increased the information on sharing by decent from 0.8 to 0.9, around the markers that gave the highest LOD scores.

FIGS. 3.1-3.33 show the genomic sequence of the LTA4H gene (SEQ ID NO: 1).

FIG. 4 shows the sequence of the LTA4H mRNA (SEQ ID NO: 2).

FIG. 5 shows the sequence of the LTA4H polypeptide (SEQ ID NO: 3).

FIGS. 6.1-6.32 show the sequences of particular SNPs of the LTA4H gene (SEQ ID NOs: 4-92).

FIGS. 7.1-7.8 show the sequences of other particular SNPs of the LTA4H gene (SEQ ID NOs: 93-117).

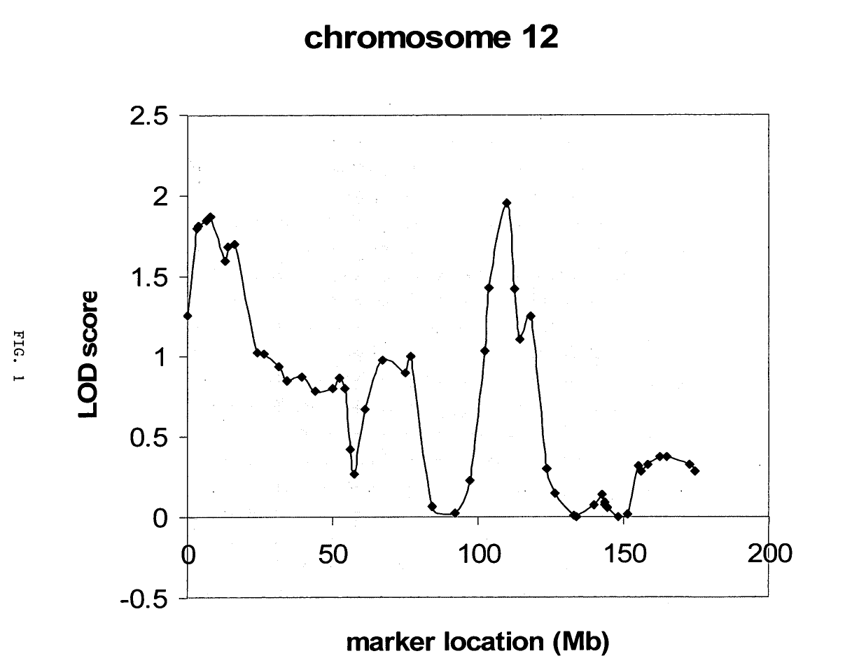

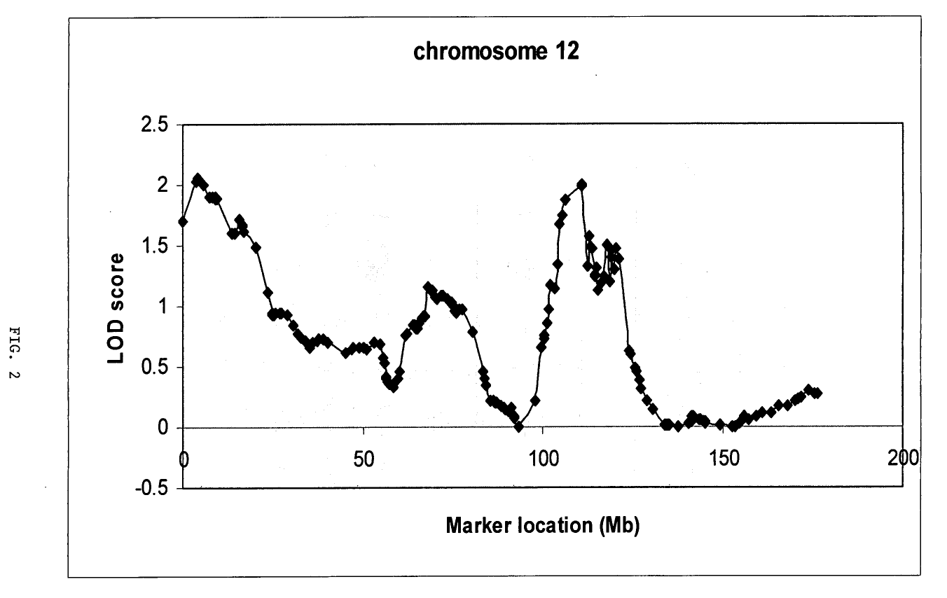

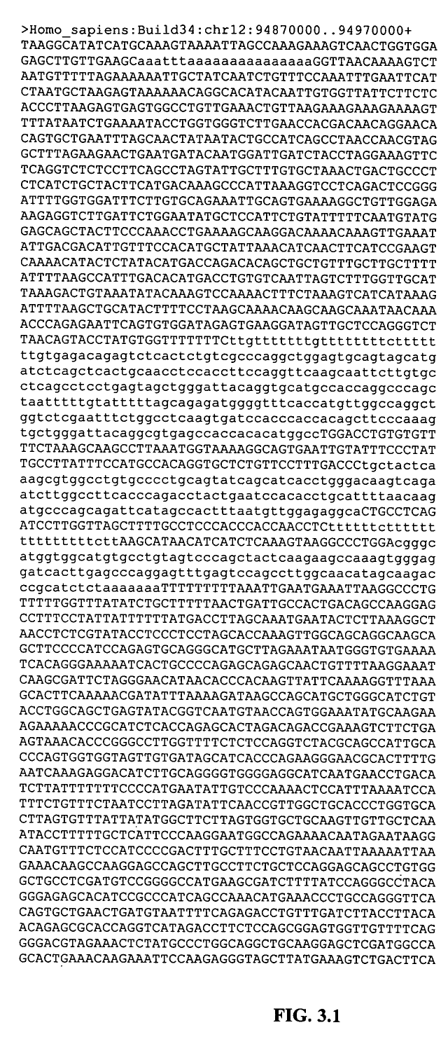

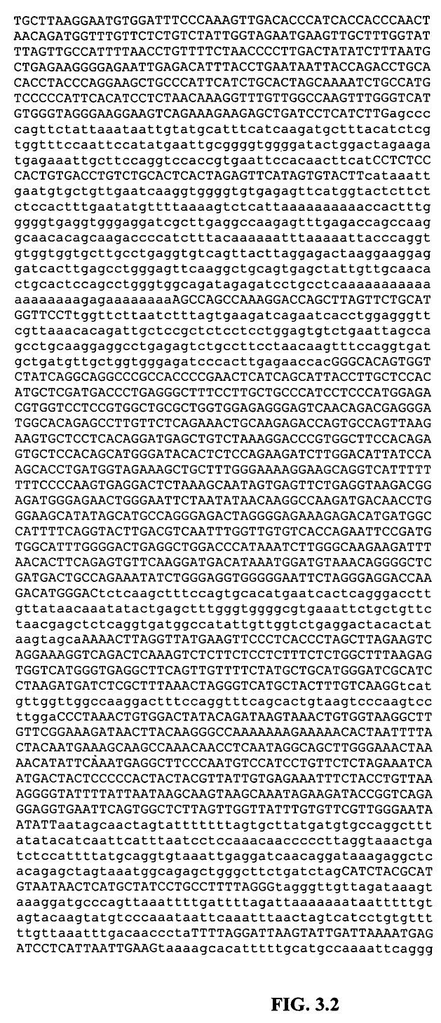

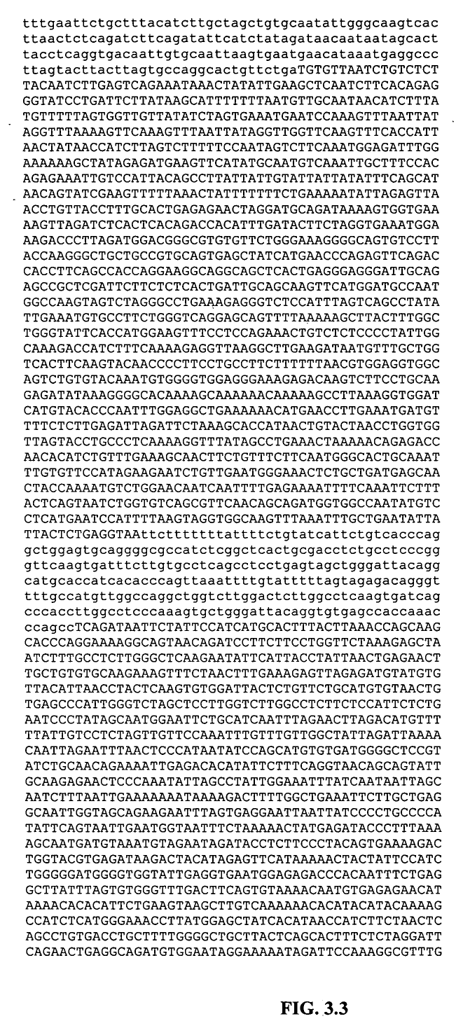

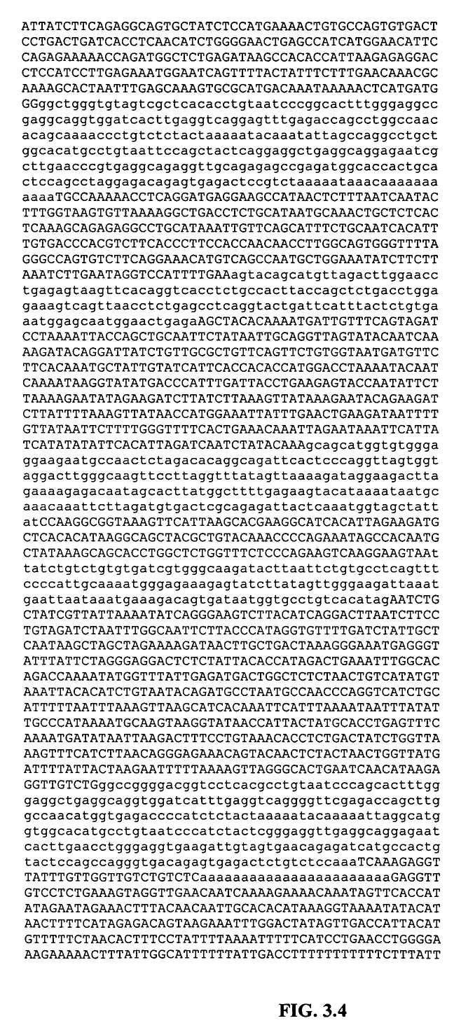

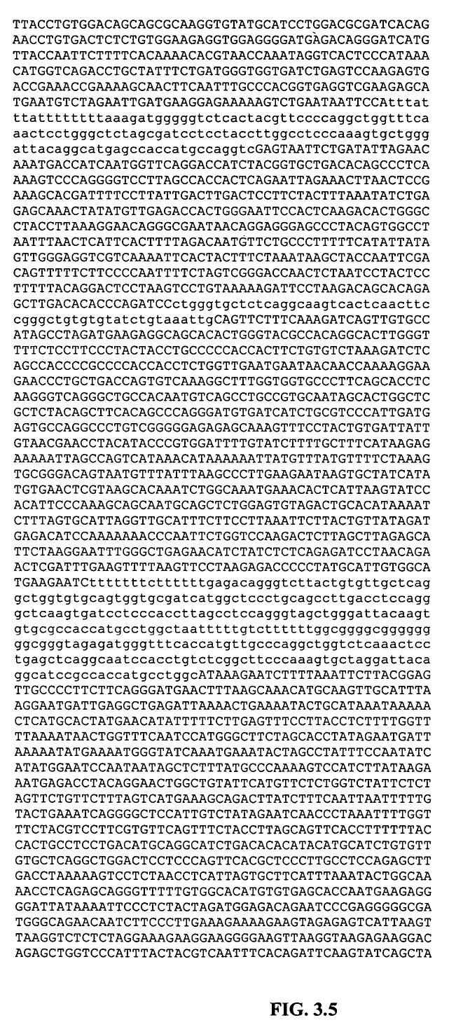

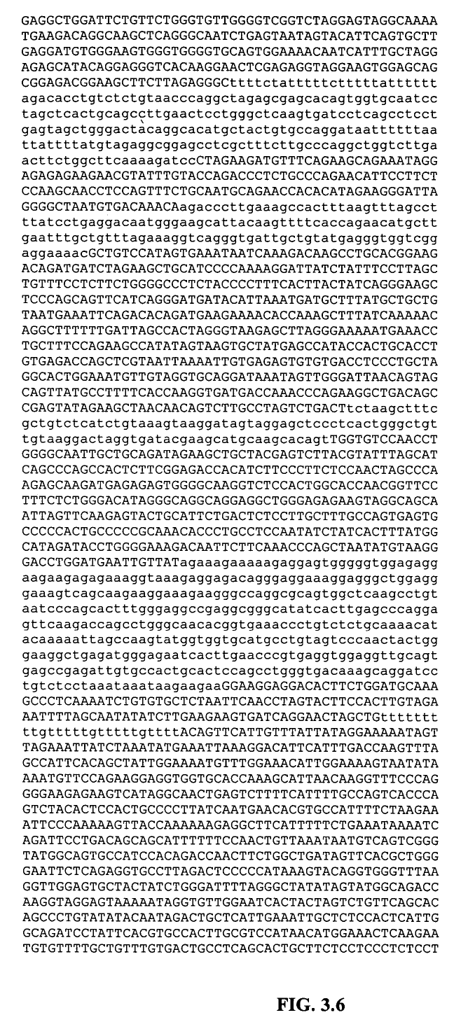

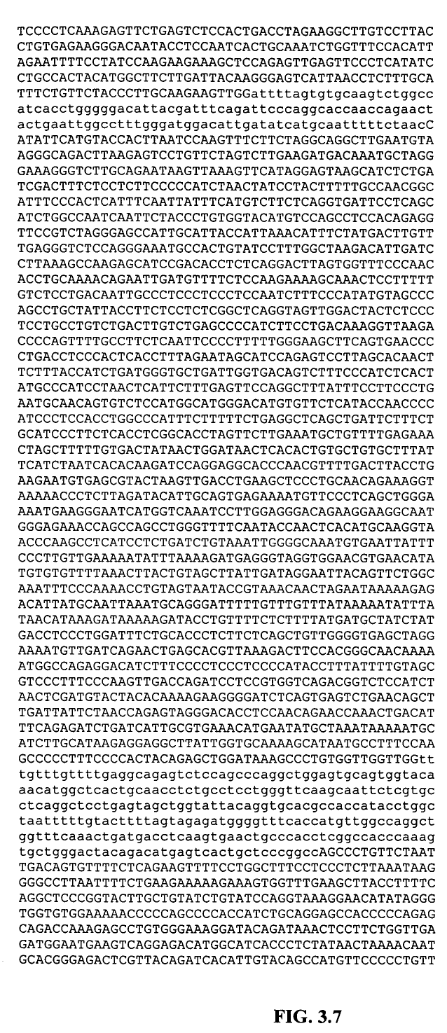

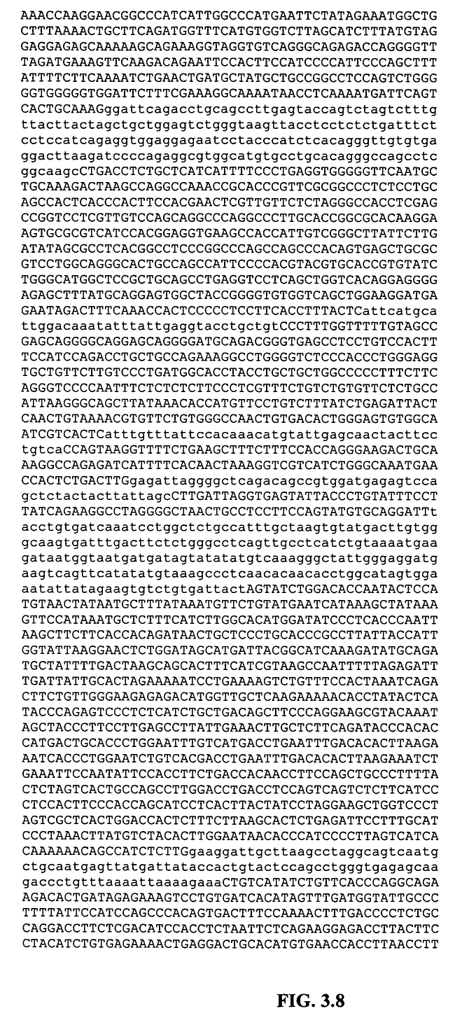

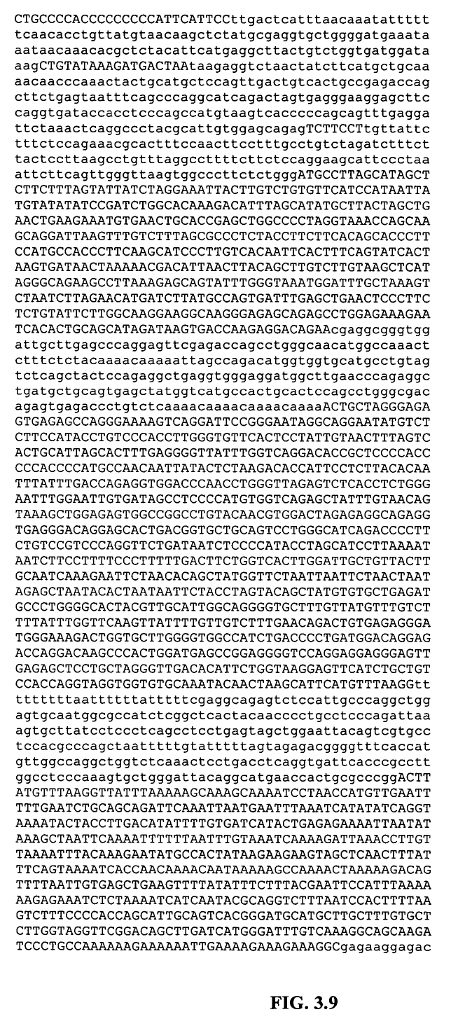

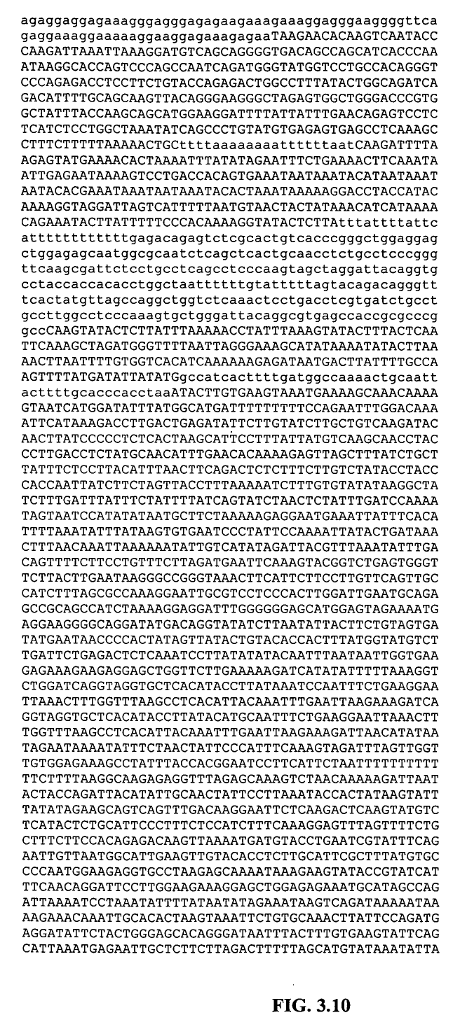

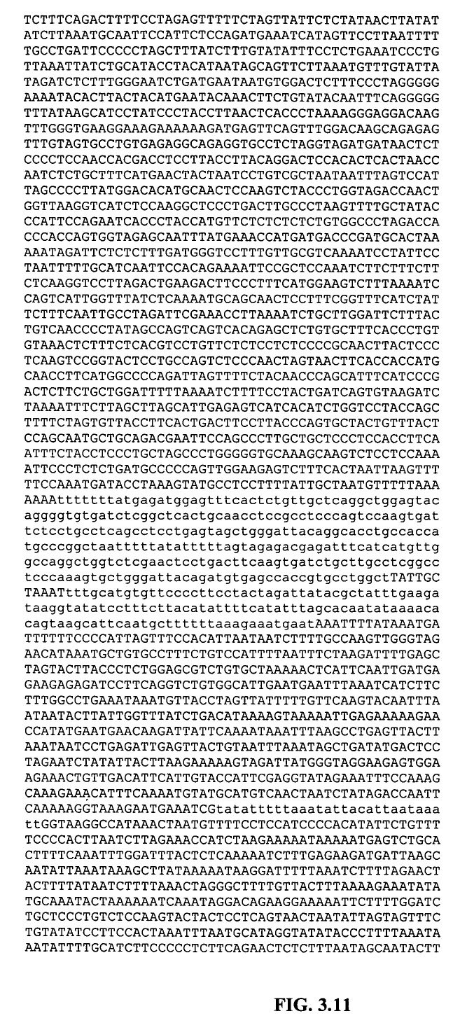

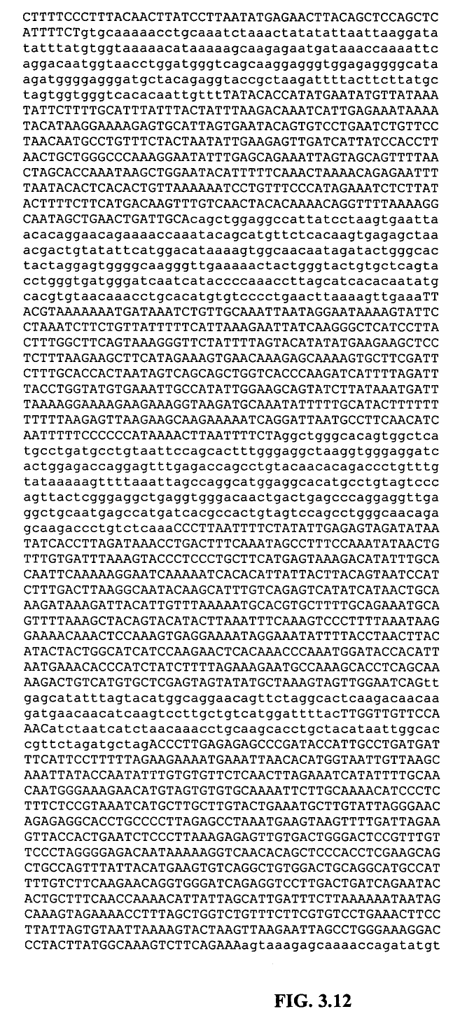

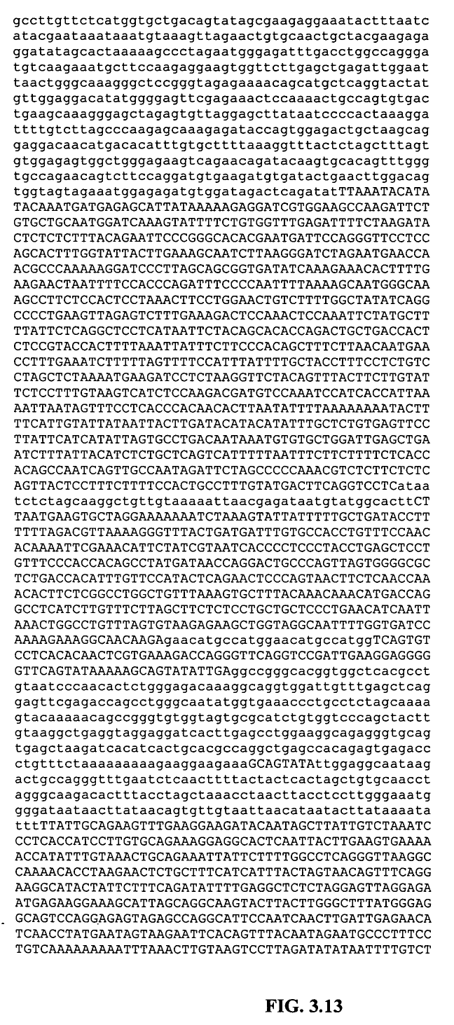

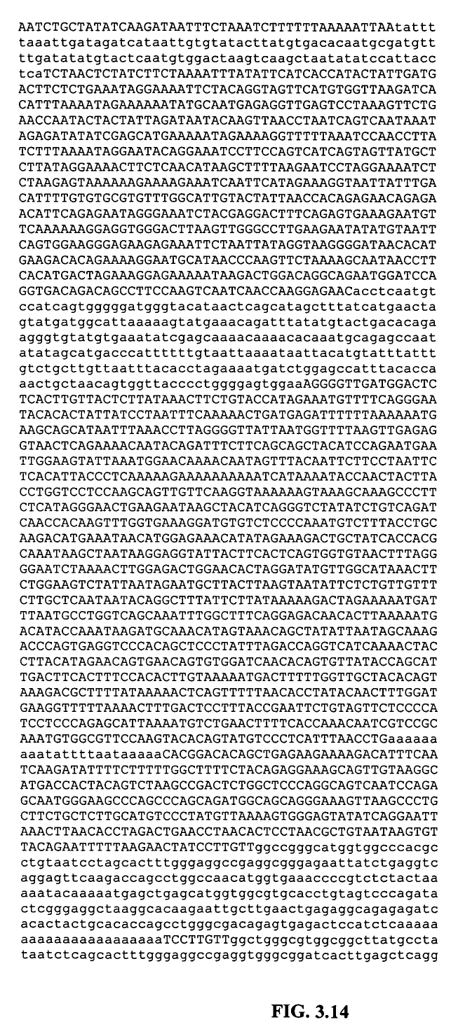

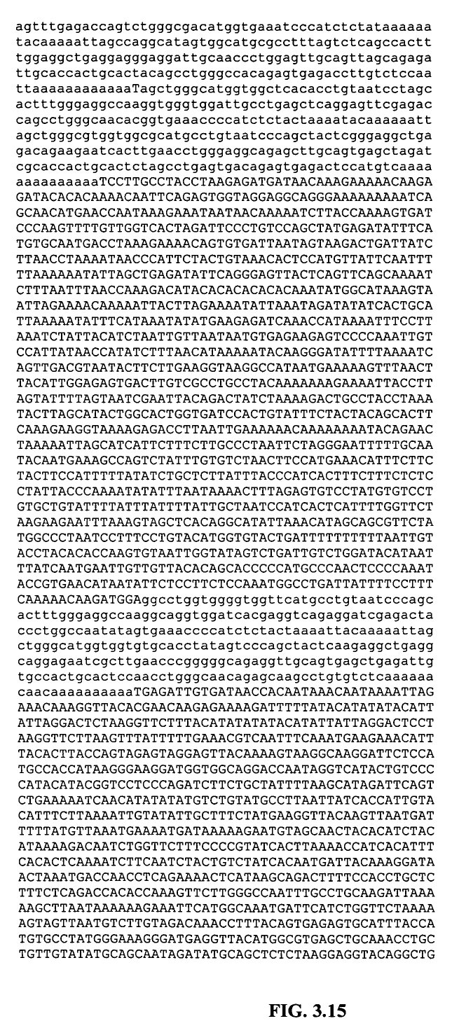

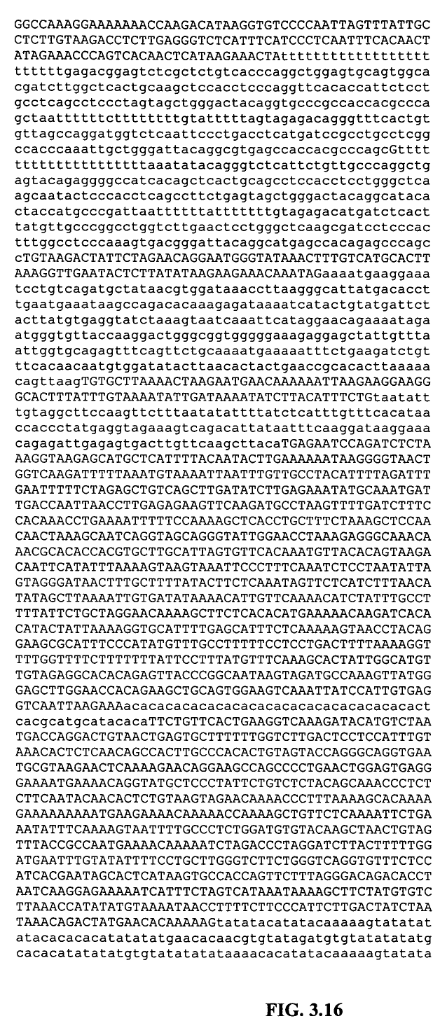

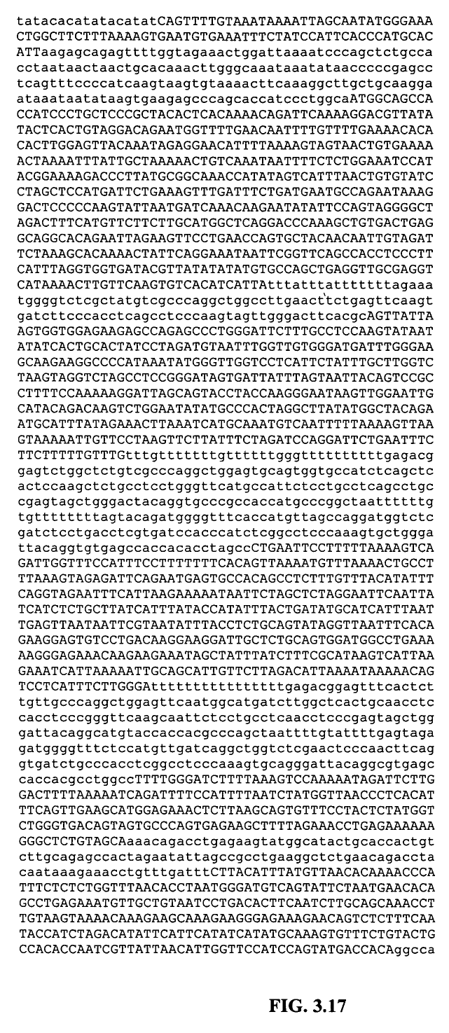

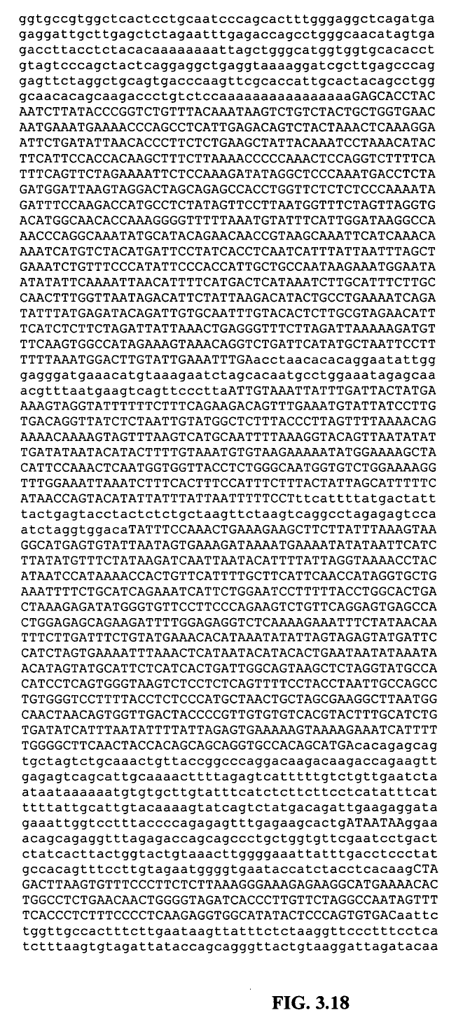

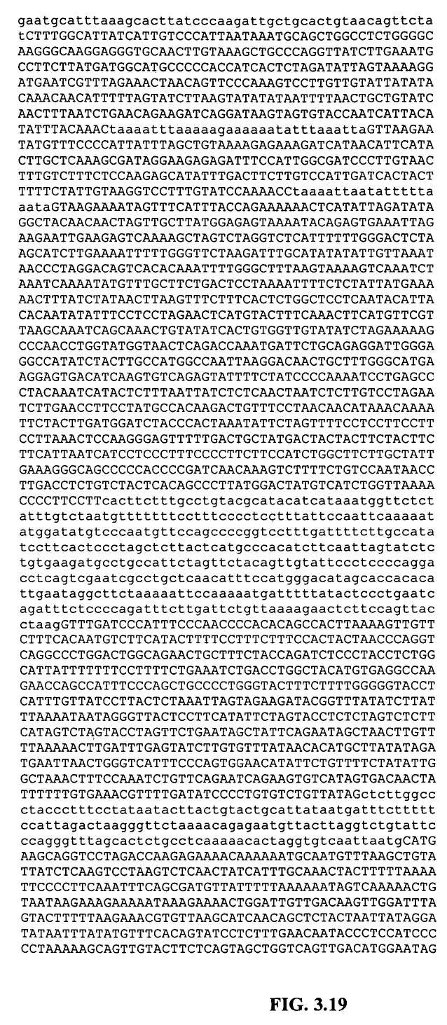

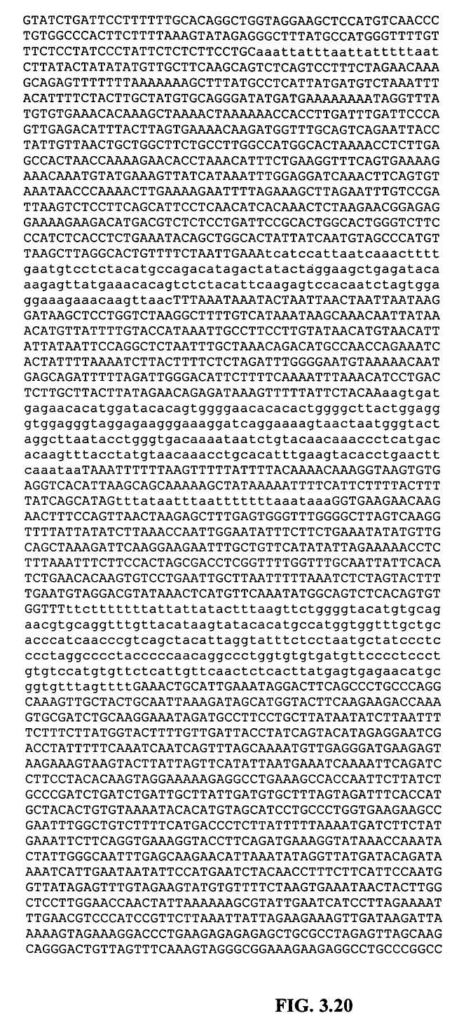

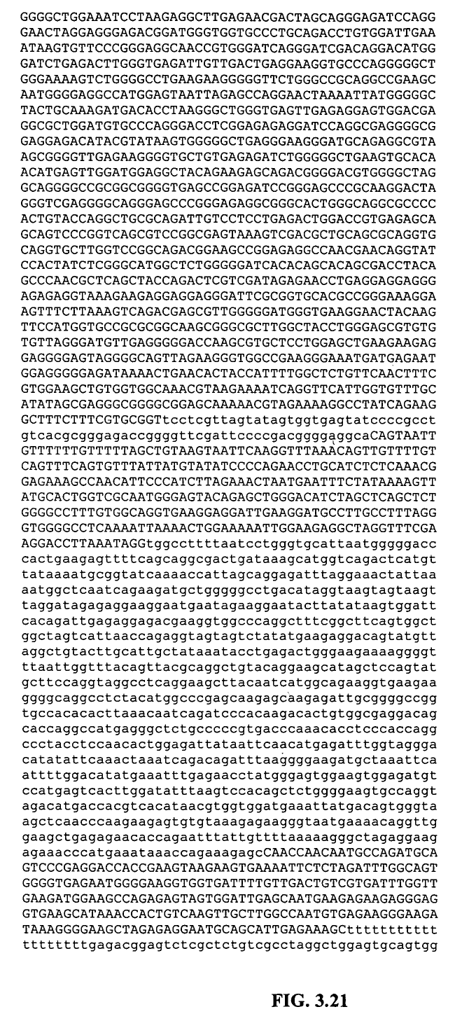

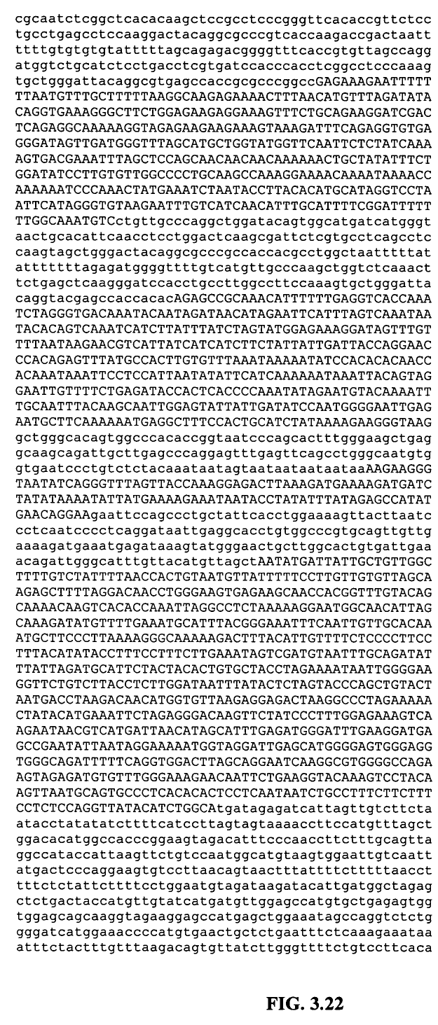

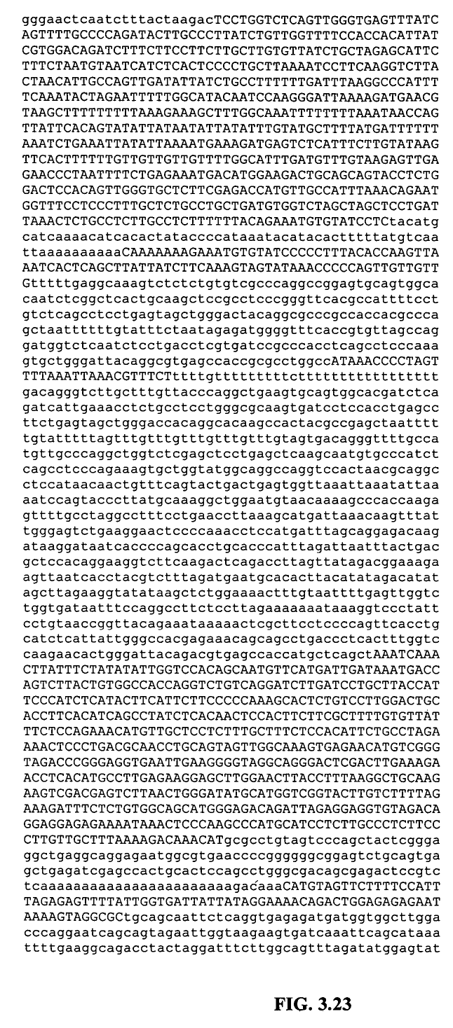

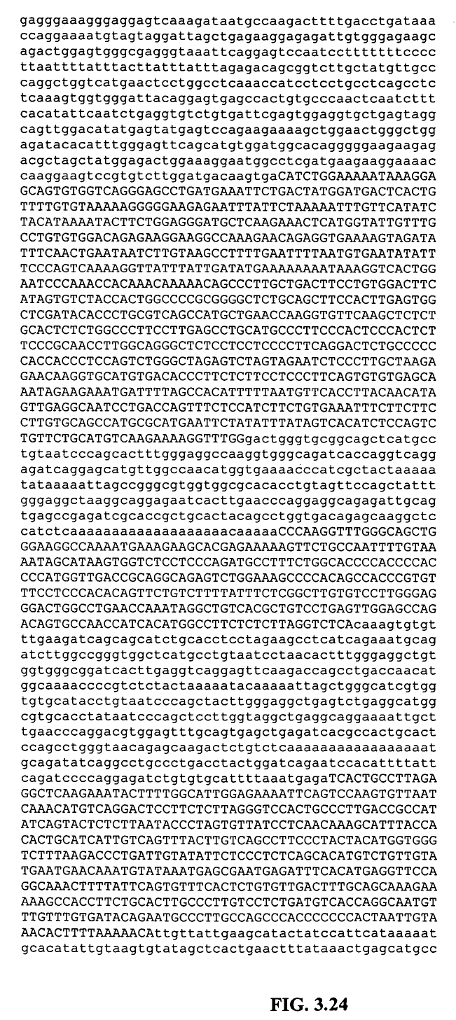

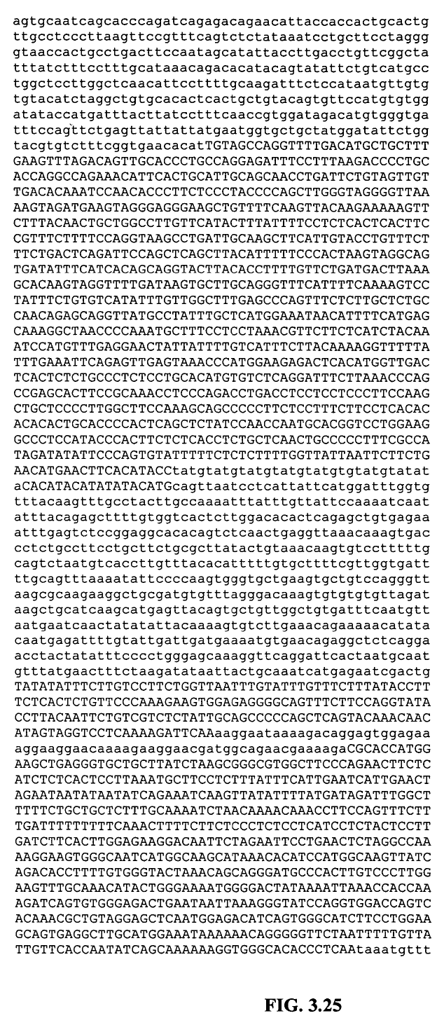

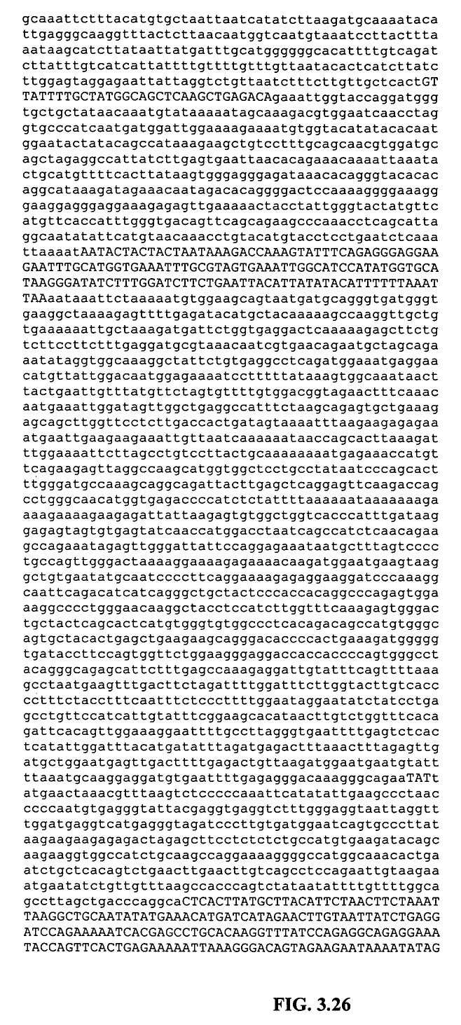

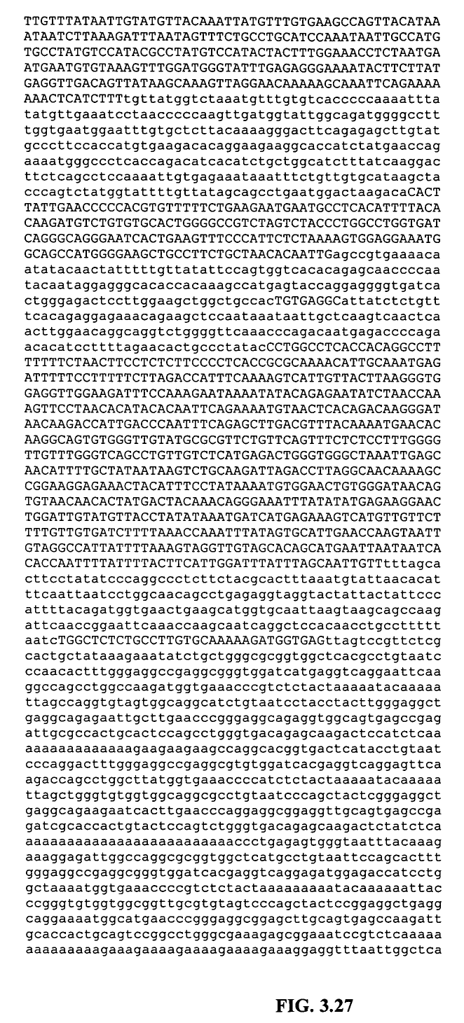

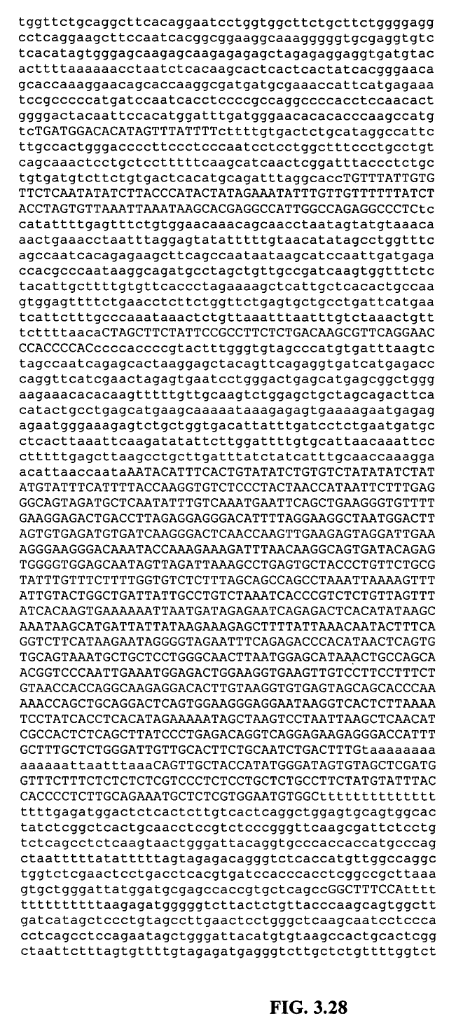

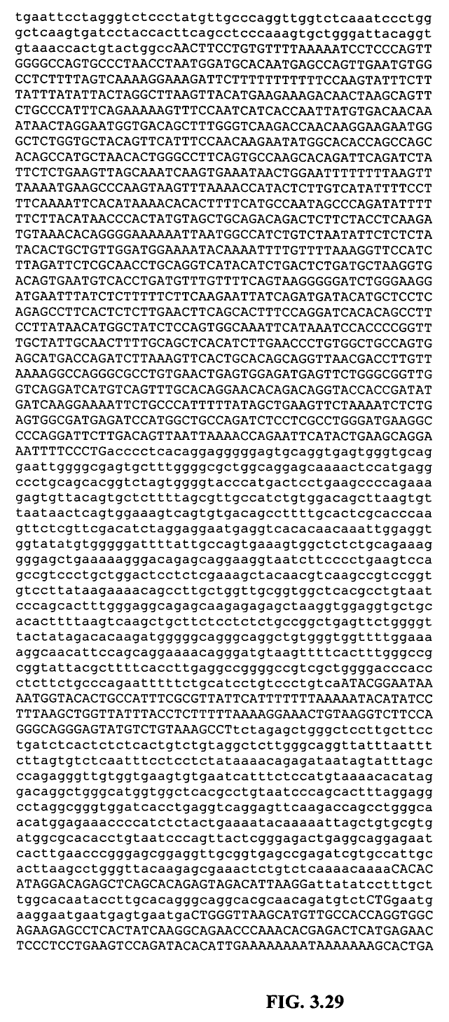

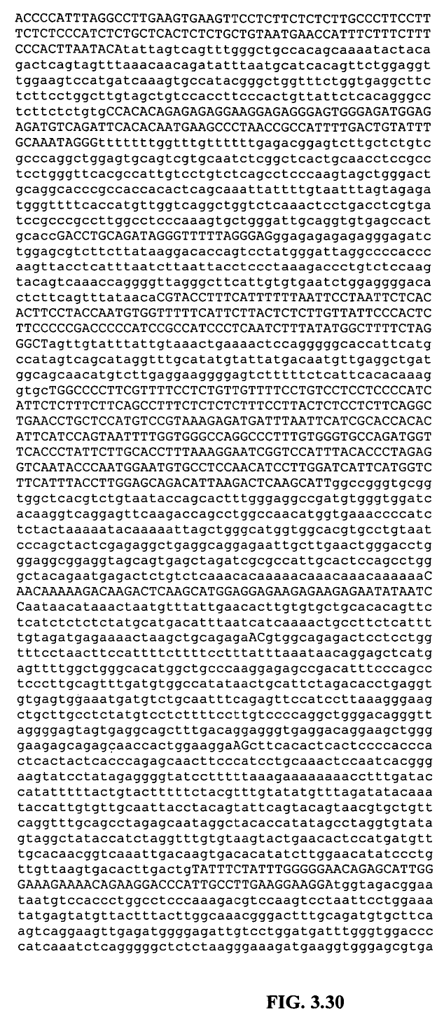

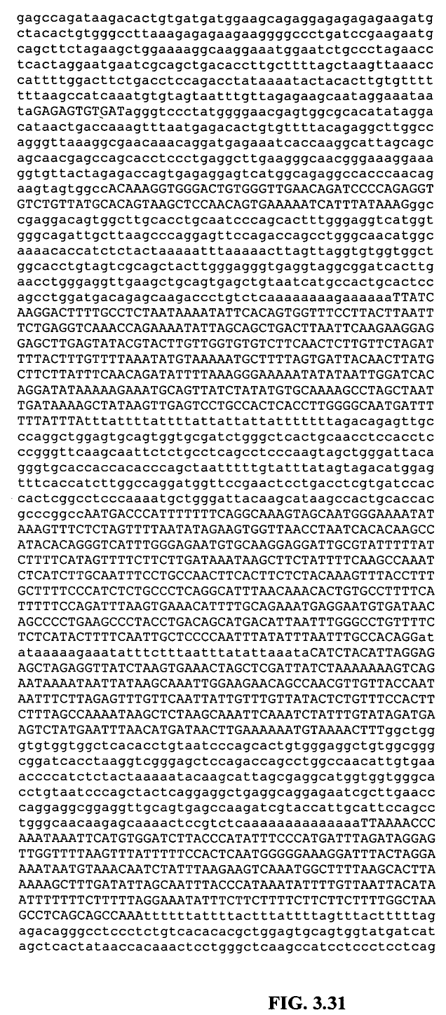

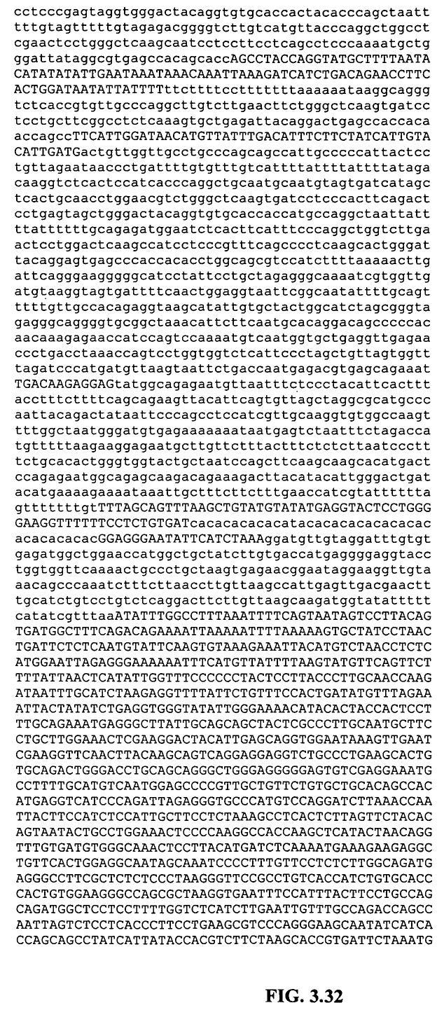

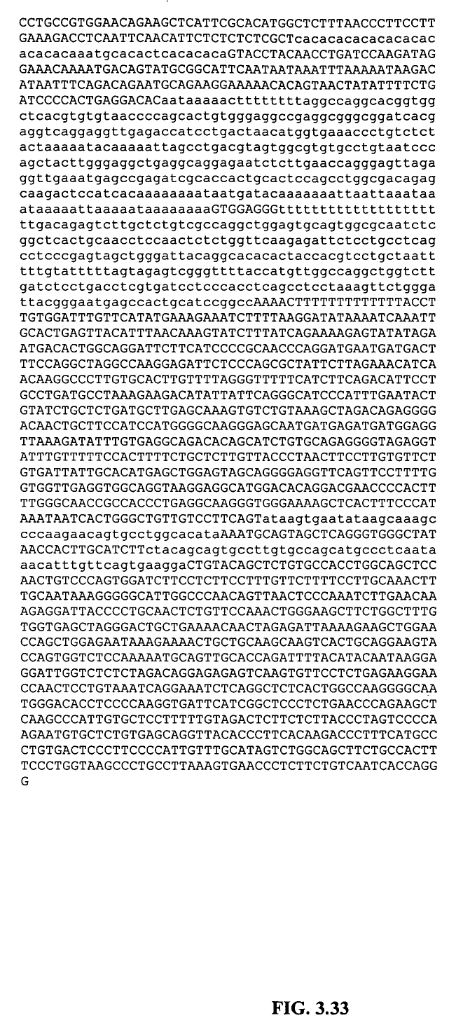

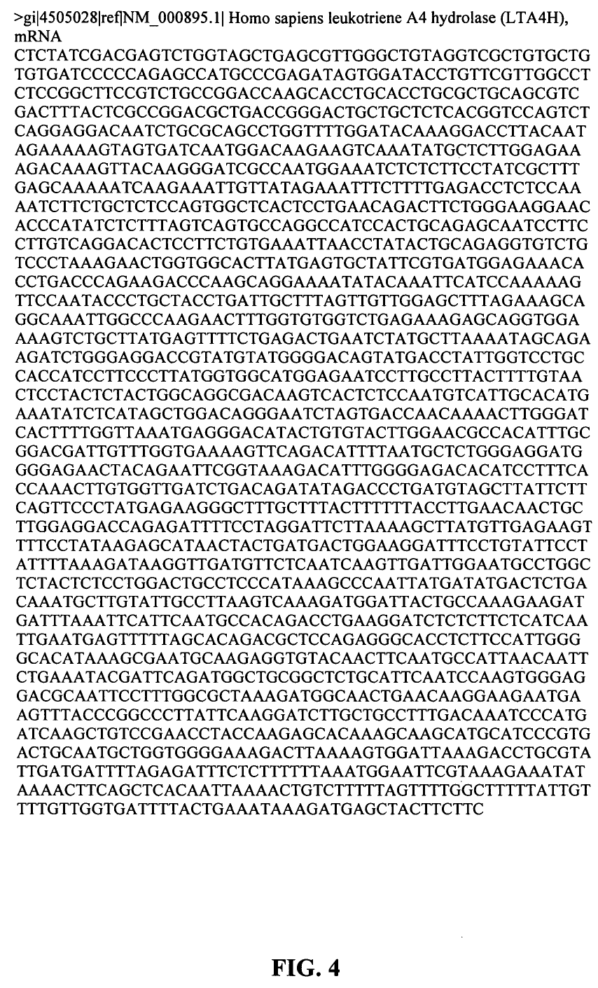

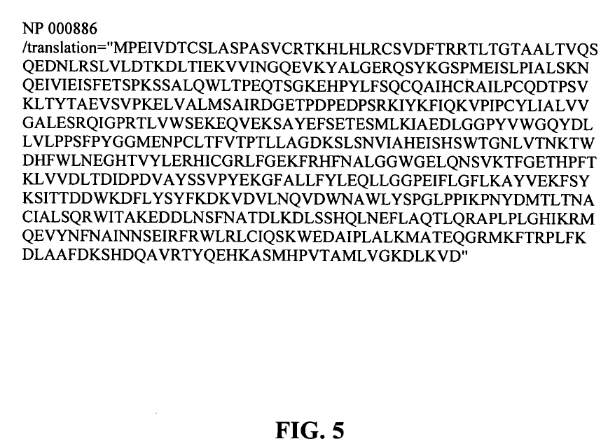

### DETAILED DESCRIPTION OF THE INVENTION

In a genome wide search for genes that cause MI using a large number of Icelandic patients and families, linkage (that is, excess sharing of a given location in the genome) was found to a locus or location on chromosome 12q23. Given our past discovery that FLAP is major gene contributing to MI risk, we noted that a candidate gene encoding a protein in the same molecular pathway as FLAP, LTA4H, resided within this locus. Three microsatellite markers and 12 SNPs spanning a 79 kb region across the LTA4H gene were genotyped in approximately 1000 patients and 460 controls.

A haplotype consisting of 2 microsatellite markers and 2 SNPs was found to be in significant excess in MI patients, compared with controls. These results strongly suggest that the LTA4H gene is a susceptibility gene for myocardial infarction and is likely involved in its pathogenesis or underlying disease process. The LTA4H nucleic acid encodes an enzyme, leukotriene A4 hydrolase, which participates in leukotriene biosynthesis. Other members of the leukotriene pathway have been shown to be associated with MI (see U.S. Provisional Application No. 60/419,432, filed on Oct. 17, 2002; U.S. patent application Ser. No. 10/829,674, filed on Apr. 22, 2004). Mutations and/or polymorphisms within the LTA4H nucleic acid that show association with the disease can potentially be used for diagnostic purposes. Furthermore, the LTA4H gene, and other members of the leukotriene pathway are therapeutic targets for myocardial infarction.

The leukotrienes are a family of highly potent biological mediators of inflammatory processes produced primarily by bone marrow derived leukocytes such as monocytes, macrophages, and neutrophils. Leukotriene biosynthetic enzymes are detected within atherosclerosis lesions, indicating that the vessel itself can be a source of leukotrienes. Increased production of leukotrienes in individuals with pre-existing atherosclerosis lesions may lead to plaque instability or friability of the fibrous cap leading to local thrombotic events. If this occurs in coronary artery arteries it leads to MI or unstable angina. If it occurs in the cerebrovasculature it leads to stroke or transient ischemic attack. If it occurs in large arteries to the limbs, it causes or exacerbates limb ischemia in persons with peripheral arterial occlusive disease (PAOD). Therefore, those with genetically influenced predisposition to produce higher leukotriene levels may be at higher risk for local thrombotic events over a pre-existing atherosclerosic lesion leading to ischemic events such as MI, stroke, and PAOD. In addition, local leukotriene production by cells within atherosclerotic plaques and the vasculature may accelerate the progression of atherosclerosis and increase the risk of clinically important atherosclerosis.

As a result of these discoveries, methods are now available for the prevention and/or treatment of myocardial infarction (MI) and acute coronary syndrome (ACS) through the use of leukotriene inhibitors, such as agents that inhibit leukotriene biosynthesis or antagonize signaling through leukotriene receptors. The term, “treatment” as used herein, refers not only to ameliorating symptoms associated with the disease or condition, but also preventing or delaying the onset of the disease or condition; preventing or delaying the occurrence of a second episode of the disease or condition; and/or also lessening the severity or frequency of symptoms of the disease or condition. In the case of atherosclerosis, “treatment” also refers to a minimization or reversal of the development of plaques. Methods are additionally available for assessing an individual's risk for MI or ACS. In preferred embodiment, the individual to be treated is an individual who is susceptible (at increased risk) for MI or ACS, such as an individual who is in one of the representative target populations described herein.

Representative Target Populations

We have defined several target populations that may especially benefit from medicaments developed against LTA4H.

In one embodiment of the invention, an individual who is at risk for MI or ACS is an individual who has an at-risk haplotype in LTA4H, as described herein. In one embodiment, the haplotype can comprise alleles 0, T, 0, and A, of markers DG12S1664, SG12S26, DG12S1666, and SG12S144, respectively, at the 12q23 locus. This LTA4H “at-risk” haplotype is detected in over 76% of male patients who have previously had an MI, conferring an increased relative risk of 1.4 fold and in 72% of female MI patients with a relative risk of 1.2. Increased risk for MI or ACS in individuals with an LTA4H at-risk haplotype is logically conferred by increased production of leukotrienes in the arterial vessel wall or in bone-marrow derived inflammatory cells within the blood and/or arterial vessel wall. In another embodiment of the invention, an individual who is at risk for MI or ACS is an individual who has a polymorphism in an LTA4H gene, in which the presence of the polymorphism is indicative of a susceptibility to MI or ACS. The term “gene,” as used herein, refers to not only the sequence of nucleic acids encoding a polypeptide, but also the promoter regions, transcription enhancement elements, splice donor/acceptor sites, and other non-transcribed nucleic acid elements. Representative polymorphisms include those presented in Table 3. Along the same lines, certain variants in the FLAP gene and other members of the leukotriene biosynthetic and response pathway (see, U.S. Provisional Application No. 60/419,432, filed on Oct. 17, 2002; U.S. patent application Ser. No. 10/829,674, filed on Apr. 22, 2004) may indicate one's increased risk for MI and ACS. Other representatibe at-risk haplotypes are shown in Table 4 and Table 5. Additional “at-risk” haplotypes can be determined using linkage disequilibrium and/or haplotype blocks, as described below.

In a further embodiment, an individual who is at risk for MI or ACS is an individual who has an elevated inflammatory marker. An “elevated inflammatory marker,” as used herein, is the presence of an amount of an inflammatory marker that is greater, by an amount that is statistically significant, than the amount that is typically found in control individual(s) or by comparison of disease risk in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). An “inflammatory marker” refers to a molecule that is indicative of the presence of inflammation in an individual, for example, C-reactive protein (CRP), serum amyloid A, myeloperoxidase (MPO), N-tyrosine, di-tyrosine, lipoprotein phospholipase A2 (Lp-PLA2), fibrinogen, leukotriene levels (e.g., LTB4, LTE4), leukotriene metabolites (e.g., 12-oxo-LTB4, 10,11,14,15-tetrahydro-12-oxo-LTB4), interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, and matrix metalloprotease type-9) or other markers (see, e.g., Doggen, C. J. M. et al., J. Internal Med., 248:406-414 (2000); Ridker, P. M. et al., New Englnd. J. Med. 1997: 336: 973-979, Rettersol, L. et al., 2002: 160:433-440; Ridker, P. M. et. al., New England. J. Med., 2002: 347: 1557-1565; Bermudez, E. A. et. al., Arterioscler. Thromb. Vasc. Biol., 2002: 22:1668-1673). In certain embodiments, the presence of such inflammatory markers can be measured in serum or urine.

In a third embodiment, an individual who is at risk for MI or ACS is an individual who has increased total cholesterol, increased LDL cholesterol and/or decreased HDL cholesterol levels. For example, the American Heart Association indicates that an LDL cholesterol level of less than 100 mg/dL is optimal; from 100-129 mg/dL is near/above optimal; from 130-159 mg/dL is borderline high; from 160-189 is high; and from 190 and up is very high. Therefore, an individual who is at risk for MI or ACS because of an increased LDL cholesterol level is, for example, an individual who has more than 100 mg/dL cholesterol, such as an individual who has a near/above optimal level, a borderline high level, a high level or a very high level. Similarly, the American Heart Association indicates that an HDL cholesterol level of less than 40 mg/dL is a major risk factor for heart disease; and an HDL cholesterol level of 60 mg/dL or more is protective against heart disease. Thus, an individual who is at risk for MI or ACS because of a decreased HDL cholesterol level is, for example, an individual who has less than 60 mg/dL HDL cholesterol, such as an individual who has less than 40 mg/dL HDL cholesterol.

In a fourth embodiment, an individual who is at risk for MI or ACS is an individual who has increased leukotriene synthesis. “Increased leukotriene synthesis,” as used herein, indicates an amount of production of leukotrienes that is greater, by an amount that is statistically significant, than the amount of production of leukotrienes that is typically found in control individual(s) or by comparison of leukotriene production in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). An individual can be assessed for the presence of increased leukotriene synthesis by a variety of methods. For example, an individual can be assessed for an increased risk of MI, ACS or atherosclerosis, by assessing the level of a leukotriene metabolite (e.g., LTB4, LTE4) in a sample (e.g., serum, plasma or urine) from the individual. An increased level of leukotriene metabolites is indicative of increased production of leukotrienes, and of an increased risk of MI, ACS or atherosclerosis.

In a further embodiment, an individual who is at risk for MI or ACS is an individual who has already experienced at least one MI or ACS event, or who has stable angina, and is therefore at risk for a second MI or ACS event. In another embodiment, an individual who is at risk for MI or ACS is an individual who has atherosclerosis or who requires treatment (e.g., angioplasty, stenting, coronary artery bypass graft) to restore blood flow in arteries.

In additional embodiments, an individual who is at risk for MI or ACS is an individual who has diabetes; hypertension; hypercholesterolemia; elevated lp(a); obesity; acute or past stroke or transient ischemic event, peripheral arterial occlusive disease, and/or is a past or current smoker.

Individuals at risk for MI or ACS may fall into more than one of these representative target populations. For example, an individual may have experienced at least one MI or ACS event, and may also have an increased level of an inflammatory marker. As used therein, the term “individual in a target population” refers to an individual who is at risk for MI or ACS who falls into at least one of the representative target populations described above.

Assessment for At-Risk Haplotypes

A “haplotype,” as described herein, refers to a combination of genetic markers (“alleles”). In a certain embodiment, the haplotype can comprise two or more alleles, three or more alleles, four or more alleles, or five or more alleles. The genetic markers are particular “alleles” at “polymorphic sites” associated with LTA4H. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference LTA4H sequence is described herein by SEQ ID NO:1. The term, “variant LTA4H”, as used herein, refers to a sequence that differs from SEQ ID NO:1, but is otherwise substantially similar. The genetic markers that make up the haplotypes described herein are LTA4H variants.

Additional variants can include changes that affect a polypeptide, e.g., the LTA4H polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by an LTA4H nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with MI or a susceptibility to MI can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

In one embodiment, haplotypes can be used to identify individuals at risk for MI OR ACS. Haplotypes are a combination of genetic markers, e.g., particular alleles at polymorphic sites. Markers can include, for example, SNPs and microsatellites. The haplotypes can comprise a combination of various genetic markers; therefore, detecting haplotypes can be accomplished by methods known in the art for detecting sequences at polymorphic sites. For example, standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. These markers and SNPs can be identified in at-risk haploptypes. Certain methods of identifying relevant markers and SNPs include the use of linkage disequilibrium (LD) and/or LOD scores.

Linkage Disequilibrium

Linkage Disequilibrium (LD) refers to a non-random assortment of two genetic elements. For example, if a particular genetic element (e.g., “alleles” at a polymorphic site) occurs in a population at a frequency of 0.25 and another occurs at a frequency of 0.25, then the predicted occurrance of a person's having both elements is 0.125, assuming a random distribution of the elements. However, if it is discovered that the two elements occur together at a frequency higher than 0.125, then the elements are said to be in linkage disequilibrium since they tend to be inherited together at a higher rate than what their independent allele frequencies would predict. Roughly speaking, LD is generally correlated with the frequency of recombination events between the two elements.

Many different measures have been proposed for assessing the strength of linkage disequilibrium (LD). Most capture the strength of association between pairs of biallelic sites. Two important pairwise measures of LD are r² (sometimes denoted ²) and |\acute{D}|. Both measures range from 0 (no disequilibrium) to 1 (‘complete’ disequilibrium), but their interpretation is slightly different. |\acute{D}| is defined in such a way that it is equal to 1 if just two or three of the possible haplotypes are present, and it is <1 if all four possible haplotypes are present. So, a value of |\acute{D}| that is <1 indicates that historical recombination has occurred between two sites (recurrent mutation can also cause |\acute{D}| to be <1, but for single nucleotide polymorphisms (SNPs) this is usually regarded as being less likely than recombination). The measure r² represents the statistical correlation between two sites, and takes the value of 1 if only two haplotypes are present. It is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

Haplotypes and LOD Score Definition of a Susceptibility Locus

In certain embodiments, haplotype analysis involves defining a candidate susceptibility locus using LOD scores. The defined regions are then ultra-fine mapped with microsatellite markers with an average spacing between markers of less than 100 kb. All usable microsatellite markers that are found in public databases and mapped within that region can be used. In addition, microsatellite markers identified within the deCODE genetics sequence assembly of the human genome can be used. The frequencies of haplotypes in the patient and the control groups can be estimated using an expectation-maximization algorithm (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values. In a preferred embodiment, a p-value of <0.05 is indicative of an at-risk haplotype.

A detailed discussion of haplotype analysis follows.

Haplotype Analysis

One general approach to haplotype analysis involves using likelihood-based inference applied to NEsted MOdels. The method is implemented in the program NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e, the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hⱼ are assumed to have the same risk, it corresponds to assuming that fᵢ pᵢ=fⱼ pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (e.g., see the information measure section below) and may require randomization to properly evaluate statistical significance. In NEMO, maximum likelihood estimates, likelihood ratios and p-values are calculated directly, with the aid of the EM algorithm, for the observed data treating it as a missing-data problem.

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes h₁=G0, h₂=GX and h₃=AX, and the focus is on comparing h₁ and h₂. The alternative grouping is [h₁], [h₂], [h₃] and the null grouping is [h₁, h₂], [h₃]. If composite alleles exist, one could collapse these alleles into one at the data processing stage, and performed the test as described. This is a perfectly valid approach, and indeed, whether we collapse or not makes no difference if there were no missing information regarding phase. But, with the actual data, if each of the alleles making up a composite correlates differently with the SNP alleles, this will provide some partial information on phase. Collapsing at the data processing stage will unnecessarily increase the amount of missing information. A nested-models/partition framework can be used in this scenario. Let h₂ be split into h₂ₐ, h_{2b}, . . . , h₂ₑ, and h₃ be split into h₃ₐ, h_{3b}, . . . , h₃ₑ. Then the alternative grouping is [h₁], [h₂ₐ, h_{2b}, . . . , h₂ₑ], [h₃ₐ, h_{3b}, . . . , h₃ₑ] and the null grouping is [h₁, h₂ₐ, h_{2b}, . . . , h₂ₑ], [h₃ₐ, h_{3b}, . . . , h₃ₑ]. The same method can be used to handle composite where collapsing at the data processing stage is not even an option since L_{C} represents multiple haplotypes constructed from multiple SNPs. Alternatively, a 3-way test with the alternative grouping of [h₁], [h₂ₐ, h_{2b}, . . . , h₂ₑ], [h₃ₐ, h_{3b}, . . . , h₃ₑ] versus the null grouping of [h₁, h₂ₐ, h_{2b}, h₂ₑ, h₃ₐ, h_{3b}, . . . h₃ₑ] could also be performed. Note that the generalized likelihood ratio test-statistic would have two degrees of freedom instead of one.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose we calculate the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose we then perform a likelihood ratio test treating these estimated haplotype counts as though they are the actual counts. We could also perform a Fisher's exact test, but we would then need to round off these estimated counts since they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than Λ, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

Statistical Analysis

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure (e.g., as described in Risch, N. & Teng, J., “The relative power of family-based and case-control designs for linkage disequilibrium studies of complex human diseases I. DNA pooling,” Genome Res. 8:1278-1288 (1998)) for sibships so that it can be applied to general familial relationships, and present both adjusted and unadjusted p-values for comparison. The differences are in general very small as expected. To assess the significance of single-marker association corrected for multiple testing we carried out a randomisation test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model), (Terwilliger, J. D. & Ott, J., Hum Hered, 42, 337-46 (1992) and Falk, C. T. & Rubinstein, P, Ann Hum Genet 51 (Pt 3), 227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes hᵢ and hⱼ, risk(hᵢ)/risk(hⱼ)=(fᵢ/pᵢ)/(fⱼ/pⱼ), where f and p denote respectively frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program called NEMO, which stands for NEsted MOdels, can be used to calculate all the haplotype results. To handle uncertainties with phase and missing genotypes, it is emphasized that we do not use a common two-step approach to association tests, where haplotype counts are first estimated, possibly with the use of the EM algorithm, Dempster, (A. P., Laird, N. M. & Rubin, D. B., Journal of the Royal Statistical Society B, 39, 1-38 (1971)) and then tests are performed treating the estimated counts as though they are true counts, a method that can sometimes be problematic and may require randomisation to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all the others. Call the selected haplotype h₁ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model we use for the test assumes h₂, . . . , hₖ to have the same risk while h₁ is allowed to have a different risk. This implies that while p₁ can be different from f₁, fᵢ(f₂+ . . . +fₖ)=pᵢ(p₂+ . . . +pₖ)=βᵢ for i=2, . . . , k. Denoting f₁ p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)]
where l denotes logₑ likelihood and ~ and \hat{ } denote maximum likelihood estimates under the null hypothesis and alternative hypothesis respectively. Λ has asymptotically a chi-square distribution with 1-df, under the null hypothesis. Slightly more complicated null and alternative hypotheses can also be used. For example, let h₁ be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁ p₁=f₂ p₂, or w=[f₁ p₁] [f₂ p₂]=1. The test statistic based on generalized likelihood ratios is
Λ=2[l(\hat{p}₁, \hat{f}₁, \hat{p}₂, ŵ)−l(\tilde{p}₁, \tilde{f}₁, \tilde{p}₂, 1)]
that again has asymptotically a chi-square distribution with 1-df under the null hypothesis. If there are composite haplotypes (for example, h₂ and h₃), that is handled in a natural manner under the nested models framework.

Linkage Disequilibrium Using NEMO

LD between pairs of SNPs can also be calculated using the standard definition of D′ and R² (Lewontin, R., Genetics 49, 49-67 (1964) and Hill, W. G. & Robertson, A. Theor. Appl. Genet. 22, 226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Statistical Methods for Linkage Analysis

Multipoint, affected-only allele-sharing methods can be used in the analyses to assess evidence for linkage. Results, both the LOD-score and the non-parametric linkage (NPL) score, can be obtained using the program Allegro (Gudbjartsson et al., Nat. Genet. 25:12-3, 2000). Our baseline linkage analysis uses the Spairs scoring function (Whittemore, A. S., Halpern, J. (1994), Biometrics 50:118-27; Kruglyak L, et al. (1996), Am J Hum Genet 58:1347-63), the exponential allele-sharing model (Kong, A. and Cox, N. J. (1997), Am J Hum Genet 61:1179-88) and a family weighting scheme that is halfway, on the log-scale, between weighting each affected pair equally and weighting each family equally. The information measure we use is part of the Allegro program output and the information value equals zero if the marker genotypes are completely uninformative and equals one if the genotypes determine the exact amount of allele sharing by decent among the affected relatives (Gretarsdottir et al., Am. J. Hom. Genet, 70:593-603, (2002)). We computed the P-values two different ways and here report the less significant result. The first P-value can be computed on the basis of large sample theory; the distribution Of Zₗᵣ=(2[logₑ(10)LOD]) approximates a standard normal variable under the null hypothesis of no linkage (Kong, A. and Cox, N. J. (1997), Am J Hum Genet 61:1179-88). The second P-value can be calculated by comparing the observed LOD-score with its complete data sampling distribution under the null hypothesis (e.g., Gudbjartsson et al., Nat. Genet. 25:12-3, 2000). When the data consist of more than a few families, these two P-values tend to be very similar.

Haplotypes and “Haplotype Block” Definition of a Susceptibility Locus

In certain embodiments, haplotype analysis involves defining a candidate susceptibility locus based on “haplotype blocks.” It has been reported that portions of the human genome can be broken into series of discrete haplotype blocks containing a few common haplotypes; for these blocks, linkage disequilibrium data provided little evidence indicating recombination (see, e.g., Wall., J. D. and Pritchard, J. K., Nature Reviews Genetics 4: 587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., PNAS SA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the term, “haplotype block” includes blocks defined by either characteristic.

Representative methods for identification of haplotype blocks are set forth, for example, in U.S. Published Patent Applications 20030099964; 20030170665; 20040023237; 20040146870. Haplotype blocks can be used readily to map associations between phenotype and haplotype status. The main haplotytpes can be identified in each haplotype block, and then a set of “tagging” SNPs or markers (the smallest set of SNPs or markers needed to distinguish among the haplotypes) can then be identified These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

Haplotypes and Diagnostics

Certain haplotypes as described herein, e.g., having markers such as those shown in Table 3, 4 or 5, have been found more frequently in individuals with MI and/or ACS than in individuals without MI and/or ACS. Therefore, these “at-risk” haplotypes have predictive value for detecting a susceptibility to MI or ACS in an individual. In addition, haplotype blocks comprising certain tagging markers, can be found more frequently in individuals with MI or ACS than in individuals without MI or ACS. Therefore, these “at-risk” tagging markers within the haplotype blocks also have predictive value for detecting a susceptibility to MI or ACS in an individual. “At-risk” tagging markers within the haplotype blocks can also include other markers that distinguish among the haplotypes, as these similarly have predictive value for detecting a susceptibility to MI or ACS in an individual.

The haplotypes and tagging markers useful herein are in some cases a combination of various genetic markers, e.g., SNPs and microsatellites. Therefore, detecting haplotypes can be accomplished by methods known in the art for detecting sequences at polymorphic sites, such as the methods described above. Furthermore, correlation between certain haplotypes or sets of tagging markers and disease phenotype can be verified using standard techniques. A representative example of a simple test for correlation would be a Fisher-exact test on a two by two table.

In specific embodiments, an at-risk haplotype in, or comprising portions of, the LTA4H gene, is one where the haplotype is more frequently present in an individual at risk for MI or ACS (affected), compared to the frequency of its presence in a healthy individual (control), and wherein the presence of the haplotype is indicative of susceptibility to MI or ACS. In other embodiments, at-risk tagging markers in a haplotype block in linkage disequilibrium with one or more markers in the LTA4H gene, are tagging markers which are more frequently present in an individual at risk for MI or ACS (affected), compared to the frequency of their presence in a healthy individual (control), and wherein the presence of the tagging markers is indicative of susceptibility to MI or ACS. In a further embodiments, at-risk markers in linkage disequilibrium with one or more markers in the LTA4H gene, are markers which are more frequently present in an individual at risk for MI or ACS (affected), compared to the frequency of their presence in a healthy individual (control), and wherein the presence of the markers is indicative of susceptibility to MI or ACS. In particularly preferred embodiments of the invention, at-risk haplotypes include haplotypes as shown in Table 4 or Table 5.

In certain methods described herein, an individual who is at risk for MI or ACS is an individual in whom an at-risk haplotype is identified, or an individual in whom at-risk tagging markers are identified. In one embodiment, the at-risk haplotype or at-risk tagging markers confer a significant risk of MI or ACS. In one embodiment, significant risk of MI or ACS is measured by an odds ratio; in another embodiment, significant risk is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95%, and 98%. In a further embodiment, a significant increase in risk is at least about 50%. In yet another embodiment, an at-risk haplotype has a p value <0.05. It is understood however, that identifying whether a risk is medically significant may also depend on a variety of factors, including the specific disease, the haplotype, and often, environmental factors.

Particular embodiments of the invention encompass methods including a method of diagnosing a susceptibility to MI or ACS in an individual, comprising assessing in an individual the presence or frequency of SNPs and/or microsatellites in, comprising portions of, the LTA4H gene, wherein an excess or higher frequency of the SNPs and/or microsatellites in the individual, compared to a healthy control individual, is indicative that the individual is susceptible to MI or ACS. See, for example, Table 3, 4 and/or 5 (below) for SNPs and markers that can form haplotypes that can be used as screening tools, as well as Tables 4 and/or 5 for haplotypes that can be used for screening tools. Other particular embodiments of the invention encompass methods of diagnosing a susceptibility to MI or ACS in an individual, comprising detecting one or more markers at one or more polymorphic sites, wherein the one or more polymomrphic sites are in linkage disequilibrium with LTA4H.

Individuals who have been identified as being susceptible to MI or ACS using the methods described herein are individuals who fall within a target population for the methods of therapy described herein.

Methods of Therapy

The present invention encompasses methods of treatment (prophylactic and/or therapeutic) for MI or ACS in individuals, such as individuals in the target populations described above, as well as for other diseases and conditions associated with LTA4H or with other members of the leukotriene pathway (e.g., for atherosclerosis). Members of the “leukotriene pathway,” as used herein, include polypeptides (e.g., enzymes, receptors) and other molecules that are associated with production of leukotrienes: for example, enzymes such as LTA4H; other leukotriene biosynthetic enzymes (e.g., FLAP, 5-LO); receptors or binding agents of the enzymes; leukotrienes such as LTA4, and LTB4; and receptors of leukotrienes (e.g., leukotriene B4 receptor 1 (BLT1), leukotriene B4 receptor 2 (BLT2)).

In particular, the invention relates to methods of treatment for myocardial infarction or susceptibility to myocardial infarction (for example, for individuals in an at-risk population such as those described above); as well as methods of treatment for acute coronary syndrome (e.g., unstable angina, non-ST-elevation myocardial infarction (NSTEMI) or ST-elevation myocardial infarction (STEMI)); for decreasing risk of a second myocardial infarction; for atherosclerosis, such as for patients requiring treatment (e.g., angioplasty, stenting, coronary artery bypass graft) to restore blood flow in arteries (e.g., coronary arteries); and/or for decreasing leukotriene synthesis (e.g., for preventing and/or treatment of MI or ACS).

The invention additionally pertains to use of one or more leukotriene synthesis inhibitors, as described herein, for the manufacture of a medicament for the treatment of MI, ACS, and/or atherosclerosis, e.g., using the methods described herein.

In the methods of the invention, a “leukotriene synthesis inhibitor” is used. In one embodiment, a “leukotriene synthesis inhibitor” is an agent that inhibits LTA4H polypeptide activity and/or LTA4H nucleic acid expression, as described herein. In another embodiment, a leukotriene synthesis inhibitor is an agent that inhibits polypeptide activity and/or nucleic acid expression of another member of the leukotriene biosynthetic pathway (e.g., FLAP, 5-LO). In still another embodiment, a leukotriene synthesis inhibitor is an agent that alters activity or metabolism of a leukotriene (e.g., an antagonist of a leukotriene; an antagonist of a leukotriene receptor). In preferred embodiments, the leukotriene synthesis inhibitor decreases activity and/or nucleic acid expression of LTA4H.

Leukotriene synthesis inhibitors can alter polypeptide activity or nucleic acid expression of a member of the leukotriene pathway by a variety of means, such as, for example, by catalytically degrading, downregulating or interfering with the expression, transcription or translation of a nucleic acid encoding the member of the leukotriene pathway; by altering posttranslational processing of the polypeptide; by altering transcription of splicing variants; or by interfering with polypeptide activity (e.g., by binding to the polypeptide, or by binding to another polypeptide that interacts with that member of the leukotriene pathway, such as an LTA4H binding agent as described herein or some other binding agent of a member of the leukotriene pathway; by altering interaction among two or more members of the leukotriene pathway (e.g., interaction between FLAP and 5-LO); or by antagonizing activity of a member of the leukotriene pathway.

Representative leukotriene synthesis inhibitors include the following:
agents that inhibit activity of a member of the leukotriene biosynthetic pathway (e.g., LTA4, FLAP, 5-LO), such as the agents presented in the Agent Table or in the Additional LTA4H Agent List below;
agents that inhibit activity of receptors of members of the leukotriene pathway, such as 5-LO receptors (e.g., FLAP), LTB4 receptors (e.g., BLT1, BLT2); agents that bind to the members of the leukotriene pathway, such as LTA4H binding agents, agents that bind to receptors of members of the leukotriene pathway (e.g., leukotriene receptor antagonists); or agents that bind to a leukotriene (e.g., to LTA4, LTB4) or otherwise affect (e.g., decrease) activity of the leukotriene;
antibodies to leukotrienes;
antisense nucleic acids or small double-stranded interfering RNA, to nucleic acids encoding LTA4H, or a leukotriene synthetase or other member of the leukotriene pathway (e.g., FLAP, 5-LO), or fragments or derivatives thereof, including antisense nucleic acids to nucleic acids encoding the LTA4H, or leukotriene synthetase polypeptides, and vectors comprising such antisense nucleic acids (e.g., nucleic acid, cDNA, and/or mRNA, double-stranded interfering RNA, or a nucleic acid encoding an active fragment or derivative thereof, or an oligonucleotide; for example, the complement of one of SEQ ID Nos. 1 or 2, or a nucleic acid complementary to the nucleic acid encoding SEQ ID NO: 3, or fragments or derivatives thereof);
peptidomimetics; fusion proteins or prodrugs thereof; ribozymes; other small molecules; and
other agents that alter (e.g., inhibit or antagonize) expression of a member of the leukotriene pathway, such as LTA4H nucleic acid expression or polypeptide activity, or that regulate transcription of LTA4H splicing variants (e.g., agents that affect which splicing variants are expressed, or that affect the amount of each splicing variant that is expressed).

More than one leukotriene synthesis inhibitor can be used concurrently, if desired.

The therapy is designed to alter activity of an LTA4H polypeptide, or another member of the leukotriene pathway in an individual, such as by inhibiting or antagonizing activity. For example, a leukotriene synthesis inhibitor can be administered in order to decrease synthesis of leukotrienes within the individual, or to downregulate or decrease the expression or availability of the LTA4H nucleic acid or specific splicing variants of the LTA4H nucleic acid. Downregulation or decreasing expression or availability of a native LTA4H nucleic acid or of a particular splicing variant could minimize the expression or activity of a defective nucleic acid or the particular splicing variant and thereby minimize the impact of the defective nucleic acid or the particular splicing variant.

The leukotriene synthesis inhibitor(s) are administered in a therapeutically effective amount (i.e., an amount that is sufficient to treat the disease or condition, such as by ameliorating symptoms associated with the disease or condition, preventing or delaying the onset of the disease or condition, and/or also lessening the severity or frequency of symptoms of the disease or condition). The amount which will be therapeutically effective in the treatment of a particular individual's disease or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In preferred embodiments of the invention, the leukotriene synthesis inhibitor agent is an agent that inhibits activity of LTA4H. Preferred agents include the following, as set forth in the Agent Table or in the Additional LTA4H Agent List:

Agent Table

| | Compound | | |
|---|---|---|---|
| Target | ID | Chemical Name | Patent/Reference |
| LTA4H | SC-57461A | 3-[methyl[3-[4- | Penning, T.D. et.al. Bioorg Med. Chem. Letters |
| Inhibitor | | (phenylmethyl)phenoxy]- | (2003), 13, 1137-1139. |
| | | propyl]amino]propionic | ibid, (2002), 12, 3383-3386 |
| | | acid | |
| LTA4H | SC-56938 | Ethyl-1-[2-[4- | Penning, T.D. et.al. Bioorg Med. Chem. Letters |
| Inhibitor | | (phenylmethyl)phenoxy]ethyl]- | (2003), 13, 1137-1139; |
| | | 4-piperidine-carboxylate | ibid, (2002), 12, 3383-3386. |
| | | | US 6506876A1 |
| LTA4H | RP 64966 | [4-[5-(3-Phenyl- | WO9627585 |
| Inhibitor | | propyl)thiophen-2- | |
| | | yl]butoxy]acetic acid | |
| LTA4H | SA 6541 | (R)-S-[[4- | WO9809943 |
| Inhibitor | | (dimethylamino)phenyl]methyl]- | |
| | | N-(3-mercapto- | |
| | | 2methyl-1-oxopropyl-L- | |
| | | cycteine | |
| LTB4 | Amelubant/ | Carbamic acid,((4-((3-((4- | U.S. Pat. No. 6,576,669 |
| Receptor | BIIL-284 | (1-(4-hydroxyphenyl)-1- | |
| Antagonist | | methylethyl)phenoxy)methyl) | |
| | | phenyl)methoxy) | |
| | | phenyl)iminomethyl)- | |
| | | ethyl ester | |
| LTB4 | BIRZ-227 | 5-Chloro-2-[3-(4-methoxy- | Journal of Organic Chemistry 1998, 63: 2(326-330). |
| Receptor | | phenyl)-2-pyridin-2-yl- | |
| Antagonist | | pyrrolidin-1-yl]- | |
| | | benzooxazole | |
| LTB4 | CP 195543 | 2-[(3S,4R)-3,4-dihydro-4- | Process: WO 98/11085 1998, priority US |
| Receptor | | hydroxy-3-(phenylmethyl)- | 60/26372 1996; J. Pharamacology and Expert. |
| Antagonist | | 2H-1-benzopyran-7-yl]-4- | Therapy, 1998, 285: 946-54 |
| | | (trifluoromethyl)benzoic | |
| | | acid | |
| LTB4 | Ebselen | 2-Phenyl- | Journal of Cerebral Blood Flow and |
| Receptor | | benzo[d]isoselenazol-3-one | Metabolism 1995, July 2-6 (S162); Drugs of |
| Antagonist | | | the Future 1995, 20: 10 (1057) |
| LTB4 | LTB 019; | 4-[5-(4-Carbamimidoyl- | ACS Meeting 1994, 207th: San Diego (MEDI |
| Receptor | CGS-25019C | phenoxy)-pentyloxy]-N,N- | 003); International Congress of the |
| Antagonist | | diisopropyl-3-methoxy- | Inflammation Research Association 1994, |
| | | benzamide maleate | 7th: White Haven (Abs W23) |
| LTB4 | LY 210073 | 5-(2-Carboxy-ethyl)-6-[6- | J Med Chem 1993 36 (12) 1726-1734 |
| Receptor | | (4-methoxy-phenyl)-hex-5- | |
| Antagonist | | enyloxy]-9-oxo-9H- | |
| | | xanthene-2-carboxylic acid | |
| LTB4 | LY 213024 | 5-(3-carboxybenzoyl)-2- | J Med Chem 1993 36 (12) 1726-1734 |
| Receptor | | (decyloxy)benzenepropanoic | |
| Antagonist | | acid | |
| LTB4 | LY 255283 | 1-[5-ethyl-2-hydroxy-4-[[6- | EP 276064 B 1990, priority US 2479 1987 |
| Receptor | | methyl-6-(1H-tetrazol-5- | |
| Antagonist | | yl)heptyl]oxy]phenyl] | |
| | | ethanone | |
| LTB4 | LY 264086 | 7-carboxy-3-(decyloxy)-9- | U.S Pat. No. 4996230 1991, priority US 481413 1990 |
| Receptor | | oxo-9H-xanthene-4- | |
| Antagonist | | propanoic acid | |
| LTB4 | LY 292728 | 7-carboxy-3-[3-[(5-ethyl-4′- | EP 743064 A 1996, priority US 443179 1995 |
| Receptor | | fluoro-2-hydroxy[1,1′- | |
| Antagonist | | biphenyl]-4-yl)oxy] | |
| | | propoxy]-9-oxo-9H- | |
| | | xanthene-4-propanoic acid | |
| | | disodium salt | |
| LTB4 | LY-293111 | Benzoic acid,2-(3-(3-((5- | Proceedings of the American Society for |
| Receptor | (VML-295) | ethyl-4-fluoro-2- | Clinical Oncology 2002, 21: 1 (Abs 343) [LY- |
| Antagonist | | hydroxy(1,1′-biphenyl)-4- | 293111 for Cancer] SCRIP |
| | | yl)oxy)propoxy)-2- | World Pharmaceutical News 1997, 2272 (13) |
| | | propylphenoxy)- | [for VML-295] |
| LTB4 | ONO 4057; | (E)-2-(4-carboxybutoxy)-6- | EP 405116 A 1991 |
| Receptor | LB 457 | [[6-(4-methoxyphenyl)-5- | |
| Antagonist | | hexenyl]oxy]benzenepropanoic | |
| | | acid | |
| LTB4 | PF 10042 | 1-[5-hydroxy-5-[8-(1- | EP 422329 B 1995, priority US 409630 1989 |
| Receptor | | hydroxy-2-phenylethyl)-2- | |
| Antagonist | | dibenzofuranyl]-1-oxo | |
| | | pentyl]pyrrolidine | |
| LTB4 | RG-14893 | 8-Benzyloxy-4-[(methyl- | SCRIP World Pharmaceutical News |
| Receptor | | phenethyl-carbamoyl)- | 1996, 2168 (20) |
| Antagonist | | methyl]-naphthalene-2- | |
| | | carboxylic acid | |
| LTB4 | SB-201993 | 3-{6-(2-Carboxy-vinyl)-5- | WO-09500487 |
| Receptor | | [8-(4-methoxy-phenyl)- | |
| Antagonist | | octyloxy]-pyridin-2- | |
| | | ylmethylsulfanylmethyl}- | |
| | | benzoic acid | |
| LTB4 | SC-52798 | 7-[3-(2-Cyclopropylmethyl- | Bioorganic and Medicinal Chemistry Letters |
| Receptor | | 3-methoxy-4-thiazol-4-yl- | 1994, 4: 6 (811-816); Journal of Medicinal |
| Antagonist | | phenoxy)-propoxy]-8- | Chemistry 1995, 38: 6 (858-868) |
| | | propyl-chroman-2- | |
| | | carboxylic acid | |
| LTB4 | SC-53228 | 3-{7-[3-(2- | International Congress of the Inflammation |
| Receptor | | Cyclopropylmethyl-3- | Research Association 1994, 7th: White Haven |
| Antagonist | | methoxy-4- | (Abs W5) |
| | | methylcarbamoyl-phenoxy)- | |
| | | propoxy]-8-propyl- | |
| | | chroman-2-yl}-propionic | |
| | | acid | |
| LTB4 | WAY 121006 | 3-fluoro-4′-(2- | Drugs under Experimental and Clinical |
| Receptor | | quinolinylmethoxy)-[1,1′- | research 1991, 17: 8 (381-387) |
| Antagonist | | biphenyl]-4-acetic acid | |
| LTB4 | ZD-2138 | 3-Amino-3-(4-methoxy- | International Symposium on Medicinal |
| Receptor | | tetrahydro-pyran-4-yl)- | Chemistry 1994, 13th: Paris (P 197) |
| Antagonist | | acrylic acid 1-methyl-2-oxo- | |
| | | 1,2-dihydro-quinolin-6- | |
| | | ylmethyl ester | |

In addition the following LTA4H inhibitors are described in USP2003/0004101A1, the teachings of which are incorporated herein by reference in their entirety:

Additional LTA4H Agent List
1. 1-[2-[4-(phenylmethyl)phenoxy]ethyl]-2-methyl-4-tetrazolylpieridine
2. 1-[2-[4-(4-oxazolyl)phenoxy)phenoxy]ethyl]pyrrolidine
3. 3-[methyl[3-[4-(2-thienylmethyl)phenoxy]propyl]amino]propionic acid
4. methyl 3-[methyl[3-[4-(2-thienylmethyl)phenoxy]propyl]amino]propionate
5. 3-[methyl[3-[4-(3-thienylmethyl)phenoxy]propyl]amino]propionic acid
6. methyl-3-[methyl[3-4-(3-theinylmethyl)phenoxy]propyl]amino]propionate
7. 3-[methyl[3-[4-(4-fluorophenoxy)phenoxy]propyl]amino]propionic acid
8. 3-[methyl[3-[4-(4-biphenyloxy)phenoxy]propyl]amino]propionic acid
9. N-[3-[[3-[4-(phenylmethyl)phenoxy]propyl]methylamino]propionyl]benzenesulfonamide
10. 1-[2-[4-(phenylmethyl)phenoxy]ethyl]-2-methyl-4-(1H-tetrazol-5-yl)piperidine
11. 1-[2-[4-(phenylmethyl)phenoxy]ethyl]-4-(1H-tetrazol-5-yl)piperidine
   Nucleic Acid Therapeutic Agents

In another embodiment, a nucleic acid of the invention; a nucleic acid complementary to a nucleic acid of the invention; or a portion of such a nucleic acid (e.g., an oligonucleotide as described below); or a nucleic acid encoding a member of the leukotriene pathway (e.g., LTA4H), can be used in “antisense” therapy, in which a nucleic acid (e.g., an oligonucleotide) which specifically hybridizes to the mRNA and/or genomic DNA of a nucleic acid is administered or generated in situ. The antisense nucleic acid that specifically hybridizes to the mRNA and/or DNA inhibits expression of the polypeptide encoded by that mRNA and/or DNA, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes the polypeptide for the member of the leukotriene pathway (e.g., LTA4H). Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Additionally, general approaches to constructing oligomers useful in antisense therapy are also described, for example, by Van der Krol et al. (Biotechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the polypeptide. The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication No. WO 88/09810) or the blood-brain barrier (see, e.g., PCT International Publication No. WO 89/10134), or hybridization-triggered cleavage agents (see, e.g., Krol et al., BioTechniques 6:958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express the member of the leukotriene pathway in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

In another embodiment of the invention, small double-stranded interfering RNA (RNA interference (RNAi)) can be used. RNAi is a post-transcription process, in which double-stranded RNA is introduced, and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al., Nature Medicine 8(7):681-686 (2002); the entire teachings of these references are incorporated herein by reference.

Endogenous expression of a member of the leukotriene pathway (e.g., LTA4H) can also be reduced by inactivating or “knocking out” the gene or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered; non-functional gene of a member of the leukotriene pathway (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of an gene in the cell, as described above. In another embodiment, targeted homologous recombination can be used to insert a DNA construct comprising a nucleic acid that encodes a polypeptide variant that differs from that present in the cell.

Alternatively, endogenous expression of a member of the leukotriene pathway can be reduced by targeting deoxyribonucleotide sequences complementary to the regulatory region of the member of the leukotriene pathway (i.e., the promoter and/or enhancers) to form triple helical structures that prevent transcription of the gene in target cells in the body. (See generally, Helene, C., Anticancer Drug Des., 6(6):569-84 (1991); Helene, C. et al., Ann. N.Y. Acad. Sci. 660:27-36 (1992); and Maher, L. J., Bioassays 14(12):807-15 (1992)). Likewise, the antisense constructs described herein, by antagonizing the normal biological activity of one of the members of the leukotriene pathway, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a nucleic acid RNA or nucleic acid sequence) can be used to investigate the role of one or more members of the leukotriene pathway in the development of disease-related conditions. Such techniques can be utilized in cell culture, but can also be used in the creation of transgenic animals.

The therapeutic agents as described herein can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. In addition, a combination of any of the above methods of treatment (e.g., administration of non-altered polypeptide in conjunction with antisense therapy targeting altered mRNA for a member of the leukotriene pathway; administration of a first splicing variant in conjunction with antisense therapy targeting a second splicing variant) can also be used.

The invention additionally pertains to use of such therapeutic agents, as described herein, for the manufacture of a medicament for the treatment of MI, ACS, and/or atherosclerosis, e.g., using the methods described herein.

Monitoring Progress of Treatment

The current invention also pertains to methods of monitoring the response of an individual, such as an individual in one of the target populations described above, to treatment with a leukotriene synthesis inhibitor. Because the level of inflammatory markers can be elevated in individuals who are in the target populations described above, an assessment of the level of inflammatory markers of the individual both before, and during, treatment with the leukotriene synthesis inhibitor will indicate whether the treatment has successfully decreased production of leukotrienes in the arterial vessel wall or in bone-marrow derived inflammatory cells.

For example, in one embodiment of the invention, an individual who is a member of a target population of individuals at risk for MI or ACS (e.g., an individual in a target population described above, such as an individual at-risk due to an LTA4H MI-haplotype) can be assessed for response to treatment with a leukotriene synthesis inhibitor, by examining leukotriene levels in the individual. Serum, plasma or urinary leukotrienes (e.g., LTB4, LTE4, LTC4, LTD4), or ex vivo production of leukotrienes (e.g., in blood samples stimulated with a calcium ionophore to produce leukotrienes) can be measured before, and during or after treatment with the leukotriene synthesis inhibitor. The leukotriene level before treatment is compared with the leukotriene level during or after treatment. The efficacy of treatment is indicated by a decrease in leukotriene production: a level of leukotriene during or after treatment that is significantly lower than the level of leukotriene before treatment, is indicative of efficacy. A level that is lower during or after treatment can be shown, for example, by decreased serum or urinary leukotrienes, or decreased ex vivo production of leukotrienes. A level that is “significantly lower”, as used herein, is a level that is less than the amount that is typically found in control individual(s), or is less in a comparison of disease risk in a population associated with the other bands of measurement (e.g., the mean or median, the highest quartile or the highest quintile) compared to lower bands of measurement (e.g., the mean or median, the other quartiles; the other quintiles).

In another embodiment of the invention, an individual who is a member of a target population of individuals at risk for MI or ACS (e.g., an individual in a target population described above, such as an individual at-risk due to elevated C-reactive protein) can be assessed for response to treatment with a leukotriene synthesis inhibitor, by examining levels of inflammatory markers in the individual. For example, levels of an inflammatory marker in an appropriate test sample (e.g., serum, plasma or urine) can be measured before, and during or after treatment with the leukotriene synthesis inhibitor. The level of the inflammatory marker before treatment is compared with the level of the inflammatory marker during or after treatment. The efficacy of treatment is indicated by a decrease in the level of the inflammatory marker, that is, a level of the inflammatory marker during or after treatment that is significantly lower than the level of inflammatory marker before treatment is indicative of efficacy. Representative inflammatory markers include: C-reactive protein (CRP), serum amyloid A, myeloperoxidase (MPO), N-tyrosine, di-tyrosine, lipoprotein phospholipase A2 (Lp-PLA2), fibrinogen, a leukotriene, a leukotriene metabolite (e.g., cysteinyl leukotrienes), interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, and matrix metalloprotease type-9. In a preferred embodiment, the marker is CRP.

Pharmaceutical Compositions

The present invention also pertains to pharmaceutical compositions comprising agents described herein, for example, an agent that is a leukotriene synthesis inhibitor as described herein. For instance, a leukotriene synthesis inhibitor can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc. Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Nucleic Acids of the Invention

LTA4H Nucleic Acids, Portions and Variants

In addition, the invention pertains to isolated nucleic acid molecules comprising a human LTA4H nucleic acid. The term, “LTA4H nucleic acid,” as used herein, refers to an isolated nucleic acid molecule encoding LTA4H polypeptide. The LTA4H nucleic acid molecules of the present invention can be RNA, for example, mRNA, or DNA, such as cDNA and genomic DNA. DNA molecules can be double-stranded or single-stranded; single stranded RNA or DNA can be either the coding, or sense strand or the non-coding, or antisense strand. The nucleic acid molecule can include all or a portion of the coding sequence of the gene or nucleic acid and can further comprise additional non-coding sequences such as introns and non-coding 3′ and 5′ sequences (including regulatory sequences, for example, as well as promoters, transcription enhancement elements, splice donor/acceptor sites, etc.).

For example, an LTA4H nucleic acid can consist of SEQ ID NOs: 1 or 2 or the complement thereof, or to a portion or fragment of such an isolated nucleic acid molecule (e.g., cDNA or the nucleic acid) that encodes LTA4H polypeptide (e.g., a polypeptide such as SEQ ID NO: 3). In a preferred embodiment, the isolated nucleic acid molecule comprises a nucleic acid molecule selected from the group consisting of SEQ ID NOs: 1 or 2, or their complement thereof.

Additionally, the nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleic acid sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. In certain embodiments, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, including but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleic acid sequence can include a nucleic acid molecule or nucleic acid sequence that is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the nucleic acid in tissue (e.g., human tissue), such as by Northern blot analysis.

The present invention also pertains to nucleic acid molecules which are not necessarily found in nature but which encode an LTA4H polypeptide (e.g., a polypeptide having an amino acid sequence comprising an amino acid sequence of SEQ ID NO: 3), or another splicing variant of an LTA4H polypeptide or polymorphic variant thereof. Thus, for example, DNA molecules that comprise a sequence that is different from the naturally occurring nucleic acid sequence but which, due to the degeneracy of the genetic code, encode an LTA4H polypeptide of the present invention are also the subjects of this invention. The invention also encompasses nucleotide sequences encoding portions (fragments), or encoding variant polypeptides such as analogues or derivatives of an LTA4H polypeptide. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of an LTA4H polypeptide. In one preferred embodiment, the nucleotide sequences are fragments that comprise one or more polymorphic microsatellite markers. In another preferred embodiment, the nucleotide sequences are fragments that comprise one or more single nucleotide polymorphisms in an LTA4H nucleic acid (e.g., the single nucleotide polymorphisms set forth in Table 3, below).

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleic acid sequence described herein (e.g., nucleic acid molecules which specifically hybridize to a nucleic acid sequence encoding polypeptides described herein, and, optionally, have an activity of the polypeptide). In one embodiment, the invention includes variants described herein which hybridize under high stringency hybridization conditions (e.g., for selective hybridization) to a nucleic acid sequence comprising a nucleic acid sequence selected from the group consisting of SEQ ID NOs: 1 or 2 or the complement thereof. In another embodiment, the invention includes variants described herein which hybridize under high stringency hybridization conditions (e.g., for selective hybridization) to a nucleic acid sequence encoding an amino acid sequence of SEQ ID NO: 3 or a polymorphic variant thereof. In a preferred embodiment, the variant that hybridizes under high stringency hybridizations has an activity of LTA4H.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity that is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200: 546-556 (1991), and in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS 4(1): 11-17 (1988). Such an algorithm is incorporated into the ALIGN program (version 2.0) which is part of the GCG sequence alignment software package (Accelrys, Cambridge, UK). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci. 10:3-5 (1994); and FASTA described in Pearson and Lipman, Proc. Natl. Acad. Sci. USA 85:2444-8 (1988).

In another embodiment, the percent identity between two amino acid sequences can be accomplished using the GAP program in the GCG software package using either a BLOSUM63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package using a gap weight of 50 and a length weight of 3.

The present invention also provides isolated nucleic acid molecules that contain a fragment or portion that hybridizes under highly stringent conditions to a nucleic acid sequence comprising SEQ ID NO: 1 or 2 or the complement of SEQ ID NO: 1 or 2, and also provides isolated nucleic acid molecules that contain a fragment or portion that hybridizes under highly stringent conditions to a nucleic acid sequence encoding an amino acid sequence of the invention or polymorphic variant thereof. The nucleic acid fragments of the invention are at least about 15, for example, at least about 18, 20, 23 or 25 nucleotides, and can be 30, 40, 50, 100, 200 or more nucleotides in length. Longer fragments, for example, 30 or more nucleotides in length, encoding antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al. (Science 254:1497-1500 (1991)).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence of SEQ ID NOs: 1 or 2 or the complement of SEQ ID Nos: 1 or 2, or a nucleic acid sequence encoding an amino acid sequence of SEQ ID NO: 3 or polymorphic variant thereof. In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated using the polymerase chain reaction and synthetic oligonucleotide primers based on one or more of SEQ ID NOs: 1 or 2, or the complement thereof, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19:4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide and the DNA encoding the polypeptide can be isolated, sequenced and further characterized.

Antisense nucleic acid molecules of the invention can be designed using the nucleotide sequences of SEQ ID NOs: 1 or 2 and/or the complement of one or more of SEQ ID NOs: 1 or 2 and/or a portion of one or more of SEQ ID NOs: 1 or 2 or the complement of one or more of SEQ ID NOs: 1 or 2 and/or a sequence encoding the amino acid sequence of SEQ ID NO: 3 or encoding a portion of SEQ ID NO: 3 or its complement. They can be constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences can also be used to compare with endogenous DNA sequences in patients to identify one or more of the disorders related to LTA4H, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions or nucleic acid regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors

Another aspect of the invention pertains to nucleic acid constructs containing a nucleic acid molecule of SEQ ID NOs: 1 or 2 or the complement thereof (or a portion thereof). Yet another aspect of the invention pertains to nucleic acid constructs containing a nucleic acid molecule encoding an amino acid of SEQ ID NO: 3 or polymorphic variant thereof. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, such as expression vectors, are capable of directing the expression of genes or nucleic acids to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” or “operatively linked” is intended to mean that the nucleic acid sequence of interest is linked to the regulatory sequence(s) in a manner which allows for expression of the nucleic acid sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, “Gene Expression Technology”, Methods in Enzymology 185, Academic Press, San Diego, Calif. (1990). Regulatory sequences include those which direct constitutive expression of a nucleic acid sequence in many types of host cell and those which direct expression of the nucleic acid sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene or nucleic acid that encodes a selectable marker (e.g., for resistance to antibiotics) is generally introduced into the host cells along with the gene or nucleic acid of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene or nucleic acid will survive, while the other cells die).

A host cell of the invention, such as a prokaryotic host cell or eukaryotic host cell in culture can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous LTA4H nucleic acid, or an exogenous nucleic acid encoding an LTA4H polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleic acid sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley, Current Opinion in BioTechnology 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature 385:810-813 (1997) and PCT Publication Nos. WO 97/07668 and WO 97/07669.

Polypeptides of the Invention

The present invention also pertains to isolated polypeptides encoded by LTA4H nucleic acids (“LTA4H polypeptides”), and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleic acid sequence selected from the group consisting of SEQ ID NO: 1 or 2, or the complement of SEQ ID NO: 1 or 2, or portions thereof, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleic acid sequence selected from the group consisting of SEQ ID NOs: 1 or 2 or their complement, or portions thereof, or having substantial homology to a polypeptide encoded by a nucleic acid molecule comprising a nucleic acid sequence selected from the group consisting of nucleotide sequences encoding SEQ ID NO: 3 and polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in others greater than about 90% or more homologous or identical. A substantially homologous amino acid sequence, according to the present invention, will be encoded by a nucleic acid molecule hybridizing to SEQ ID NO: 1 or 2 or portion thereof, under stringent conditions as more particularly described above, or will be encoded by a nucleic acid molecule hybridizing to a nucleic acid sequence encoding SEQ ID NO: 3 or a portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol. 224:899-904 (1992); de Vos et al., Science 255:306-312 (1992)).

The invention also includes fragments of the polypeptides of the invention. Fragments can be derived from a polypeptide encoded by a nucleic acid molecule comprising SEQ ID NO: 1 or 2, or the complement of SEQ ID NO: 1 or 2 (or other variants). However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a ligand) in biological fluids. The polypeptides can also be used as markers for cells or tissues in which the corresponding polypeptide is preferentially expressed, either constitutively, during tissue differentiation, or in diseased states. The polypeptides can be used to isolate a corresponding binding agent, e.g., ligand, such as, for example, in an interaction trap assay, and to screen for peptide or small molecule antagonists or agonists of the binding interaction. For example, because members of the leukotriene pathway including LTA4H bind to receptors, the leukotriene pathway polypeptides can be used to isolate such receptors.

Antibodies of the Invention

Polyclonal and/or monoclonal antibodies that specifically bind one form of the polypeptide or nucleic acid product (e.g., a polypeptide encoded by a nucleic acid having a SNP as set forth in Table 3), but not to another form of the polypeptide or nucleic acid product, are also provided. Antibodies are also provided which bind a portion of either polypeptide encoded by nucleic acids of the invention (e.g., SEQ ID NO: 1 or SEQ ID NO:2, or the complement of SEQ ID NO: 1 or SEQ ID NO:2), or to a polypeptide encoded by nucleic acids of the invention that contain a polymorphic site or sites. The invention also provides antibodies to the polypeptides and polypeptide fragments of the invention, or a portion thereof, or having an amino acid sequence encoded by a nucleic acid molecule comprising all or a portion of SEQ ID NOs: 1 or 2, or the complement thereof, or another variant or portion thereof. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)); the EBV-hybridoma technique (Cole et al., Monoclonal Antibodies and Cancer Therapy, Alan R. Liss, 1985, Inc., pp. 77-96); or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246:1275-1281 (1989); Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin and aequorin, and examples of suitable radioactive material include ¹²⁵I, ¹³¹I, ³⁵S or ³H.

As described above, antibodies to leukotrienes can be used in the methods of the invention. The methods described herein can be used to generate such antibodies for use in the methods.

Diagnostic Assays

The nucleic acids, probes, primers, polypeptides and antibodies described herein can be used in methods of diagnosis of MI or diagnosis of a susceptibility to MI or to a disease or condition associated with an MI gene, such as LTA4H, as well as in kits useful for diagnosis of MI or a susceptibility to MI or to a disease or condition associated with LTA4H. In one embodiment, the kit useful for diagnosis of MI or susceptibility to MI, or to a disease or condition associated with LTA4H comprises primers as described herein, wherein the primers contain one or more of the SNPs identified in Table 3.

In one embodiment of the invention, diagnosis of MI or susceptibility to MI (or diagnosis of or susceptibility to a disease or condition associated with LTA4H), is made by detecting a polymorphism in an LTA4H nucleic acid as described herein. The polymorphism can be an alteration in an LTA4H nucleic acid, such as the insertion or deletion of a single nucleotide, or of more than one nucleotide, resulting in a frame shift alteration; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene or nucleic acid; duplication of all or a part of the gene or nucleic acid; transposition of all or a part of the gene or nucleic acid; or rearrangement of all or a part of the gene or nucleic acid. More than one such alteration may be present in a single gene or nucleic acid. Such sequence changes cause an alteration in the polypeptide encoded by an LTA4H nucleic acid. For example, if the alteration is a frame shift alteration, the frame shift can result in a change in the encoded amino acids, and/or can result in the generation of a premature stop codon, causing generation of a truncated polypeptide. Alternatively, a polymorphism associated with a disease or condition associated with an LTA4H nucleic acid or a susceptibility to a disease or condition associated with an LTA4H nucleic acid can be a synonymous alteration in one or more nucleotides (i.e., an alteration that does not result in a change in the polypeptide encoded by an LTA4H nucleic acid). Such a polymorphism may alter splicing sites, affect the stability or transport of mRNA, or otherwise affect the transcription or translation of the nucleic acid. An LTA4H nucleic acid that has any of the alteration described above is referred to herein as an “altered nucleic acid.”

In a first method of diagnosing MI or a susceptibility to MI, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, a susceptibility to a disease or condition associated with an LTA4H nucleic acid (the “test individual”). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in an MI nucleic acid is present, and/or to determine which splicing variant(s) encoded by the LTA4H nucleic acid is present. The presence of the polymorphism or splicing variant(s) can be indicated by hybridization of the nucleic acid in the genomic DNA, RNA, or cDNA to a nucleic acid probe. A “nucleic acid probe”, as used herein, can be a DNA probe or an RNA probe; the nucleic acid probe can contain at least one polymorphism in an LTA4H nucleic acid or contains a nucleic acid encoding a particular splicing variant of an LTA4H nucleic acid. The probe can be any of the nucleic acid molecules described above (e.g., the nucleic acid, a fragment, a vector comprising the nucleic acid, a probe or primer, etc.).

To diagnose MI or a susceptibility to MI (or a disease or condition associated with LTA4H), the test sample containing an LTA4H nucleic acid is contacted with at least one nucleic acid probe to form a hybridization sample. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of one of SEQ ID NOs: 1 or 2, or the complement thereof or a portion thereof; or can be a nucleic acid encoding all or a portion of SEQ ID NO: 3. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to an LTA4H nucleic acid. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and LTA4H nucleic acid in the test sample, then the LTA4H has the polymorphism, or is the splicing variant, that is present in the nucleic acid probe. More than one nucleic acid probe can also be used concurrently in this method. Specific hybridization of any one of the nucleic acid probes is indicative of a polymorphism in the LTA4H nucleic acid, or of the presence of a particular splicing variant encoding the LTA4H nucleic acid, and is therefore diagnostic for a disease or condition associated with LTA4H or a susceptibility to a disease or condition associated with LTA4H (e.g., MI).

In Northern analysis (see Current Protocols in Molecular Biology, Ausubel, F. et al., eds., John Wiley & Sons, supra) the hybridization methods described above are used to identify the presence of a polymorphism or a particular splicing variant, associated with a disease or condition associated with or a susceptibility to a disease or condition associated with LTA4H (e.g., MI). For Northern analysis, a test sample of RNA is obtained from the individual by appropriate means. Specific hybridization of a nucleic acid probe, as described above, to RNA from the individual is indicative of a polymorphism in an LTA4H nucleic acid, or of the presence of a particular splicing variant encoded by an LTA4H nucleic acid, and is therefore diagnostic for the disease or condition associated with LTA4H, or for susceptibility to a disease or condition associated with LTA4H (e.g., MI).

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a nucleic acid having a polymorphism associated with a disease or condition associated with LTA4H or associated with a susceptibility to a disease or condition associated with LTA4H (e.g., MI). Hybridization of the PNA probe to an LTA4H nucleic acid as described herein is diagnostic for the disease or condition or the susceptibility to the disease or condition.

In another method of the invention, mutation analysis by restriction digestion can be used to detect an altered nucleic acid, or nucleic acids containing a polymorphism(s), if the mutation or polymorphism in the nucleic acid results in the creation or elimination of a restriction site. A test sample containing genomic DNA is obtained from the individual. Polymerase chain reaction (PCR) can be used to amplify an LTA4H nucleic acid (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the alteration or polymorphism in the LTA4H nucleic acid, and therefore indicates the presence or absence of a disease or condition associated with LTA4H or the susceptibility to a disease or condition associated with LTA4H (e.g., MI).

Sequence analysis can also be used to detect specific polymorphisms in the LTA4H nucleic acid. A test sample of DNA or RNA is obtained from the test individual. PCR or other appropriate methods can be used to amplify the nucleic acid, and/or its flanking sequences, if desired. The sequence of an LTA4H nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the nucleic acid, such as cDNA or mRNA (e.g., one or more of SEQ ID NOs: 1 or 2, and/or the complement of SEQ ID NO: 1 or 2), or a nucleic acid sequence encoding SEQ ID NO: 3 or a fragment thereof) or other DNA, as appropriate. The presence of a polymorphism in the LTA4H nucleic acid indicates that the individual has disease or a susceptibility to a disease associated with LTA4H (e.g., MI).

Allele-specific oligonucleotides can also be used to detect the presence of polymorphism(s) in the LTA4H nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, for example, approximately 15-30 base pairs, that specifically hybridizes to an LTA4H nucleic acid, and that contains a polymorphism associated with a disease or condition associated with LTA4H or a susceptibility to a disease or condition associated with LTA4H (e.g., MI). An allele-specific oligonucleotide probe that is specific for particular polymorphisms in an LTA4H nucleic acid can be prepared, using standard methods (see Current Protocols in Molecular Biology, supra). To identify polymorphisms in the nucleic acid associated with disease or susceptibility to disease, a test sample of DNA is obtained from the individual. PCR can be used to amplify all or a fragment of an LTA4H nucleic acid, and its flanking sequences. The DNA containing the amplified LTA4H nucleic acid (or fragment of the nucleic acid) is dot-blotted, using standard methods (see Current Protocols in Molecular Biology, supra), and the blot is contacted with the oligonucleotide probe. The presence of specific hybridization of the probe to the amplified LTA4H is then detected. Specific hybridization of an allele-specific oligonucleotide probe to DNA from the individual is indicative of a polymorphism in the LTA4H, and is therefore indicative of a disease or condition associated with LTA4H or a susceptibility to a disease or condition associated with LTA4H (e.g., MI).

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from an individual, can be used to identify polymorphisms in an LTA4H nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and WO 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991); Pirrung et al., U.S. Pat. No. 5,143,854; (see also PCT Application WO 90/15070); Fodor et al., PCT Publication WO 92/10092; and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified using well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array. In a reverse method, a probe, containing a polymorphism, can be coupled to a solid surface and PCR amplicons are then added to hybridize to these probes.

Although primarily described in terms of a single detection block, e.g., detection of a single polymorphism arrays can include multiple detection blocks, and thus be capable of analyzing multiple, specific polymorphisms. It will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for detection of polymorphisms can be found, for example, in U.S. Pat. Nos. 5,858,659 and 5,837,832, the entire teachings of which are incorporated by reference herein. Other methods of nucleic acid analysis can be used to detect polymorphisms in a nucleic acid described herein, or variants encoded by a nucleic acid described herein. Representative methods include direct manual sequencing (Church and Gilbert, Proc. Natl. Acad. Sci. USA 81:1991-1995 (1988); Sanger, F. et al., Proc. Natl. Acad. Sci., USA 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad. Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one embodiment of the invention, diagnosis of a disease or condition associated with LTA4H (e.g., MI) or a susceptibility to a disease or condition associated with LTA4H (e.g., MI) can also be made by expression analysis by quantitative PCR (kinetic thermal cycling). This technique utilizing TaqMan® can be used to allow the identification of polymorphisms and whether a patient is homozygous or heterozygous. The technique can assess the presence of an alteration in the expression or composition of the polypeptide encoded by an LTA4H nucleic acid or splicing variants encoded by an LTA4H nucleic acid. Further, the expression of the variants can be quantified as physically or functionally different.

In another embodiment of the invention, diagnosis of MI or a susceptibility to MI (or of another disease or condition associated with LTA4H) can also be made by examining expression and/or composition of an LTA4H polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by an LTA4H nucleic acid, or for the presence of a particular variant encoded by an LTA4H nucleic acid. An alteration in expression of a polypeptide encoded by an LTA4H nucleic acid can be, for example, an alteration in the quantitative polypeptide expression (i.e., the amount of polypeptide produced); an alteration in the composition of a polypeptide encoded by an LTA4H nucleic acid is an alteration in the qualitative polypeptide expression (e.g., expression of an altered LTA4H polypeptide or of a different splicing variant). In a preferred embodiment, diagnosis of disease or condition associated with LTA4H or a susceptibility to a disease or condition associated with LTA4H is made by detecting a particular splicing variant encoded by that LTA4H variant, or a particular pattern of splicing variants.

Both such alterations (quantitative and qualitative) can also be present. An “alteration” in the polypeptide expression or composition, refers to an alteration in expression or composition in a test sample, as compared with the expression or composition of polypeptide by an LTA4H nucleic acid in a control sample. A control sample is a sample that corresponds to the test sample (e.g., is from the same type of cells), and is from an individual who is not affected by the disease or a susceptibility to a disease or condition associated with an LTA4H nucleic acid. An alteration in the expression or composition of the polypeptide in the test sample, as compared with the control sample, is indicative of disease or condition associated with LTA4H or a susceptibility to a disease or condition associated with LTA4H (e.g., MI). Similarly, the presence of one or more different splicing variants in the test sample, or the presence of significantly different amounts of different splicing variants in the test sample, as compared with the control sample, is indicative of a susceptibility to a disease or condition associated with an LTA4H nucleic acid. Various means of examining expression or composition of the polypeptide encoded by an LTA4H nucleic acid can be used, including: spectroscopy, colorimetry, electrophoresis, isoelectric focusing and immunoassays (e.g., David et al., U.S. Pat. No. 4,376,110) such as immunoblotting (see also Current Protocols in Molecular Biology, particularly Chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by an altered LTA4H (e.g., by an LTA4H having a SNP as shown in Table 3), or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered LTA4H, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for disease or condition associated with LTA4H or a susceptibility to a disease or condition associated with, as is the presence (or absence) of particular splicing variants encoded by the LTA4H nucleic acid.

In one embodiment of this method, the level or amount of polypeptide encoded by an LTA4H nucleic acid in a test sample is compared with the level or amount of the polypeptide encoded by the LTA4H in a control sample. A level or amount of the polypeptide in the test sample that is higher or lower than the level or amount of the polypeptide in the control sample, such that the difference is statistically significant, is indicative of an alteration in the expression of the polypeptide encoded by the LTA4H, and is diagnostic for disease or condition, or for a susceptibility to a disease or condition, associated with that LTA4H. Alternatively, the composition of the polypeptide encoded by an LTA4H nucleic acid in a test sample is compared with the composition of the polypeptide encoded by the LTA4H in a control sample (e.g., the presence of different splicing variants). A difference in the composition of the polypeptide in the test sample, as compared with the composition of the polypeptide in the control sample, is diagnostic for a disease or condition, or for a susceptibility to a disease or condition, associated with that LTA4H. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a disease or condition, or a susceptibility to a disease or condition, associated with LTA4H (e.g., MI).

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) LTA4H polypeptide, means for amplification of nucleic acids comprising an LTA4H, or means for analyzing the nucleic acid sequence of a nucleic acid described herein, or for analyzing the amino acid sequence of a polypeptide as described herein, etc. In one embodiment, a kit for diagnosing MI or susceptibility to MI can comprise primers for nucleic acid amplification of a region in the LTA4H nucleic acid comprising an at-risk haplotype that is more frequently present in an individual having MI or susceptible to MI. The primers can be designed using portions of the nucleic acids flanking SNPs that are indicative of MI. In a particularly preferred embodiment, the primers are designed to amplify regions of the LTA4H nucleic acid associated with an at-risk haplotype for MI, as shown in Table 4 or Table 5, or more particularly the haplotype defined by the microsatellite markers and SNPs at the locus on chromosome 12q23.

Screening Assays and Agents Identified Therby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of one of SEQ ID NOs: 1 or 2 or the complement thereof, or a nucleic acid encoding an amino acid having the sequence of SEQ ID NO: 3, or a fragment or variant of such nucleic acids), under stringent conditions as described above, and then assessing the sample for the presence (or absence) of hybridization. In a preferred embodiment, high stringency conditions are conditions appropriate for selective hybridization. In another embodiment, a sample containing a nucleic acid molecule of interest is contacted with a nucleic acid containing a contiguous nucleic acid sequence (e.g., a primer or a probe as described above) that is at least partially complementary to a part of the nucleic acid molecule of interest (e.g., an LTA4H nucleic acid), and the contacted sample is assessed for the presence or absence of hybridization. In a preferred embodiment, the nucleic acid containing a contiguous nucleic acid sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes which alter (e.g., increase or decrease) the activity of the polypeptides described herein, or which otherwise interact with the polypeptides herein. For example, such agents can be agents which bind to polypeptides described herein (e.g., binding agent for members of the leukotriene pathway, such as LTA4H binding agents); which have a stimulatory or inhibitory effect on, for example, activity of polypeptides of the invention; or which change (e.g., enhance or inhibit) the ability of the polypeptides of the invention to interact with members of the leukotriene pathway binding agents (e.g., receptors or other binding agents); or which alter posttranslational processing of the leukotriene pathway member polypeptide, such as an LTA4H polypeptide (e.g., agents that alter proteolytic processing to direct the polypeptide from where it is normally synthesized to another location in the cell, such as the cell surface; agents that alter proteolytic processing such that more polypeptide is released from the cell, etc.)

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S., Anticancer Drug Des. 12:145 (1997)).

In one embodiment, to identify agents which alter the activity of an LTA4H polypeptide, a cell, cell lysate, or solution containing or expressing an LTA4H polypeptide (e.g., SEQ ID NO: 3 or another splicing variant encoded by an LTA4H nucleic acid, such as a nucleic acid comprising a SNP as shown in Table 3), or a fragment or derivative thereof (as described above), can be contacted with an agent to be tested; alternatively, the polypeptide can be contacted directly with the agent to be tested. The level (amount) of LTA4H activity is assessed (e.g., the level (amount) of LTA4H activity is measured, either directly or indirectly), and is compared with the level of activity in a control (i.e., the level of activity of the LTA4H polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of an LTA4H polypeptide. An increase in the level of LTA4H activity in the presence of the agent relative to the activity in the absence of the agent, indicates that the agent is an agent that enhances (stimulates) LTA4H activity. Similarly, a decrease in the level of LTA4H activity in the presence of the agent, relative to the activity in the absence of the agent, indicates that the agent is an agent that inhibits LTA4H activity. In another embodiment, the level of activity of an LTA4H polypeptide or derivative or fragment thereof in the presence of the agent to be tested, is compared with a control level that has previously been established. A statistically significant difference in the level of the activity in the presence of the agent from the control level indicates that the agent alters LTA4H activity.

The present invention also relates to an assay for identifying agents which alter the expression of an LTA4H nucleic acid (e.g., antisense nucleic acids, fusion proteins, polypeptides, peptidomimetics, prodrugs, receptors, binding agents, antibodies, small molecules or other drugs, or ribozymes); which alter (e.g., increase or decrease) expression (e.g., transcription or translation) of the nucleic acid or which otherwise interact with the nucleic acids described herein, as well as agents identifiable by the assays. For example, a solution containing a nucleic acid encoding an LTA4H polypeptide (e.g., an LTA4H nucleic acid) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of LTA4H expression (e.g., the level and/or pattern of mRNA or of protein expressed, such as the level and/or pattern of different splicing variants) is assessed, and is compared with the level and/or pattern of expression in a control (i.e., the level and/or pattern of the LTA4H expression in the absence of the agent to be tested). If the level and/or pattern in the presence of the agent differ, by an amount or in a manner that is statistically significant, from the level and/or pattern in the absence of the agent, then the agent is an agent that alters the expression of the LTA4H nucleic acid. Enhancement of LTA4H expression indicates that the agent is an activator of LTA4H transcription. Similarly, inhibition of LTA4H expression indicates that the agent is a repressor of LTA4H transcription.

In another embodiment, the level and/or pattern of LTA4H polypeptide(s) (e.g., different splicing variants) in the presence of the agent to be tested, is compared with a control level and/or pattern that have previously been established. A level and/or pattern in the presence of the agent that differs from the control level and/or pattern by an amount or in a manner that is statistically significant indicates that the agent alters LTA4H expression.

In another embodiment of the invention, agents which alter the expression of an LTA4H nucleic acid or which otherwise interact with the nucleic acids described herein, can be identified using a cell, cell lysate, or solution containing a nucleic acid encoding the promoter region of the LTA4H nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of the LTA4H nucleic acid, as indicated by its ability to alter expression of a nucleic acid that is operably linked to the LTA4H nucleic acid promoter.

Enhancement of the expression of the reporter indicates that the agent is an activator of LTA4H transcription. Similarly, inhibition of the expression of the reporter indicates that the agent is a repressor of LTA4H transcription. In another embodiment, the level of expression of the reporter in the presence of the test agent, is compared with a control level that has previously been established. A level in the presence of the agent that differs from the control level by an amount or in a manner that is statistically significant indicates that the agent alters expression.

Agents which alter the amounts of different splicing variants encoded by an LTA4H nucleic acid (e.g., an agent which enhances activity of a first splicing variant, and which inhibits activity of a second splicing variant), as well as agents which are agonists of activity of a first splicing variant and antagonists of activity of a second splicing variant, can easily be identified using these methods described above.

In other embodiments of the invention, assays can be used to assess the impact of a test agent on the activity of a polypeptide relative to an LTA4H binding agent. For example, a cell that expresses a compound that interacts with LTA4H (herein referred to as a “LTA4H binding agent”, which can be a polypeptide or other molecule that interacts with LTA4H, such as a receptor, or another molecule) is contacted with LTA4H in the presence of a test agent, and the ability of the test agent to alter the interaction between LTA4H and the LTA4H binding agent is determined. Alternatively, a cell lysate or a solution containing the LTA4H binding agent, can be used. An agent which binds to LTA4H or the LTA4H binding agent can alter the interaction by interfering with, or enhancing the ability of LTA4H to bind to, associate with, or otherwise interact with the LTA4H binding agent. Determining the ability of the test agent to bind to LTA4H or an LTA4H binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with LTA4H or an LTA4H binding agent without the labeling of either the test agent, LTA4H, or the LTA4H binding agent. McConnell, H. M. et al., Science 257:1906-1912 (1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

Thus, these receptors can be used to screen for compounds that are agonists for use in treating a disease or condition associated with LTA4H or a susceptibility to a disease or condition associated with LTA4H, or antagonists for studying a susceptibility to a disease or condition associated with LTA4H (e.g., MI). Drugs can be designed to regulate LTA4H activation, which in turn can be used to regulate signaling pathways and transcription events of genes downstream or of proteins or polypeptides interacting with LTA4H.

In another embodiment of the invention, assays can be used to identify polypeptides that interact with one or more LTA4H polypeptides as described herein. For example, a yeast two-hybrid system such as that described by Fields and Song (Fields, S. and Song, O., Nature 340:245-246 (1989)) can be used to identify polypeptides that interact with one or more LTA4H polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also an LTA4H polypeptide, splicing variant, or fragment or derivative thereof, and a second vector is used which includes a nucleic acid encoding a transcription activation domain and also a nucleic acid encoding a polypeptide which potentially may interact with the LTA4H polypeptide, splicing variant, or fragment or derivative thereof (e.g., an LTA4H polypeptide binding agent or receptor). Incubation of yeast containing the first vector and the second vector under appropriate conditions (e.g., mating conditions such as used in the Matchmaker™ system from Clontech (Palo Alto, Calif., USA)) allows identification of colonies that express the markers of interest. These colonies can be examined to identify the polypeptide(s) that interact with the LTA4H polypeptide or fragment or derivative thereof. Such polypeptides may be useful as agents that alter the activity of expression of an LTA4H polypeptide, as described above.

In more than one embodiment of the above assay methods of the present invention, it may be desirable to immobilize either the LTA4H, the LTA4H binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows LTA4H or an LTA4H binding agent to be bound to a matrix or other solid support.

In another embodiment, modulators of expression of nucleic acid molecules of the invention are identified in a method wherein a cell, cell lysate, or solution containing a nucleic acid encoding LTA4H is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

In yet another embodiment, the invention provides methods for identifying agents (e.g., fusion proteins, polypeptides, peptidomimetics, prodrugs, receptors, binding agents, antibodies, small molecules or other drugs, or ribozymes) which alter (e.g., increase or decrease) the activity of a member of the leukotriene pathway binding agent, such as an LTA4H binding agent, as described herein. For example, such agents can be agents which have a stimulatory or inhibitory effect on, for example, the activity of a member of the leukotriene pathway binding agent, such as an LTA4H binding agent; which change (e.g., enhance or inhibit) the ability a member of the leukotriene pathway binding agents, (e.g., receptors or other binding agents) to interact with the polypeptides of the invention; or which alter posttranslational processing of the member of the leukotriene pathway binding agent, (e.g., agents that alter proteolytic processing to direct the member of the leukotriene pathway binding agent from where it is normally synthesized to another location in the cell, such as the cell surface; agents that alter proteolytic processing such that more active binding agent is released from the cell, etc.).

For example, the invention provides assays for screening candidate or test agents that bind to or modulate the activity of a member of the leukotriene pathway (or enzymatically active portion(s) thereof), as well as agents identifiable by the assays. As described above, test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. Anticancer Drug Des., 12:145 (1997)).

In one embodiment, to identify agents which alter the activity of a member of the leukotriene pathway (such as an LTA4H binding agent, or an agent which binds to a member of the leukotriene pathway (a “binding agent”)), a cell, cell lysate, or solution containing or expressing a binding agent (e.g., a leukotriene pathway member receptor, or other binding agent), or a fragment (e.g., an enzymatically active fragment) or derivative thereof, can be contacted with an agent to be tested; alternatively, the binding agent (or fragment or derivative thereof) can be contacted directly with the agent to be tested. The level (amount) of binding agent activity is assessed (either directly or indirectly), and is compared with the level of activity in a control (i.e., the level of activity in the absence of the agent to be tested). If the level of the activity in the presence of the agent differs, by an amount that is statistically significant, from the level of the activity in the absence of the agent, then the agent is an agent that alters the activity of the member of the leukotriene pathway. An increase in the level of the activity relative to a control, indicates that the agent is an agent that enhances the activity. Similarly, a decrease in the level of activity relative to a control, indicates that the agent is an agent that inhibits the activity. In another embodiment, the level of activity in the presence of the agent to be tested, is compared with a control level that has previously been established. A level of the activity in the presence of the agent that differs from the control level by an amount that is statistically significant indicates that the agent alters the activity.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent.

Furthermore, this invention pertains to uses of novel agents identified by the above-described screening assays for treatments as described herein. In addition, an agent identified as described herein can be used to alter activity of a polypeptide encoded by an LTA4H nucleic acid, or to alter expression of an LTA4H nucleic acid, by contacting the polypeptide or the nucleic acid (or contacting a cell comprising the polypeptide or the nucleic acid) with the agent identified as described herein.

The present invention is now illustrated by the following Examples, which are not intended to be limiting in any way.

### EXAMPLE 1

### Identification of Haplotypes Associated with MI Subjects and Methods

Study Population

Patients entering the study were defined from a myocardial infarction (MI) registry that includes all MIs (over 8,000 patients) in Iceland from 1981 to 2002. This registry is a part of the World Health Organization MONICA Project (The World Health Organization MONICA Project (monitoring trends and determinants in cardiovascular disease): a major international collaboration. WHO MONICA Project Principal Investigators. J. Clin. Epidemiol. 1988; 41:105-14). Diagnosis of all patients in the registry follow strict diagnostic rules based on symptoms, electrocardiograms, cardiac enzymes, and necropsy findings.

Blood samples from over 1500 MI patients, both cases with a family history and sporadic cases were collected. For each patient that participated, blood was collected from 2 relatives (unaffected or affected). Their genotypes were used to help with construction of haplotypes. Blood samples from over 950 controls were also collected. The control cohort was population based.

Linkage Analysis

In an effort to enrich for those patients who had stronger genetic factors contributing to their risk for MI, we fractionated the MI cohort to those patients with earlier onset MI. We chose different age cutoffs for male and females since the average age of MI in females is 10 years older than for males. Using MI onset at age less than 50 in males and less than 60 in females, 196 patients were clustered within 67 Pedigrees. These pedigrees included related earlier onset MI patients such that each patient is related to at least one other patient up to and including six meiotic events. The information regarding the relatedness of patients was obtained from an encrypted genealogy database that covers the entire Icelandic nation (Gulcher et al., Eur. J. Hum. Genet. 8: 739-742 (2000)). A genome-wide scan was performed using a framework map of 1000 microsatellite markers, using protocols described elsewhere (Gretarsdottir S., et al. Am. J. Hum. Genet., 70: 593-603, 2002)). The marker order and positions were obtained from deCODE genetic's high resolution genetic map (Kong A, et al., Nat. genet., 31: 241-247 (2002)). All markers used in the linkage analysis are publicly available microsatellite markers. The population-based allele frequencies were constructed from a cohort of more than 30,000 Icelanders who have participated in genetic studies of various disease projects.

For statistical analysis, multipoint, affected only allele-sharing methods were used to assess evidence for linkage. All results, both the LOD and the non-parametric linkage (NPL) score, were obtained using the program ALLEGRO (Gudbjartsson D. F., et al., Nat Genet., 25: 12-13 (2000)). The baseline linkage analysis (Gretarsdottir S., et al., Am. J. Hum. Genet. 70: 593-603, (2002)) uses the Spairs scoring function (Whittermore A S, and Haplern J A., Biometrics 50: 118-127 (1994)) and Kruglyak et al., Am. J. Hum. Genet., 58:1347-1363 (1996)) the exponential allele-sharing model (Kong A., and Cox N. J., Am. J. Hum. Genet. 61:1179-1188 (1997)), and a family weighting scheme which is halfway, on the log-scale, between weighing each affected pairs equally and weighing each family equally.

Fine Mapping:

A candidate susceptibility locus was defined as the region under the LOD score curve where the score was one lower than the highest lod score ((peak lod score −1)\one lod drop). This region (approx. 12 Mb) was finemapped with microsatellite markers with an average spacing between markers of approximately 1.5 cM.

Case-Control Haplotype Association Analysis

A large case-control analysis was initially carried out using over 560 male MI patients and 338 female MI patients and 480 population-based controls in an effort to find the MI gene within the linkage peak on chromosome 12 found in genome-wide linkage analysis. Given that a member of the leukotriene biosynthetic pathway, LTA4H, was near the peak microsatellite marker, an effort was made to identify microsatellite markers positioned close to, or within, the LTA4H gene. Three microsatellite markers were identified within the deCODE genetics modified assembly of the public UCSC human genome sequence assembly and they were subsequently genotyped. In addition, SNPs were identified within the LTA4H gene by sequencing 93 patients. Out of the 90 SNPs that were identified 12 were selected to genotype 894 patients and 462 controls. These three microsatellite markers and 12 SNPs, were subsequently used for haplotype analysis. Results from the initial haplotype analysis are shown in Table 4 and Table 5.

We then typed a subset of the markers on more MI patients and controls. This subset included 8 SNPs and 3 microsatellite markers. In addition, we typed 9 new SNPs on the total cohort which now included 1560 MI patients and 953 controls. Results from the haplotype association analysis, using the extended cohort and a total of 17 SNPs and 3 microsatellite markers, are shown in Table 5.

The frequencies of haplotypes in the patient and the control groups using an expectation-maximization algorithm were estimated (Dempster A. P. et al., J. R. Stat. Soc. B. 39: 1-389 (1977)). An implementation of this algorithm that can handle missing genotypes and uncertainty with the phase was used. Under the null hypothesis, the patients and the controls are assumed to have identical frequencies. Using a likelihood approach, an alternative hypothesis where a candidate at-risk-haplotype is allowed to have a higher frequency in patients than controls, while the ratios of the frequencies of other haplotypes are assumed to be the same in both groups was tested. Likelihoods are maximized separately under both hypothesis and a corresponding 1-df likelihood ratio statistics is used to evaluate the statistic significance.

To assess the significance of the haplotype association corrected for multiple testing, we carried out a randomisation test using the same genotype data. We randomised the cohorts of patients and controls and repeated the analysis. This procedure was repeated up to 500 times and the adjusted P value is the fraction of replications that produced a P value for some haplotype tested that is lower than or equal to the P value we observed using the original patient and control cohorts.

Results:

Table 1 shows the results of the first step of the linkage analysis; multipoint non-parametric LOD scores for a framework marker map on chromosome 12. A LOD score suggestive of linkage of 1.95 was found at marker D12S2081. This linkage peak was one of the highest peaks found for the earlier onset MI phenotype. Table 2 shows the results of the second step of the linkage analysis; multipoint non-parametric LOD scores for the families after adding 20 fine mapping markers to the candidate region. The inclusion of additional microsatellite markers increased the information on sharing by decent from 0.8 to 0.9, around the markers that gave the highest LOD scores. The lodscore in this locus increased to 2.01 and the peak marker was D12S348 at centimorgin distance 110.6. Thus the locus remained suggestive for linkage suggesting that a gene conferring risk for MI was within the 10 million bases defined by the width of the linkage peak.

One of the genes close to the peak marker at this linkage peak (that is, the marker with the highest sharing or lodscore) was LTA4H. Our previous genetic work with FLAP showed that the leukotriene biosynthetic pathway plays a major role in MI risk. Since LTA4H encodes a major member of the leukotriene biosynthetic pathway converting Leukotriene A to Leukotriene B, we chose to test it for association to MI in a case-control study using 894 MI patients and 462 population-based controls.

Table 3 shows SNPs that were found by sequencing the LTA4H gene. One of the SNPs, LTA4H_{—}31334, is in the coding region. The polymorphism, A\G, does not change the amino acid sequence in the protein. The rest of the SNPs were outside the coding exons of LTA4H and were within introns or flanking regions of LTA4H.

Table 4 shows results from the initial haplotype association analysis using 894 MI patients and 462 controls that were typed with 3 microsatellite markers and 12 SNPs. The following markers show a significant association with MI in males: DG12S1664, SG12S16, SG12S17, SG12S18, SG12S21, SG12S22, SG12S23, SG12S24, SG12S25, SG12S26, DG12S1666, SG12S100, SG12S28, and SG12S144, with alleles 0, C, A, T, G, G, T, T, A, T, 0, and T, T, and A, respectively. The allelic frequency of a shorter version of this haplotype including markers DG12S1664, SG12S26, DG12S1666, and SG12S144, with alleles 0, T, 0, and A, respectively, is 51% in male MI patients and 43% in controls (carried by 76% of male patients and 67% of controls). Allelic frequency of this haplotype is higher, or 56%, in a subgroup of patients that have had more than one MI (see Table 4).

Table 5 shows the results of the haplotype association analysis using 1560 unrelated MI patients and 953 unrelated population controls. A haplotype comprised of the consecutive markers was highly significant in MI patients that had also had either stroke or peripheral arterial occlusive disease (PAOD) (P-value adjusted for multiple comparisons=0.007). The fact that the haplotypes shown in Table 5 are more significant in MI patients that have more than one clinically evident cardiovascular complication might indicate that the gene played a role in clinical activity or severity of the atherosclerotic disease. The significantly associated haplotype is comprised of the following consecutive markers; SG12S438, DG12S1664, SG12S16, SG12S21, SG12S23, SG12S25, SG12S26, DG12S1666, SG12S100, SG12S28, SG12S143, SG12S144, SG12S221, SG12S222, SG12S223, SG12S225, SG12S226, SG12S233, SG12S237, and DG12S1668 with alleles C, 0, C, G, T, A, T, 0, T, T, T, A, G, C, C, G, G, C, T, and 0. Also shown in Table 5 is a shorter version of the consecutive haplotype and a haplotype that shows a significant protection against MI involving more than one clinically evident cardiovascular complication.

In summary, it has been shown for the first time that genetic varians of LTA4H show significant association to MI. The results complement previous work showing that variants in FLAP are significantly associated with MI. In both cases the risk ratio is similar to or higher than the conventional and well-known risk factors for MI including smoking, hypercholesterolemia, hypertension and diabetes among others.

TABLE 1
The marker map for chromosome 12 and LOD scores in the
first step of the linkage analysis.

| location | LOD | dhat | NPL | Zlr | Info | marker |
|---|---|---|---|---|---|---|
| 0 | 1.2574 | −0.4865 | −1.6783 | −2.4063 | 0.5456 | D12S352 |
| 3.083 | 1.7993 | −0.5525 | −2.1441 | −2.8786 | 0.6374 | D12S1608 |
| 3.554 | 1.8107 | −0.5494 | −2.1696 | −2.8877 | 0.6472 | D12S1656 |
| 6.566 | 1.8434 | −0.5493 | −2.2066 | −2.9136 | 0.6591 | D12S1626 |
| 7.956 | 1.8748 | −0.5527 | −2.2239 | −2.9383 | 0.6638 | D12S372 |
| 12.93 | 1.5997 | −0.4719 | −2.166 | −2.7142 | 0.7291 | D12S1725 |
| 13.761 | 1.6842 | −0.4859 | −2.2249 | −2.785 | 0.732 | D12S314 |
| 16.166 | 1.6989 | −0.5279 | −2.0948 | −2.7971 | 0.6467 | D12S374 |
| 24.078 | 1.0258 | −0.4043 | −1.5861 | −2.1734 | 0.6036 | D12S336 |
| 26.254 | 1.0166 | −0.3907 | −1.6163 | −2.1637 | 0.6338 | D12S1697 |
| 31.288 | 0.9373 | −0.3846 | −1.5323 | −2.0775 | 0.6 | D12S364 |
| 34.202 | 0.8469 | −0.3806 | −1.4006 | −1.9748 | 0.5518 | D12S1728 |
| 39.399 | 0.8692 | −0.4163 | −1.3441 | −2.0007 | 0.4871 | D12S1682 |
| 44.135 | 0.7789 | −0.3786 | −1.306 | −1.894 | 0.5121 | D12S1591 |
| 49.974 | 0.7977 | −0.3819 | −1.3162 | −1.9166 | 0.5166 | D12S1640 |
| 52.254 | 0.8638 | −0.3759 | −1.4437 | −1.9945 | 0.5749 | D12S1704 |
| 53.951 | 0.8005 | −0.3442 | −1.4441 | −1.92 | 0.6191 | D12S1681 |
| 55.792 | 0.4155 | −0.2301 | −1.0815 | −1.3833 | 0.6554 | D12S345 |
| 57.468 | 0.2695 | −0.1842 | −0.8653 | −1.114 | 0.6382 | D12S1668 |
| 61.09 | 0.6674 | −0.3134 | −1.2999 | −1.7531 | 0.6074 | D12S85 |
| 67.239 | 0.9722 | −0.3854 | −1.5762 | −2.116 | 0.6203 | D12S368 |
| 74.802 | 0.8922 | −0.3971 | −1.4186 | −2.027 | 0.5412 | D12S83 |
| 76.789 | 0.9969 | −0.4272 | −1.4897 | −2.1426 | 0.5351 | D12S329 |
| 84.363 | 0.0618 | −0.103 | −0.3514 | −0.5333 | 0.4367 | D12S313 |
| 92.292 | 0.0266 | 0.052 | 0.2826 | 0.3497 | 0.6444 | D12S326 |
| 96.995 | 0.2219 | 0.1438 | 0.8312 | 1.0108 | 0.6496 | D12S1708 |
| 102.426 | 1.0345 | 0.2707 | 2.0001 | 2.1827 | 0.7615 | D12S351 |
| 103.746 | 1.4296 | 0.3119 | 2.3732 | 2.5659 | 0.7625 | D12S95 |
| 109.914 | 1.9537 | 0.3537 | 2.8183 | 2.9995 | 0.7796 | D12S2081 |
| 112.689 | 1.4231 | 0.2984 | 2.4796 | 2.56 | 0.84 | D12S346 |
| 114.367 | 1.1079 | 0.2685 | 2.1563 | 2.2588 | 0.8307 | D12S1727 |
| 117.962 | 1.2498 | 0.2916 | 2.2133 | 2.3991 | 0.7773 | D12S78 |
| 123.398 | 0.2995 | 0.1592 | 1.012 | 1.1744 | 0.7055 | D12S1613 |
| 126.542 | 0.1457 | 0.1139 | 0.6968 | 0.819 | 0.6986 | D12S1583 |
| 132.981 | 0.0058 | 0.0232 | 0.1392 | 0.1631 | 0.7222 | D12S354 |
| 133.655 | 0.0011 | 0.0106 | 0.0607 | 0.0725 | 0.6962 | D12S369 |
| 133.964 | 0.0012 | 0.0107 | 0.0608 | 0.0728 | 0.6913 | D12S79 |
| 139.646 | 0.0742 | 0.0823 | 0.4953 | 0.5844 | 0.701 | D12S366 |
| 142.505 | 0.1383 | 0.1088 | 0.694 | 0.7979 | 0.7292 | D12S395 |
| 143.459 | 0.0732 | 0.0795 | 0.5072 | 0.5805 | 0.7417 | D12S2073 |
| 143.698 | 0.0886 | 0.0875 | 0.5572 | 0.6387 | 0.7369 | D12S1349 |
| 144.394 | 0.0604 | 0.0727 | 0.4591 | 0.5275 | 0.7376 | D12S378 |
| 148.306 | 0 | 0.0013 | 0.0084 | 0.0096 | 0.7673 | D12S1614 |
| 151.275 | 0.0125 | 0.0351 | 0.1985 | 0.2397 | 0.6764 | D12S324 |
| 155.308 | 0.3155 | 0.1758 | 0.9568 | 1.2054 | 0.6008 | D12S2075 |
| 156.144 | 0.2797 | 0.1706 | 0.8734 | 1.1348 | 0.5679 | D12S1675 |
| 158.207 | 0.3194 | 0.1834 | 0.9265 | 1.2128 | 0.5549 | D12S1679 |
| 162.448 | 0.3706 | 0.1872 | 1.0567 | 1.3063 | 0.6156 | D12S1659 |
| 164.59 | 0.368 | 0.1876 | 1.0474 | 1.3019 | 0.6084 | D12S367 |
| 172.615 | 0.3231 | 0.1872 | 0.9214 | 1.2199 | 0.5371 | D12S1723 |
| 174.333 | 0.2827 | 0.1781 | 0.847 | 1.1411 | 0.5229 | D12S1638 |

TABLE 2
The marker map for chromosome 12 and LOD scores, in the
second step of the linkage analysis.

| location | LOD | dhat | NPL | Zlr | Info | marker |
|---|---|---|---|---|---|---|
| 0 | 1.6956 | −0.6253 | −1.8379 | −2.7944 | 0.4963 | D12S352 |
| 3.758 | 2.024 | −0.6098 | −2.2287 | −3.053 | 0.6154 | D12S1608 |
| 4.239 | 2.0532 | −0.6089 | −2.262 | −3.0749 | 0.6257 | D12S1656 |
| 4.899 | 2.0351 | −0.6062 | −2.2476 | −3.0614 | 0.6244 | D12S100 |
| 4.949 | 2.0335 | −0.6059 | −2.2466 | −3.0601 | 0.6243 | D12S1694 |
| 5.825 | 1.9982 | −0.5969 | −2.2337 | −3.0335 | 0.6278 | D12S1615 |
| 7.41 | 1.895 | −0.5609 | −2.2259 | −2.9541 | 0.6556 | D12S1626 |
| 8.241 | 1.9046 | −0.5627 | −2.2255 | −2.9616 | 0.6556 | D12S372 |
| 9.071 | 1.8945 | −0.5659 | −2.197 | −2.9537 | 0.6463 | D12S835 |
| 9.239 | 1.8908 | −0.5659 | −2.1919 | −2.9509 | 0.6452 | D12S1050 |
| 9.628 | 1.8804 | −0.5648 | −2.1812 | −2.9427 | 0.6435 | D12S1652 |
| 13.786 | 1.6009 | −0.4751 | −2.1492 | −2.7152 | 0.7218 | D12S1725 |
| 14.624 | 1.596 | −0.4767 | −2.1379 | −2.7111 | 0.7157 | D12S314 |
| 15.679 | 1.7102 | −0.5249 | −2.1113 | −2.8064 | 0.6569 | D12S328 |
| 15.729 | 1.7111 | −0.5255 | −2.1102 | −2.8071 | 0.656 | D12S93 |
| 15.917 | 1.7113 | −0.5272 | −2.1062 | −2.8073 | 0.6527 | D12S99 |
| 16.495 | 1.6721 | −0.5331 | −2.0411 | −2.7749 | 0.6266 | D12S1673 |
| 16.684 | 1.6562 | −0.5339 | −2.0199 | −2.7617 | 0.6192 | D12S356 |
| 17.131 | 1.6124 | −0.5336 | −1.9702 | −2.725 | 0.6035 | D12S374 |
| 20.18 | 1.4787 | −0.5541 | −1.7482 | −2.6095 | 0.5214 | D12S1625 |
| 23.545 | 1.1182 | −0.4645 | −1.5402 | −2.2693 | 0.5229 | D12S397 |
| 24.869 | 0.9441 | −0.4038 | −1.4682 | −2.0852 | 0.5568 | D12S1695 |
| 24.979 | 0.9297 | −0.3985 | −1.4625 | −2.0692 | 0.5606 | D12S336 |
| 25.269 | 0.9337 | −0.399 | −1.4663 | −2.0736 | 0.5617 | D12S1674 |
| 25.559 | 0.9367 | −0.3992 | −1.4704 | −2.077 | 0.5632 | D12S1690 |
| 25.772 | 0.9384 | −0.3989 | −1.4735 | −2.0788 | 0.5648 | D12S1696 |
| 25.793 | 0.9385 | −0.3989 | −1.4738 | −2.0789 | 0.5649 | D12S77 |
| 26.767 | 0.9395 | −0.3946 | −1.4893 | −2.08 | 0.5758 | D12S827 |
| 27.155 | 0.937 | −0.3915 | −1.4961 | −2.0773 | 0.5821 | D12S1697 |
| 27.325 | 0.938 | −0.3939 | −1.4894 | −2.0784 | 0.5766 | D12S89 |
| 28.883 | 0.9248 | −0.4057 | −1.4313 | −2.0636 | 0.5411 | D12S391 |
| 30.851 | 0.8473 | −0.39 | −1.3665 | −1.9754 | 0.5299 | D12S1581 |
| 31.936 | 0.7765 | −0.3651 | −1.3345 | −1.891 | 0.5429 | D12S1580 |
| 32.188 | 0.7575 | −0.3576 | −1.3274 | −1.8677 | 0.5489 | D12S320 |
| 32.238 | 0.7536 | −0.356 | −1.326 | −1.863 | 0.5503 | D12S364 |
| 32.735 | 0.7445 | −0.3581 | −1.3038 | −1.8516 | 0.538 | D12S308 |
| 34.013 | 0.7073 | −0.3557 | −1.2478 | −1.8048 | 0.5172 | D12S2210 |
| 34.335 | 0.6949 | −0.3532 | −1.2338 | −1.7889 | 0.5143 | D12S1303 |
| 35.153 | 0.6582 | −0.3436 | −1.1984 | −1.741 | 0.5108 | D12S1728 |
| 36.074 | 0.693 | −0.3705 | −1.1841 | −1.7864 | 0.4727 | D12S1715 |
| 37.358 | 0.7161 | −0.3917 | −1.1671 | −1.816 | 0.4445 | D12S310 |
| 37.716 | 0.723 | −0.3955 | −1.1681 | −1.8247 | 0.4414 | D12S1669 |
| 39.199 | 0.7267 | −0.3952 | −1.1753 | −1.8294 | 0.4443 | D12S1650 |
| 40.35 | 0.7034 | −0.3777 | −1.1844 | −1.7998 | 0.4644 | D12S1682 |
| 45.086 | 0.6102 | −0.3149 | −1.1956 | −1.6764 | 0.5509 | D12S1591 |
| 46.757 | 0.645 | −0.3251 | −1.2237 | −1.7234 | 0.5509 | D12S1057 |
| 47.216 | 0.6504 | −0.3287 | −1.2219 | −1.7307 | 0.5449 | D12S1617 |
| 49.098 | 0.6565 | −0.332 | −1.2227 | −1.7387 | 0.5404 | D12S1596 |
| 50.007 | 0.6508 | −0.3269 | −1.2292 | −1.7312 | 0.5503 | D12S1034 |
| 50.925 | 0.6382 | −0.3169 | −1.2391 | −1.7144 | 0.5696 | D12S1640 |
| 53.204 | 0.7066 | −0.3153 | −1.3729 | −1.8039 | 0.6362 | D12S1704 |
| 53.205 | 0.7066 | −0.3153 | −1.373 | −1.8039 | 0.6362 | D12S1643 |
| 54.901 | 0.6809 | −0.2936 | −1.4087 | −1.7708 | 0.695 | D12S1681 |
| 55.526 | 0.5731 | −0.2654 | −1.301 | −1.6245 | 0.6994 | D12S1648 |
| 55.827 | 0.5217 | −0.2504 | −1.25 | −1.55 | 0.7065 | D12S61 |
| 56.499 | 0.4119 | −0.2146 | −1.1385 | −1.3772 | 0.737 | ATA73C05 |
| 56.549 | 0.4041 | −0.2119 | −1.1303 | −1.3641 | 0.7401 | D12S1621 |
| 56.793 | 0.3671 | −0.1986 | −1.0906 | −1.3002 | 0.7572 | D12S345 |
| 57.118 | 0.3602 | −0.1959 | −1.0835 | −1.288 | 0.7615 | D12S2080 |
| 58.072 | 0.3416 | −0.1881 | −1.0664 | −1.2542 | 0.7782 | D12S1048 |
| 58.469 | 0.3345 | −0.1849 | −1.0609 | −1.2411 | 0.7867 | D12S1668 |
| 59.057 | 0.3671 | −0.1944 | −1.1109 | −1.3002 | 0.7874 | D12S1589 |
| 59.716 | 0.4056 | −0.2045 | −1.1706 | −1.3667 | 0.7932 | D12S291 |
| 60.054 | 0.4612 | −0.221 | −1.2374 | −1.4573 | 0.7826 | D12S1301 |
| 61.826 | 0.7555 | −0.2833 | −1.6011 | −1.8652 | 0.8213 | D12S1713 |
| 62.09 | 0.7752 | −0.2879 | −1.6189 | −1.8894 | 0.819 | D12S85 |
| 63.701 | 0.8433 | −0.309 | −1.6549 | −1.9707 | 0.7867 | D12S1701 |
| 64.377 | 0.8374 | −0.3088 | −1.6463 | −1.9637 | 0.7819 | D12S2199 |
| 64.888 | 0.821 | −0.3047 | −1.6355 | −1.9445 | 0.785 | D12S1590 |
| 65.096 | 0.8096 | −0.3025 | −1.6239 | −1.9309 | 0.784 | D12S1627 |
| 65.665 | 0.8586 | −0.3194 | −1.6441 | −1.9884 | 0.756 | D12S1620 |
| 65.666 | 0.8587 | −0.3194 | −1.6441 | −1.9885 | 0.7561 | D12S1635 |
| 66.235 | 0.8957 | −0.3295 | −1.6678 | −2.031 | 0.7474 | D12S1633 |
| 66.236 | 0.8958 | −0.3295 | −1.6678 | −2.0311 | 0.7473 | D12S1629 |
| 66.838 | 0.9205 | −0.3325 | −1.6967 | −2.0589 | 0.7558 | D12S347 |
| 67.205 | 0.9208 | −0.3307 | −1.7028 | −2.0592 | 0.7633 | D12S1677 |
| 68.24 | 1.1611 | −0.3656 | −1.9527 | −2.3124 | 0.8101 | D12S368 |
| 68.854 | 1.1354 | −0.3678 | −1.9021 | −2.2867 | 0.7842 | D12S96 |
| 69.118 | 1.1237 | −0.3682 | −1.8815 | −2.2749 | 0.7746 | D12S398 |
| 70.315 | 1.0649 | −0.3662 | −1.7961 | −2.2145 | 0.7407 | D12S1604 |
| 70.523 | 1.0539 | −0.3653 | −1.7827 | −2.2031 | 0.7365 | D12S359 |
| 70.637 | 1.0579 | −0.3678 | −1.7787 | −2.2072 | 0.7304 | D12S1651 |
| 71.597 | 1.0794 | −0.3844 | −1.7459 | −2.2296 | 0.6917 | D12S1724 |
| 71.8 | 1.0813 | −0.3867 | −1.7392 | −2.2315 | 0.6859 | D12S1707 |
| 72.252 | 1.0822 | −0.3904 | −1.7247 | −2.2324 | 0.6753 | D12S2191 |
| 73.451 | 1.0636 | −0.3917 | −1.6882 | −2.2132 | 0.6601 | D12S1632 |
| 74.528 | 1.0229 | −0.3828 | −1.6582 | −2.1704 | 0.6601 | D12S90 |
| 74.775 | 1.0106 | −0.3795 | −1.6517 | −2.1573 | 0.6617 | D12S305 |
| 74.919 | 1.0029 | −0.3773 | −1.648 | −2.1491 | 0.6631 | D12S1298 |
| 75.69 | 0.9563 | −0.363 | −1.6289 | −2.0985 | 0.6753 | D12S1700 |
| 75.691 | 0.9562 | −0.3629 | −1.6288 | −2.0984 | 0.6756 | D12S1056 |
| 75.744 | 0.9527 | −0.3618 | −1.6276 | −2.0946 | 0.6767 | D12S1662 |
| 75.802 | 0.9487 | −0.3605 | −1.6262 | −2.0902 | 0.6779 | D12S83 |
| 75.803 | 0.9487 | −0.3605 | −1.6262 | −2.0902 | 0.6779 | D12S1655 |
| 76.339 | 0.9582 | −0.3657 | −1.6221 | −2.1006 | 0.6682 | D12S298 |
| 76.916 | 0.9668 | −0.3701 | −1.62 | −2.1101 | 0.6606 | D12S1726 |
| 77.789 | 0.9767 | −0.3743 | −1.621 | −2.1209 | 0.6546 | D12S329 |
| 80.622 | 0.7896 | −0.3801 | −1.2958 | −1.9068 | 0.5155 | D12S1649 |
| 83.513 | 0.4582 | −0.2911 | −0.9752 | −1.4527 | 0.4746 | D12S1601 |
| 84.007 | 0.3957 | −0.2648 | −0.9209 | −1.35 | 0.4851 | D12S1294 |
| 84.428 | 0.3441 | −0.2407 | −0.8746 | −1.2588 | 0.5003 | D12S335 |
| 85.558 | 0.2207 | −0.1753 | −0.75 | −1.0081 | 0.573 | D12S313 |
| 86.414 | 0.2075 | −0.1672 | −0.7361 | −0.9775 | 0.5883 | D12S375 |
| 86.588 | 0.2051 | −0.1658 | −0.7331 | −0.9718 | 0.5905 | D12S1680 |
| 87.042 | 0.198 | −0.1615 | −0.7253 | −0.9549 | 0.5991 | D12S1693 |
| 88.586 | 0.1683 | −0.1407 | −0.7008 | −0.8803 | 0.6584 | D12S1040 |
| 89.237 | 0.1545 | −0.1303 | −0.6917 | −0.8436 | 0.6988 | D12S299 |
| 89.238 | 0.1545 | −0.1303 | −0.6917 | −0.8435 | 0.6987 | D12S92 |
| 89.781 | 0.143 | −0.1214 | −0.6848 | −0.8116 | 0.7399 | D12S1052 |
| 90.368 | 0.131 | −0.1118 | −0.6779 | −0.7767 | 0.7921 | D12S337 |
| 91.289 | 0.155 | −0.1175 | −0.7641 | −0.8449 | 0.8534 | D12S1660 |
| 91.913 | 0.087 | −0.0886 | −0.5648 | −0.6331 | 0.8225 | D12S1684 |
| 92.02 | 0.0761 | −0.0831 | −0.5262 | −0.5921 | 0.8142 | D12S350 |
| 93.288 | 0.0009 | −0.0089 | −0.0583 | −0.0652 | 0.8082 | D12S326 |
| 97.989 | 0.2109 | 0.123 | 0.9332 | 0.9855 | 0.8597 | D12S1297 |
| 97.99 | 0.2119 | 0.1234 | 0.9351 | 0.9879 | 0.8588 | D12S106 |
| 97.991 | 0.213 | 0.1237 | 0.9371 | 0.9903 | 0.8578 | D12S1708 |
| 99.524 | 0.6535 | 0.201 | 1.7426 | 1.7347 | 0.9295 | D12S1667 |
| 99.525 | 0.6535 | 0.201 | 1.7427 | 1.7348 | 0.9296 | D12S319 |
| 100.397 | 0.7234 | 0.208 | 1.8684 | 1.8252 | 0.9553 | D12S323 |
| 100.398 | 0.7235 | 0.208 | 1.8686 | 1.8253 | 0.955 | D12S88 |
| 100.399 | 0.7301 | 0.2091 | 1.8758 | 1.8336 | 0.9533 | D12S1719 |
| 100.519 | 0.7536 | 0.2127 | 1.9016 | 1.8629 | 0.947 | D12S1593 |
| 101.064 | 0.8567 | 0.2269 | 2.0196 | 1.9863 | 0.9341 | D12S853 |
| 101.841 | 0.9732 | 0.2384 | 2.1747 | 2.117 | 0.951 | D12S1710 |
| 102.131 | 1.1754 | 0.2589 | 2.4086 | 2.3266 | 0.9561 | D12S1717 |
| 103.423 | 1.1442 | 0.2555 | 2.379 | 2.2955 | 0.9588 | D12S351 |
| 104.343 | 1.341 | 0.2756 | 2.5694 | 2.485 | 0.9479 | D12S311 |
| 104.743 | 1.6769 | 0.3035 | 2.8993 | 2.7789 | 0.952 | D12S95 |
| 105.266 | 1.7384 | 0.3095 | 2.9441 | 2.8294 | 0.9441 | D12S1345 |
| 106.345 | 1.8647 | 0.326 | 2.9793 | 2.9304 | 0.8988 | D12S1346 |
| 110.627 | 2.0063 | 0.3408 | 3.0437 | 3.0397 | 0.8726 | D12S348 |
| 110.908 | 1.9856 | 0.337 | 3.0533 | 3.0239 | 0.8861 | D12S1716 |
| 110.909 | 1.9854 | 0.337 | 3.053 | 3.0238 | 0.886 | D12S1657 |
| 112.477 | 1.3244 | 0.2754 | 2.5394 | 2.4696 | 0.9375 | D12S393 |
| 112.658 | 1.5716 | 0.2988 | 2.7576 | 2.6903 | 0.9246 | D12S1706 |
| 113.456 | 1.482 | 0.2868 | 2.7191 | 2.6125 | 0.9569 | D12S1600 |
| 113.686 | 1.4654 | 0.2856 | 2.7011 | 2.5978 | 0.9556 | D12S346 |
| 114.583 | 1.2538 | 0.2643 | 2.5203 | 2.4029 | 0.9739 | D12S1641 |
| 114.628 | 1.2491 | 0.2637 | 2.5166 | 2.3984 | 0.9748 | D12S306 |
| 114.674 | 1.2445 | 0.2632 | 2.5127 | 2.3939 | 0.9759 | D12S332 |
| 115.043 | 1.3131 | 0.271 | 2.5676 | 2.4591 | 0.9635 | D12S1041 |
| 115.364 | 1.1318 | 0.2546 | 2.3621 | 2.283 | 0.956 | D12S1727 |
| 116.299 | 1.1829 | 0.2606 | 2.4032 | 2.334 | 0.9477 | D12S1607 |
| 116.948 | 1.2361 | 0.2691 | 2.4273 | 2.3859 | 0.9221 | IGF1 |
| 116.949 | 1.2361 | 0.2691 | 2.4273 | 2.3859 | 0.9219 | D12S1030 |
| 117.75 | 1.5059 | 0.2956 | 2.6701 | 2.6334 | 0.9082 | PAH |
| 118.61 | 1.2001 | 0.2629 | 2.4192 | 2.3509 | 0.9435 | D12S360 |
| 118.899 | 1.4558 | 0.2869 | 2.6729 | 2.5893 | 0.9393 | D12S78 |
| 119.188 | 1.399 | 0.2838 | 2.5969 | 2.5382 | 0.9253 | D12S338 |
| 120.067 | 1.3032 | 0.2727 | 2.5213 | 2.4498 | 0.943 | D12S1647 |
| 120.068 | 1.2993 | 0.2723 | 2.5179 | 2.4461 | 0.9436 | D12S317 |
| 120.348 | 1.4722 | 0.2886 | 2.6798 | 2.6038 | 0.9378 | D12S1597 |
| 121.195 | 1.3839 | 0.2842 | 2.5548 | 2.5245 | 0.9127 | D12S1683 |
| 124.023 | 0.6306 | 0.2003 | 1.693 | 1.7041 | 0.9045 | D12S1342 |
| 124.297 | 0.6069 | 0.198 | 1.6474 | 1.6718 | 0.8927 | D12S1613 |
| 125.597 | 0.483 | 0.183 | 1.4221 | 1.4915 | 0.8432 | D12S1605 |
| 126.055 | 0.451 | 0.1786 | 1.3612 | 1.4411 | 0.8293 | D12S84 |
| 126.796 | 0.3855 | 0.1683 | 1.2383 | 1.3324 | 0.8059 | D12S105 |
| 127.545 | 0.3132 | 0.1527 | 1.1129 | 1.2009 | 0.8072 | D12S1583 |
| 129.188 | 0.2211 | 0.1354 | 0.8864 | 1.009 | 0.7362 | D12S1344 |
| 130.64 | 0.141 | 0.1122 | 0.6858 | 0.8058 | 0.6977 | D12S1616 |
| 133.986 | 0.0109 | 0.0313 | 0.1941 | 0.2238 | 0.742 | D12S354 |
| 134.268 | 0.0114 | 0.0321 | 0.1973 | 0.2287 | 0.7353 | D12S1023 |
| 134.818 | 0.0122 | 0.0336 | 0.2027 | 0.237 | 0.7233 | D12S369 |
| 134.959 | 0.0122 | 0.0336 | 0.2019 | 0.2365 | 0.7205 | D12S1602 |
| 135.149 | 0.0121 | 0.0335 | 0.2006 | 0.2356 | 0.7164 | D12S79 |
| 135.367 | 0.0102 | 0.0312 | 0.1829 | 0.217 | 0.7035 | D12S1665 |
| 137.617 | 0.0008 | 0.0093 | 0.0498 | 0.0617 | 0.6492 | D12S1718 |
| 140.815 | 0.0287 | 0.0511 | 0.3109 | 0.3633 | 0.7212 | D12S366 |
| 141.527 | 0.0431 | 0.0638 | 0.374 | 0.4458 | 0.6902 | D12S349 |
| 141.528 | 0.0879 | 0.0897 | 0.5377 | 0.6361 | 0.6935 | D12S1619 |
| 141.755 | 0.0867 | 0.0892 | 0.5334 | 0.6317 | 0.6917 | D12S385 |
| 143.676 | 0.0629 | 0.073 | 0.476 | 0.5383 | 0.7618 | D12S395 |
| 143.677 | 0.0629 | 0.073 | 0.4759 | 0.5382 | 0.7615 | D12S321 |
| 143.678 | 0.0629 | 0.073 | 0.4759 | 0.5381 | 0.7613 | D12S1721 |
| 143.824 | 0.0588 | 0.0707 | 0.4601 | 0.5205 | 0.7614 | D12S1666 |
| 144.632 | 0.0428 | 0.0604 | 0.3929 | 0.444 | 0.7652 | D12S2073 |
| 144.962 | 0.0437 | 0.0611 | 0.3961 | 0.4485 | 0.7621 | D12S1349 |
| 145.291 | 0.037 | 0.0563 | 0.3644 | 0.4128 | 0.7628 | D12S1603 |
| 145.426 | 0.0331 | 0.0534 | 0.3446 | 0.3907 | 0.7623 | D12S378 |
| 149.447 | 0.0134 | −0.0352 | −0.2159 | −0.2483 | 0.7658 | D12S1614 |
| 149.448 | 0.0134 | −0.0352 | −0.2158 | −0.2483 | 0.7656 | D12S342 |
| 152.517 | 0.0049 | −0.0224 | −0.124 | −0.1505 | 0.6847 | D12S324 |
| 153.404 | 0.0009 | −0.0099 | −0.0509 | −0.064 | 0.6328 | D12S1634 |
| 153.405 | 0.0009 | −0.0098 | −0.0507 | −0.0638 | 0.6382 | D12S307 |
| 154.88 | 0.0244 | 0.0534 | 0.2534 | 0.3353 | 0.561 | D12S1658 |
| 155.819 | 0.0768 | 0.0941 | 0.447 | 0.5948 | 0.549 | GATA41E12 |
| 155.94 | 0.0855 | 0.0991 | 0.472 | 0.6275 | 0.5489 | D12S2078 |
| 157.397 | 0.0566 | 0.0832 | 0.3729 | 0.5104 | 0.5228 | D12S1675 |
| 159.342 | 0.0829 | 0.0973 | 0.4654 | 0.6179 | 0.5526 | D12S1679 |
| 161.157 | 0.1143 | 0.1111 | 0.5609 | 0.7255 | 0.5776 | D12S1609 |
| 163.425 | 0.1165 | 0.1067 | 0.5964 | 0.7324 | 0.6407 | D12S834 |
| 163.559 | 0.1167 | 0.1063 | 0.5993 | 0.733 | 0.6461 | D12S1659 |
| 165.72 | 0.175 | 0.1287 | 0.7383 | 0.8977 | 0.6479 | D12S1714 |
| 165.721 | 0.175 | 0.1287 | 0.7383 | 0.8978 | 0.648 | D12S367 |
| 168.245 | 0.1739 | 0.132 | 0.7137 | 0.8949 | 0.6107 | D12S2069 |
| 168.246 | 0.1739 | 0.132 | 0.7138 | 0.8949 | 0.6105 | D12S97 |
| 170.298 | 0.2145 | 0.1514 | 0.7627 | 0.9938 | 0.5626 | D12S343 |
| 170.824 | 0.2262 | 0.156 | 0.78 | 1.0207 | 0.5566 | D12S1599 |
| 171.817 | 0.2496 | 0.1638 | 0.8178 | 1.0722 | 0.5531 | D12S392 |
| 173.734 | 0.2978 | 0.1751 | 0.9099 | 1.171 | 0.5715 | D12S1723 |
| 175.333 | 0.2667 | 0.1709 | 0.8351 | 1.1083 | 0.5393 | D12S357 |
| 175.456 | 0.2648 | 0.1707 | 0.8307 | 1.1043 | 0.5372 | D12S1638 |
| 176.211 | 0.2665 | 0.1772 | 0.8027 | 1.1079 | 0.4984 | D12S2343 |

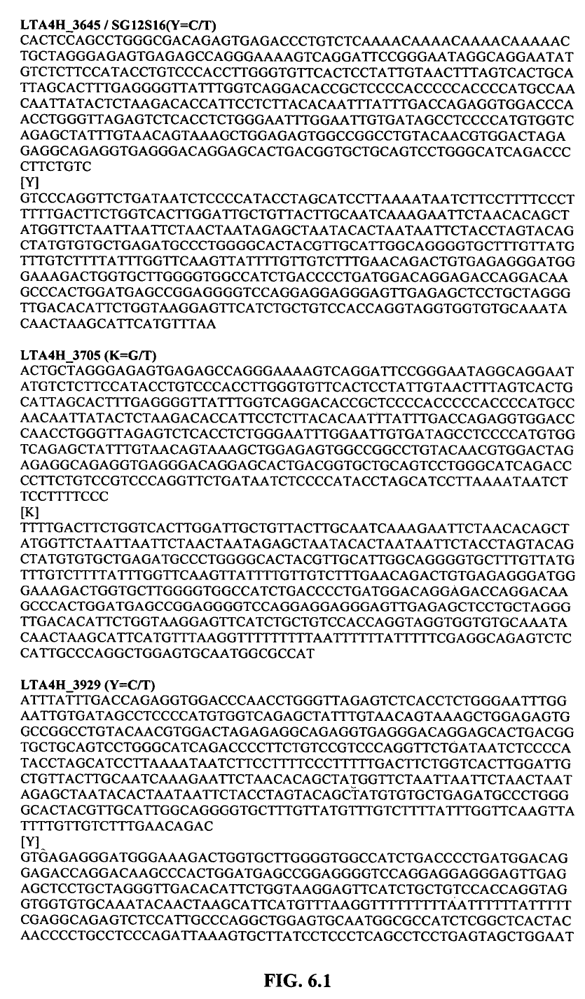

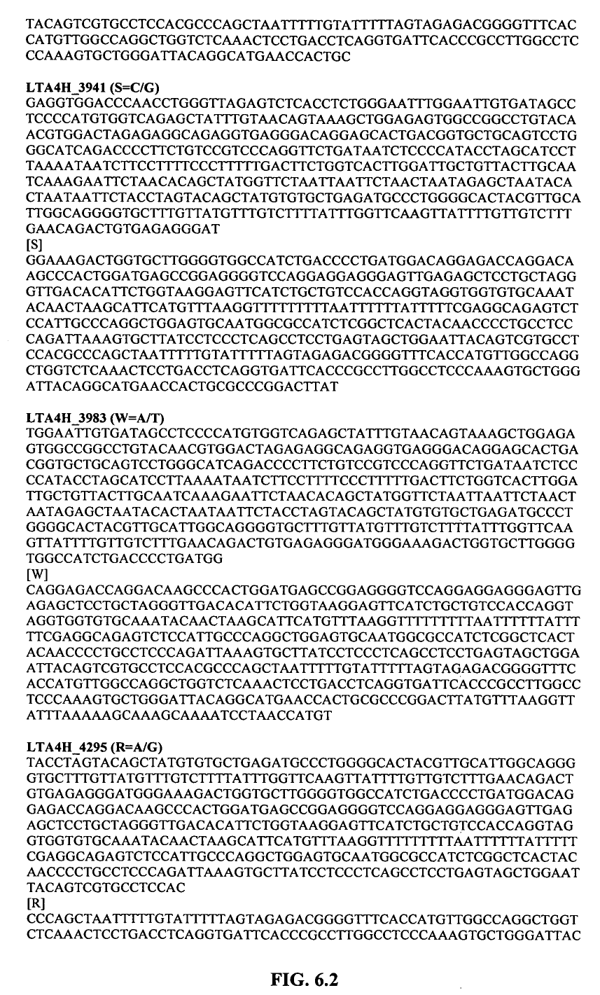

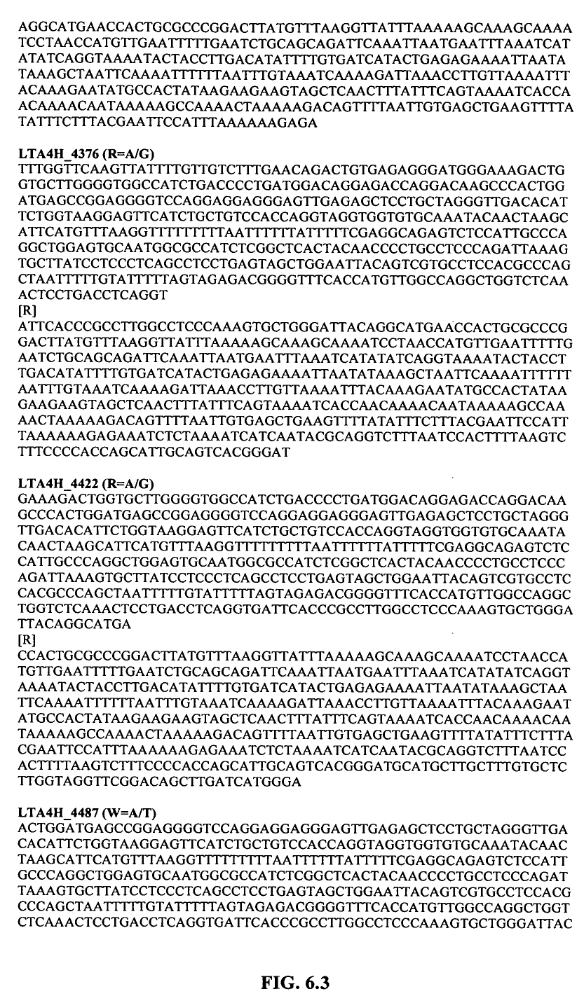

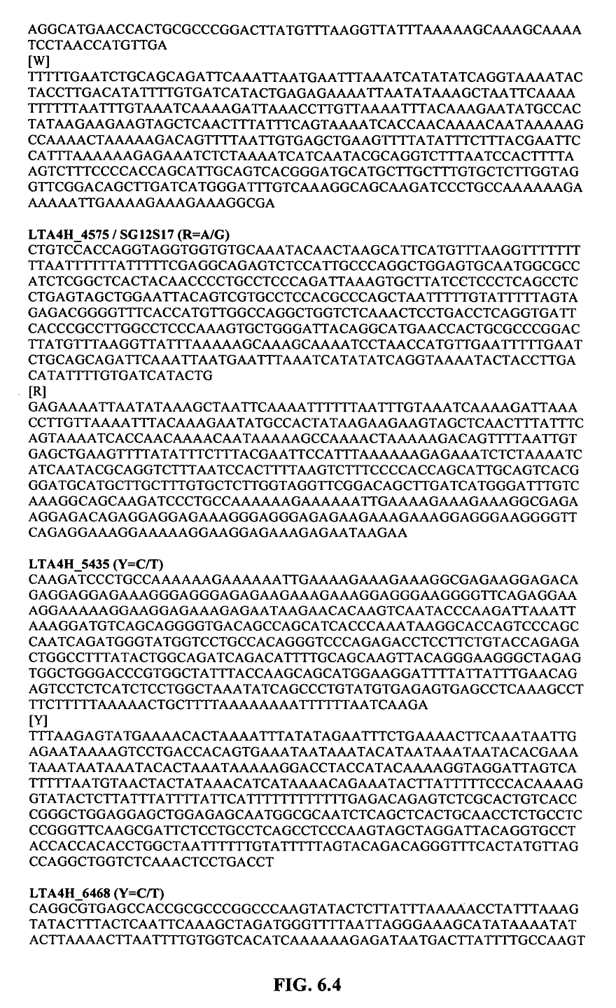

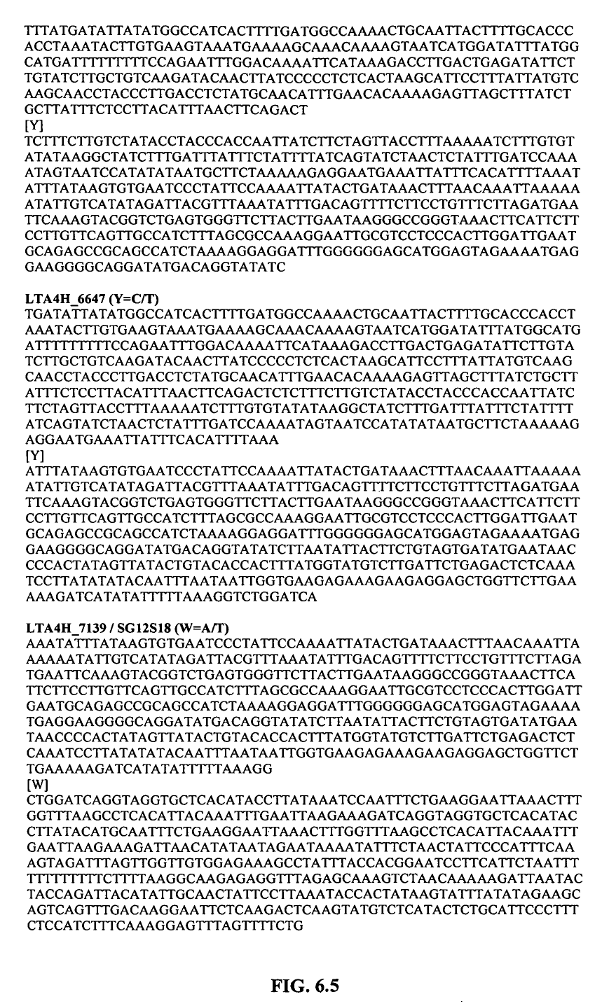

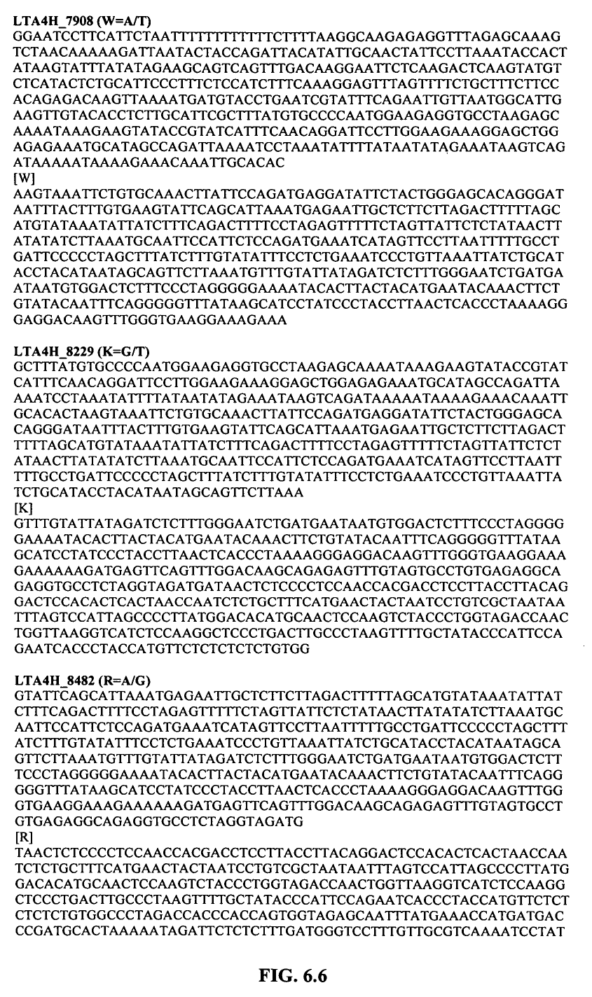

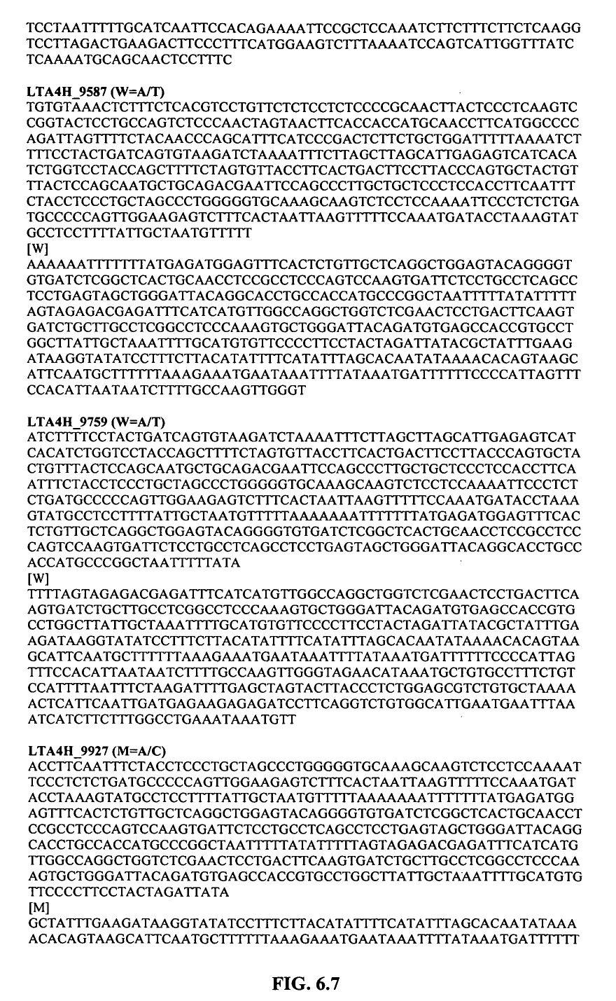

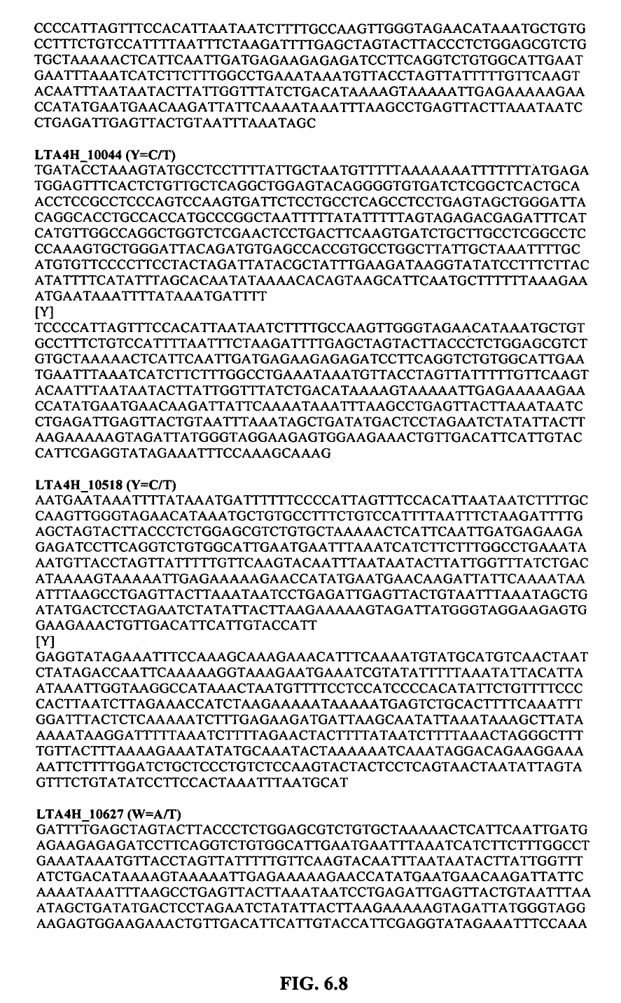

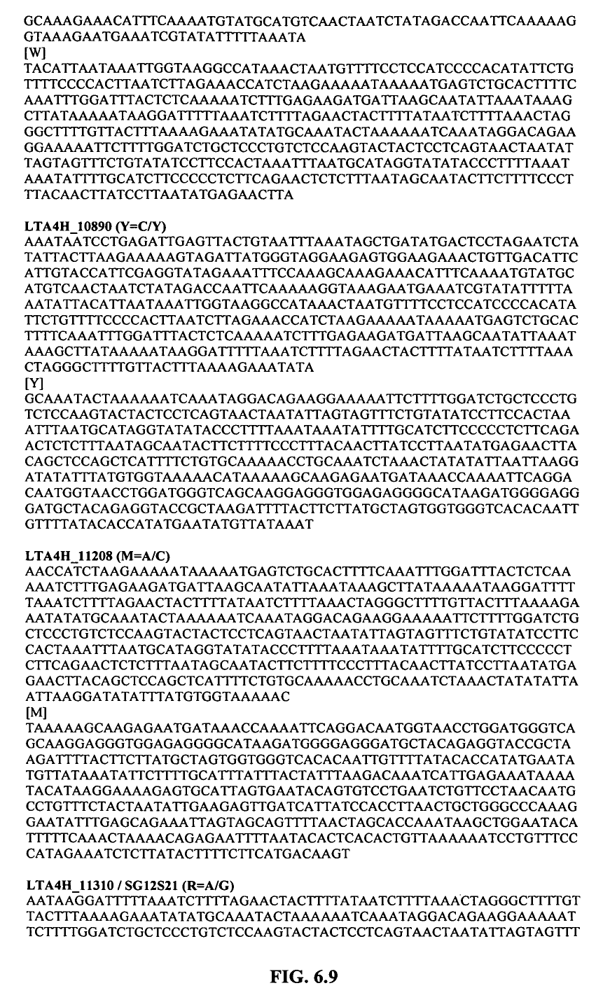

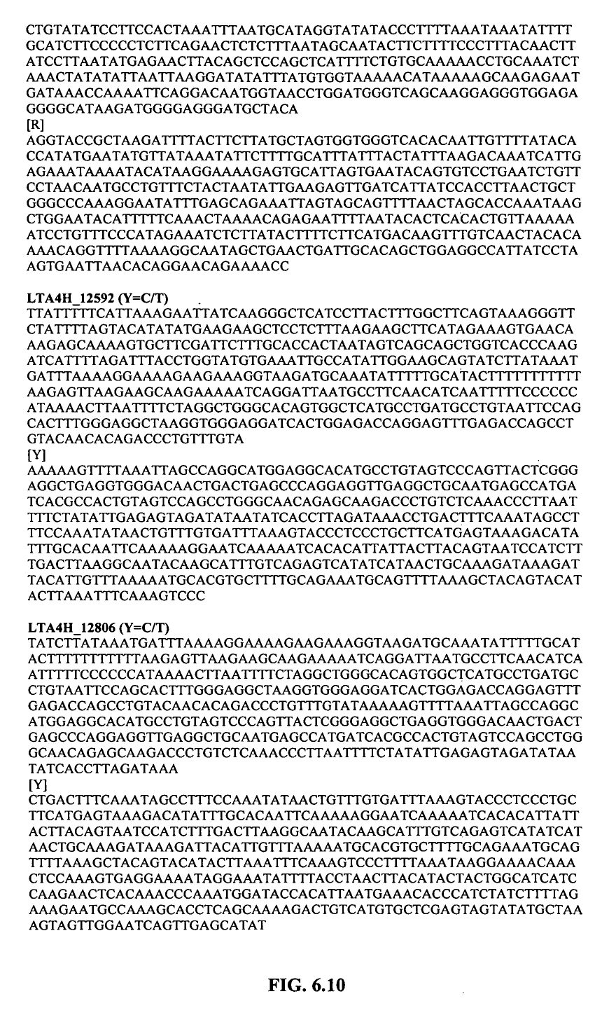

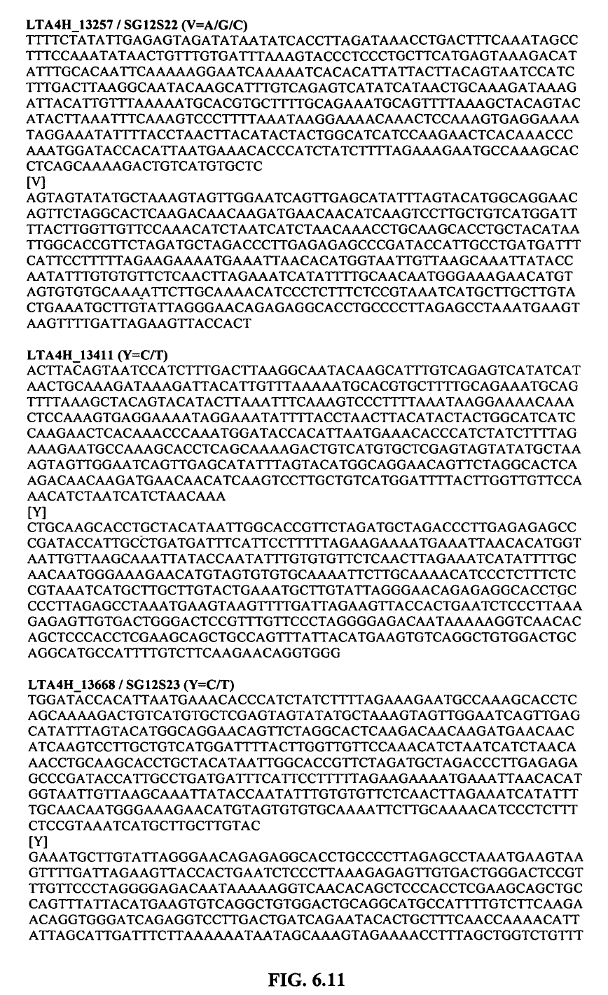

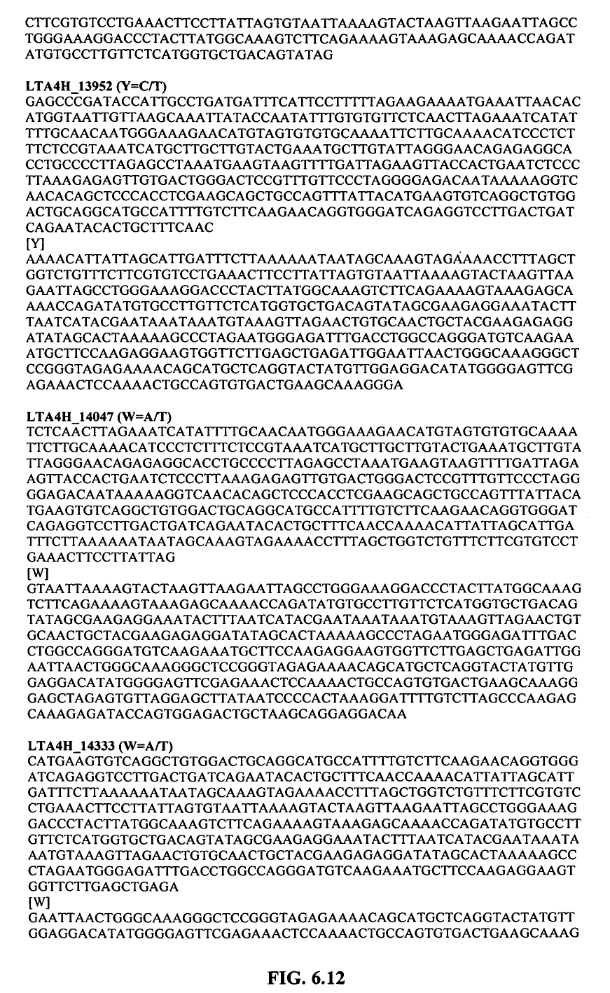

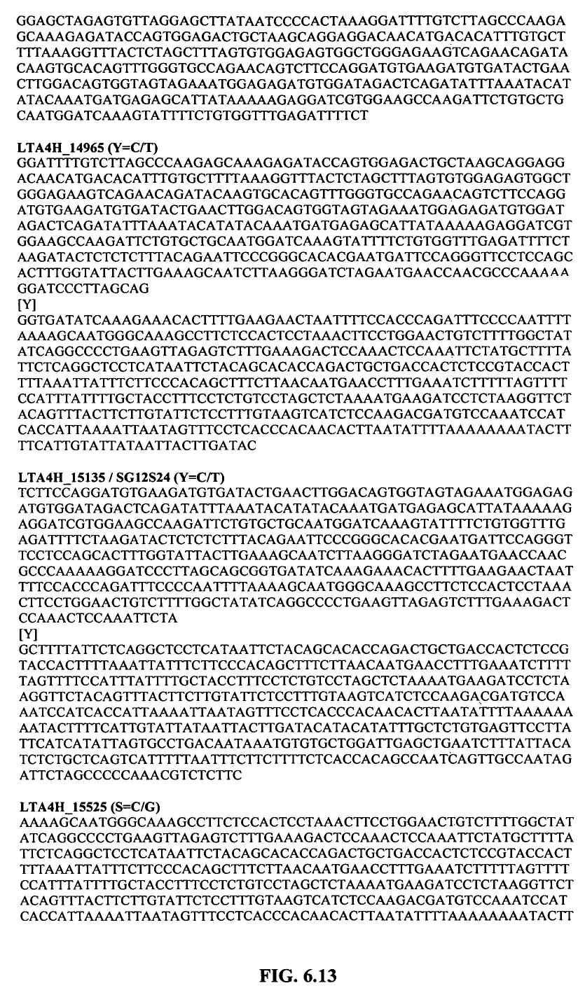

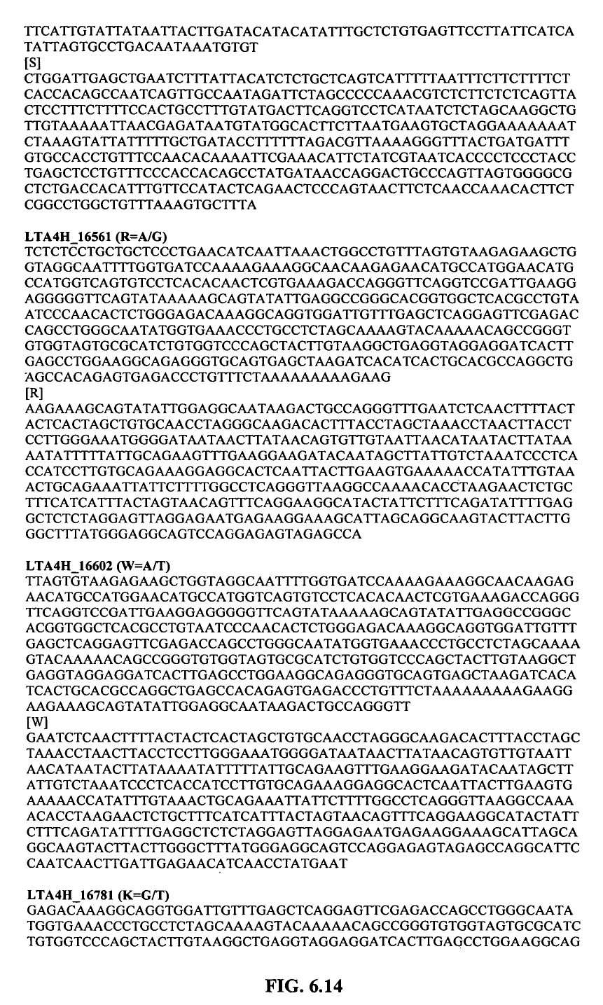

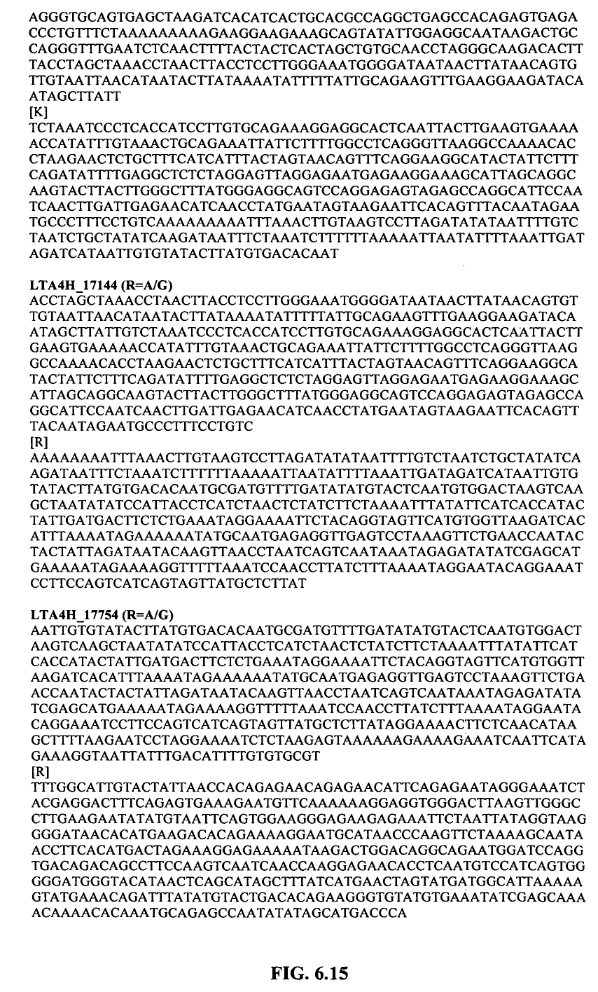

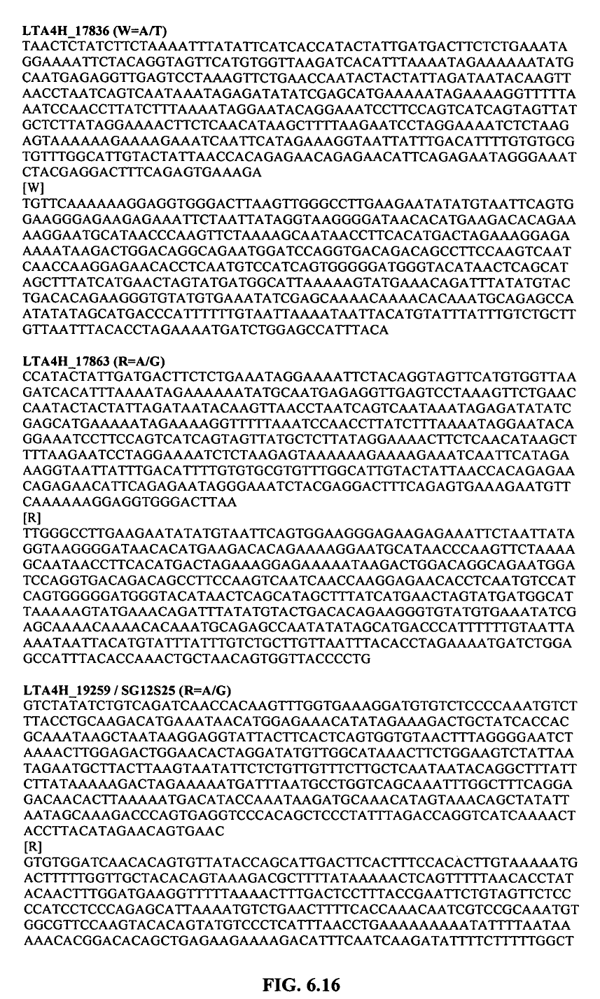

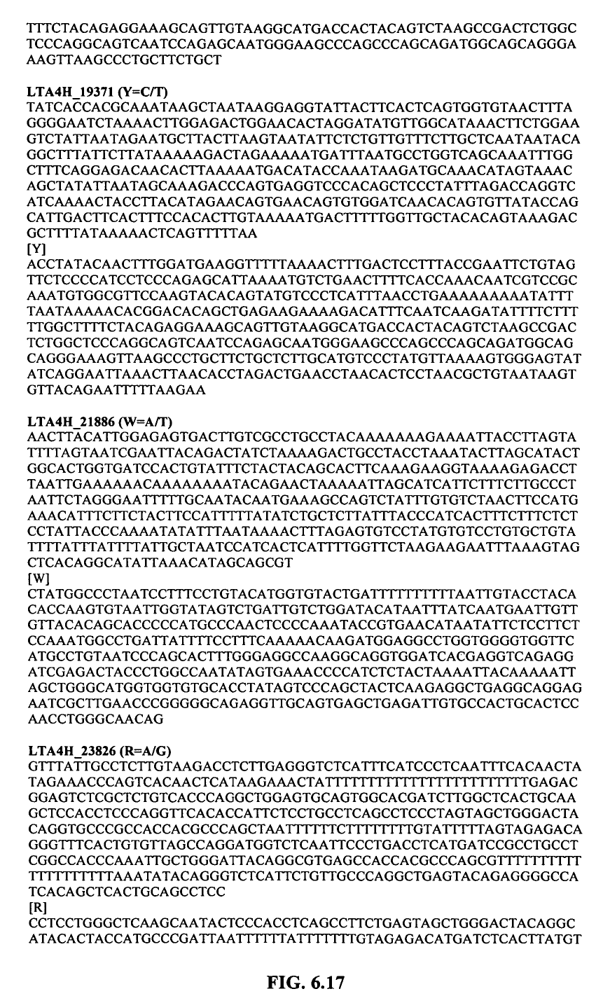

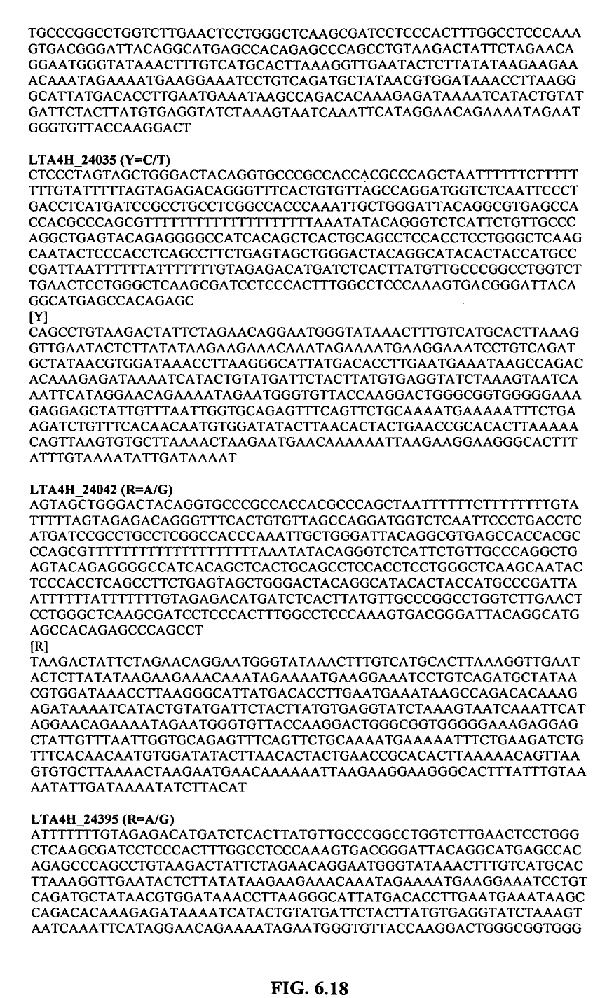

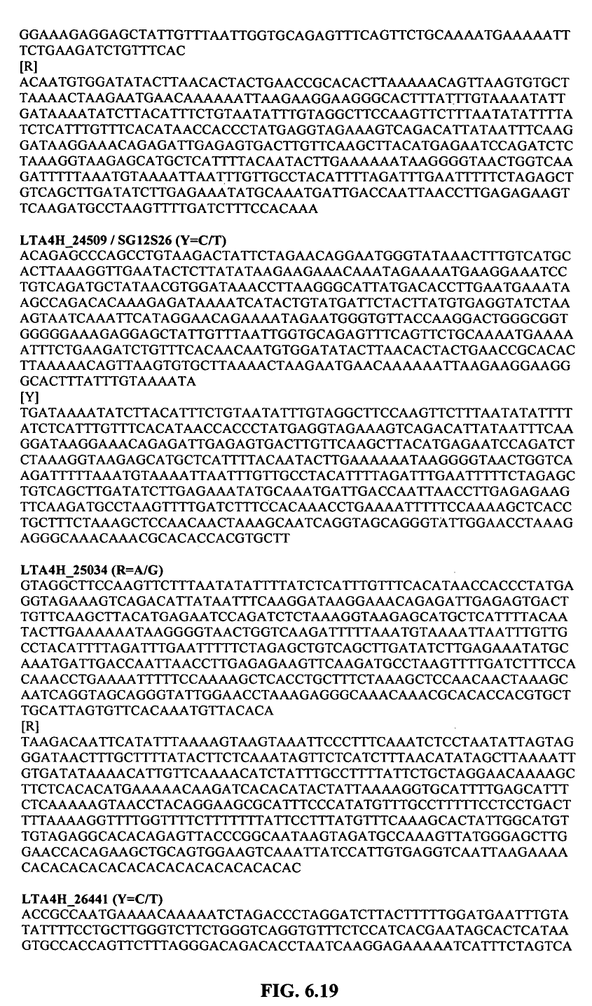

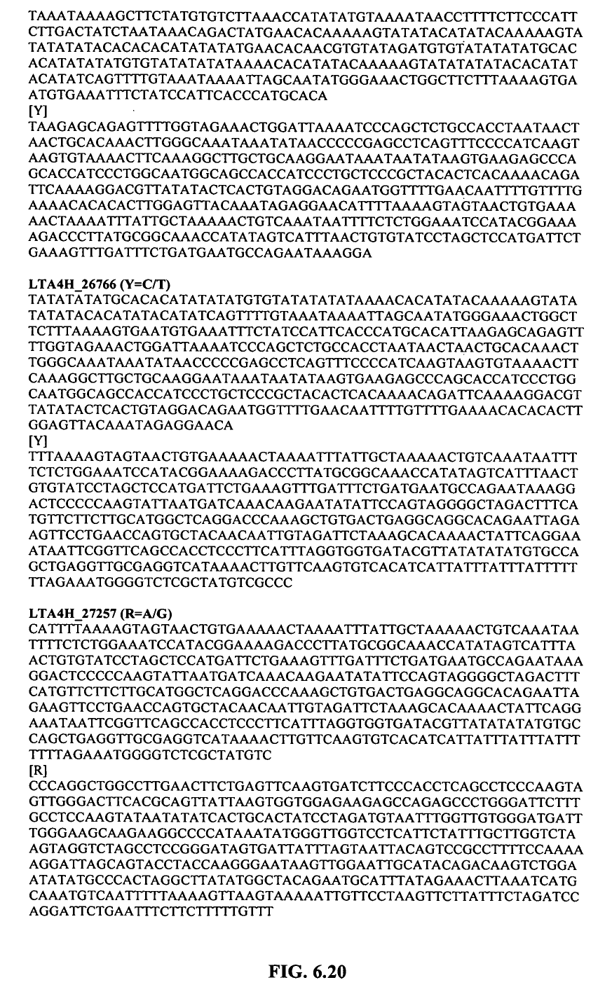

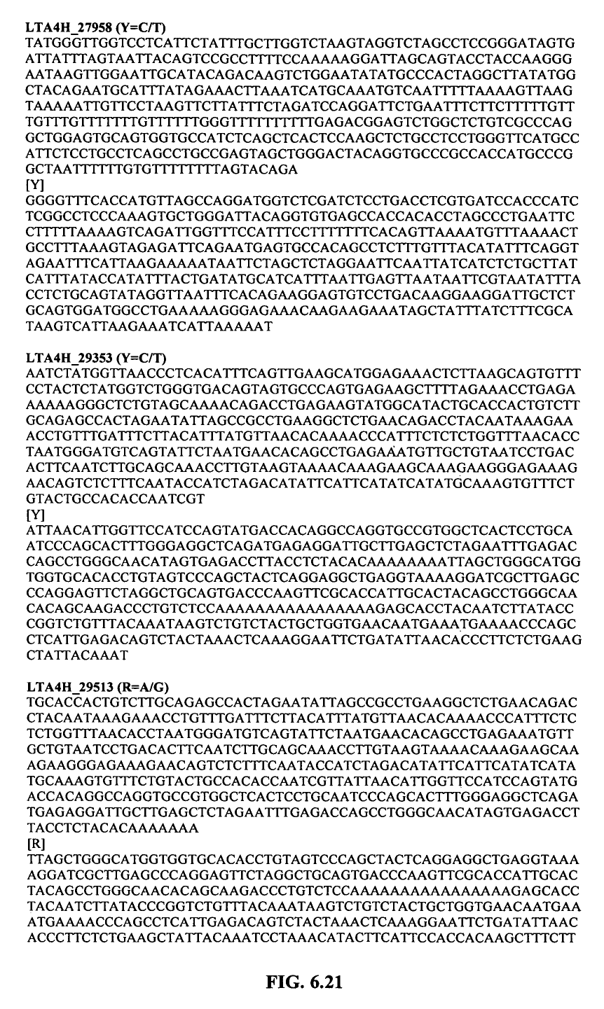

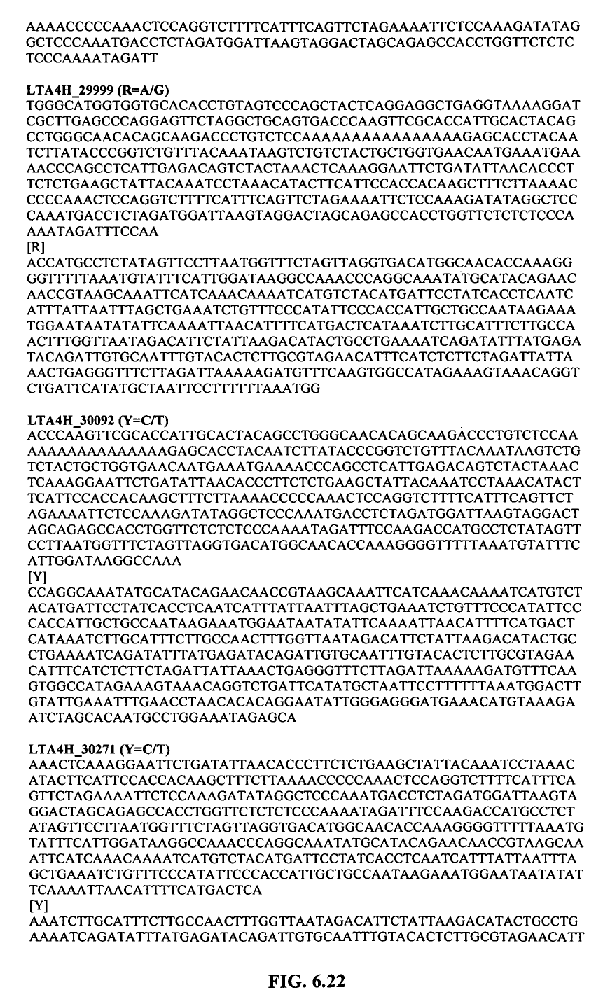

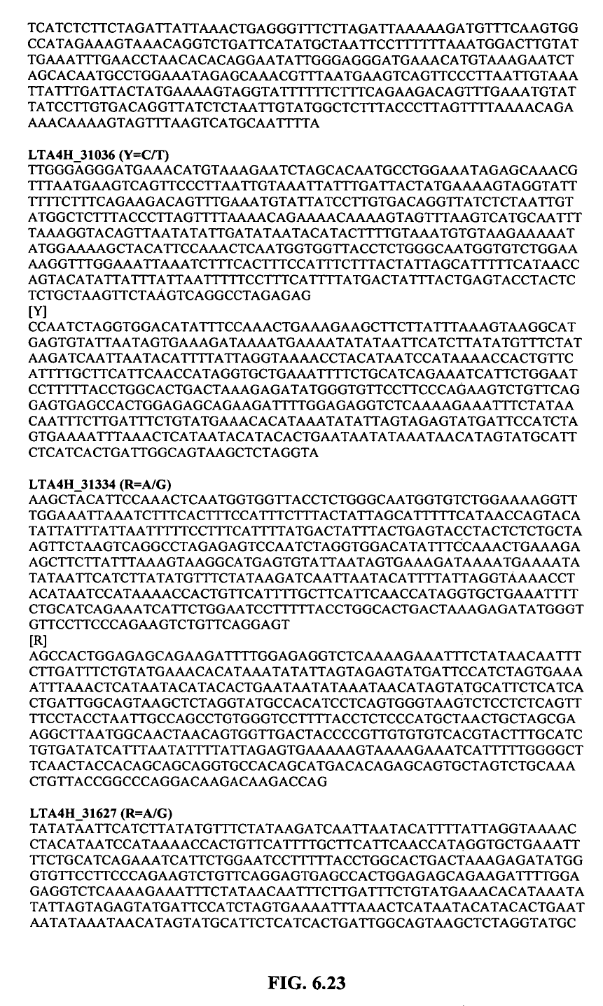

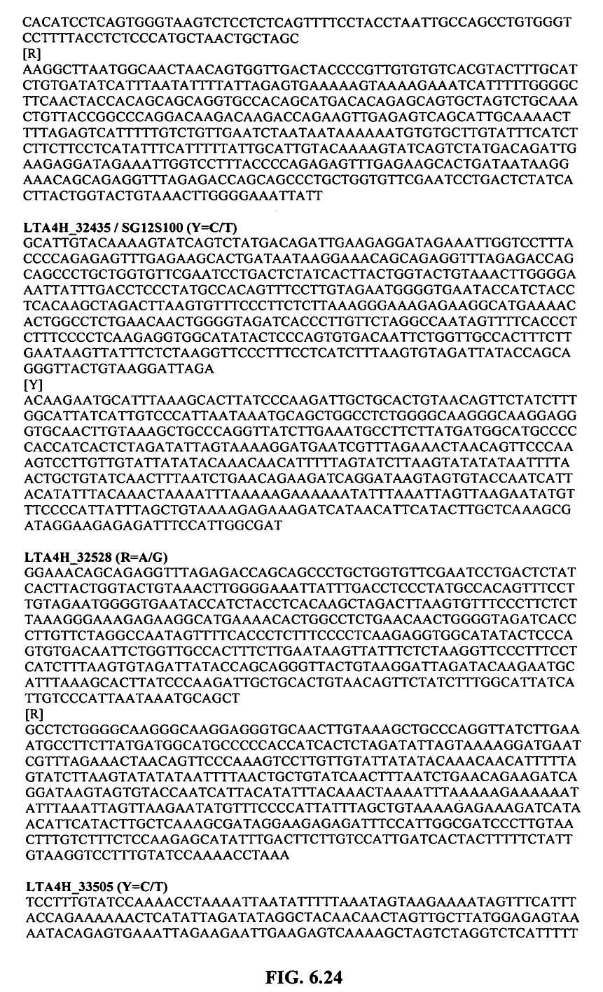

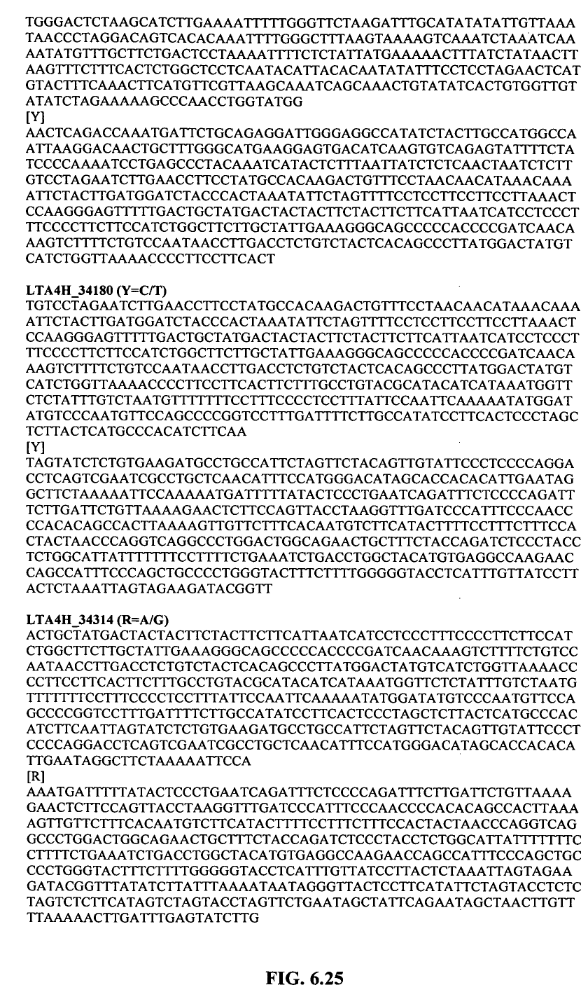

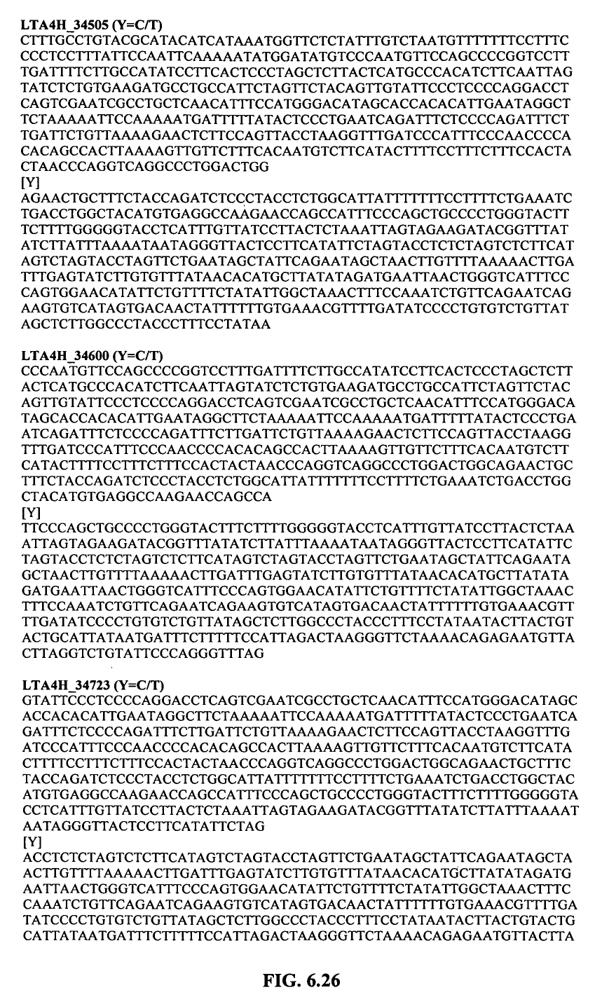

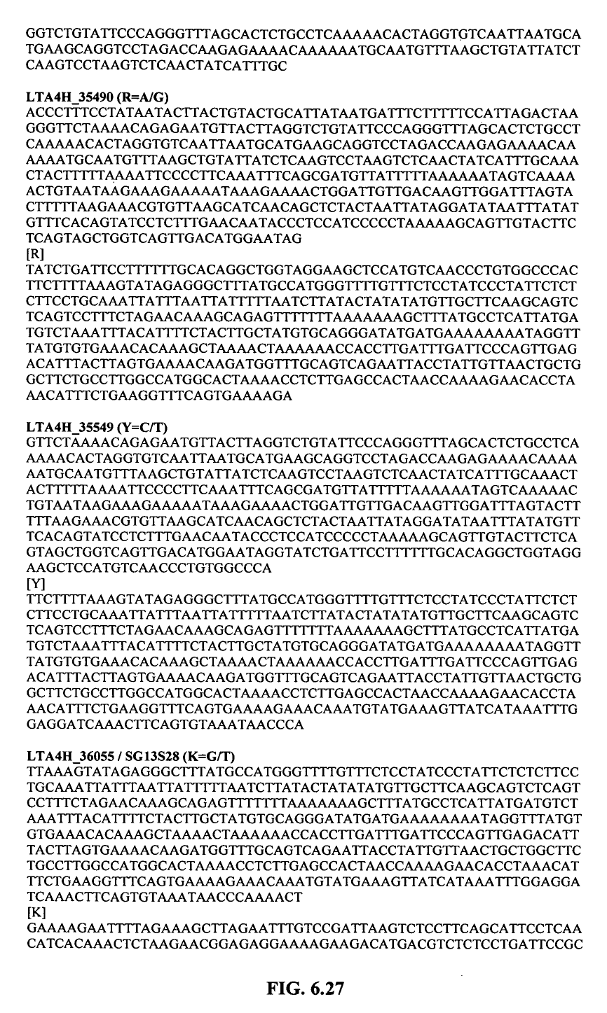

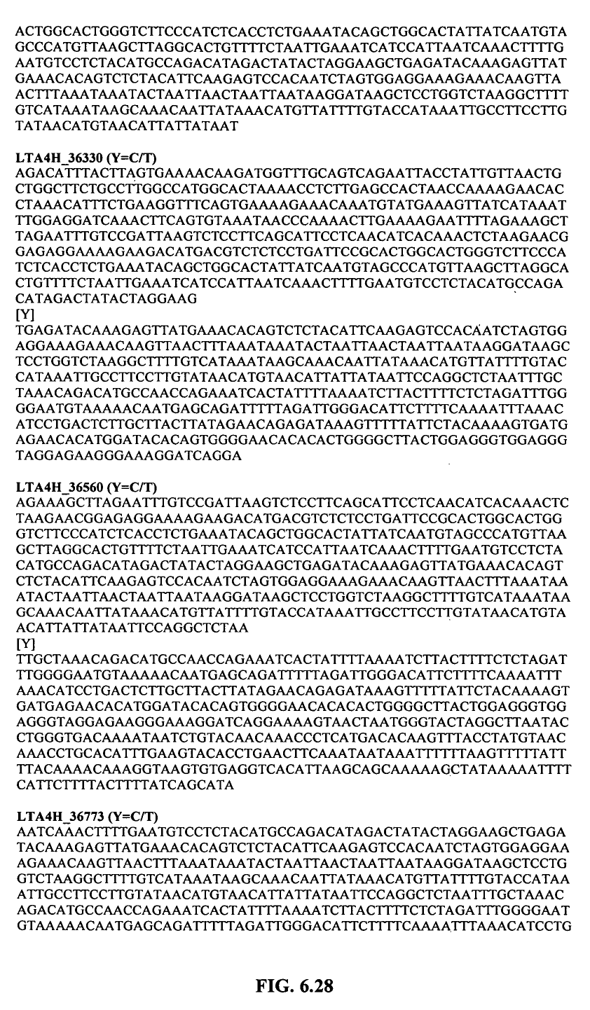

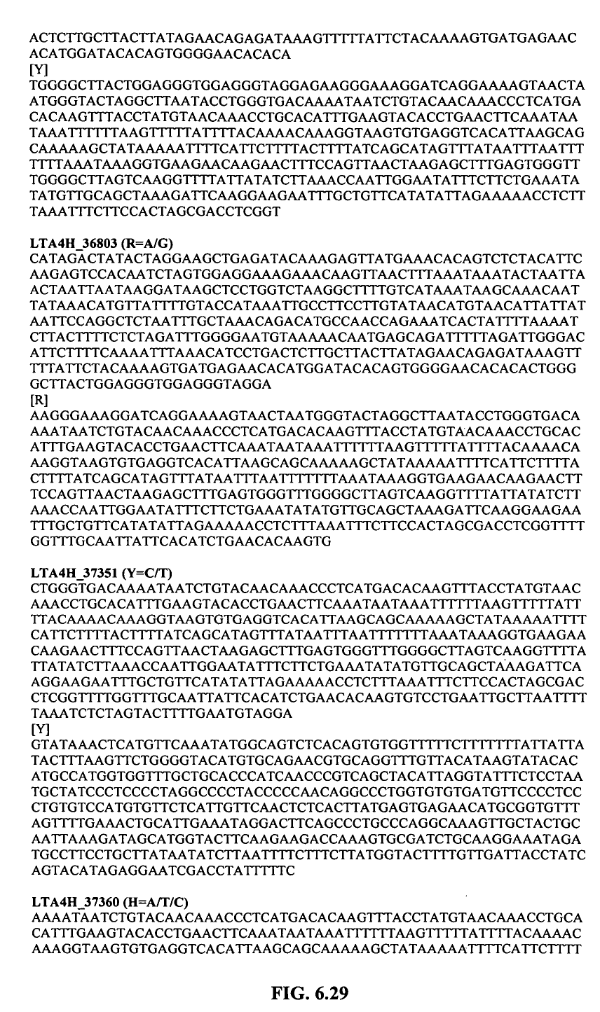

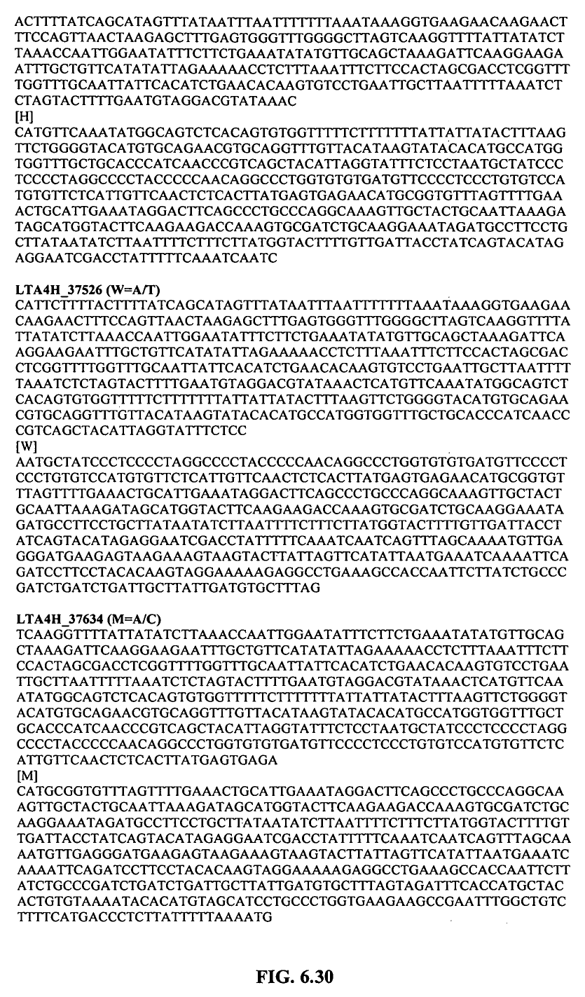

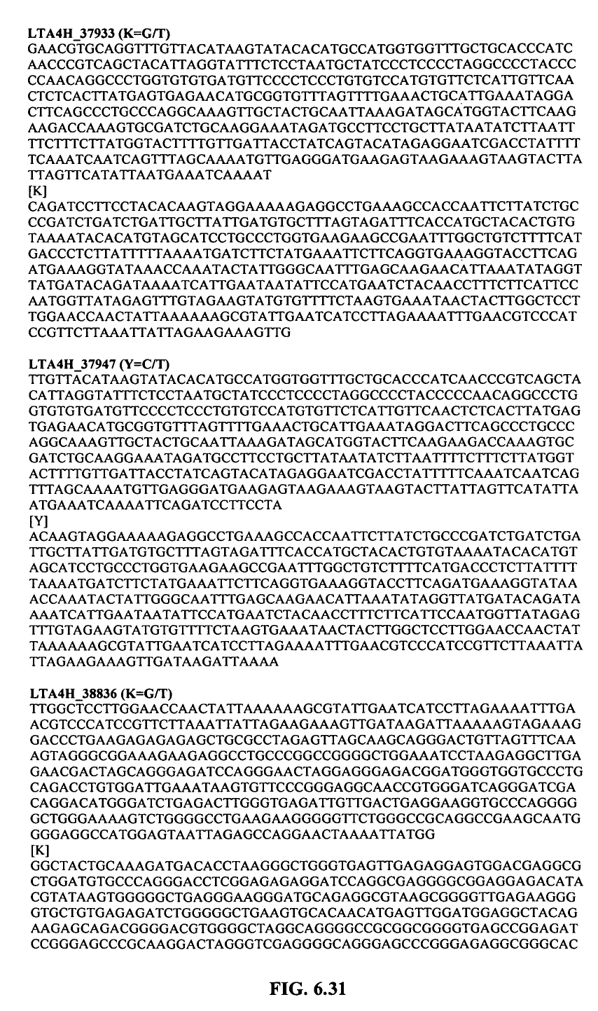

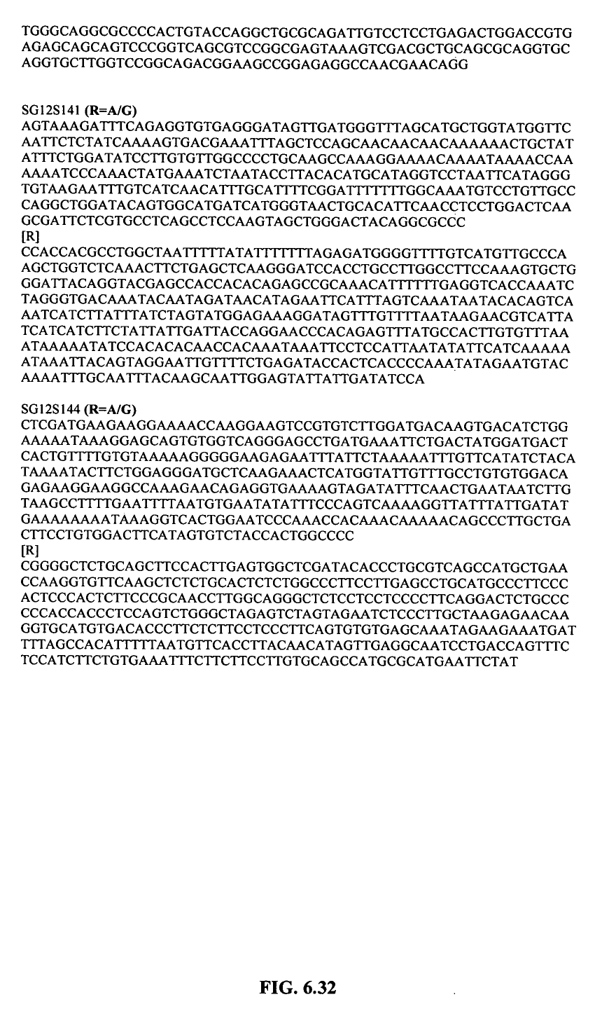

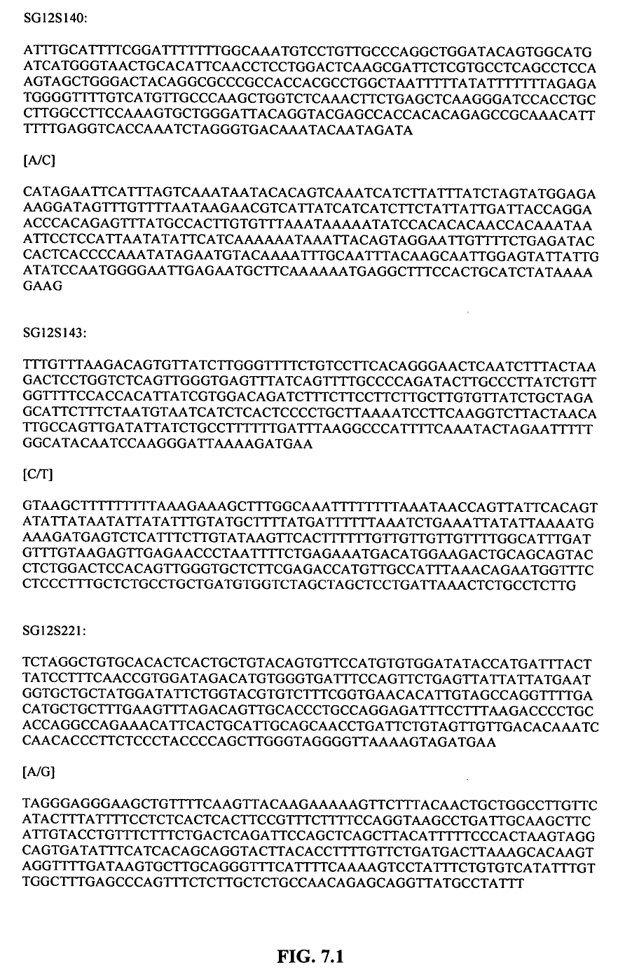

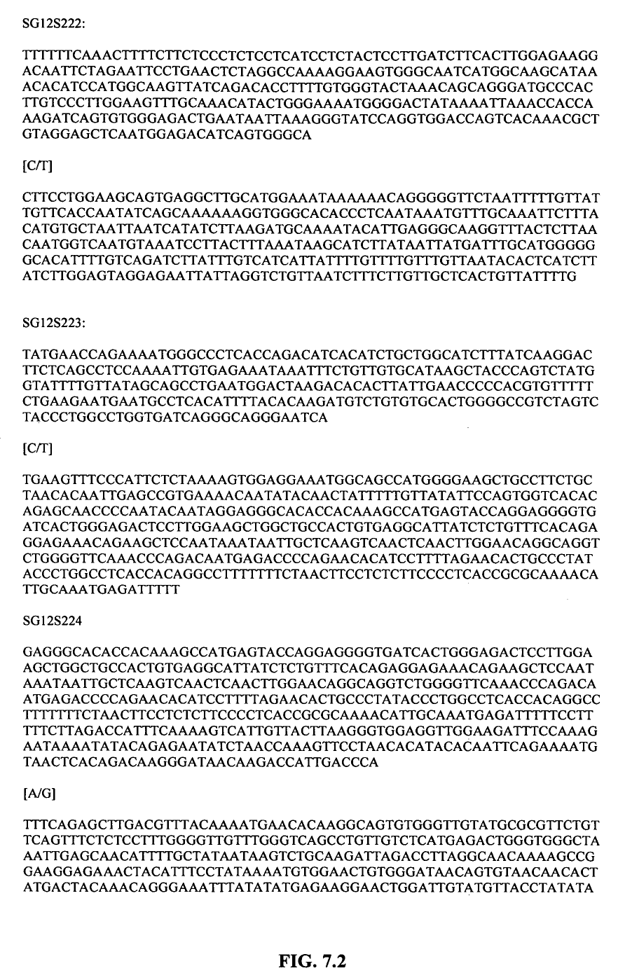

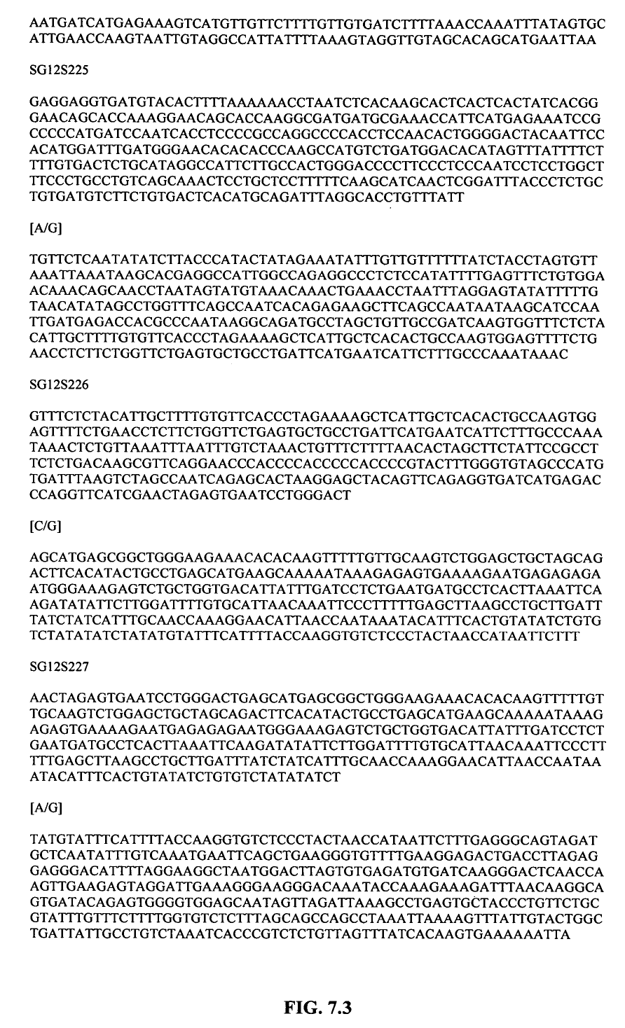

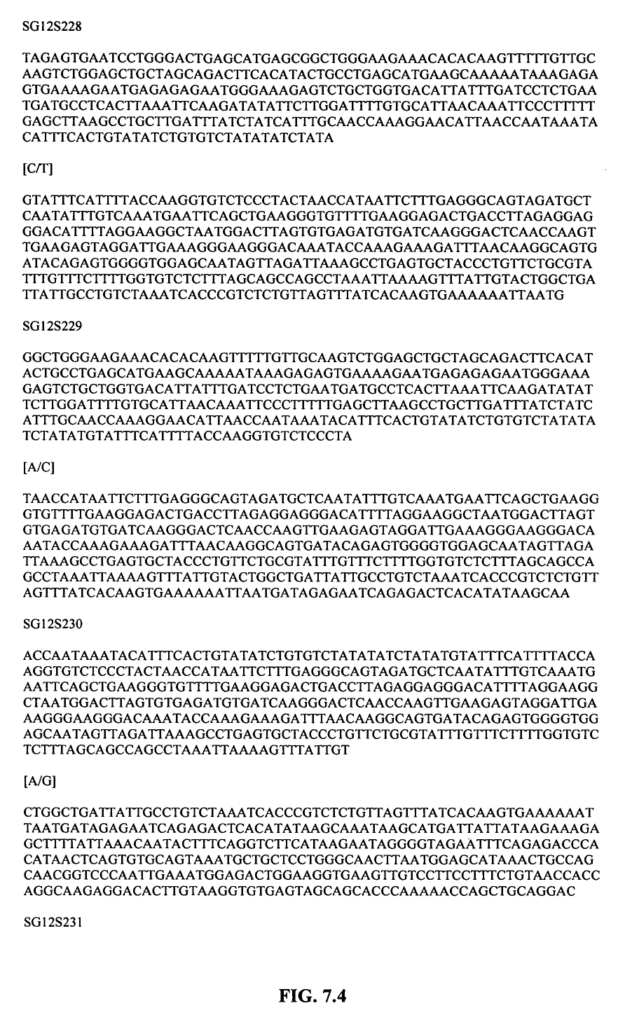

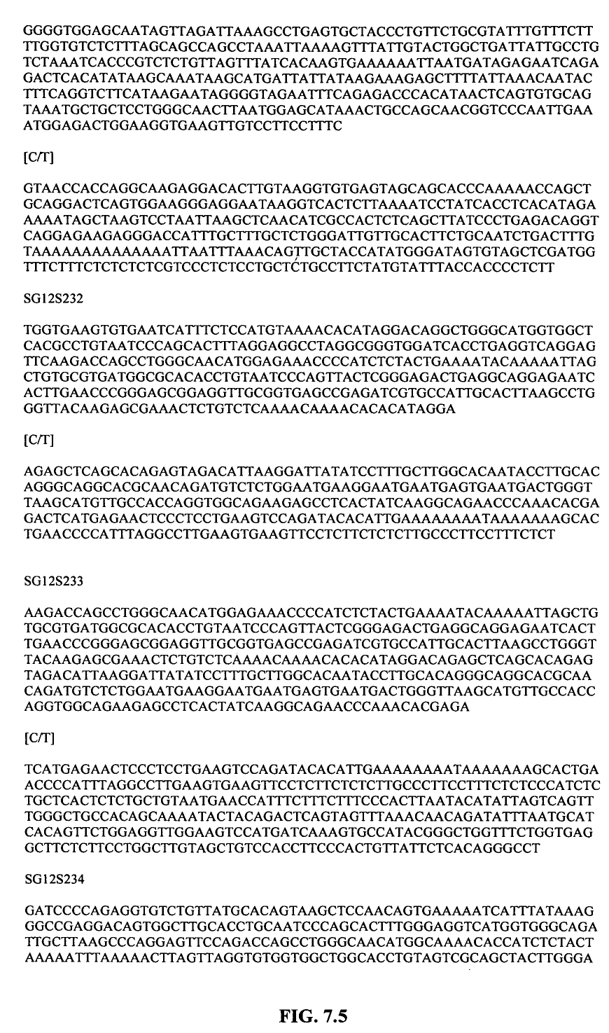

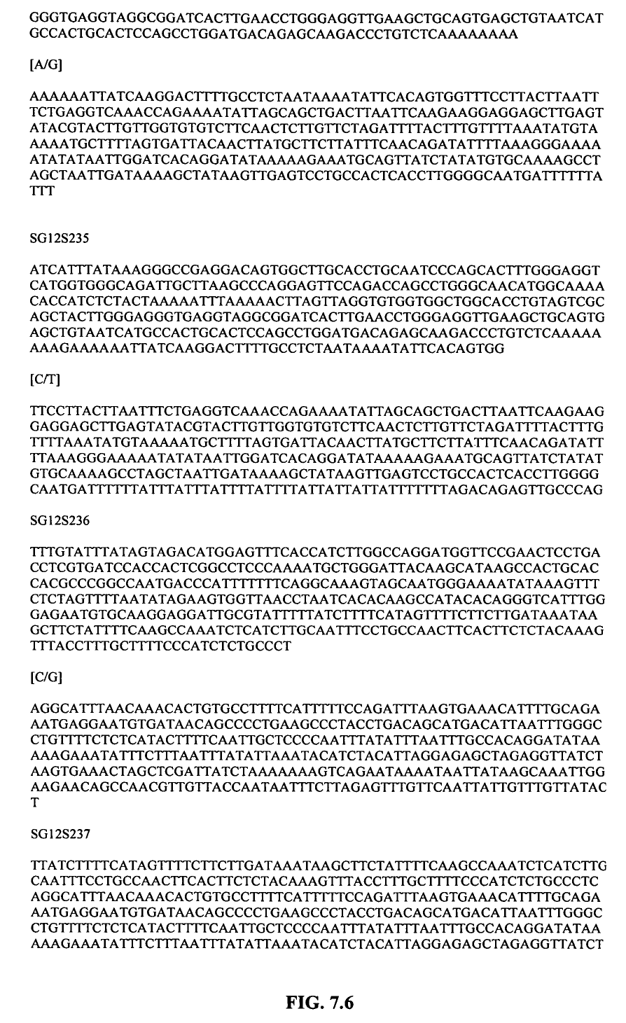

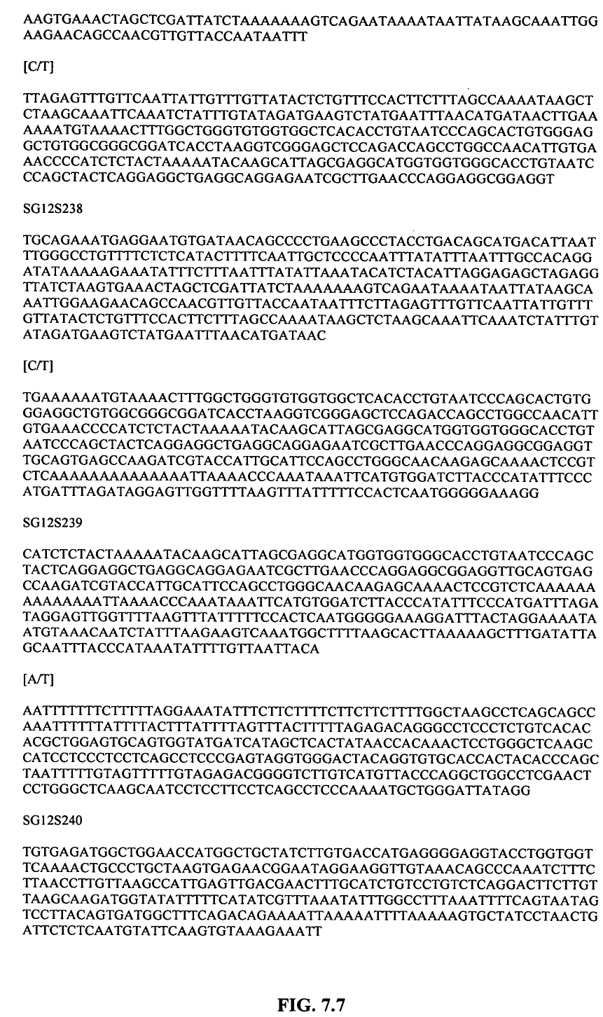

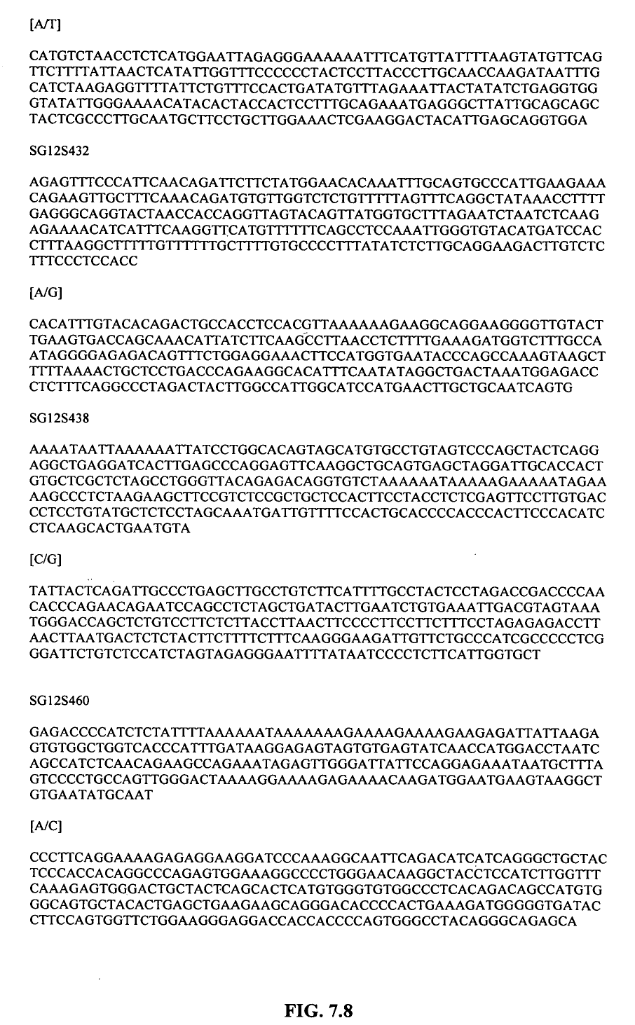

TABLE 3
Table 3 shows the SNPs identified within the genomic sequence, by the methods described herein.
Position of the SNPs refers to SEQ ID NO 1. Sequences of the SNPs are shown in FIG. 6 or FIG. 7.

| Build34 | Build34 | Marker | Marker | | Public | | Minor | Minor | position in |
|---|---|---|---|---|---|---|---|---|---|
| start | stop | name | alias | IUPAC | SNP | Variation | allele | allele % | Sequence |
| 94877218 | 94877218 | SG12S432 | | R | rs2270318 | A/G | A | 12.75 | 7218 |
| 94885285 | 94885285 | SG12S438 | | S | rs2268517 | C/G | G | 9.36 | 15285 |
| 94896055 | 94896055 | SG12S16 | LTA4H_3645 | Y | | C/T | T | 22.64 | 26055 |
| 94896115 | 94896115 | SG12S56 | LTA4H_3705 | K | | G/T | G | 4.14 | 26115 |
| 94896339 | 94896339 | SG12S57 | LTA4H_3929 | Y | | C/T | C | 2.5 | 26339 |
| 94896351 | 94896351 | SG12S58 | LTA4H_3941 | S | | C/G | C | 0.85 | 26351 |
| 94896393 | 94896393 | SG12S37 | LTA4H_3983 | W | | A/T | T | 9.3 | 26393 |
| 94896705 | 94896705 | SG12S59 | LTA4H_4295 | R | | A/G | A | 4.5 | 26705 |
| 94896786 | 94896786 | SG12S60 | LTA4H_4376 | R | | A/G | A | 2.87 | 26786 |
| 94896832 | 94896832 | SG12S61 | LTA4H_4422 | R | | A/G | G | 1.56 | 26832 |
| 94896897 | 94896897 | SG12S29 | LTA4H_4487 | W | | A/T | T | 4.26 | 26897 |
| 94896985 | 94896985 | SG12S17 | LTA4H_4575 | R | rs11108372 | A/G | A | 41.41 | 26985 |
| 94897845 | 94897845 | SG12S62 | LTA4H_5435 | Y | | C/T | C | 1.17 | 27845 |
| 94898878 | 94898878 | SG12S63 | LTA4H_6468 | Y | | C/T | T | 4.46 | 28878 |
| 94899057 | 94899057 | SG12S64 | LTA4H_6647 | Y | | C/T | C | 2.99 | 29057 |
| 94899549 | 94899549 | SG12S18 | LTA4H_7139 | W | | A/T | A | 21.72 | 29549 |
| 94900318 | 94900318 | SG12S19 | LTA4H_7908 | W | | A/T | A | 10.9 | 30318 |
| 94900639 | 94900639 | SG12S65 | LTA4H_8229 | K | | G/T | G | 5.09 | 30639 |
| 94900892 | 94900892 | SG12S66 | LTA4H_8482 | R | | A/G | G | 0.59 | 30892 |
| 94901997 | 94901997 | SG12S68 | LTA4H_9587 | W | | A/T | T | 3.63 | 31997 |
| 94902169 | 94902169 | SG12S69 | LTA4H_9759 | W | | A/T | A | 0.88 | 32169 |
| 94902337 | 94902337 | SG12S70 | LTA4H_9927 | M | | A/C | A | 24.09 | 32337 |
| 94902454 | 94902454 | SG12S71 | LTA4H_10044 | Y | | C/T | C | 20.93 | 32454 |
| 94902928 | 94902928 | SG12S72 | LTA4H_10518 | Y | | C/T | T | 1.35 | 32928 |
| 94903037 | 94903037 | SG12S30 | LTA4H_10627 | W | rs2540498 | A/T | A | 22.36 | 33037 |
| 94903300 | 94903300 | SG12S73 | LTA4H_10890 | Y | rs2300559 | C/T | C | 2.33 | 33300 |
| 94903618 | 94903618 | SG12S20 | LTA4H_11208 | M | | A/C | C | 39.08 | 33618 |
| 94903720 | 94903720 | SG12S21 | LTA4H_11310 | R | rs2660880 | A/G | A | 5.95 | 33720 |
| 94905002 | 94905002 | SG12S38 | LTA4H_12592 | Y | rs2110762 | C/T | C | 34.92 | 35002 |
| 94905216 | 94905216 | SG12S74 | LTA4H_12806 | Y | | C/T | T | 0.8 | 35216 |
| 94905667 | 94905667 | SG12S22 | LTA4H_13257 | R | rs2072510 | A/G | A | 36.88 | 35667 |
| 94905821 | 94905821 | SG12S75 | LTA4H_13411 | Y | | C/T | T | 1.39 | 35821 |
| 94906078 | 94906078 | SG12S23 | LTA4H_13668 | Y | | C/T | C | 7.06 | 36078 |
| 94906362 | 94906362 | SG12S31 | LTA4H_13952 | Y | | C/T | T | 5.67 | 36362 |
| 94906457 | 94906457 | SG12S76 | LTA4H_14047 | W | rs10492226 | A/T | A | 1.18 | 36457 |
| 94906743 | 94906743 | SG12S77 | LTA4H_14333 | W | | A/T | A | 24.77 | 36743 |
| 94907375 | 94907375 | SG12S78 | LTA4H_14965 | Y | | C/T | T | 2.48 | 37375 |
| 94907545 | 94907545 | SG12S24 | LTA4H_15135 | Y | rs2660900 | C/T | C | 23.76 | 37545 |
| 94907935 | 94907935 | SG12S79 | LTA4H_15525 | S | | C/G | C | 0.83 | 37935 |
| 94908971 | 94908971 | SG12S32 | LTA4H_16561 | R | rs2540496 | A/G | A | 31.11 | 38971 |
| 94909012 | 94909012 | SG12S80 | LTA4H_16602 | W | | A/T | A | 0.74 | 39012 |
| 94909191 | 94909191 | SG12S39 | LTA4H_16781 | K | rs2540495 | G/T | T | 30.74 | 39191 |
| 94909554 | 94909554 | SG12S81 | LTA4H_17144 | R | rs12319438 | A/G | G | 4.12 | 39554 |
| 94910164 | 94910164 | SG12S82 | LTA4H_17754 | R | | A/G | A | 0.4 | 40164 |
| 94910246 | 94910246 | SG12S83 | LTA4H_17836 | W | | A/T | T | 1.21 | 40246 |
| 94910273 | 94910273 | SG12S84 | LTA4H_17863 | R | | A/G | A | 2.82 | 40273 |
| 94911669 | 94911669 | SG12S25 | LTA4H_19259 | R | rs1978331 | A/G | G | 31.68 | 41669 |
| 94911781 | 94911781 | SG12S85 | LTA4H_19371 | Y | | C/T | T | 1.25 | 41781 |
| 94914296 | 94914296 | SG12S40 | LTA4H_21886 | W | rs7959337 | A/T | A | 5.29 | 44296 |
| 94916236 | 94916236 | SG12S86 | LTA4H_23826 | R | | A/G | G | 4.71 | 46236 |
| 94916445 | 94916445 | SG12S87 | LTA4H_24035 | Y | | C/T | T | 1.27 | 46445 |
| 94916452 | 94916452 | SG12S88 | LTA4H_24042 | R | rs1990611 | A/G | A | 33.76 | 46452 |
| 94916805 | 94916805 | SG12S89 | LTA4H_24395 | R | rs7981011 | A/G | G | 4.91 | 46805 |
| 94916919 | 94916919 | SG12S26 | LTA4H_24509 | Y | | C/T | C | 17.16 | 46919 |
| 94917444 | 94917444 | SG12S90 | LTA4H_25034 | R | | A/G | A | 0.84 | 47444 |
| 94918851 | 94918851 | SG12S91 | LTA4H_26441 | Y | rs2660838 | C/T | C | 25 | 48851 |
| 94919176 | 94919176 | SG12S92 | LTA4H_26766 | Y | | C/T | C | 20.44 | 49176 |
| 94919667 | 94919667 | SG12S93 | LTA4H_27257 | R | rs2268516 | A/G | A | 2.44 | 49667 |
| 94920368 | 94920368 | SG12S94 | LTA4H_27958 | Y | rs2660839 | C/T | C | 31.82 | 50368 |
| 94921763 | 94921763 | SG12S41 | LTA4H_29353 | Y | | C/T | C | 20.35 | 51763 |
| 94921923 | 94921923 | SG12S95 | LTA4H_29513 | R | rs4441106 | A/G | G | 7.07 | 51923 |
| 94922409 | 94922409 | SG12S96 | LTA4H_29999 | R | rs763875 | A/G | A | 5.92 | 52409 |
| 94922502 | 94922502 | SG12S97 | LTA4H_30092 | Y | rs763876 | C/T | T | 2.1 | 52502 |
| 94922681 | 94922681 | SG12S98 | LTA4H_30271 | Y | rs763874 | C/T | C | 32.42 | 52681 |
| 94923446 | 94923446 | SG12S42 | LTA4H_31036 | Y | rs2660892 | C/T | C | 27.41 | 53446 |
| 94923744 | 94923744 | SG12S55 | LTA4H_31334 | R | | A/G | A | 0.27 | 53744 |
| 94924037 | 94924037 | SG12S99 | LTA4H_31627 | R | | A/G | A | 4.37 | 54037 |
| 94924845 | 94924845 | SG12S100 | LTA4H_32435 | Y | rs2247570 | C/T | C | 27.79 | 54845 |
| 94924938 | 94924938 | SG12S101 | LTA4H_32528 | R | | A/G | A | 1.5 | 54938 |
| 94925915 | 94925915 | SG12S33 | LTA4H_33505 | Y | rs2660895 | C/T | C | 30.71 | 55915 |
| 94926590 | 94926590 | SG12S34 | LTA4H_34180 | Y | rs2247330 | C/T | C | 30.9 | 56590 |
| 94926724 | 94926724 | SG12S102 | LTA4H_34314 | R | rs2247323 | A/G | G | 31.85 | 56724 |
| 94926915 | 94926915 | SG12S103 | LTA4H_34505 | Y | rs2247313 | C/T | T | 32.74 | 56915 |
| 94927010 | 94927010 | SG12S104 | LTA4H_34600 | Y | rs2247309 | C/T | C | 32.74 | 57010 |
| 94927133 | 94927133 | SG12S27 | LTA4H_34723 | Y | rs2247304 | C/T | C | 25.57 | 57133 |
| 94927900 | 94927900 | SG12S35 | LTA4H_35490 | R | rs2660897 | A/G | A | 35.93 | 57900 |
| 94927959 | 94927959 | SG12S105 | LTA4H_35549 | Y | rs11108381 | C/T | T | 2.4 | 57959 |
| 94928465 | 94928465 | SG12S28 | LTA4H_36055 | K | rs2660898 | G/T | G | 29.36 | 58465 |
| 94928740 | 94928740 | SG12S36 | LTA4H_36330 | Y | rs2540490 | C/T | T | 31 | 58740 |
| 94928970 | 94928970 | SG12S106 | LTA4H_36560 | Y | rs2540489 | C/T | C | 30.89 | 58970 |
| 94929183 | 94929183 | SG12S107 | LTA4H_36773 | Y | rs11108382 | C/T | T | 2.58 | 59183 |
| 94929213 | 94929213 | SG12S108 | LTA4H_36803 | R | rs2540488 | A/G | A | 26.28 | 59213 |
| 94929761 | 94929761 | SG12S109 | LTA4H_37351 | Y | rs2300557 | C/T | T | 4.76 | 59761 |
| 94929770 | 94929770 | SG12S110 | LTA4H_37360 | W | rs2246990 | A/T | A | 28.57 | 59770 |
| 94929936 | 94929936 | SG12S111 | LTA4H_37526 | W | | A/T | A | 2.81 | 59936 |
| 94930044 | 94930044 | SG12S112 | LTA4H_37634 | M | | A/C | C | 46.15 | 60044 |
| 94930343 | 94930343 | SG12S43 | LTA4H_37933 | K | rs2246973 | G/T | G | 32.93 | 60343 |
| 94930357 | 94930357 | SG12S113 | LTA4H_37947 | Y | rs2246972 | C/T | T | 33.54 | 60357 |
| 94931246 | 94931246 | SG12S114 | LTA4H_38836 | K | | G/T | T | 7.55 | 61246 |
| 94934775 | 94934775 | SG12S141 | | R | rs10777768 | A/G | | | 64775 |
| 94934975 | 94934975 | SG12S140 | | M | rs2660840 | A/C | C | 29.77 | 64975 |
| 94937348 | 94937348 | SG12S143 | | Y | rs2540482 | C/T | C | 17.02 | 67348 |
| 94941021 | 94941021 | SG12S144 | | R | rs2660845 | A/G | G | 19.43 | 71021 |
| 94943761 | 94943761 | SG12S221 | | R | rs2540475 | A/G | A | 16.92 | 73761 |
| 94946089 | 94946089 | SG12S222 | | Y | rs2660850 | C/T | C | 15.47 | 76089 |
| 94948016 | 94948016 | SG12S460 | | M | rs2660852 | A/C | A | 37.22 | 78016 |
| 94949965 | 94949965 | SG12S223 | | Y | rs2660875 | C/T | C | 43.79 | 79965 |
| 94950568 | 94950568 | SG12S224 | | R | rs2540473 | A/G | G | 6.12 | 80568 |
| 94952847 | 94952847 | SG12S225 | | R | rs2540472 | A/G | A | 5.63 | 82847 |
| 94953483 | 94953483 | SG12S226 | | S | rs2540471 | C/G | C | 37.7 | 83483 |
| 94953798 | 94953798 | SG12S227 | | R | | A/G | | | 83798 |
| 94953801 | 94953801 | SG12S228 | | Y | rs2660890 | C/T | T | 46.96 | 83801 |
| 94953831 | 94953831 | SG12S229 | | M | rs2660889 | A/C | | | 83831 |
| 94954155 | 94954155 | SG12S230 | | R | rs2660888 | A/G | A | 35.68 | 84155 |
| 94954449 | 94954449 | SG12S231 | | Y | rs4762661 | C/T | | | 84449 |
| 94958156 | 94958156 | SG12S232 | | Y | | C/T | | | 88156 |
| 94958339 | 94958339 | SG12S233 | | Y | rs2660885 | C/T | T | 15.18 | 88339 |
| 94962388 | 94962388 | SG12S234 | | R | rs5800242 | A/G | | | 92388 |
| 94962435 | 94962435 | SG12S235 | | Y | rs759391 | C/T | | | 92435 |
| 94963320 | 94963320 | SG12S236 | | S | rs2540467 | C/G | | | 93320 |
| 94963655 | 94963655 | SG12S237 | | Y | rs2540466 | C/T | T | 37.05 | 93655 |
| 94963774 | 94963774 | SG12S238 | | Y | rs10492225 | C/T | | | 93774 |
| 94964298 | 94964298 | SG12S239 | | W | rs2660874 | A/T | | | 94298 |
| 94966584 | 94966584 | SG12S240 | | W | rs2540461 | A/T | | | 96584 |

TABLE 4A
Haplotype association analysis including SNPs and microsatellite markers across the LTA4H gene.

| | | | | | | | | |
|---|---|---|---|---|---|---|---|---|
| DG12S1664 | SG12S16 | SG12S17 | SG12S18 | SG12S21 | SG12S22 | SG12S23 | SG12S24 | SG12S25 |

All MI vs controls

| | | | | | | | | | |
|---|---|---|---|---|---|---|---|---|---|
| short | 0 | C | A | T | G | G | T | T | A |
| form | 0 | | | | | | | | |

MI males vs controls

| | | | | | | | | | |
|---|---|---|---|---|---|---|---|---|---|
| short | 0 | C | A | T | G | G | T | T | A |
| form | 0 | | | | | | | | |

MI females vs controls

| | | | | | | | | | |
|---|---|---|---|---|---|---|---|---|---|
| short | 0 | C | A | T | G | G | T | T | A |
| form | 0 | | | | | | | | |

Recurrent MI vs controls

| | | | | | | | | | |
|---|---|---|---|---|---|---|---|---|---|
| short | 0 | C | A | T | G | G | T | T | A |
| form | 0 | | | | | | | | |

| | | | | | | | | | | |
|---|---|---|---|---|---|---|---|---|---|---|
| SG12S26 | DG12S1666 | SG12S100 | SG12S28 | SG12S144 | p-val | r | #aff | aff.frq | #con | con.frq. |

All MI vs controls

| | | | | | | | | | | | |
|---|---|---|---|---|---|---|---|---|---|---|---|
| short | T | 0 | T | T | A | 1.67E−02 | 1.24 | 590 | 0.49 | 481 | 0.44 |
| form | T | 0 | | | A | 3.20E−03 | 1.32 | 590 | 0.5 | 480 | 0.43 |

MI males vs controls

| | | | | | | | | | | | |
|---|---|---|---|---|---|---|---|---|---|---|---|
| short | T | 0 | T | T | A | 5.10E−03 | 1.34 | 361 | 0.51 | 481 | 0.44 |
| form | T | 0 | | | A | 1.50E−03 | 1.4 | 361 | 0.51 | 480 | 0.43 |

MI females vs controls

| | | | | | | | | | | | |
|---|---|---|---|---|---|---|---|---|---|---|---|
| short | T | 0 | T | T | A | 3.80E−01 | 1.11 | 229 | 0.46 | 481 | 0.44 |
| form | T | 0 | | | A | 1.35E−01 | 1.2 | 229 | 0.47 | 480 | 0.43 |

Recurrent MI vs controls

| | | | | | | | | | | | |
|---|---|---|---|---|---|---|---|---|---|---|---|
| short | T | 0 | T | T | A | 1.50E−02 | 1.51 | 88 | 0.54 | 481 | 0.44 |
| form | T | 0 | | | A | 2.40E−03 | 1.69 | 88 | 0.56 | 480 | 0.43 |

p-val = p-value.
r = Relative risk.
#aff = Number of patients.
#con = number of controls.
aff.frq = haplotype/allelic frequency in patients.
con.frq = haplotype/allelic frequency in controls.

TABLE 4B

Information on microsatellite markers that were used
in the haplotype association analysis shown in Table 4A.

| | |
|---|---|
| Marker Name | DG12S1664 |

| | |
|---|---|
| Chr | 12 |
| Cytoband | q23.1 |
| Start in SEQ_ID_NO_1 (bp) | 7855 |
| NCBI_build33Start (Mb) | 96.317853 |
| Size | 238 |
| CEPH standard | 245 |
| (reference allele) | |
| Polymorphism type | SNP |
| Polymorphism class | in-del |
| Heterozygosity ratio | 0.23 |

| | | |
|---|---|---|
| Forward primer | GGAAGGAGGACACTTCTGGA | (SEQ ID NO:118) |
| Reverse primer | GCTGTGAATGGCTAAACTTGG | (SEQ ID NO:119) |

| | |
|---|---|
| Marker Name | DG12S1666 |

| | |
|---|---|
| Chr | 12 |
| Cytoband | q23.1 |
| Start in SEQ_ID_NO_1 (bp) | 38342 |
| NCBI_build33Start (Mb) | 96.34834 |
| Size | 188 |
| CEPH standard | 193 |
| (reference allele) | |
| Polymorphism type | Microsatellite |
| Polymorphism class | Di |
| Heterozygosity ratio | 0.52 |

| | | |
|---|---|---|
| Forward primer | CACAGAAGCTGCAGTGGAAG | (SEQ ID NO:120) |
| Reverse primer | CAAATGGAGGAGTCAAGACCA | (SEQ ID NO:121) |

| | |
|---|---|
| Marker Name | DG12S1668 |

| | |
|---|---|
| Chr | 12 |
| Cytoband | q23.1 |
| Start in SEQ_ID_NO_1 (bp) | 86595 |
| NCBI_build33Start (Mb) | 96.396593 |
| Size | 398 |
| CEPH standard | 398 |
| (reference allele) | |
| Polymorphism type | Microsatellite |
| Polymorphism class | Di |
| Heterozygosity ratio | 0.72 |

| | | |
|---|---|---|
| Forward primer | GCAGTTTAAGCTGTATGTATATGAGG | (SEQ ID NO:122) |
| Reverse primer | TGAAAGCCATCACTGTAAGGA | (SEQ ID NO:123) |

TABLE 5
Haplotype association analysis including SNPs and microsatellite markers in the LTA4H gene region.

| | | | | | | | | |
|---|---|---|---|---|---|---|---|---|
| SG12S438 | DG12S1664 | SG12S16 | SG12S21 | SG12S23 | SG12S25 | SG12S26 | DG12S1666 | SG12S100 |

All MI vs controls

| | | | | | | | | | |
|---|---|---|---|---|---|---|---|---|---|
| Consecutive | C | 0 | C | G | T | A | T | 0 | T |
| Short version | C | 0 | | | | | | | |
| Protective variant | C | | C | | | | T | | |

MI males vs controls

| | | | | | | | | | |
|---|---|---|---|---|---|---|---|---|---|
| Consecutive | C | 0 | C | G | T | A | T | 0 | T |
| Short version | C | 0 | | | | | | | |
| Protective variant | C | | C | | | | T | | |

MI females vs controls

| | | | | | | | | | |
|---|---|---|---|---|---|---|---|---|---|
| Consecutive | C | 0 | C | G | T | A | T | 0 | T |
| Short version | C | 0 | | | | | | | |
| Protective variant | C | | C | | | | T | | |

Recurrent MI vs controls

| | | | | | | | | | |
|---|---|---|---|---|---|---|---|---|---|
| Consecutive | C | 0 | C | G | T | A | T | 0 | T |
| Short version | C | 0 | | | | | | | |
| Protective variant | C | | C | | | | T | | |

MI plus stroke or PAOD vs controls

| | | | | | | | | | |
|---|---|---|---|---|---|---|---|---|---|
| Consecutive | C | 0 | C | G | T | A | T | 0 | T |
| Short version | C | 0 | | | | | | | |
| Protective variant | C | | C | | | | T | | |

| | | | | | | | | |
|---|---|---|---|---|---|---|---|---|
| SG12S28 | SG12S143 | SG12S144 | SG12S221 | SG12S222 | SG12S223 | SG12S225 | SG12S226 | SG12S233 |

All MI vs controls

| | | | | | | | | | |
|---|---|---|---|---|---|---|---|---|---|
| Consecutive | T | T | A | G | C | C | G | G | C |
| Short version | | | | | C | | | G | |
| Protective variant | | T | | | | | | | |

MI males vs controls

| | | | | | | | | | |
|---|---|---|---|---|---|---|---|---|---|
| Consecutive | T | T | A | G | C | C | G | G | C |
| Short version | | | | | C | | | G | |
| Protective variant | | T | | | | | | | |

MI females vs controls

| | | | | | | | | | |
|---|---|---|---|---|---|---|---|---|---|
| Consecutive | T | T | A | G | C | C | G | G | C |
| Short version | | | | | C | | | G | |
| Protective variant | | T | | | | | | | |

Recurrent MI vs controls

| | | | | | | | | | |
|---|---|---|---|---|---|---|---|---|---|
| Consecutive | T | T | A | G | C | C | G | G | C |
| Short version | | | | | C | | | G | |
| Protective variant | | T | | | | | | | |

MI plus stroke or PAOD vs controls

| | | | | | | | | | |
|---|---|---|---|---|---|---|---|---|---|
| Consecutive | T | T | A | G | C | C | G | G | C |
| Short version | | | | | C | | | G | |
| Protective variant | | T | | | | | | | |

| | | | | | | | | |
|---|---|---|---|---|---|---|---|---|
| SG12S237 | DG12S1668 | p-val | P-val adj. | r | #aff | aff.frq. | #con | con.frq. |

All MI vs controls

| | | | | | | | | |
|---|---|---|---|---|---|---|---|---|
| Consecutive | T | 0 | 6.2E−02 | 1.34 | 1560 | 0.051 | 953 | 0.039 |
| Short version | | 0 | 1.5E−03 | 1.63 | 1556 | 0.071 | 951 | 0.045 |
| Protective variant | C | | 7.5E−02 | 0.88 | 1557 | 0.290 | 951 | 0.317 |

MI males vs controls

| | | | | | | | | |
|---|---|---|---|---|---|---|---|---|
| Consecutive | T | 0 | 2.2E−02 | 1.49 | 1096 | 0.051 | 953 | 0.035 |
| Short version | | 0 | 3.1E−03 | 1.66 | 1093 | 0.069 | 951 | 0.043 |
| Protective variant | C | | 6.3E−02 | 0.86 | 1094 | 0.283 | 951 | 0.314 |

MI females vs controls

| | | | | | | | | |
|---|---|---|---|---|---|---|---|---|
| Consecutive | T | 0 | 4.3E−01 | 1.19 | 464 | 0.046 | 953 | 0.039 |
| Short version | | 0 | 1.6E−02 | 1.60 | 463 | 0.073 | 951 | 0.047 |
| Protective variant | C | | 3.1E−01 | 0.91 | 463 | 0.301 | 951 | 0.322 |

Recurrent MI vs controls

| | | | | | | | | |
|---|---|---|---|---|---|---|---|---|
| Consecutive | T | 0 | 7.7E−02 | 1.52 | 273 | 0.060 | 953 | 0.040 |
| Short version | | 0 | 7.5E−02 | 1.54 | 272 | 0.067 | 951 | 0.045 |
| Protective variant | C | | 9.8E−02 | 0.82 | 273 | 0.274 | 951 | 0.316 |

MI plus stroke or PAOD vs controls

| | | | | | | | | | |
|---|---|---|---|---|---|---|---|---|---|
| Consecutive | T | 0 | 1.5E−03 | 0.007 | 1.97 | 325 | 0.073 | 953 | 0.038 |
| Short version | | 0 | 2.4E−05 | 0.038 | 2.39 | 325 | 0.099 | 951 | 0.044 |
| Protective variant | C | | 4.1E−05 | | 0.61 | 325 | 0.220 | 951 | 0.315 |

p-val = p-value.
P-val adj: P-value adjusted for multiple comparisons.
r = Relative risk.
#aff = Number of patients.
#con = number of controls.
aff.frq = haplotype/allelic frequency in patients.
con.frq = haplotype/allelic frequency in controls.

Discussion

In a genome wide search for susceptibility genes for MI, a gene was mapped to 12q23. This locus was fine mapped with microsatellite markers. Haplotype analysis in a large case-control association study using markers spanning a 79 kb region across the LTA4H gene, shows that LTA4H is a significant susceptibility gene for MI.

The LTA4H gene encodes a protein that is required for leukotriene B4 synthesis. The leukotrienes are potent inflammatory lipid mediators derived from arachidonic acid. Given that our data shows that LTA4H shows significant association to MI, it may contribute to development of atherosclerosis in coronary arteries and/or to the destabilization of existing coronary atherosclerotic plaques through lipid oxidation and/or proinflammatory effects. In support of our discovery, Dashwood and coworkers have studied expression of the enzymes that control the formation of leukotrienes in coronary arteries. They showed that cells showing positive antibody binding to 5-LO, FLAP (5-lipoxygenase activating protein), and leukotriene A4 hydrolase were present in the coronary arteries and had a similar distribution to macrophages. (Dashwood, et al., Circulation 1998 Jun. 23; 97(24):2406-13). Thus, LTA4H and other members of the leukotriene pathway are expressed within cell types found in atherosclerotic lesions that form the basis for the final event of myocardial infarction. Their potential role in plaque instability may explain why many patients have stable angina for years without suffering a myocardial infarction (and therefore presumably have atherosclerotic lesions without the instability that leads to overriding thrombosis and MI) while others suffer MI with little or no period of stable angina. Those patients with elevated LTA4H enzymatic activity in atherosclerotic lesions may have more unstable plaques and higher MI rates. In addition, increased LTA4H activity may accelerate atherosclerosis lesion formation and progression.

Our work on LTA4H is supported by our previous work on the gene that encodes FLAP, which works with 5-LO to produce Leukotriene A4; that is, it is upstream of LTA4H. We found that variants in the FLAP gene more than double the risk of MI. LTA4H represents the second member of the leukotriene biosynthetic pathway that we have been the first to show confers substantially higher risk for MI.

Further work in animals which supports our discovery that LTA4H is a disease gene for MI comes from Aiello and coworkers. They have shown that leukotriene B4 receptor antagonism reduces monocytic foam cells in mice, suggesting that LTB4 has a role in the pathogenesis of atherosclerosis in mice. (Aiello, et al., Arteriosclerosis, Thrombosis and Vascular Biology. 2002; 22:443.)

Finally, additional support of our human validation of the leukotriene pathways role in MI in general, and for LTA4H, in particular, comes from Mehrabian et al. who described the identification of 5-Lipoxygenase (5-LO) as a major gene contributing to atherosclerosis susceptibility in mice. Mehrabian et al. described that heterozygous deficiency for the enzyme in a knockout model decreased the atherosclerotic lesion size in LDL−/− mice by about 95%. Mehrabian et al show that the enzyme is expressed abundantly in macrophage-rich regions of atherosclerotic lesions, and suggested that 5-LO and/or its products might act locally to promote lesion development (Mehrabian et al., Circulation Research. 91:120 (2002)).

These results suggest that the Leukotriene B4 branch of the leukotriene pathway (as opposed to the other main end products of the leukotriene biosynthetic pathway: leukotriene C4, leukotriene D4, and leukotriene E4) may be more specifically involved in MI risk. If so, then medicants acting on this branch or blocking the effects of LTB4 may be more effective in preventing/treating MI than those acting on the other branches of the pathway or that block the effects of LTC4, LTD4, or LTE4. However, our current data do not exclude these other branches of the leukotriene pathway; the data do suggest that at least the LTB4 side of the leukotriene pathway is important for MI.

Mutations and/or polymorphisms within or near the LTA4H nucleic acid, and other members of the same pathway (i.e., leukotriene B4 receptor 1 and 2, leukotriene B4 omega-hydroxylase, leukotriene B4 12-hydroxydehydrogenase), that show association with the disease, may be used as a diagnostic test to predict those at risk for MI and ACS as well as those who might benefit from medicants directed against members of the leukotriene pathway. Therefore, there may be other members of the leukotriene pathway that may be valuable therapeutic targets for myocardial infarction in addition to LTA4H and FLAP.

### EXAMPLE 2

### MRNA Expression of the LTA4 Hydrolase Gene in White Blood Cells of MI Patients vs Control

mRNA expression was compared in white blood cells from patients with history of myocardial infarction (MI) and in age and sex matched controls without MI. The leucocyte population was separated into: 1) neutrophils and 2) peripheral blood mononuclear cells prior to RNA extraction using standardized methods as previously described (Helgadottir et al, Nature Genetics, 2004; Hakonarson et al, J Immunol, 2001).

RNA was isolated from PBM cells obtainted from 43 MI patients and 35 controls. RNA was separately analyzed from granulocytes from the same subjects. Sufficient amount for RNA was obtained from all PBM cell preparations, and granulocyte preparations from 35 MI patients and 29 controls. RNA was converted into cDNA using the protocol below. PCR was then run on the cDNA with the LTA4H Assay-on-Demand and Beta Actin Pre-Developed Assay Reagent from Applied Biosystems using the PCR parameters below.

TABLE 6
PCR Parameters
RT Reaction

| | | | |
|---|---|---|---|
| TaqMan RT Buffer | 1× | | |
| MgCl2 | 5.5 mM | | |
| dNTP | 0.5 mM per | 25° C. | 10′ |
| | dNTP | | |
| Random Hexamers | 2.5 uM | 48° C. | 30′ |
| Rnase Inhibitor | 0.4 U/uL | 95° C. | 5′ |
| MultiScribe Reverse Transcriptase | 1.25 U/uL | | |
| RNA | 2 ng/uL | | |
| | 50 uL Reaction | | |
| | Volume | | |

PCR Reaction

| | | | |
|---|---|---|---|
| TaqMan Universal Master Mix | 1× | 95° C. | 10′ |
| TaqManAssay (20×) | 1× | 40 cycles: | |
| cDNA | 2 ng/ul (original | 95° C. | 15″ |
| | RNA) | | |
| | 10 uL Reaction | 60° C. | 60″ |
| | Volume | | |

All PCR reactions run in duplicates.

ABI7900 instrument was used to calculate CT (Threshold Cycle) values. Samples displaying a greater than 1 deltaCT between duplicates were not used in our analysis. Quantity was obtained using the formula 2′-deltaCT where deltaCT represents the difference of CT values between target and housekeeping assay. mRNA expression was subsequently compared between patients and controls. To determine if there were differences between the groups, we used standardized Mann-Whitney analysis as well as Standard t tests, with p<0.05 considered significant. Moreover, given our hypothesis of enhanced expression of the LTA4 hydrolase gene in patients compared to controls, we report both unpaired two-sided and unpaired one-sided t tests with Welch correction.

TABLE 7
Results
Analysis

| | # | # 5% extr. | Ave Q −5% extr. |
|---|---|---|---|
| PBMC | | | |
| Patients | 43 | 2.15 | 1.954317191 |
| Controls | 35 | 1.75 | 1.72766267 |
| Granulocytes | | | |
| Patients | 35 | 1.75 | 0.401265947 |
| Controls | 29 | 1.45 | 0.331226464 |

Statistics Granulocytes MI patients vs controls
P = 0.0868 Mann-Whitney two-sided test
P = 0.0635 Unpaired two-sided t test
P = 0.0318 Unpaired one-sided t test
P = 0.0556 Unpaired two-sided t test with Welch correction
P = 0.0278 Unpaired one-sided t test with Welch correction
Statistics PBMC Patients vs Control
P = 0.0456 Mann-Whitney two-sided test
P = 0.0591 Unpaired two-sided t test
P = 0.0296 Unpaired one-sided t test
P = 0.0656 Unpaired two-sided t test with Welch correction
P = 0.0328 Unpaired one-sided t test with Welch correction

Relative to cells isolated from control subjects, mRNA expression of LTA4 hydrolase gene is significantly enhanced in both PBM cells and granulocytes isolated from patients with MI. These data further confirmed the role of this gene in MI.

All references cited herein are incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1-102. (canceled)

103. A method of assessing a susceptibility to myocardial infarction (MI) or acute coronary syndrome (ACS) in a human individual, comprising screening nucleic acid of the individual for at least one polymorphism in a leukotriene A4 hydrolase (LTA4H) nucleic acid, or a LTA4H haplotype, that correlates with increased occurrence of myocardial infarction in a human population,
wherein the presence of the at least one polymorphism or the haplotype in the nucleic acid identifies the individual as having elevated susceptibility to MI or ACS, and
wherein the absence of the at least one polymporphism or the haplotype in the nucleic acid identifies the individual as not having the elevated susceptibility.

104. The method of claim 103, wherein the screening further comprises measuring C-reactive protein (CRP) in the human individual, and
wherein the presence of elevated CRP and the presence of the at least one polymorphism or haplotype identifies the individual as having elevated susceptibility to MI or ACS.

105. A method of selecting a human individual for prophylactic treatment against myocardial infarction (MI) or acute coronary syndrome (ACS) using an inhibitor of leukotriene synthesis, said method comprising:
screening nucleic acid of the individual for presence or absence of at least one polymorphism in a leukotriene A4 hydrolase (LTA4H) nucleic acid, or a LTA4H haplotype, that correlates with increased occurrence of myocardial infarction in a human population, and
selecting for said prophylactic treatment an individual having the polymorphism or the haplotype present in the nucleic acid.

106. The method of claim 105, wherein the screening further comprises measuring at least one inflammatory marker in the human individual, said at least one marker selected from the group consisting of C-reactive protein (CRP), serum amyloid A, fibrinogen, interleukin-6, tissue necrosis factor-alpha (TNF-alpha), soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, myeloperoxidase (MPO), lipoprotein phospholipase A2, N-tyrosine and di-tyrosine, fibrinogen, leukotrienes, and leukotriene metabolites; and
wherein the selecting step comprises selecting for said prophylactic treatment an individual identified having the presence of an elevated inflammatory marker measurement and having the presence of the at least one polymorphism or haplotype.

107. The method of claim 105, wherein the screening further comprising assessing at least one family or medical history risk factor selected from the group consisting of past or current smoker, diabetes, hypertension, increased total cholesterol, increased serum LDL cholesterol, decreased serum HDL cholesterol, increased leukotriene synthesis, hypercholesterolemia, elevated triglycerides, elevated lp(a), obesity, acute coronary syndrome (ACS), angina, atherosclerosis, periphereal arterial occlusive disease and surgery to restore coronary artery blood flow; and
wherein the selecting step comprises selecting for said prophylactic treatment an individual identified as having the presence of at least one of said risk factors and having the presence of the at least one polymorphism or haplotype.

108. The method of claim 105, wherein the screening further comprises measuring C-reactive protein (CRP) in the human individual, and
wherein the selecting step comprises selecting for said prophylactic treatment an individual identified as having elevated CRP and having the presence of the polymorphism or haplotype.

109. A method according to claim 105, further comprising a step of prescribing for the subject a medicament that comprises an inhibitor of leukotriene synthesis.

110. The method of claim 103 or 105, wherein the screening comprises screening the nucleic acid of the individual for at least one polymorphism in a leukotriene A4 hydrolase (LTA4H) nucleic acid listed in Table 3 that correlates with increased occurrence of myocardial infarction in a human population.

111. The method of claim 110, wherein the at least one polymorphism is selected from the group consisting of SG12S438, DG12S1664, SG12S16, SG12S21, SG12S23, SG12S25, SG12S26, DG12S1666, SG12S100, SG12S28, SG12S143, SG12S144, SG12S221, SG12S222, SG12S223, SG12S225, SG12S226, SG12S233, SG12S237, and DG12S1668.

112. The method of claim 103 or 105, wherein the screening comprises screening the nucleic acid of the individual for a haplotype comprised of at least two polymorphisms in a leukotriene A4 hydrolase (LTA4H) nucleic acid listed in Table 3, wherein the haplotype correlates with increased occurrence of myocardial infarction in a human population.

113. The method of claim 103 or 105, wherein the correlation is an increased risk of MI of at least 20%.

114. The method of claim 103 or 105, wherein the correlation is an odds ratio of at least about 1.2.

115. The method of claim 103 or 105, wherein the haplotype in the LTA4H nucleic acids comprises markers DG12S1664, SG12S26, DG12S1666, and SG12S144, with alleles 0, T, 0, and A, respectively.

116. The method of claim 115, wherein the haplotype in the LTA4H nucleic acids further comprises markers SG12S16, SG12S17, SG12S18, SG12S21, SG12S22, SG12S23, SG12S24, SG12S25, SG12S100, SG12S28, and, with alleles C, A, T, G, G, T, T, A, T, and T, respectively.

117. The method of claim 103 or 105, comprising screening for the presence or absence in the individual of a haplotype using one or more of the markers DG12S1664, SG12S26, DG12S1666, and SG12S144, with alleles 0, T, 0, and A, respectively, wherein the presence of the haplotype is diagnostic of susceptibility to MI or ACS.

118. The method of claim 103, wherein the screening comprises at least one procedure selected from the group consisting of: (a) enzymatic amplification of nucleic acid from the individual; (b) electrophoretic analysis; (c) restriction fragment length polymorphism analysis; and (d) nucleotide sequence analysis.

119. The method of claim 103, comprising a step of obtaining a nucleic acid sample from said individual for the screening step.


SEQUENCE LISTING (129 sequences)

SEQ ID NO: 1 - DNA, 100001 bp, Homo sapiens
taaggcatatcatgcaaagtaaaattagccaaagaaagtcaactggtggagagcttgttgaagcaaatttaaaaaaaaaaaaaaaggttaacaaaagtctaatgtttttagaaaaaattgctatcaatctgtttccaaatttgaattcatctaatgctaagagtaaaaaacaggcacatacaattgtggttattcttctcacccttaagagtgagtggcctgttgaaactgttaagaaagaaagaaaagttttataatctgaaaatacctggtgggtcttgaaccacgacaacaggaacacagtgctgaatttagcaactataatactgccatcagcctaaccaacgtaggctttagaagaactgaatgatacaatggattgatctacctaggaaagttctcaggtctctccttcagcctagtattgctttgtgctaaactgactgccctctcatctgctacttcatgacaaagcccattaaaggtcctcagactccgggattttggtggatttcttgtgcagaaattgcagtgaaaaggctgttggagaaagaggtcttgattctggaatatgctccattctgtatttttcaatgtatggagcagctacttcccaaacctgaaaagcaaggacaaaacaaagttgaaatattgacgacattgtttccacatgctattaaacatcaacttcatccgaagtcaaaacatactctatacatgaccagacacagctgctgtttgcttgcttttattttaagccatttgacacatgacctgtgtcaattagtctttggttgcattaaagactgtaaatatacaaagtccaaaactttctaaagtcatcataaagattttaagctgcatacttttcctaagcaaaacaagcaagcaaataacaaaacccagagaattcagtgtggatagagtgaaggatagttgctccagggtcttaacagtacctatgtggtttttttcttgtttttttgttttttttctttttttgtgagacagagtctcactctgtcgcccaggctggagtgcagtagcatgatctcagctcactgcaacctccaccttccaggttcaagcaattcttgtgcctcagcctcctgagtagctgggattacaggtgcatgccaccaggcccagctaatttttgtatttttagcagagatggggtttcaccatgttggccaggctggtctcgaatttctggcctcaagtgatccacccaccacagcttcccaaagtgctgggattacaggcgtgagccaccacacatggcctggacctgtgtgttttctaaagcaagccttaaatggtaaaaggcagtgaattgtatttccctattgccttatttccatgccacaggtgctctgttcctttgaccctgctactcaaagcgtggcctgtgcccctgcagtatcagcatcacctgggacaagtcagaatcttggccttcacccagacctactgaatccacacctgcattttaacaagatgcccagcagattcatagccactttaatgttggagaggcactgcctcagatccttggttagcttttgcctcccacccaccaacctcttttttctttttttttttttttcttaagcataacatcatctcaaagtaaggccctggacgggcatggtggcatgtgcctgtagtcccagctactcaagaagccaaagtgggaggatcacttgagcccaggagtttgagtccagccttggcaacatagcaagacccgcatctctaaaaaaatttttttttaaattgaatgaaattaaggccctgtttttggtttatatctgctttttaactgattgccactgacagccaaggagcctttcctattattttttatgaccttagcaaatgaatactcttaaaggctaacctctcgtatacctccctcctagcaccaaagttggcagcaggcaagcagcttccccatccagagtgcagggcatgcttagaaataatgggtgtgaaaatcacagggaaaaatcactgccccagagcagagcaactgttttaaggaaatcaagcgattctagggaacataacacccacaagttattcaaaaggtttaaagcacttcaaaaacgatatttaaaagataagccagcatgctgggcatctgtacctggcagctgagtatacggtcaatgtaaccagtggaaatatgcaagaaagaaaaacccgcatctcaccagagcactagacagaccgaaagtcttctgaagtaaacacccgggccttggttttctctccaggtctacgcagccattgcacccagtggtggtagttgtgatagcatcacccagaagggaacgcacttttgaatcaaagaggacatcttgcaggggtggggaggcatcaatgaacctgacatcttatttttttccccatgaatattgtcccaaaactccatttaaaatccatttctgtttctaatccttagatattcaaccgttggctgcaccctggtgcacttagtgtttattatatggcttcttagtggtgctgcaagttgttgctcaaatacctttttgctcattcccaaggaatggccagaaaacaatagaataaggcaatgtttctccatccccgactttgctttcctgtaacaattaaaaattaagaaacaagccaaggagccagcttgccttctgctccaggagcagcctgtgggctgcctcgatgtccggggccatgaagcgatcttttatccagggcctacagggagagcacatccgcccatcagccaaacatgaaaccctgccagggttcacagtgctgaactgatgtaattttcagagacctgtttgatcttaccttacaacagagcgcaccaggtcatagaccttctccagcggagtggttgttttcaggggacgtagaaactctatgccctggcaggctgcaaggagctcgatggccagcactgaaacaagaaattccaagagggtagcttatgaaagtctgacttcatgcttaaggaatgtggatttcccaaagttgacacccatcaccacccaactaacagatggtttgttctctgtctattggtagaatgaagttgctttggtatttagttgccattttaacctgttttctaaccccttgactatatctttaatgctgagaaggggagaattgagacatttacctgaataattaccagacctgcacacctacccaggaagctgcccattcatctgcactagcaaaatctgccatgtcccccattcacatcctctaacaaaggtttgttggccaagtttgggtcatgtgggtagggaaggaagtcagaaagaagagctgatcctcatcttgagccccagttctattaaataattgtatgcatttcatcaagatgctttacatctcgtggtttccaattccatatgaattgcggggtggggatactggactagaagatgagaaattgcttccaggtccaccgtgaattccacaacttcatcctctcccactgtgacctgtctgcactcactagagttcatagtgtacttcataaattgaatgtgctgttgaatcaaggtggggtgtgagagttcatggtactcttctctccactttgaatatgttttaaaagtctcattaaaaaaaaaaccactttgggggtgaggtgggaggatcgcttgaggccaagagtttgagaccagccaaggcaacacagcaagaccccatctttacaaaaaatttaaaaattacccaggtgtggtggtgcttgcctgaggtgtcagttacttaggagactaaggaaggaggatcacttgagcctgggagttcaaggctgcagtgagctattgttgcaacactgcactccagcctgggtggcagatagagatcctgcctcaaaaaaaaaaaaaaaaaaagagaaaaaaaaagccagccaaaggaccagcttagttctgcatggttccttggttcttaatctttagtgaagatcagaatcacctggagggttcgttaaacacagattgctccgctctcctcctggagtgtctgaattagccagcctgcaaggaggcctgagagtctgccttcctaacaagtttccaggtgatgctgatgttgctggtgggagatcccacttgagaaccacgggcacagtggtctatcaggcaggcccgccaccccgaactcatcagcattaccttgctccacatgctcgatgaccctgagggctttccttgctgcccatcctcccatggagacgtggtcctccgtggctgcgctggtggagagggagtcaacagacgagggatggcacagagccttgttctcagaaactgcaagagaccagtgccagttaagaagtgctcctcacaggatgagctgtctaaaggacccgtggcttccacagagtgctccacagcatgggatacactctccagaagatcttggacattatccaagcacctgatggtagaaagctgctttgggaaaaggaagcaggtcattttttttccccaagtgaggactctaaagcaatagtgagttctgaggtaagacggagatgggagaactgggaattctaatataacaaggccaagatgacaacctgggaagcatatagcatgccagggagactaggggagaaagagacatgatggccattttcaggtacttgacgtcaatttggttgtgtcaccagaattccgatgtggcatttggggactgaggctggacccataaatcttgggcaagaagatttaacacttcagagtgttcaaggatgacataaatggatgtaaacaggggctcgatgactgccagaaatatctgggaggtgggggaattctagggaggaccaagacatgggactctcaagctttccagtgcacatgaatcactcagggaccttgttataacaaatatactgagctttgggtggggcgtgaaattctgctgttctaacgagctctcaggtgatggccatattgttggtctgaggactacactataagtagcaaaaacttaggttatgaagttccctcaccctagcttagaagtcaggaaaggtcagactcaaagtctcttctcctctttctctggctttaagagtggtcatgggtgaggcttcagttgttttctatgctgcatgggatcgcatcctaagatgatctcgctttaaactagggtcatgctactttgtcaaggtcatgttggttggccaaggactttccaggtttcagcactgtaagtcccaagtccttggaccctaaactgtggactatacagataagtaaactgtggtaaggcttgttcggaaagataacttacaagggccaaaaaaagaaaaacactaattttactacaatgaaagcaagccaaacaacctcaataggcagcttgggaaactaaaacatattcaaatgaggcttcccaatgtccatcctgttctctagaaatcaatgactactcccccactactacgttattgtgagaaatttctacctgttaaaggggtattttattaataagcaagtaagcaaatagaagataccggtcagaggaggtgaattcagtggctcttagttggttatttgtgttcgttgggaataatattaatagcaactagtatttttttagtgcttatgatgtgccaggctttatatacatcaattcatttaatcctccaaacaacccccttaggtaaactgatctccattttatgcaggtgtaaattgaggatcaacaggataaagaggctcacagagctagtaaatggcagagctgggcttctgatctagcatctacgcatgtaataactcatgctatcctgccttttagggtagggttgttagataaagtaaaggatgcccagttaaattttgattttagattaaaaaaataatttttgtagtacaagtatgtcccaaataattcaaatttaactagtcatcctgtgtttttgttaaatttgacaaccctattttaggattaagtattgattaaaatgagatcctcattaattgaagtaaaagcacatttttgcatgccaaaattcagggtttgaattctgctttacatcttgctagctgtgcaatattgggcaagtcacttaactctcagatcttcagatattcatctatagataacaataatagcacttacctcaggtgacaattgtgcaattaagtgaatgaacataaatgaggcccttagtacttacttagtgccaggcactgttctgatgtgttaatctgtctcttacaatcttgagtcagaaataaactatattgaagctcaatcttcacagagggtatcctgattcttataagcatttttttaatgttgcaataacatctttatgtttttagtggttgttatatctagtgaaatgaatccaaagtttaattataggtttaaaagttcaaagtttaattataggttggttcaagtttcaccattaactataaccatcttagtctttttccaatagtcttcaaatggagatttggaaaaaagctatagagatgaagttcatatgcaatgtcaaattgctttccacagagaaattgtccattacagccttattattgtattattatatttcagcataacagtatcgaagtttttaaactatttttttctgaaaaatattagagttaacctgttacctttgcactgagagaactaggatgcagataaaagtggtgaaaagttagatctcactcacagaccacatttgatacttctaggtgaaatggaaagacccttagatggacgggcgtgtgttctgggaaaggggcagtgtccttaccaagggctgctgccgtgcagtgagctatcatgaacccagagttcagaccaccttcagccaccaggaaggcaggcagctcactgagggagggattgcagagccgctcgattcttctctcactgattgcagcaagttcatggatgccaatggccaagtagtctagggcctgaaagagggtctccatttagtcagcctatattgaaatgtgccttctgggtcaggagcagttttaaaaagcttactttggctgggtattcaccatggaagtttcctccagaaactgtctctcccctattggcaaagaccatctttcaaaagaggttaaggcttgaagataatgtttgctggtcacttcaagtacaaccccttcctgccttcttttttaacgtggaggtggcagtctgtgtacaaatgtggggtggagggaaagagacaagtcttcctgcaagagatataaaggggcacaaaagcaaaaaacaaaaagccttaaaggtggatcatgtacacccaatttggaggctgaaaaaacatgaaccttgaaatgatgttttctcttgagattagattctaaagcaccataactgtactaacctggtggttagtacctgccctcaaaaggtttatagcctgaaactaaaaacagagaccaacacatctgtttgaaagcaacttctgtttcttcaatgggcactgcaaatttgtgttccatagaagaatctgttgaatgggaaactctgctgatgagcaactaccaaaatgtctggaacaatcaattttgagaaaattttcaaattctttactcagtaatctggtgtcagcgttcaacagcagatggtggccaatatgtcctcatgaatccattttaagtaggtggcaagtttaaatttgctgaatattattactctgaggtaattctttttttattttctgtatcattctgtcacccaggctggagtgcaggggcgccatctcggctcactgcgacctctgcctcccgggttcaagtgatttcttgtgcctcagcctcctgagtagctgggattacaggcatgcaccatcacacccagttaaattttgtatttttagtagagacagggttttgccatgttggccaggctggtcttggactcttggcctcaagtgatcagcccaccttggcctcccaaagtgctgggattacaggtgtgagccaccaaacccagcctcagataattctattccatcatgcactttacttaaaccagcaagcacccaggaaaaggcagtaacagatccttcttcctggttctaaagagctaatctttgcctcttgggctcaagaatattcattacctattaactgagaacttgctgtgtgcaagaaagtttctaactttgaaagagttagagatgtatgtgttacattaacctactcaagtgtggattactctgttctgcatgtgtaactgtgagcccattgggtctagctccttggtcttggcctcttctccattctctgaatccctatagcaatggaattctgcatcaatttagaacttagacatgtttttattgtcctctagttgttccaaatttgtttgttggctattagattaaaacaattagaatttaactcccataatatccagcatgtgtgatggggctccgtatctgcaacagaaaattgagacacatattctttcaggtaacagcagtattgcaagagaactcccaaatattagcctattggaaatttatcaataattagcaatctttaattgaaaaaaataaaagacttttggctgaaattcttgctgaggcaattggtagcagaagaatttagtgaggaattaattatcccctgccccatattcagtaattgaatggtaatttctaaaaactatgagataccctttaaaagcaatgatgtaaatgtagaatagatacctcttccctacagtgaaaagactggtacgtgagataagactacatagagttcataaaaactactattccatctgggggatggggtggtattgaggtgaatggagagacccacaatttctgaggcttatttagtgtgggtttgacttcagtgtaaaacaatgtgagagaacataaaacacacattctgaagtaagcttgtcaaaaaacacatacatacaaaagccatctcatgggaaaccttatggagctatcacataaccatcttctaactcagcctgtgacctgcttttggggctgcttactcagcactttctctaggattcagaactgaggcagatgtggaataggaaaaatagattccaaaggcgtttgattatcttcagaggcagtgctatctccatgaaaactgtgccagtgtgactcctgactgatcacctcaacatctggggaactgagccatcatggaacattccagagaaaaaccagatggctctgagataagccacaccattaagagaggacctccatccttgagaaatggaatcagttttactatttctttgaacaaacgcaaaagcactaatttgagcaaagtgcgcatgacaaataaaaactcatgatgggggctgggtgtagtcgctcacacctgtaatcccggcactttgggaggccgaggcaggtggatcacttgaggtcaggagtttgagaccagcctggccaacacagcaaaaccctgtctctactaaaaatacaaatattagccaggcctgctggcacatgcctgtaattccagctactcaggaggctgaggcaggagaatcgcttgaacccgtgaggcagaggttgcagagagccgagatggcaccactgcactccagcctaggagacagagtgagactccgtctaaaaataaacaaaaaaaaaaatgccaaaaacctcaggatgaggaagccataactctttaatcaatactttggtaagtgttaaaaggctgacctctgcataatgcaaactgctctcactcaaagcagagaggcctgcataaattgttcagcatttctgcaatcacatttgtgacccacgtcttcacccttccaccaacaaccttggcagtgggttttagggccagtgtcttcaggaaacatgtcagccaatgctggaaatatcttcttaaatcttgaataggtccattttgaaagtacagcatgttagacttggaacctgagagtaagttcacaggtcacctctgccacttaccagctctgacctggagaaagtcagttaacctctgagcctcaggtactgattcatttactctgtgaaatggagcaatggaactgagaagctacacaaaatgattgtttcagtagatcctaaaattaccagctgcaattctataattgcaggttagtatacaatcaaaagatacaggattatctgttgcgctgttcagttctgtggtaatgatgttcttcacaaatgctattgtatcattcaccacaccatggacctaaaatacaatcaaaataaggtatatgacccatttgattacctgaagagtaccaatattcttaaaagaatatagaagatcttatcttaaagttataaagaatacagaagatcttattttaaagttataaccatggaaattatttgaactgaagataattttgttataattcttttgggttttcactgaaacaaattagaataaattcattatcatatatattcacattagatcaatctatacaaagcagcatggtgtgggaggaagaatgccaactctagacacaggcagattcactcccaggttagtggtaggacttgggcaagttccttaggtttatagttaaaagataggaagacttagaaaagagacaatagcacttatggcttttgagaagtacataaaataatgcaaacaaattcttagatgtgactcgcagagattactcaaatggtagctattatccaaggcggtaaagttcattaagcacgaaggcatcacattagaagatgctcacacataaggcagctacgctgtacaaaccccagaaatagccacaatgctataaagcagcacctggctctggtttctcccagaagtcaaggaagtaattatctgtctgtgtgatcgtgggcaagatacttaattctgtgcctcagtttccccattgcaaaatgggagaaagagtatcttatagttgggaagattaaatgaattaataaatgaaagacagtgataatggtgcctgtcacatagaatctgctatcgttattaaaatatcagggaagtcttacatcaggacttaatcttcctgtagatctaatttggcaattcttacccataggtgttttgatctattgctcaataagctagctagaaaagataacttgctgactaaagggaaatgagggtatttattctagggaggactctctattacaccatagactgaaatttggcacagaccaaaatatggtttattgagatgactggctctctaactgtcatatgtaaattacacatctgtaatacagatgcctaatgccaacccaggtcatctgcatttttaatttaaagttaagcatcacaaattcatttaaaataatttatattgcccataaaatgcaagtaaggtataaccattactatgcacctgagtttcaaaatgatataattaagactttcctgtaaacacctctgactatctggttaaagtttcatcttaacagggagaaacagtacaactctactaactggttatgattttattactaagaatttttaaaagttagggcactgaatcaacataagaggttgtctgggccggggacggtcctcacgcctgtaatcccagcactttgggaggctgaggcaggtggatcatttgaggtcaggggttcgagaccagcttggccaacatggtgagaccccatctctactaaaaatacaaaaattaggcatggtggcacatgcctgtaatcccatctactcgggaggttgaggcaggagaatcacttgaacctgggaggtgaagattgtagtgaacagagatcatgccactgtactccagccagggtgacagagtgagactctgtctccaaatcaaagaggttatttgttggttgtctgtctcaaaaaaaaaaaaaaaaaaaaaaagaggttgtcctctgaaagtaggttgaacaatcaaaagaaaacaaatagttcaccatatagaatagaaactttacaacaattgcacacataaaggtaaaatatacataacttttcatagagacagtaagaaatttggactatagttgaccattacatgtttttctaacactttcctattttaaaatttttcatcctgaacctggggaaagaaaaactttattggcattttttattgacctttttttttttctttattttacctgtggacagcagcgcaaggtgtatgcatcctggacgcgatcacagaacctgtgactctctgtggaagaggtggaggggatgagacagggatcatgttaccaattcttttcacaaaacacgtaaccaaataggtcactcccataaacatggtcagacctgctatttctgatgggtggtgatctgagtccaagagtgaccgaaaccgaaaagcaacttcaatttgcccacggtgaggtcgaagagcatgaatgtctagaattgatgaaggagaaaaagtctgaataattccatttatttattttttttaaagatgggggtctcactacgttccccaggctggtttcaaactcctgggctctagcgatcctcctaccttggcctcccaaagtgctgggattacaggcatgagccaccatgccaggtcgagtaattctgatattagaacaaatgaccatcaatggttcaggaccatctacggtgctgacacagccctcaaaagtcccaggggtccttagccaccactcagaattagaaacttaactccgaaagcacgattttccttattgacttgactccttctactttaaatatctgagagcaaactatatgttgagaccactgggaattccactcaagacactgggcctaccttaaaggaacagggcgaataacaggagggagccctacagtggcctaatttaactcattcacttttagacaatgttctgccctttttcatattatagttgggaggtcgtcaaaattcactactttctaaataagctaccaattcgacagtttttcttccccaattttctagtcgggaccaactctaatcctactcctttttacaggactcctaagtcctgtaaaaagattcctaagacagcacagagcttgacacacccagatccctgggtgctctcaggcaagtcactcaacttccgggctgtgtgtatctgtaaattgcagttctttcaaagatcagttgtgccatagcctagatgaagaggcagcacactgggtacgccacaggcacttgggttttctccttccctactacctgcccccaccacttctgtgtctaaagatctcagccaccccgccccaccacctctggttgaatgaataacaaccaaaaggaagaaccctgctgaccagtgtcaaaggctttggtggtgcccttcagcacctcaagggtcagggctgccacaatgtcagcctgccgtgcaatagcactggctcgctctacagcttcacagcccagggatgtgatcatctgcgtcccattgatgagtgccaggccctgtcgggggagagagcaaagtttcctactgtgattattgtaacgaacctacatacccgtggattttgtatcttttgctttcataagagaaaaattagccagtcataaacataaaaaattatgtttatgttttctaaagtgcgggacagtaatgtttatttaagcccttgaagaataagtgctatcatatgtgaactcgtaagcacaaatctggcaaatgaaacactcattaagtatccacattcccaaagcagcaatgcagctctggagtgtagactgcacataaaatctttagtgcattaggttgcatttcttccttaaattcttactgttatagatgagacatccaaaaaaacccaattctggtccaagactcttagcttagagcattctaaggaatttgggctgagaacatctatctctcagagatcctaacagaactcgatttgaagttttaagttcctaagagaccccctatgcattgtggcatgaagaatctttttttcttttttgagacagggtcttactgtgttgctcaggctggtgtgcagtggtgcgatcatggctccctgcagccttgacctccagggctcaagtgatcctcccaccttagcctccagggtagctgggattacaagtgtgcgccaccatgcctggctaatttttgtcttttttggcggggcggggggggcgggtagagatgggtttcaccatgttgcccaggctggtctcaaactcctgagctcaggcaatccacctgtctcggcttcccaaagtgctaggattacaggcatccgccaccatgcctggcataaagaatcttttaaattcttacggagttgccccttcttcagggatgaactttaagcaaacatgcaagttgcatttaaggaatgattgaggctgagattaaaactgaaaatactgcataaataaaaactcatgcactatgaacatatttttcttgagtttccttacctcttttggttttaaaataactggtttcaatccatgggcttctagcacctatagaatgattaaaaatatgaaaatgggtatcaaatgaaatactagcctatttccaatatcatatggaatccaataatagctctttatgcccaaaagtccatcttataagaaatgagacctacaggaactggctgtattcatgttctctggtctattctctagttctgttctttagtcatgaaagcagacttatctttcaattaatttttgtactgaaatcaggggctccattgtctatagaatcaaccctaaattttggtttctacgtccttcgtgttcagtttctaccttagcagttcaccttttttaccactgcctcctgacatgcaggcatctgacacacatacatgcatctgtgttgtgctcaggctggactcctcccagttcacgctcccttgcctccagagcttgacctaaaaagtcctctaacctcattagtgcttcatttaaatactggcaaaacctcagagcagggtttttgtggcacatgtgtgagcaccaatgaagaggggattataaaattccctctactagatggagacagaatcccgagggggcgatgggcagaacaatcttcccttgaaagaaaagaagtagagagtcattaagttaaggtctctctaggaaagaaggaaggggaagttaaggtaagagaaggacagagctggtcccatttactacgtcaatttcacagattcaagtatcagctagaggctggattctgttctgggtgttggggtcggtctaggagtaggcaaaatgaagacaggcaagctcagggcaatctgagtaatagtacattcagtgcttgaggatgtgggaagtgggtggggtgcagtggaaaacaatcatttgctaggagagcatacaggagggtcacaaggaactcgagaggtaggaagtggagcagcggagacggaagcttcttagagggcttttctatttttctttttattttttagacacctgtctctgtaacccaggctagagcgagcacagtggtgcaatcctagctcactgcagccttgaactcctgggctcaagtgatcctcagcctcctgagtagctgggactacaggcacatgctactgtgccaggataattttttaattattttatgtagaggcggagcctcgctttcttgcccaggctggtcttgaacttctggcttcaaaagatccctagaagatgtttcagaagcagaaataggagagagaagaacgtatttgtaccagaccctctgcccagaacattccttctccaagcaacctccagtttctgcaatgcagaaccacacatagaagggattaggggctaatgtgacaaacaagacccttgaaagccactttaagtttagcctttatcctgaggacaatgggaagcattacaagttttcaccagaacatgcttgaatttgctgtttagaaaggtcagggtgattgctgtatgagggtggtcggaggaaaacgctgtccatagtgaaataatcaaagacaagcctgcacggaagacagatgatctagaagctgcatccccaaaaggattatctatttccttagctgtttcctcttctggggccctctacccctttcacttactatcagggaagctcccagcagttcatcagggatgatacattaaatgatgctttatgctgctgtaatgaaattcagacacagatgaagaaaacaccaaagctttatcaaaaacaggcttttttgattagccactagggtaagagcttagggaaaaatgaaacctgctttccagaagccatatagtaagtgctatgagccataccactgcacctgtgagaccagctcgtaattaaaattgtgagagtgtgtgacctccctgctaggcactggaaatgttgtaggtgcaggataaatagttgggattaacagtagcagttatgccttttcaccaaggtgatgaccaaacccagaaggctgacagccgagtatagaagctaacaacagtcttgcctagtctgacttctaagctttcgctgtctcatctgtaaagtaaggatagtaggagctccctcactgggctgttgtaaggactaggtgatacgaagcatgcaagcacagttggtgtccaacctggggcaattgctgcagatagaagctgctacgagtcttacgtatttagcatcagcccagccactcttcggagaccacatcttcccttctccaactagcccaagagcaagatgagagagtggggcaaggtctccactggcaccaacggttcctttctctgggacatagggcaggcaggaggctgggagagaagtaggcagcaattagttcaagagtactgcattctgactctccttgctttgccagtgagtgcccccactgcccccgcaaacaccctgcctccaatatctatcactttatggcatagatacctggggaaagacaattcttcaaacccagctaatatgtaagggacctggatgaattgttatagaaagaaaaagaggagtgggggtggagaggaagaagagagaaaggtaaagaggagacagggaggaaaggagggctggagggaaagtcagcaagaaggaaagaagggccaggcgcagtggctcaagcctgtaatcccagcactttgggaggccgaggcgggcatatcacttgagcccaggagttcaagaccagcctgggcaacacggtgaaaccctgtctctgcaaaacatacaaaaattagccaagtatggtggtgcatgcctgtagtcccaactactgggaaggctgagatgggagaatcacttgaacccgtgaggtggaggttgcagtgagccgagattgtgccactgcactccagcctgggtgacaaagcaggatcctgtctcctaaataaataagaagaaggaaggaggacacttctggatgcaaagccctcaaaatctgtgtgctctaattcaacctagtacttccacttgtagaaattttagcaatatatcttgaagaagtgatcaggaactagctgtttttttttgtttttgtttttgttttacagttcattgtttattataggaaaaatagttagaaattatctaaatatgaaattaaaggacattcatttgaccaagtttagccattcacagctattggaaaatgtttggaaacattggaaaagtaatataaaatgttccagaaggaggtggtgcaccaaagcattaacaaggtttcccaggggaagagaagtcataggcaactgagtcttttcattttgccagtcacccagtctacactccactgccccttatcaatgaacacgtgccattttctaagaaattcccaaaaagttaccaaaaaagaggcttcatttttctgaaataaaatcagattcctgacagcagcattttttccaactgttaaataatgtcagtcgggtatggcagtgccatccacagaccaacttctggctgatagttcacgctggggaattctcagaggtgccttagactcccccataaagtacaggtgggtttaaggttggagtgctactatctgggattttagggctatatagtatggcagaccaaggtaggagtaaaaataggtgttggaatcactactagtctgttcagcacagccctgtatatacaatagactgctcattgaaattgctctccactcattggcagatcctattcacgtgccacttgcgtccataacatggaaactcaagaatgtgttttgctgtttgtgactgcctcagcactgcttctcctccctctccttcccctcaaagagttctgagtctccactgacctagaaggcttgtccttacctgtgagaagggacaatacctccaatcactgcaaatctggtttccacattagaattttcctatccaagaagaaagctccagagttgagttccctcatatcctgccactacatggcttcttgattacaagggagtcattaacctctttgcatttctgttctacccttgcaagaagttggattttagtgtgcaagtctggccatcacctgggggacattacgatttcagattcccaggcaccaaccagaactactgaattggcctttgggatggacattgatatcatgcaatttttctaaccatattcatgtaccacttaatccaagtttcttctaggcaggcttgaatgtaagggcagacttaagagtcctgttctagtcttgaagatgacaaatgctagggaaagggtcttgcagaataagttaaagttcataggagtaagcatctctgatcgactttctcctcttcccccatctaactatcctactttttgccaacggcatttcccactcatttcaattatttcatgtcttctcaggtgattcctcagcatctggccaatcaattctaccctgtggtacatgtccagcctccacagaggttccgtctagggagccattgcattaccattaaacatttctatgacttgtttgagggtctccagggaaatgccactgtatcctttggctaagacattgatccttaaagccaagagcatccgacacctctcaggacttagtggtttcccaacacctgcaaaacagaattgatgttttctccaagaaaagcaaactcctttttgtctcctgacaattgccctccctccctccaatctttcccatatgtagcccagcctgctattaccttctcctctcggctcaggtagttggactactctccctcctgcctgtctgacttgtctgagccccatcttcctgacaaaggttaagaccccagttttgccttctcaattcccctttttgggaagcttcagtgaacccctgacctcccactcacctttagaatagcatccagagtccttagcacaacttctttaccatctgatgggtgctgattggtgacagtctttcccatctcactatgcccatcctaactcattctttgagttccaggctttatttccttccctgaatgcaacagtgtctccatggcatgggacatgtgttctcataccaaccccatccctccacctggcccatttctttttctgaggctcagctgattctttctgcatcccttctcacctcggcacctagttcttgaaatgctgttttgagaaactagctttttgtgactataactggataactcacactgtgctgtgctttattcatctaatcacacaagatccaggaggcacccaacgttttgacttacctgaagaatgtgagcgtactaagttgacctgaagctccctgcaacagaaaggtaaaaaccctcttagatacattgcagtgagaaaatgttccctcagctgggaaaatgaagggaatcatggtcaaatccttggagggacagaaggaaggcaatgggagaaaccagccagcctgggttttcaataccaactcacatgcaaggtaacccaagcctcatcctctgatctgtaaattggggcaaatgtgaattatttcccttgttgaaaaatatttaaaagatgagggtaggtggaacgtgaacatatgtgtgttttaaacttactgtagcttattgataggaattacagttctggcaaatttcccaaaacctgtagtaataccgtaaacaactagaataaaaagagacattatgcaattaaatgcagggatttttgtttgtttataaaaatatttataacataaagataaaaagatacctgttttctcttttatgatgctatctatgacctccctggatttctgcaccctcttctcagctgttggggtgagctaggaaaatgttgatcagaactgagcacgttaaagacttccacgggcaacaaaaatggccagaggacatctttcccctccctccccatacctttattttgtagcgtccctttcccaagttgaccagatcctccgtggtcagacggtctccatctaactcgatgtactacacaaaagaaggggatctcagtgagtctgaacagcttgattattctaaccagagtagggacacctccaacagaaccaaactgacatttcagagatctgatcattgcgtgaaacatgaatatgctaaataaaaatgcatcttgcataagaggaggcttattggtgcaaaagcataatgcctttccaagccccctttccccactacagagctggataaagccctgtggttggttggtttgtttgttttgaggcagagtctccagcccaggctggagtgcagtggtacaaacatggctcactgcaacctctgcctcctgggttcaagcaattctcgtgcctcaggctcctgagtagctggtattacaggtgcacgccaccatacctggctaatttttgtacttttagtagagatggggtttcaccatgttggccaggctggtttcaaactgatgacctcaagtgaactgcccacctcggccacccaaagtgctgggactacagacatgagtcactgctcccggccagccctgttctaattgacagtgttttctcagaagttttcctggctttcctccctcttaaataaggggccttaattttctgaagaaaaagaaagtggtttgaagcttaccttttcaggctcccggtacttgctgtatctgtatccaggtaaaggaacatatagggtggtgtggaaaaacccccagccccaccatctgcaggagccacccccagagcagaccaaagagcctgtgggaaaggatacagataaactccttctggttgagatggaatgaagtcaggagacatggcatcaccctctataactaaaacaatgcacgggagactcgttacagatcacattgtacagccatgttccccctgttaaaccaaggaacggcccatcattggcccatgaattctatagaaatggctgctttaaaactgcttcagatggtttcatgtggtcttagcatctttatgtaggaggagagcaaaaagcagaaaggtaggtgtcagggcagagaccaggggtttagatgaaagttcaagacagaattccacttccatccccattcccagctttattttcttcaaaatctgaactgatgctatgctgccggcctccagtctgggggtgggggtggattctttcgaaaggcaaaataacctcaaaatgattcagtcactgcaaagggattcagacctgcagccttgagtaccagtctagtctttgttacttactagctgctggagtctgggtaagttacctcctctctgatttctcctccatcagaggtggaggagaatcctacccatctcacagggttgtgtgaggacttaagatccccagaggcgtggcatgtgcctgcacagggccagcctcggcaagcctgacctctgctcatcattttccctgaggtgggggttcaatgctgcaaagactaagccaggccaaaccgcacccgttcgcggccctctcctgcagccactcacccacttccacgaactcgttgttctctagggccacctcgagccggtcctcgttgtccagcaggcccaggcccttgcaccggcgcacaaggaagtgcgcgtcatccacggaggtgaagccaccattgtcgggcttattcttgatatagcgcctcacggcctcccggcccagccagcccacagtgagctgcgcgtcctggcagggcactgccagccattccccacgtacgtgcaccgtgtatctgggcatggctccgctgcagcctgaggtcctcagctggtcacaggaggggagagctttatgcaggagtggctaccggggtgtggtcagctggaaggatgagaatagactttcaaaccactccccctccttcacctttactcattcatgcattggacaaatatttattgaggtacctgctgtccctttggtttttgtagccgagcaggggcaggagcaggggatgcagacgggtgagcctcctgtccactttccatccagacctgctgccagaaaggcctggggtctcccaccctgggaggtgctgttcttgtccctgatggcacctacctgctgctggccccctttcttcagggtccccaatttctctctcttccctcgtttctgtctgtgttctctgccattaagggcagcttataaacaccatgttcctgtctttatctgagattactcaactgtaaaacgtgttctgtgggccaactgtgacactgggagtgtggcaatcgtcactcatttgtttattccacaaacatgtattgagcaactacttcctgtcaccagtaaggttttctgaagctttctttccaccagggaagactgcaaaggccagagatcattttcacaactaaaggtcgtcatctgggcaaatgaaccactctgacttggagattaggggctcagacagccgtggatgagagtccagctctactacttattagccttgattaggtgagtattaccctgtatttccttatcagaaggcctaggggctaactgcctccttccagtatgtgcaggatttacctgtgatcaaatcctggctctgccatttgctaagtgtatgacttgtgggcaagtgatttgacttctctgggcctcagttgcctcatctgtaaaatgaagataatggtaatgatgatagtatatatgtcaaagggctattgggaggatgaagtcagttcatatatgtaaagccctcaacacaacacctggcatagtggaaatattatagaagtgtctgtgattactagtatctggacaccaatactccatgtaactataatgctttataaatgttctgtatgaatcataaagctataaagttccataaatgctctttcatcttggcacatggatatccctcacccaattaagcttcttcaccacagataactgctccctgcacccgccttattaccattggtattaaggaactctggatagcatgattacggcatcaaagatatgcagatgctattttgactaagcagcactttcatcgtaagccaatttttagagatttgattattgcactagaaaaatcctgaaaagtctgtttccactaaatcagacttctgttgggaagagagacatggttgctcaagaaaaacacctatactcatacccagagtccctctcatctgctgacagcttcccaggaagcgtacaaatagctacccttccttgagccttattgaaacttgctcttcagatacccacaccatgactgcaccctggaatttgtcatgacctgaatttgacacacttaagaaatcaccctggaatctgtcacgacctgaatttgacacacttaagaaatctgaaattccaatattccaccttctgaccacaaccttccagctgcccttttactctagtcactgccagccttggacctgacctccagtcagtctcttcatccctccacttcccaccagcatcctcacttactatcctaggaagctggtccctagtcgctcactggaccactctttcttaagcactctgagattcctttgcatccctaaacttatgtctacacttggaataacacccatccccttagtcatcacaaaaaacagccatctcttggaaggattgcttaagcctaggcagtcaatgctgcaatgagttatgattataccactgtactccagcctgggtgagagcaagaccctgtttaaaattaaaagaaactgtcatatctgttcacccaggcagaagacactgatagagaaagtcctgtgatcacatagtttgatggtattgcccttttattccatccagcccacagtgactttccaaaactttgacccctctgccaggaccttctcgacatccacctctaattctcagaaggagaccttacttcctacatctgtgagaaaactgaggactgcacatgtgaaccaccttaaccttctgccccacccccccccattcattccttgactcatttaacaaatattttttcaacacctgttatgtaacaagctctatgcgaggtgctggggatgaaataaataacaaacacgctctacattcatgaggcttactgtctggtgatggataaagctgtataaagatgactaataagaggtctaactatcttcatgctgcaaaacaacccaaactactgcatgctccagttgactgtcactgccgagaccagcttctgagtaatttcagcccaggcatcagactagtgagggaaggagcttccaggtgataccacctcccagccatgtaagtcacccccagcagtttgaggattctaaactcaggccctacgcattgtggagcagagtcttccttgttattctttctccagaaacgcactttccaacttcctttgcctgtctagatctttcttactccttaagcctgtttaggccttttcttctccaggaagcattccctaaattcttcagttgggttaagtggcccttctctgggatgccttagcatagctcttctttagtattatctaggaaattacttgtctgtgttcatccataattatgtatatatccgatctggcacaaagacatttagcatatgcttactagctgaactgaagaaatgtgaactgcaccgagctggcccctaggtaaaccagcaagcaggattaagtttgtctttagcgccctctaccttcttcacagcacccttccatgccacccttcaagcatcccttgtcacaattcactttcagtatcactaagtgataactaaaaacgacattaacttacagcttgtcttgtaagctcatagggcagaagccttaaagagcagtatttgggtaaatggatttgctaaagtctaatcttagaacatgatcttatgccagtgatttgagctgaactcccttctctgtattcttggcaaggaaggcaagggagagcagagcctggagaaagaatcacactgcagcatagataagtgaccaagaggacagaacgaggcgggtggattgcttgagcccaggagttcgagaccagcctgggcaacatggccaaactctttctctacaaaacaaaaattagccagacatggtggtgcatgcctgtagtctcagctactccagaggctgaggtgggaggatggcttgaacccagaggctgatgctgcagtgagctatggtcatgccactgcactccagcctgggcgacagagtgagaccctgtctcaaaacaaaacaaaacaaaaactgctagggagagtgagagccagggaaaagtcaggattccgggaataggcaggaatatgtctcttccatacctgtcccaccttgggtgttcactcctattgtaactttagtcactgcattagcactttgaggggttatttggtcaggacaccgctccccacccccaccccatgccaacaattatactctaagacaccattcctcttacacaatttatttgaccagaggtggacccaacctgggttagagtctcacctctgggaatttggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtccgtcccaggttctgataatctccccatacctagcatccttaaaataatcttccttttccctttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagactgtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgcgcccggacttatgtttaaggttatttaaaaagcaaagcaaaatcctaaccatgttgaatttttgaatctgcagcagattcaaattaatgaatttaaatcatatatcaggtaaaatactaccttgacatattttgtgatcatactgagagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagagaaatctctaaaatcatcaatacgcaggtctttaatccacttttaagtctttccccaccagcattgcagtcacgggatgcatgcttgctttgtgctcttggtaggttcggacagcttgatcatgggatttgtcaaaggcagcaagatccctgccaaaaaagaaaaaattgaaaagaaagaaaggcgagaaggagacagaggaggagaaagggagggagagaagaaagaaaggagggaaggggttcagaggaaaggaaaaaggaaggagaaagagaataagaacacaagtcaatacccaagattaaattaaaggatgtcagcaggggtgacagccagcatcacccaaataaggcaccagtcccagccaatcagatgggtatggtcctgccacagggtcccagagacctccttctgtaccagagactggcctttatactggcagatcagacattttgcagcaagttacagggaagggctagagtggctgggacccgtggctatttaccaagcagcatggaaggattttattatttgaacagagtcctctcatctcctggctaaatatcagccctgtatgtgagagtgagcctcaaagcctttctttttaaaaactgcttttaaaaaaaattttttaatcaagattttaagagtatgaaaacactaaaatttatatagaatttctgaaaacttcaaataattgagaataaaagtcctgaccacagtgaaataataaatacataataaataatacacgaaataaataataaatacactaaataaaaaggacctaccatacaaaaggtaggattagtcatttttaatgtaactactataaacatcataaaacagaaatacttatttttcccacaaaaggtatactcttatttattttattcattttttttttttgagacagagtctcgcactgtcacccgggctggaggagctggagagcaatggcgcaatctcagctcactgcaacctctgcctcccgggttcaagcgattctcctgcctcagcctcccaagtagctaggattacaggtgcctaccaccacacctggctaattttttgtatttttagtacagacagggtttcactatgttagccaggctggtctcaaactcctgacctcgtgatctgcctgccttggcctcccaaagtgctgggattacaggcgtgagccaccgcgcccggcccaagtatactcttatttaaaaacctatttaaagtatactttactcaattcaaagctagatgggttttaattagggaaagcatataaaatatacttaaaacttaattttgtggtcacatcaaaaaagagataatgacttattttgccaagttttatgatattatatggccatcacttttgatggccaaaactgcaattacttttgcacccacctaaatacttgtgaagtaaatgaaaagcaaacaaaagtaatcatggatatttatggcatgatttttttttccagaatttggacaaaattcataaagaccttgactgagatattcttgtatcttgctgtcaagatacaacttatccccctctcactaagcattcctttattatgtcaagcaacctacccttgacctctatgcaacatttgaacacaaaagagttagctttatctgcttatttctccttacatttaacttcagactctctttcttgtctatacctacccaccaattatcttctagttacctttaaaaatctttgtgtatataaggctatctttgatttatttctattttatcagtatctaactctatttgatccaaaatagtaatccatatataatgcttctaaaaagaggaatgaaattatttcacattttaaatatttataagtgtgaatccctattccaaaattatactgataaactttaacaaattaaaaaatattgtcatatagattacgtttaaatatttgacagttttcttcctgtttcttagatgaattcaaagtacggtctgagtgggttcttacttgaataagggccgggtaaacttcattcttccttgttcagttgccatctttagcgccaaaggaattgcgtcctcccacttggattgaatgcagagccgcagccatctaaaaggaggatttggggggagcatggagtagaaaatgaggaaggggcaggatatgacaggtatatcttaatattacttctgtagtgatatgaataaccccactatagttatactgtacaccactttatggtatgtcttgattctgagactctcaaatccttatatatacaatttaataattggtgaagagaaagaagaggagctggttcttgaaaaagatcatatatttttaaaggtctggatcaggtaggtgctcacataccttataaatccaatttctgaaggaattaaactttggtttaagcctcacattacaaatttgaattaagaaagatcaggtaggtgctcacataccttatacatgcaatttctgaaggaattaaactttggtttaagcctcacattacaaatttgaattaagaaagattaacatataatagaataaaatatttctaactattcccatttcaaagtagatttagttggttgtggagaaagcctatttaccacggaatccttcattctaattttttttttttcttttaaggcaagagaggtttagagcaaagtctaacaaaaagattaatactaccagattacatattgcaactattccttaaataccactataagtatttatatagaagcagtcagtttgacaaggaattctcaagactcaagtatgtctcatactctgcattccctttctccatctttcaaaggagtttagttttctgctttcttccacagagacaagttaaaatgatgtacctgaatcgtatttcagaattgttaatggcattgaagttgtacacctcttgcattcgctttatgtgccccaatggaagaggtgcctaagagcaaaataaagaagtataccgtatcatttcaacaggattccttggaagaaaggagctggagagaaatgcatagccagattaaaatcctaaatattttataatatagaaataagtcagataaaaataaaagaaacaaattgcacactaagtaaattctgtgcaaacttattccagatgaggatattctactgggagcacagggataatttactttgtgaagtattcagcattaaatgagaattgctcttcttagactttttagcatgtataaatattatctttcagacttttcctagagtttttctagttattctctataacttatatatcttaaatgcaattccattctccagatgaaatcatagttccttaatttttgcctgattccccctagctttatctttgtatatttcctctgaaatccctgttaaattatctgcatacctacataatagcagttcttaaatgtttgtattatagatctctttgggaatctgatgaataatgtggactctttccctagggggaaaatacacttactacatgaatacaaacttctgtatacaatttcagggggtttataagcatcctatccctaccttaactcaccctaaaagggaggacaagtttgggtgaaggaaagaaaaaagatgagttcagtttggacaagcagagagtttgtagtgcctgtgagaggcagaggtgcctctaggtagatgataactctcccctccaaccacgacctccttaccttacaggactccacactcactaaccaatctctgctttcatgaactactaatcctgtcgctaataatttagtccattagccccttatggacacatgcaactccaagtctaccctggtagaccaactggttaaggtcatctccaaggctccctgacttgccctaagttttgctatacccattccagaatcaccctaccatgttctctctctctgtggccctagaccacccaccagtggtagagcaatttatgaaaccatgatgacccgatgcactaaaaatagattctctctttgatgggtcctttgttgcgtcaaaatcctattcctaatttttgcatcaattccacagaaaattccgctccaaatcttctttcttctcaaggtccttagactgaagacttccctttcatggaagtctttaaaatccagtcattggtttatctcaaaatgcagcaactcctttcggtttcatctattctttcaattgcctagattcgaaaccttaaaatctgcttggattctttactgtcaacccctatagccagtcagtcacagagctctgtgctttcaccctgtgtaaactctttctcacgtcctgttctctcctctccccgcaacttactccctcaagtccggtactcctgccagtctcccaactagtaacttcaccaccatgcaaccttcatggccccagattagttttctacaacccagcatttcatcccgactcttctgctggatttttaaaatcttttcctactgatcagtgtaagatctaaaatttcttagcttagcattgagagtcatcacatctggtcctaccagcttttctagtgttaccttcactgacttccttacccagtgctactgtttactccagcaatgctgcagacgaattccagcccttgctgctccctccaccttcaatttctacctccctgctagccctgggggtgcaaagcaagtctcctccaaaattccctctctgatgcccccagttggaagagtctttcactaattaagtttttccaaatgatacctaaagtatgcctccttttattgctaatgtttttaaaaaaatttttttatgagatggagtttcactctgttgctcaggctggagtacaggggtgtgatctcggctcactgcaacctccgcctcccagtccaagtgattctcctgcctcagcctcctgagtagctgggattacaggcacctgccaccatgcccggctaatttttatatttttagtagagacgagatttcatcatgttggccaggctggtctcgaactcctgacttcaagtgatctgcttgcctcggcctcccaaagtgctgggattacagatgtgagccaccgtgcctggcttattgctaaattttgcatgtgttccccttcctactagattatacgctatttgaagataaggtatatcctttcttacatattttcatatttagcacaatataaaacacagtaagcattcaatgcttttttaaagaaatgaataaattttataaatgattttttccccattagtttccacattaataatcttttgccaagttgggtagaacataaatgctgtgcctttctgtccattttaatttctaagattttgagctagtacttaccctctggagcgtctgtgctaaaaactcattcaattgatgagaagagagatccttcaggtctgtggcattgaatgaatttaaatcatcttctttggcctgaaataaatgttacctagttatttttgttcaagtacaatttaataatacttattggtttatctgacataaaagtaaaaattgagaaaaagaaccatatgaatgaacaagattattcaaaataaatttaagcctgagttacttaaataatcctgagattgagttactgtaatttaaatagctgatatgactcctagaatctatattacttaagaaaaagtagattatgggtaggaagagtggaagaaactgttgacattcattgtaccattcgaggtatagaaatttccaaagcaaagaaacatttcaaaatgtatgcatgtcaactaatctatagaccaattcaaaaaggtaaagaatgaaatcgtatatttttaaatattacattaataaattggtaaggccataaactaatgttttcctccatccccacatattctgttttccccacttaatcttagaaaccatctaagaaaaataaaaatgagtctgcacttttcaaatttggatttactctcaaaaatctttgagaagatgattaagcaatattaaataaagcttataaaaataaggatttttaaatcttttagaactacttttataatcttttaaactagggcttttgttactttaaaagaaatatatgcaaatactaaaaaatcaaataggacagaaggaaaaattcttttggatctgctccctgtctccaagtactactcctcagtaactaatattagtagtttctgtatatccttccactaaatttaatgcataggtatatacccttttaaataaatattttgcatcttccccctcttcagaactctctttaatagcaatacttcttttccctttacaacttatccttaatatgagaacttacagctccagctcattttctgtgcaaaaacctgcaaatctaaactatatattaattaaggatatatttatgtggtaaaaacataaaaagcaagagaatgataaaccaaaattcaggacaatggtaacctggatgggtcagcaaggagggtggagaggggcataagatggggagggatgctacagaggtaccgctaagattttacttcttatgctagtggtgggtcacacaattgttttatacaccatatgaatatgttataaatattcttttgcatttatttactatttaagacaaatcattgagaaataaaatacataaggaaaagagtgcattagtgaatacagtgtcctgaatctgttcctaacaatgcctgtttctactaatattgaagagttgatcattatccaccttaactgctgggcccaaaggaatatttgagcagaaattagtagcagttttaactagcaccaaataagctggaatacatttttcaaactaaaacagagaattttaatacactcacactgttaaaaaatcctgtttcccatagaaatctcttatacttttcttcatgacaagtttgtcaactacacaaaacaggttttaaaaggcaatagctgaactgattgcacagctggaggccattatcctaagtgaattaacacaggaacagaaaaccaaatacagcatgttctcacaagtgagagctaaacgactgtatattcatggacataaaagtggcaacaatagatactgggcactactaggagtggggcaagggttgaaaaactactgggtactgtgctcagtacctgggtgatgggatcaatcataccccaaaccttagcatcacacaatatgcacgtgtaacaaacctgcacatgtgtcccctgaacttaaaagttgaaattacgtaaaaaaatgataaatctgttgcaaattaataggaataaaagtattcctaaatcttctgttatttttcattaaagaattatcaagggctcatccttactttggcttcagtaaagggttctattttagtacatatatgaagaagctcctctttaagaagcttcatagaaagtgaacaaagagcaaaagtgcttcgattctttgcaccactaatagtcagcagctggtcacccaagatcattttagatttacctggtatgtgaaattgccatattggaagcagtatcttataaatgatttaaaaggaaaagaagaaaggtaagatgcaaatatttttgcatactttttttttttaagagttaagaagcaagaaaaatcaggattaatgccttcaacatcaatttttccccccataaaacttaattttctaggctgggcacagtggctcatgcctgatgcctgtaattccagcactttgggaggctaaggtgggaggatcactggagaccaggagtttgagaccagcctgtacaacacagaccctgtttgtataaaaagttttaaattagccaggcatggaggcacatgcctgtagtcccagttactcgggaggctgaggtgggacaactgactgagcccaggaggttgaggctgcaatgagccatgatcacgccactgtagtccagcctgggcaacagagcaagaccctgtctcaaacccttaattttctatattgagagtagatataatatcaccttagataaacctgactttcaaatagcctttccaaatataactgtttgtgatttaaagtaccctccctgcttcatgagtaaagacatatttgcacaattcaaaaaggaatcaaaaatcacacattattacttacagtaatccatctttgacttaaggcaatacaagcatttgtcagagtcatatcataactgcaaagataaagattacattgtttaaaaatgcacgtgcttttgcagaaatgcagttttaaagctacagtacatacttaaatttcaaagtcccttttaaataaggaaaacaaactccaaagtgaggaaaataggaaatattttacctaacttacatactactggcatcatccaagaactcacaaacccaaatggataccacattaatgaaacacccatctatcttttagaaagaatgccaaagcacctcagcaaaagactgtcatgtgctcgagtagtatatgctaaagtagttggaatcagttgagcatatttagtacatggcaggaacagttctaggcactcaagacaacaagatgaacaacatcaagtccttgctgtcatggattttacttggttgttccaaacatctaatcatctaacaaacctgcaagcacctgctacataattggcaccgttctagatgctagacccttgagagagcccgataccattgcctgatgatttcattcctttttagaagaaaatgaaattaacacatggtaattgttaagcaaattataccaatatttgtgtgttctcaacttagaaatcatattttgcaacaatgggaaagaacatgtagtgtgtgcaaaattcttgcaaaacatccctctttctccgtaaatcatgcttgcttgtactgaaatgcttgtattagggaacagagaggcacctgccccttagagcctaaatgaagtaagttttgattagaagttaccactgaatctcccttaaagagagttgtgactgggactccgtttgttccctaggggagacaataaaaaggtcaacacagctcccacctcgaagcagctgccagtttattacatgaagtgtcaggctgtggactgcaggcatgccattttgtcttcaagaacaggtgggatcagaggtccttgactgatcagaatacactgctttcaaccaaaacattattagcattgatttcttaaaaaataatagcaaagtagaaaacctttagctggtctgtttcttcgtgtcctgaaacttccttattagtgtaattaaaagtactaagttaagaattagcctgggaaaggaccctacttatggcaaagtcttcagaaaagtaaagagcaaaaccagatatgtgccttgttctcatggtgctgacagtatagcgaagaggaaatactttaatcatacgaataaataaatgtaaagttagaactgtgcaactgctacgaagagaggatatagcactaaaaagccctagaatgggagatttgacctggccagggatgtcaagaaatgcttccaagaggaagtggttcttgagctgagattggaattaactgggcaaagggctccgggtagagaaaacagcatgctcaggtactatgttggaggacatatggggagttcgagaaactccaaaactgccagtgtgactgaagcaaagggagctagagtgttaggagcttataatccccactaaaggattttgtcttagcccaagagcaaagagataccagtggagactgctaagcaggaggacaacatgacacatttgtgcttttaaaggtttactctagctttagtgtggagagtggctgggagaagtcagaacagatacaagtgcacagtttgggtgccagaacagtcttccaggatgtgaagatgtgatactgaacttggacagtggtagtagaaatggagagatgtggatagactcagatatttaaatacatatacaaatgatgagagcattataaaaagaggatcgtggaagccaagattctgtgctgcaatggatcaaagtattttctgtggtttgagattttctaagatactctctctttacagaattcccgggcacacgaatgattccagggttcctccagcactttggtattacttgaaagcaatcttaagggatctagaatgaaccaacgcccaaaaaggatcccttagcagcggtgatatcaaagaaacacttttgaagaactaattttccacccagatttccccaattttaaaagcaatgggcaaagccttctccactcctaaacttcctggaactgtcttttggctatatcaggcccctgaagttagagtctttgaaagactccaaactccaaattctatgcttttattctcaggctcctcataattctacagcacaccagactgctgaccactctccgtaccacttttaaattatttcttcccacagctttcttaacaatgaacctttgaaatctttttagttttccatttattttgctacctttcctctgtcctagctctaaaatgaagatcctctaaggttctacagtttacttcttgtattctcctttgtaagtcatctccaagacgatgtccaaatccatcaccattaaaattaatagtttcctcacccacaacacttaatattttaaaaaaaatacttttcattgtattataattacttgatacatacatatttgctctgtgagttccttattcatcatattagtgcctgacaataaatgtgtgctggattgagctgaatctttattacatctctgctcagtcatttttaatttcttcttttctcaccacagccaatcagttgccaatagattctagcccccaaacgtctcttctctcagttactcctttcttttccactgcctttgtatgacttcaggtcctcataatctctagcaaggctgttgtaaaaattaacgagataatgtatggcacttcttaatgaagtgctaggaaaaaaatctaaagtattatttttgctgataccttttttagacgttaaaagggtttactgatgatttgtgccacctgtttccaacacaaaattcgaaacattctatcgtaatcacccctccctacctgagctcctgtttcccaccacagcctatgataaccaggactgcccagttagtggggcgctctgaccacatttgttccatactcagaactcccagtaacttctcaaccaaacacttctcggcctggctgtttaaagtgctttacaaacaaacatgaccaggcctcatcttgtttcttagcttctctcctgctgctccctgaacatcaattaaactggcctgtttagtgtaagagaagctggtaggcaattttggtgatccaaaagaaaggcaacaagagaacatgccatggaacatgccatggtcagtgtcctcacacaactcgtgaaagaccagggttcaggtccgattgaaggagggggttcagtataaaaagcagtatattgaggccgggcacggtggctcacgcctgtaatcccaacactctgggagacaaaggcaggtggattgtttgagctcaggagttcgagaccagcctgggcaatatggtgaaaccctgcctctagcaaaagtacaaaaacagccgggtgtggtagtgcgcatctgtggtcccagctacttgtaaggctgaggtaggaggatcacttgagcctggaaggcagagggtgcagtgagctaagatcacatcactgcacgccaggctgagccacagagtgagaccctgtttctaaaaaaaaagaaggaagaaagcagtatattggaggcaataagactgccagggtttgaatctcaacttttactactcactagctgtgcaacctagggcaagacactttacctagctaaacctaacttacctccttgggaaatggggataataacttataacagtgttgtaattaacataatacttataaaatatttttattgcagaagtttgaaggaagatacaatagcttattgtctaaatccctcaccatccttgtgcagaaaggaggcactcaattacttgaagtgaaaaaccatatttgtaaactgcagaaattattcttttggcctcagggttaaggccaaaacacctaagaactctgctttcatcatttactagtaacagtttcaggaaggcatactattctttcagatattttgaggctctctaggagttaggagaatgagaaggaaagcattagcaggcaagtacttacttgggctttatgggaggcagtccaggagagtagagccaggcattccaatcaacttgattgagaacatcaacctatgaatagtaagaattcacagtttacaatagaatgccctttcctgtcaaaaaaaaatttaaacttgtaagtccttagatatataattttgtctaatctgctatatcaagataatttctaaatcttttttaaaaattaatattttaaattgatagatcataattgtgtatacttatgtgacacaatgcgatgttttgatatatgtactcaatgtggactaagtcaagctaatatatccattacctcatctaactctatcttctaaaatttatattcatcaccatactattgatgacttctctgaaataggaaaattctacaggtagttcatgtggttaagatcacatttaaaatagaaaaaatatgcaatgagaggttgagtcctaaagttctgaaccaatactactattagataatacaagttaacctaatcagtcaataaatagagatatatcgagcatgaaaaatagaaaaggtttttaaatccaaccttatctttaaaataggaatacaggaaatccttccagtcatcagtagttatgctcttataggaaaacttctcaacataagcttttaagaatcctaggaaaatctctaagagtaaaaaagaaaagaaatcaattcatagaaaggtaattatttgacattttgtgtgcgtgtttggcattgtactattaaccacagagaacagagaacattcagagaatagggaaatctacgaggactttcagagtgaaagaatgttcaaaaaaggaggtgggacttaagttgggccttgaagaatatatgtaattcagtggaagggagaagagaaattctaattataggtaaggggataacacatgaagacacagaaaaggaatgcataacccaagttctaaaagcaataaccttcacatgactagaaaggagaaaaataagactggacaggcagaatggatccaggtgacagacagccttccaagtcaatcaaccaaggagaacacctcaatgtccatcagtgggggatgggtacataactcagcatagctttatcatgaactagtatgatggcattaaaaagtatgaaacagatttatatgtactgacacagaagggtgtatgtgaaatatcgagcaaaacaaaacacaaatgcagagccaatatatagcatgacccattttttgtaattaaaataattacatgtatttatttgtctgcttgttaatttacacctagaaaatgatctggagccatttacaccaaactgctaacagtggttacccctggggagtggaaaggggttgatggactctcacttgttactcttataaacttctgtaccatagaaatgttttcagggaatacacactattatcctaatttcaaaaactgatgagattttttaaaaaatgaagcagcataatttaaaccttaggggttattaatggttttaagttgagaggtaactcagaaaacaatacagatttcttcagcagctacatccagaatgaattggaagtattaaatggaacaaaacaatagtttacaattcttcctaattctcacattaccctcaaaaagaaaaaaaaaatcataaaataccaactacttacctggtcctccaagcagttgttcaaggtaaaaaagtaaagcaaagcccttctcatagggaactgaagaataagctacatcagggtctatatctgtcagatcaaccacaagtttggtgaaaggatgtgtctccccaaatgtctttacctgcaagacatgaaataacatggagaaacatatagaaagactgctatcaccacgcaaataagctaataaggaggtattacttcactcagtggtgtaactttaggggaatctaaaacttggagactggaacactaggatatgttggcataaacttctggaagtctattaatagaatgcttacttaagtaatattctctgttgtttcttgctcaataatacaggctttattcttataaaaagactagaaaaatgatttaatgcctggtcagcaaatttggctttcaggagacaacacttaaaaatgacataccaaataagatgcaaacatagtaaacagctatattaatagcaaagacccagtgaggtcccacagctccctatttagaccaggtcatcaaaactaccttacatagaacagtgaacagtgtggatcaacacagtgttataccagcattgacttcactttccacacttgtaaaaatgactttttggttgctacacagtaaagacgcttttataaaaactcagtttttaacacctatacaactttggatgaaggtttttaaaactttgactcctttaccgaattctgtagttctccccatcctcccagagcattaaaatgtctgaacttttcaccaaacaatcgtccgcaaatgtggcgttccaagtacacagtatgtccctcatttaacctgaaaaaaaaatattttaataaaaacacggacacagctgagaagaaaagacatttcaatcaagatattttctttttggcttttctacagaggaaagcagttgtaaggcatgaccactacagtctaagccgactctggctcccaggcagtcaatccagagcaatgggaagcccagcccagcagatggcagcagggaaagttaagccctgcttctgctcttgcatgtccctatgttaaaagtgggagtatatcaggaattaaacttaacacctagactgaacctaacactcctaacgctgtaataagtgttacagaatttttaagaactatccttgttggccgggcatggtggcccacgcctgtaatcctagcactttgggaggccgaggcgggagaattatctgaggtcaggagttcaagaccagcctggccaacatggtgaaaccccgtctctactaaaaatacaaaaatgagctgagcatggtggcgtgcacctgtagtcccagatactcgggaggctaaggcacaagaattgcttgaactgagaggcagagagatcacactactgcacaccagcctgggcgacagagtgagactccatctcaaaaaaaaaaaaaaaaaaaaaatccttgttggctgggcgtggcggcttatgcctataatctcagcactttgggaggccgaggtgggcggatcacttgagctcaggagtttgagaccagtctgggcgacatggtgaaatcccatctctataaaaaatacaaaaattagccaggcatagtggcatgcgcctttagtctcagccactttggaggctgaggagggaggattgcaaccctggagttgcagttagcagagattgcaccactgcactacagcctgggccacagagtgagaccttgtctccaattaaaaaaaaaaaatagctgggcatggtggctcacacctgtaatcctagcactttgggaggccaaggtgggtggattgcctgagctcaggagttcgagaccagcctgggcaacacggtgaaaccccatctctactaaaatacaaaaaattagctgggcgtggtggcgcatgcctgtaatcccagctactcgggaggctgagacagaagaatcacttgaacctgggaggcagagcttgcagtgagctagatcgcaccactgcactctagcctgagtgacagagtgagactccatgtcaaaaaaaaaaaaaatccttgcctacctaagagatgataacaaagaaaaacaagagatacacacaaaacaattcagagtggtaggaggcagggaaaaaaaaatcagcaacatgaaccaataaagaaataataacaaaaatcttaccaaaagtgatcccaagttttgttggtcactagattccctgtccagctatgagatatttcatgtgcaatgacctaaagaaaacagtgtgattaatagtaagactgattatcttaacctaaaataacccattctactgtaaacactccatgttattcaatttttaaaaaatattagctgagatattcagggagttactcagttcagcaaaatctttaatttaaccaaagacatacacacacacacaaatatggcataaagtaattagaaaacaaaaattacttagaaaatattaaatagatatatcactgcattaaaaatatttcataaatatatgaagagatcaaaccataaaatttccttaaatctattacatctaattgttaataatgtgagaagagtccccaaattgtccattataaccatatctttaacataaaaatacaagggatattttaaaatcagttgacgtaatacttcttgaaggtaaggccataatgaaaaagtttaacttacattggagagtgacttgtcgcctgcctacaaaaaaagaaaattaccttagtattttagtaatcgaattacagactatctaaaagactgcctacctaaatacttagcatactggcactggtgatccactgtatttctactacagcacttcaaagaaggtaaaagagaccttaattgaaaaaacaaaaaaaatacagaactaaaaattagcatcattctttcttgccctaattctagggaatttttgcaatacaatgaaagccagtctatttgtgtctaacttccatgaaacatttcttctacttccatttttatatctgctcttatttacccatcactttctttctctcctattacccaaaatatatttaataaaactttagagtgtcctatgtgtcctgtgctgtattttatttattttattgctaatccatcactcattttggttctaagaagaatttaaagtagctcacaggcatattaaacatagcagcgttctatggccctaatcctttcctgtacatggtgtactgattttttttttaattgtacctacacaccaagtgtaattggtatagtctgattgtctggatacataatttatcaatgaattgttgttacacagcacccccatgcccaactccccaaataccgtgaacataatattctccttctccaaatggcctgattattttcctttcaaaaacaagatggaggcctggtggggtggttcatgcctgtaatcccagcactttgggaggccaaggcaggtggatcacgaggtcagaggatcgagactaccctggccaatatagtgaaaccccatctctactaaaattacaaaaattagctgggcatggtggtgtgcacctatagtcccagctactcaagaggctgaggcaggagaatcgcttgaacccgggggcagaggttgcagtgagctgagattgtgccactgcactccaacctgggcaacagagcaagcctgtgtctcaaaaaacaacaaaaaaaaaatgagattgtgataaccacaataaacaataaaattagaaacaaaggttacacgaacaagagaaaagatttttatacatatatacattattaggactctaaggttctttacatatatatacatattattaggactcctaaggttcttaagtttatttttgaaacgtcaatttcaaatgaagaaacatttacacttaccagtagagtaggagttacaaaagtaaggcaaggattctccatgccaccataagggaaggatggtggcaggaccaataggtcatactgtccccatacatacggtcctcccagatcttctgctattttaagcatagattcagtctgaaaaatcaacatatatatgtctgtatgccttaattatcaccattgtacatttcttaaaattgtatattgctttctatgaaggttacaagttaatgatttttatgttaaatgaaaatgataaaaagaatgtagcaactacacatctacataaaagacaatctggttctttccccgtatcacttaaaaccatcacatttcacactcaaaatcttcaatctactgtctatcacaatgattacaaaggataactaaatgaccaacctcagaaaactcataagcagacttttccacctgctctttctcagaccacaccaaagttcttgggccaatttgcctgcaagattaaaaagcttaataaaaaagaaattcatggcaaatgattcatctggttctaaaaagtagttaatgtcttgtagacaaacctttacagtgagagtgcatttaccatgtgcctatgggaaagggatgaggttacatggcgtgagctgcaaacctgctgttgtatatgcagcaatagatatgcagctctctaaggaggtacaggctgggccaaaggaaaaaaaccaagacataaggtgtccccaattagtttattgcctcttgtaagacctcttgagggtctcatttcatccctcaatttcacaactatagaaacccagtcacaactcataagaaactattttttttttttttttttttttttgagacggagtctcgctctgtcacccaggctggagtgcagtggcacgatcttggctcactgcaagctccacctcccaggttcacaccattctcctgcctcagcctccctagtagctgggactacaggtgcccgccaccacgcccagctaattttttcttttttttgtatttttagtagagacagggtttcactgtgttagccaggatggtctcaattccctgacctcatgatccgcctgcctcggccacccaaattgctgggattacaggcgtgagccaccacgcccagcgttttttttttttttttttttaaatatacagggtctcattctgttgcccaggctgagtacagaggggccatcacagctcactgcagcctccacctcctgggctcaagcaatactcccacctcagccttctgagtagctgggactacaggcatacactaccatgcccgattaattttttatttttttgtagagacatgatctcacttatgttgcccggcctggtcttgaactcctgggctcaagcgatcctcccactttggcctcccaaagtgacgggattacaggcatgagccacagagcccagcctgtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggactgggcggtgggggaaagaggagctattgtttaattggtgcagagtttcagttctgcaaaatgaaaaatttctgaagatctgtttcacaacaatgtggatatacttaacactactgaaccgcacacttaaaaacagttaagtgtgcttaaaactaagaatgaacaaaaaattaagaaggaagggcactttatttgtaaaatattgataaaatatcttacatttctgtaatatttgtaggcttccaagttctttaatatattttatctcatttgtttcacataaccaccctatgaggtagaaagtcagacattataatttcaaggataaggaaacagagattgagagtgacttgttcaagcttacatgagaatccagatctctaaaggtaagagcatgctcattttacaatacttgaaaaaataaggggtaactggtcaagatttttaaatgtaaaattaatttgttgcctacattttagatttgaatttttctagagctgtcagcttgatatcttgagaaatatgcaaatgattgaccaattaaccttgagagaagttcaagatgcctaagttttgatctttccacaaacctgaaaatttttccaaaagctcacctgctttctaaagctccaacaactaaagcaatcaggtagcagggtattggaacctaaagagggcaaacaaacgcacaccacgtgcttgcattagtgttcacaaatgttacacagtaagacaattcatatttaaaagtaagtaaattccctttcaaatctcctaatattagtagggataactttgcttttatacttctcaaatagttctcatctttaacatatagcttaaaattgtgatataaaacattgttcaaaacatctatttgccttttattctgctaggaacaaaagcttctcacacatgaaaaacaagatcacacatactattaaaaggtgcattttgagcatttctcaaaaagtaacctacaggaagcgcatttcccatatgtttgcctttttcctcctgacttttaaaaggttttggttttctttttttattcctttatgtttcaaagcactattggcatgttgtagaggcacacagagttacccggcaataagtagatgccaaagttatgggagcttggaaccacagaagctgcagtggaagtcaaattatccattgtgaggtcaattaagaaaacacacacacacacacacacacacacacacacacactcacgcatgcatacacattctgttcactgaaggtcaaagatacatgtctaatgaccaggactgtaactgagtgcttttttggtcttgactcctccatttgtaaacactctcaacagccacttgcccacactgtagtaccagggcaggtgaatgcgtaagaactcaaaagaacaggaagccagcccctgaactggagtgagggaaaatgaaaacaggtatgctccctattctgtctctacagcaaaccctctcttcaatacaacactctgtaagtagaacaaaaccctttaaaagcacaaaagaaaaaaaaatgaagaaaacaaaaaccaaaagctgttctcaaaattctgaaatatttcaaaagtaattttgccctctggatgtgtacaagctaactgtagtttaccgccaatgaaaacaaaaatctagaccctaggatcttactttttggatgaatttgtatattttcctgcttgggtcttctgggtcaggtgtttctccatcacgaatagcactcataagtgccaccagttctttagggacagacacctaatcaaggagaaaaatcatttctagtcataaataaaagcttctatgtgtcttaaaccatatatgtaaaataaccttttcttcccattcttgactatctaataaacagactatgaacacaaaaagtatatacatatacaaaaagtatatatatacacacacatatatatgaacacaacgtgtatagatgtgtatatatatgcacacatatatatgtgtatatatataaaacacatatacaaaaagtatatatatacacatatacatatcagttttgtaaataaaattagcaatatgggaaactggcttctttaaaagtgaatgtgaaatttctatccattcacccatgcacattaagagcagagttttggtagaaactggattaaaatcccagctctgccacctaataactaactgcacaaacttgggcaaataaatataacccccgagcctcagtttccccatcaagtaagtgtaaaacttcaaaggcttgctgcaaggaataaataatataagtgaagagcccagcaccatccctggcaatggcagccaccatccctgctcccgctacactcacaaaacagattcaaaaggacgttatatactcactgtaggacagaatggttttgaacaattttgttttgaaaacacacacttggagttacaaatagaggaacattttaaaagtagtaactgtgaaaaactaaaatttattgctaaaaactgtcaaataattttctctggaaatccatacggaaaagacccttatgcggcaaaccatatagtcatttaactgtgtatcctagctccatgattctgaaagtttgatttctgatgaatgccagaataaaggactcccccaagtattaatgatcaaacaagaatatattccagtaggggctagactttcatgttcttcttgcatggctcaggacccaaagctgtgactgaggcaggcacagaattagaagttcctgaaccagtgctacaacaattgtagattctaaagcacaaaactattcaggaaataattcggttcagccacctcccttcatttaggtggtgatacgttatatatatgtgccagctgaggttgcgaggtcataaaacttgttcaagtgtcacatcattatttatttatttttttagaaatggggtctcgctatgtcgcccaggctggccttgaacttctgagttcaagtgatcttcccacctcagcctcccaagtagttgggacttcacgcagttattaagtggtggagaagagccagagccctgggattctttgcctccaagtataatatatcactgcactatcctagatgtaatttggttgtgggatgatttgggaagcaagaaggccccataaatatgggttggtcctcattctatttgcttggtctaagtaggtctagcctccgggatagtgattatttagtaattacagtccgccttttccaaaaaggattagcagtacctaccaagggaataagttggaattgcatacagacaagtctggaatatatgcccactaggcttatatggctacagaatgcatttatagaaacttaaatcatgcaaatgtcaatttttaaaagttaagtaaaaattgttcctaagttcttatttctagatccaggattctgaatttcttctttttgtttgtttgtttttttgttttttgggttttttttttgagacggagtctggctctgtcgcccaggctggagtgcagtggtgccatctcagctcactccaagctctgcctcctgggttcatgccattctcctgcctcagcctgccgagtagctgggactacaggtgcccgccaccatgcccggctaattttttgtgttttttttagtacagatggggtttcaccatgttagccaggatggtctcgatctcctgacctcgtgatccacccatctcggcctcccaaagtgctgggattacaggtgtgagccaccacacctagccctgaattcctttttaaaagtcagattggtttccatttcctttttttcacagttaaaatgtttaaaactgcctttaaagtagagattcagaatgagtgccacagcctctttgtttacatatttcaggtagaatttcattaagaaaaataattctagctctaggaattcaattatcatctctgcttatcatttataccatatttactgatatgcatcatttaattgagttaataattcgtaatatttacctctgcagtataggttaatttcacagaaggagtgtcctgacaaggaaggattgctctgcagtggatggcctgaaaaagggagaaacaagaagaaatagctatttatctttcgcataagtcattaagaaatcattaaaaattgcagcattgttcttagacattaaaataaaaacagtcctcatttcttgggattttttttttttttttgagacggagtttcactcttgttgcccaggctggagttcaatggcatgatcttggctcactgcaacctccacctcccgggttcaagcaattctcctgcctcaacctcccgagtagctgggattacaggcatgtaccaccacgcccagctaattttgtattttgagtagagatggggtttctccatgttgatcaggctggtctcgaactcccaacttcaggtgatctgcccacctcggcctcccaaagtgcagggattacaggcgtgagccaccacgcctggccttttgggatcttttaaagtccaaaaatagattcttggacttttaaaaatcagattttccattttaatctatggttaaccctcacatttcagttgaagcatggagaaactcttaagcagtgtttcctactctatggtctgggtgacagtagtgcccagtgagaagcttttagaaacctgagaaaaaagggctctgtagcaaaacagacctgagaagtatggcatactgcaccactgtcttgcagagccactagaatattagccgcctgaaggctctgaacagacctacaataaagaaacctgtttgatttcttacatttatgttaacacaaaacccatttctctctggtttaacacctaatgggatgtcagtattctaatgaacacagcctgagaaatgttgctgtaatcctgacacttcaatcttgcagcaaaccttgtaagtaaaacaaagaagcaaagaagggagaaagaacagtctctttcaataccatctagacatattcattcatatcatatgcaaagtgtttctgtactgccacaccaatcgttattaacattggttccatccagtatgaccacaggccaggtgccgtggctcactcctgcaatcccagcactttgggaggctcagatgagaggattgcttgagctctagaatttgagaccagcctgggcaacatagtgagaccttacctctacacaaaaaaaattagctgggcatggtggtgcacacctgtagtcccagctactcaggaggctgaggtaaaaggatcgcttgagcccaggagttctaggctgcagtgacccaagttcgcaccattgcactacagcctgggcaacacagcaagaccctgtctccaaaaaaaaaaaaaaaagagcacctacaatcttatacccggtctgtttacaaataagtctgtctactgctggtgaacaatgaaatgaaaacccagcctcattgagacagtctactaaactcaaaggaattctgatattaacacccttctctgaagctattacaaatcctaaacatacttcattccaccacaagctttcttaaaacccccaaactccaggtcttttcatttcagttctagaaaattctccaaagatataggctcccaaatgacctctagatggattaagtaggactagcagagccacctggttctctctcccaaaatagatttccaagaccatgcctctatagttccttaatggtttctagttaggtgacatggcaacaccaaaggggtttttaaatgtatttcattggataaggccaaacccaggcaaatatgcatacagaacaaccgtaagcaaattcatcaaacaaaatcatgtctacatgattcctatcacctcaatcatttattaatttagctgaaatctgtttcccatattcccaccattgctgccaataagaaatggaataatatattcaaaattaacattttcatgactcataaatcttgcatttcttgccaactttggttaatagacattctattaagacatactgcctgaaaatcagatatttatgagatacagattgtgcaatttgtacactcttgcgtagaacatttcatctcttctagattattaaactgagggtttcttagattaaaaagatgtttcaagtggccatagaaagtaaacaggtctgattcatatgctaattccttttttaaatggacttgtattgaaatttgaacctaacacacaggaatattgggagggatgaaacatgtaaagaatctagcacaatgcctggaaatagagcaaacgtttaatgaagtcagttcccttaattgtaaattatttgattactatgaaaagtaggtattttttctttcagaagacagtttgaaatgtattatccttgtgacaggttatctctaattgtatggctctttacccttagttttaaaacagaaaacaaaagtagtttaagtcatgcaattttaaaggtacagttaatatattgatataatacatacttttgtaaatgtgtaagaaaaatatggaaaagctacattccaaactcaatggtggttacctctgggcaatggtgtctggaaaaggtttggaaattaaatctttcactttccatttctttactattagcatttttcataaccagtacatattatttattaatttttcctttcattttatgactatttactgagtacctactctctgctaagttctaagtcaggcctagagagtccaatctaggtggacatatttccaaactgaaagaagcttcttatttaaagtaaggcatgagtgtattaatagtgaaagataaaatgaaaatatataattcatcttatatgtttctataagatcaattaatacattttattaggtaaaacctacataatccataaaaccactgttcattttgcttcattcaaccataggtgctgaaattttctgcatcagaaatcattctggaatcctttttacctggcactgactaaagagatatgggtgttccttcccagaagtctgttcaggagtgagccactggagagcagaagattttggagaggtctcaaaagaaatttctataacaatttcttgatttctgtatgaaacacataaatatattagtagagtatgattccatctagtgaaaatttaaactcataatacatacactgaataatataaataacatagtatgcattctcatcactgattggcagtaagctctaggtatgccacatcctcagtgggtaagtctcctctcagttttcctacctaattgccagcctgtgggtccttttacctctcccatgctaactgctagcgaaggcttaatggcaactaacagtggttgactaccccgttgtgtgtcacgtactttgcatctgtgatatcatttaatattttattagagtgaaaaagtaaaagaaatcatttttggggcttcaactaccacagcagcaggtgccacagcatgacacagagcagtgctagtctgcaaactgttaccggcccaggacaagacaagaccagaagttgagagtcagcattgcaaaacttttagagtcatttttgtctgttgaatctaataataaaaaatgtgtgcttgtatttcatctcttcttcctcatatttcatttttattgcattgtacaaaagtatcagtctatgacagattgaagaggatagaaattggtcctttaccccagagagtttgagaagcactgataataaggaaacagcagaggtttagagaccagcagccctgctggtgttcgaatcctgactctatcacttactggtactgtaaacttggggaaattatttgacctccctatgccacagtttccttgtagaatggggtgaataccatctacctcacaagctagacttaagtgtttcccttctcttaaagggaaagagaaggcatgaaaacactggcctctgaacaactggggtagatcacccttgttctaggccaatagttttcaccctctttcccctcaagaggtggcatatactcccagtgtgacaattctggttgccactttcttgaataagttatttctctaaggttccctttcctcatctttaagtgtagattataccagcagggttactgtaaggattagatacaagaatgcatttaaagcacttatcccaagattgctgcactgtaacagttctatctttggcattatcattgtcccattaataaatgcagctggcctctggggcaagggcaaggagggtgcaacttgtaaagctgcccaggttatcttgaaatgccttcttatgatggcatgcccccaccatcactctagatattagtaaaaggatgaatcgtttagaaactaacagttcccaaagtccttgttgtattatatacaaacaacatttttagtatcttaagtatatataattttaactgctgtatcaactttaatctgaacagaagatcaggataagtagtgtaccaatcattacatatttacaaactaaaatttaaaaagaaaaaatatttaaattagttaagaatatgtttccccattatttagctgtaaaagagaaagatcataacattcatacttgctcaaagcgataggaagagagatttccattggcgatcccttgtaactttgtctttctccaagagcatatttgacttcttgtccattgatcactactttttctattgtaaggtcctttgtatccaaaacctaaaattaatatttttaaatagtaagaaaatagtttcatttaccagaaaaaactcatattagatataggctacaacaactagttgcttatggagagtaaaatacagagtgaaattagaagaattgaagagtcaaaagctagtctaggtctcattttttgggactctaagcatcttgaaaatttttgggttctaagatttgcatatatattgttaaataaccctaggacagtcacacaaattttgggctttaagtaaaagtcaaatctaaatcaaaatatgtttgcttctgactcctaaaattttctctattatgaaaaactttatctataacttaagtttctttcactctggctcctcaatacattacacaatatatttcctcctagaactcatgtactttcaaacttcatgttcgttaagcaaatcagcaaactgtatatcactgtggttgtatatctagaaaaagcccaacctggtatggtaactcagaccaaatgattctgcagaggattgggaggccatatctacttgccatggccaattaaggacaactgctttgggcatgaaggagtgacatcaagtgtcagagtattttctatccccaaaatcctgagccctacaaatcatactctttaattatctctcaactaatctcttgtcctagaatcttgaaccttcctatgccacaagactgtttcctaacaacataaacaaaattctacttgatggatctacccactaaatattctagttttcctccttccttccttaaactccaagggagtttttgactgctatgactactacttctacttcttcattaatcatcctccctttccccttcttccatctggcttcttgctattgaaagggcagcccccaccccgatcaacaaagtcttttctgtccaataaccttgacctctgtctactcacagcccttatggactatgtcatctggttaaaaccccttccttcacttctttgcctgtacgcatacatcataaatggttctctatttgtctaatgtttttttcctttcccctcctttattccaattcaaaaatatggatatgtcccaatgttccagccccggtcctttgattttcttgccatatccttcactccctagctcttactcatgcccacatcttcaattagtatctctgtgaagatgcctgccattctagttctacagttgtattccctccccaggacctcagtcgaatcgcctgctcaacatttccatgggacatagcaccacacattgaataggcttctaaaaattccaaaaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggcagaactgctttctaccagatctccctacctctggcattatttttttccttttctgaaatctgacctggctacatgtgaggccaagaaccagccatttcccagctgcccctgggtactttcttttgggggtacctcatttgttatccttactctaaattagtagaagatacggtttatatcttatttaaaataatagggttactccttcatattctagtacctctctagtctcttcatagtctagtacctagttctgaatagctattcagaatagctaacttgttttaaaaacttgatttgagtatcttgtgtttataacacatgcttatatagatgaattaactgggtcatttcccagtggaacatattctgttttctatattggctaaactttccaaatctgttcagaatcagaagtgtcatagtgacaactattttttgtgaaacgttttgatatcccctgtgtctgttatagctcttggccctaccctttcctataatacttactgtactgcattataatgatttctttttccattagactaagggttctaaaacagagaatgttacttaggtctgtattcccagggtttagcactctgcctcaaaaacactaggtgtcaattaatgcatgaagcaggtcctagaccaagagaaaacaaaaaatgcaatgtttaagctgtattatctcaagtcctaagtctcaactatcatttgcaaactactttttaaaattccccttcaaatttcagcgatgttatttttaaaaaatagtcaaaaactgtaataagaaagaaaaataaagaaaactggattgttgacaagttggatttagtactttttaagaaacgtgttaagcatcaacagctctactaattataggatataatttatatgtttcacagtatcctctttgaacaataccctccatccccctaaaaagcagttgtacttctcagtagctggtcagttgacatggaataggtatctgattccttttttgcacaggctggtaggaagctccatgtcaaccctgtggcccacttcttttaaagtatagagggctttatgccatgggttttgtttctcctatccctattctctcttcctgcaaattatttaattatttttaatcttatactatatatgttgcttcaagcagtctcagtcctttctagaacaaagcagagtttttttaaaaaaagctttatgcctcattatgatgtctaaatttacattttctacttgctatgtgcagggatatgatgaaaaaaaataggtttatgtgtgaaacacaaagctaaaactaaaaaaccaccttgatttgattcccagttgagacatttacttagtgaaaacaagatggtttgcagtcagaattacctattgttaactgctggcttctgccttggccatggcactaaaacctcttgagccactaaccaaaagaacacctaaacatttctgaaggtttcagtgaaaagaaacaaatgtatgaaagttatcataaatttggaggatcaaacttcagtgtaaataacccaaaacttgaaaagaattttagaaagcttagaatttgtccgattaagtctccttcagcattcctcaacatcacaaactctaagaacggagaggaaaagaagacatgacgtctctcctgattccgcactggcactgggtcttcccatctcacctctgaaatacagctggcactattatcaatgtagcccatgttaagcttaggcactgttttctaattgaaatcatccattaatcaaacttttgaatgtcctctacatgccagacatagactatactaggaagctgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataattccaggctctaatttgctaaacagacatgccaaccagaaatcactattttaaaatcttacttttctctagatttggggaatgtaaaaacaatgagcagatttttagattgggacattcttttcaaaatttaaacatcctgactcttgcttacttatagaacagagataaagtttttattctacaaaagtgatgagaacacatggatacacagtggggaacacacactggggcttactggagggtggagggtaggagaagggaaaggatcaggaaaagtaactaatgggtactaggcttaatacctgggtgacaaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtgtcctgaattgcttaatttttaaatctctagtacttttgaatgtaggacgtataaactcatgttcaaatatggcagtctcacagtgtggtttttctttttttattattatactttaagttctggggtacatgtgcagaacgtgcaggtttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatcagtttagcaaaatgttgagggatgaagagtaagaaagtaagtacttattagttcatattaatgaaatcaaaattcagatccttcctacacaagtaggaaaaagaggcctgaaagccaccaattcttatctgcccgatctgatctgattgcttattgatgtgctttagtagatttcaccatgctacactgtgtaaaatacacatgtagcatcctgccctggtgaagaagccgaatttggctgtcttttcatgaccctcttatttttaaaatgatcttctatgaaattcttcaggtgaaaggtaccttcagatgaaaggtataaaccaaatactattgggcaatttgagcaagaacattaaatataggttatgatacagataaaatcattgaataatattccatgaatctacaacctttcttcattccaatggttatagagtttgtagaagtatgtgttttctaagtgaaataactacttggctccttggaaccaactattaaaaaagcgtattgaatcatccttagaaaatttgaacgtcccatccgttcttaaattattagaagaaagttgataagattaaaaagtagaaaggaccctgaagagagagagctgcgcctagagttagcaagcagggactgttagtttcaaagtagggcggaaagaagaggcctgcccggccggggctggaaatcctaagaggcttgagaacgactagcagggagatccagggaactaggagggagacggatgggtggtgccctgcagacctgtggattgaaataagtgttcccgggaggcaaccgtgggatcagggatcgacaggacatgggatctgagacttgggtgagattgttgactgaggaaggtgcccagggggctgggaaaagtctggggcctgaagaagggggttctgggccgcaggccgaagcaatggggaggccatggagtaattagagccaggaactaaaattatgggggctactgcaaagatgacacctaagggctgggtgagttgagaggagtggacgaggcgctggatgtgcccagggacctcggagagaggatccaggcgaggggcggaggagacatacgtataagtgggggctgagggaagggatgcagaggcgtaagcggggttgagaaggggtgctgtgagagatctgggggctgaagtgcacaacatgagttggatggaggctacagaagagcagacggggacgtggggctaggcaggggccgcggcggggtgagccggagatccgggagcccgcaaggactagggtcgaggggcagggagcccgggagaggcgggcactgggcaggcgccccactgtaccaggctgcgcagattgtcctcctgagactggaccgtgagagcagcagtcccggtcagcgtccggcgagtaaagtcgacgctgcagcgcaggtgcaggtgcttggtccggcagacggaagccggagaggccaacgaacaggtatccactatctcgggcatggctctgggggatcacacagcacagcgacctacagcccaacgctcagctaccagactcgtcgatagagaacctgaggaggagggagagaggtaaagaagaggaggagggattcgcggtgcacgccgggaaaggaagtttcttaaagtcagacgagcgttgggggatgggtgaaggaactacaagttccatggtgccgcgcggcaagcgggcgcttggctacctgggagcgtgtgtgttagggatgttgagggggaccaagcgtgctcctggagctgaagaagaggaggggagtaggggcagttagaagggtggccgaagggaaatgatgagaatggagggggagataaaactgaacactaccattttggctctgttcaactttcgtggaagctgtggtggcaaacgtaagaaaatcaggttcattggtgtttgcatatagcgagggcggggcggagcaaaaacgtagaaaaggcctatcagaaggctttctttcgtgcggttcctcgttagtatagtggtgagtatccccgcctgtcacgcgggagaccggggttcgattccccgacggggaggcacagtaattgttttttgtttttagctgtaagtaattcaaggtttaaacagttgttttgtcagtttcagtgtttattatgtatatccccagaacctgcatctctcaaacggagaaagccaacattcccatcttagaaactaatgaatttctataaaagttatgcactggtcgcaatgggagtacagagctgggacatctagctcagctctggggcctttgtggcaggtgaaggaggattgaaggatgccttgcctttagggtggggcctcaaaattaaaactggaaaaattggaagaggctaggtttcgaaggaccttaaataggtggccttttaatcctgggtgcattaatgggggacccactgaagagttttcagcaggcgactgataaagcatggtcagactcatgttataaaatgcggtatcaaaaccattagcaggagatttaggaaactattaaaatggctcaatcagaagatgctgggggcctgacataggtaagtagtaagttaggatagagaggaaggaatgaatagaaggaatacttatataagtggattcacagattgagaggagacgaaggtggcccaggctttcggcttcagtggctggctagtcattaaccagaggtagtagtctatatgaagaggacagtatgttaggctgtacttgcattgctataaatacctgagactgggaagaaaaggggtttaattggtttacagttacgcaggctgtacaggaagcatagctccagtatgcttccaggtaggcctcaggaagcttacaatcatggcagaaggtgaagaaggggcaggcctctacatggcccgagcaagagcaagagattgcggggccggtgccacacacttaaacaatcagatcccacaagacactgtggcgaggacagcaccaggccatgagggctctgcccccgtgacccaaacacctcccaccaggccctacctccaacactggagattataattcaacatgagatttggtagggacatatattcaaactaaatcagacagatttaaggggaagatgctaaattcaattttggacatatgaaatttgagaacctatgggagtggaagtggagatgtccatgagtcacttggatatttaagtccacagctctggggaagtgccaggtagacatgaccacgtcacataacgtggtggatgaaattatgacagtgggtaagctcaacccaagaagagtgtgtaaagagaagggtaatgaaaacaggttggaagctgagagaacaccagaatttattgttttaaaaagggctagaggaagagaaacccatgaaataaaccagaaagagccaaccaacaatgccagatgcagtcccgaggaccaccgaagtaagaagtgaaaattctctagatttggcagtggggtgagaatggggaaggtggtgattttgttgactgtcgtgatttggttgaagatggaagccagagagtagtggattgagcaatgaagagaagagggaggtgaagcataaaccactgtcaagttgcttggccaatgtgagaagggaagataaaggggaagctagagaggaatgcagcattgagaaagctttttttttttttttttttgagacggagtctcgctctgtcgcctaggctggagtgcagtggcgcaatctcggctcacacaagctccgcctcccgggttcacaccgttctcctgcctgagcctccaaggactacaggcgcccgtcaccaagaccgactaattttttgtgtgtgtatttttagcagagacggggtttcaccgtgttagccaggatggtctgcatctcctgacctcgtgatccacccacctcggcctcccaaagtgctgggattacaggcgtgagccaccgcgcccggccgagaaagaatttttttaatgtttgctttttaaggcaagagaaaactttaacatgtttagatatacaggtgaaagggcttctggagaagaggaaagtttctgcagaaggatcgactcagaggcaaaaaggtagagaagaagaaagtaaagatttcagaggtgtgagggatagttgatgggtttagcatgctggtatggttcaattctctatcaaaagtgacgaaatttagctccagcaacaacaacaaaaaactgctatatttctggatatccttgtgttggcccctgcaagccaaaggaaaacaaaataaaaccaaaaaatcccaaactatgaaatctaataccttacacatgcataggtcctaattcatagggtgtaagaatttgtcatcaacatttgcattttcggatttttttggcaaatgtcctgttgcccaggctggatacagtggcatgatcatgggtaactgcacattcaacctcctggactcaagcgattctcgtgcctcagcctccaagtagctgggactacaggcgcccgccaccacgcctggctaatttttatatttttttagagatggggttttgtcatgttgcccaagctggtctcaaacttctgagctcaagggatccacctgccttggccttccaaagtgctgggattacaggtacgagccaccacacagagccgcaaacattttttgaggtcaccaaatctagggtgacaaatacaatagataacatagaattcatttagtcaaataatacacagtcaaatcatcttatttatctagtatggagaaaggatagtttgttttaataagaacgtcattatcatcatcttctattattgattaccaggaacccacagagtttatgccacttgtgtttaaataaaaatatccacacacaaccacaaataaattcctccattaatatattcatcaaaaaataaattacagtaggaattgttttctgagataccactcaccccaaatatagaatgtacaaaatttgcaatttacaagcaattggagtattattgatatccaatggggaattgagaatgcttcaaaaaatgaggctttccactgcatctataaaagaagggtaaggctgggcacagtggcccacaccggtaatcccagcactttgggaagctgaggcaagcagattgcttgagcccaggagtttgagttcagcctgggcaatgtggtgaatccctgtctctacaaataatagtaataataataataaaagaagggtaatatcagggtttagttaccaaaggagacttaaagatgaaaagatgatctatataaaatattatgaaaagaaataatacctatatttatagagccatatgaacaggaagaattccagccctgctattcacctggaaaagttacttaatccctcaatcccctcaggataattgaggcacctgtggcccgtgcagttgttgaaaagatgaaatgagataaagtatgggaactgcttggcactgtgattgaaacagattgggcatttgttacatgttagctaatatgattattgctgttggcttttgtctattttaaccactgtaatgttatttttccttgttgtgttagcaagagcttttaggacaacctgggaagtgagaagcaaccacggtttgtacagcaaaacaagtcacaccaaattaggcctctaaaaaggaatggcaacattagcaaagatatgttttgaaatgcatttacgggaaatttcaattgttgcacaaatgcttcccttaaaagggcaaaaagactttacattgttttctccccttcctttacatatacctttcctttcttgaaatagtcgatgtaatttgcagatatttattagatgcattctactacactgtgctacctagaaaataattggggaaggttctgtcttacctcttggataatttatactctagtacccagctgtactaatgacctaagacaacatggtgttaagaggagactaaggccctagaaaaactatacatgaaattctagagggacaagttctatccctttggagaaagtcaagaataacgtcatgattaacatagcatttgagatgggatttgaaggatgagccgaatattaataggaaaaatggtaggattgagcatggggagtgggaggtgggcagatttttcaggtggacttagcaggaatcaaggcgtggggccagaagtagagatgtgtttgggaaagaacaattctgaaggtacaaagtcctacaagttaatgcagtgccctcacacactcctcaataatctgcctttcttctttcctctccaggttatacatctggcatgatagagatcattagttgtcttctaatacctatatatcttttcatccttagtagtaaaaccttccatgtttagctggacacatggccacccggaagtagacatttcccaaccttctttgcagttaggccataccattaagttctgtccaatggcatgtaagtggaattgtcaattatgactcccaggaagtgtccttaacagtaactttattttctttttaaccttttctctattcttttcctggaatgtagataagatacattgatggctagagctctgactaccatgttgtatcatgatgttggagccatgtgctgagagtggtggagcagcaaggtagaaggagccatgagctggaaatagccaggtctctggggatcatggaaaccccatgtgaactgctctgaatttctcaaagaaataaatttctactttgtttaagacagtgttatcttgggttttctgtccttcacagggaactcaatctttactaagactcctggtctcagttgggtgagtttatcagttttgccccagatacttgcccttatctgttggttttccaccacattatcgtggacagatctttcttccttcttgcttgtgttatctgctagagcattctttctaatgtaatcatctcactcccctgcttaaaatccttcaaggtcttactaacattgccagttgatattatctgccttttttgatttaaggcccattttcaaatactagaatttttggcatacaatccaagggattaaaagatgaacgtaagctttttttttaaagaaagctttggcaaattttttttaaataaccagttattcacagtatattataatattatatttgtatgcttttatgattttttaaatctgaaattatattaaaatgaaagatgagtctcatttcttgtataagttcacttttttgttgttgttgttttggcatttgatgtttgtaagagttgagaaccctaattttctgagaaatgacatggaagactgcagcagtacctctggactccacagttgggtgctcttcgagaccatgttgccatttaaacagaatggtttcctccctttgctctgcctgctgatgtggtctagctagctcctgattaaactctgcctcttgcctcttttttacagaaatgtgtatcctctacatgcatcaaaacatcacactataccccataaatacatacactttttatgtcaattaaaaaaaaaacaaaaaaagaaatgtgtatccccctttacaccaagttaaatcactcagcttattatcttcaaagtagtataaacccccagttgttgttgtttttgaggcaaagtctctctgtgtcgcccaggccggagtgcagtggcacaatctcggctcactgcaagctccgcctcccgggttcacgccattttcctgtctcagcctcctgagtagctgggactacaggcgcccgccaccacgcccagctaattttttgtatttctaatagagatggggtttcaccgtgttagccaggatggtctcaatctcctgacctcgtgatccgcccacctcagcctcccaaagtgctgggattacaggcgtgagccaccgcgcctggccataaacccctagttttaaattaaacgtttctttttgtttttttttctttttttttttttttttgacagggtcttgctttgttacccaggctgaagtgcagtggcacgatctcagatcattgaaacctctgcctcctgggcgcaagtgatcctccacctgagccttctgagtagctgggaccacaggcacaagccactacgccgagctaatttttgtatttttagtttgtttgtttgtttgtttgtagtgacagggttttgccatgttgcccaggctggtctcgagctcctgagctcaagcaatgtgcccatctcagcctcccagaaagtgctggtatggcaggccaggtccactaacgcaggcctccataacaactgtttcagtactgactgagtggttaaattaaatattaaaatccagtacccttatgcaaaggctggaatgtaacaaaagcccaccaagagttttgcctaggcctttcctgaaccttaaagcatgattaaacaagtttattgggagtctgaaggaactccccaaacctccatgatttagcaggagacaagataaggataatcaccccagcacctgcacccatttagattaatttactgacgctccacaggaaggtcttcaagactcagaccttagttatagacggaaagaagttaatcacctacgtctttagatgaatgcacacttacatatagacatatagcttagaaggtatataagctctggaaaactttgtaattttgagttggtctggtgataatttccaggccttctccttagaaaaaaataaaggtccctattcctgtaaccggttacagaaataaaaactcgcttcctccccagttcacctgcatctcattattgggccacgagaaacagcagcctgaccctcactttggtccaagaacactgggattacagacgtgagccaccatgctcagctaaatcaaacttatttctatatattggtccacagcaatgttcatgattgataaatgaccagtcttactgtggccaccaggtctgtcaggatcttgatcctgcttaccattcccatctcatacttcattcttcccccaaagcactctgtccttggactgcaccttcacatcagcctatctcacaactccacttcttcgcttttgtgttatttctccagaaacatgttgctcctctttgctttctccacattctgcctagaaaactccctgacgcaacctgcagtagttggcaaagtgagaacatgtcgggtagacccgggaggtgaattgaaggggtaggcagggactcgacttgaaagaacctcacatgccttgagaaggagcttggaacttacctttaaggctgcaagaagtcgacgagtcttaactgggatatgcatggtcggtacttgtcttttagaaagatttctctgtggcagcatgggagacagattagaggaggtgtagacaggaggagagaaaataaactcccaagcccatgcatcctcttgccctcttcccttgttgctttaaaagacaaacatgcgcctgtagtcccagctactcgggaggctgaggcaggagaatggcgtgaaccccggggggcggagtctgcagtgagctgagatcgagccactgcactccagcctgggcgacagcgagactccgtctcaaaaaaaaaaaaaaaaaaaaaaagacaaacatgtagttcttttccatttagagagttttattggtgattattataggaaaacagactggagagagaataaaagtaggcgctgcagcaattctcaggtgagagatgatggtggcttggacccaggaatcagcagtagaattggtaagaagtgatcaaattcagcataaattttgaaggcagacctactaggatttcttggcagtttagatatggagtatgagggaaagggaggagtcaaagataatgccaagacttttgacctgataaaccaggaaaatgtagtaggattagctgagaaggagagattgtgggagaagcagactggagtgggcgagggtaaattcaggagtccaatcctttttttccccttaattttatttacttatttatttagagacagcggtcttgctatgttgcccaggctggtcatgaactcctggcctcaaaccatcctcctgcctcagcctctcaaagtggtgggattacaggagtgagccactgtgcccaactcaatctttcacatattcaatctgaggtgtctgtgattcgagtggaggtgctgagtaggcagttggacatatgagtatgagtccagaagaaaagctggaactgggctggagatacacatttgggagttcagcatgtggatggcacagggggaagaagagacgctagctatggagactggaaaggaatggcctcgatgaagaaggaaaaccaaggaagtccgtgtcttggatgacaagtgacatctggaaaaataaaggagcagtgtggtcagggagcctgatgaaattctgactatggatgactcactgttttgtgtaaaaagggggaagagaatttattctaaaaatttgttcatatctacataaaatacttctggagggatgctcaagaaactcatggtattgtttgcctgtgtggacagagaaggaaggccaaagaacagaggtgaaaagtagatatttcaactgaataatcttgtaagccttttgaattttaatgtgaatatatttcccagtcaaaaggttatttattgatatgaaaaaaaataaaggtcactggaatcccaaaccacaaacaaaaacagcccttgctgacttcctgtggacttcatagtgtctaccactggccccgcggggctctgcagcttccacttgagtggctcgatacaccctgcgtcagccatgctgaaccaaggtgttcaagctctctgcactctctggcccttccttgagcctgcatgcccttcccactcccactcttcccgcaaccttggcagggctctcctcctccccttcaggactctgccccccaccaccctccagtctgggctagagtctagtagaatctcccttgctaagagaacaaggtgcatgtgacacccttctcttcctcccttcagtgtgtgagcaaatagaagaaatgattttagccacatttttaatgttcaccttacaacatagttgaggcaatcctgaccagtttctccatcttctgtgaaatttcttcttccttgtgcagccatgcgcatgaattctatatttatagtcacatctccagtctgttctgcatgtcaagaaaaggtttgggactgggtgcggcagctcatgcctgtaatcccagcactttgggaggccaaggtgggcagatcaccaggtcaggagatcaggagcatgttggccaacatggtgaaaacccatcgctactaaaaatataaaaattagccgggcgtggtggcgcacacctgtagttccagctatttgggaggctaaggcaggagaatcacttgaacccaggaggcagagattgcagtgagccgagatcgcaccgctgcactacagcctggtgacagagcaaggctccatctcaaaaaaaaaaaaaaaaaaacaaaaacccaaggtttgggcagctgggaaggccaaaatgaaagaagcacgagaaaaagttctgccaattttgtaaaatagcataagtggtctcctcccagatgcctttctggcaccccaccccaccccatggttgaccgcaggcagagtctggaaagccccacagccacccgtgtttcctcccacacagttctgtcttttatttctcggcttgtgtccttgggagggactggcctgaaccaaataggctgtcacgctgtcctgagttggagccagacagtgccaaccatcacatggccttctctcttaggtctcacaaagtgtgtttgaagatcagcagcatctgcacctcctagaagcctcatcagaaatgcagatcttggccgggtggctcatgcctgtaatcctaacactttgggaggctgtggtgggcggatcacttgaggtcaggagttcaagaccagcctgaccaacatggcaaaaccccgtctctactaaaaatacaaaaattagctgggcatcgtggtgtgcatacctgtaatcccagctacttgggaggctgagtctgaggcatggcgtgcacctataatcccagctccttggtaggctgaggcaggaaaattgcttgaacccaggacgtggagtttgcagtgagctgagatcacgccactgcactccagcctgggtaacagagcaagactctgtctcaaaaaaaaaaaaaaaaatgcagatatcaggcctgccctgacctactggatcagaatccacattttattcagatccccaggagatctgtgtgcattttaaatgagatcactgccttagaggctcaagaaatacttttggcattggagaaaattcagtccaagtgttaatcaaacatgtcaggactccttctcttagggtccactgcccttgaccgccatatcagtactctcttaataccctagtgttatcctcaacaaagcatttaccacactgcatcattgtcagtttacttgtcagccttccctactacatggtgggtctttaagaccctgattgtatattctccctctcagcacatgtctgttgtatgaatgaacaaatgtataaatgagcgaatgagatttcacatgaggttccaggcaaacttttattcagtgtttcactctgtgttgactttgcagcaaagaaaaagccaccttctgcacttgcccttgtcctctgatgtcaccaggcaatgtttgtttgtgatacagaatgcccttgccagcccacccccccactaattgtaaacacttttaaaaacattgttattgaagcatactatccattcataaaaatgcacatattgtaagtgtatagctcactgaactttataaactgagcatgccagtgcaatcagcacccagatcagagacagaacattaccaccactgcactgttgcctcccttaagttccgtttcagtctctataaatcctgcttcctaggggtaaccactgcctgacttccaatagcatattaccttgacctgttcggctatttatctttcctttgcataaacagacacatacagtatattctgtcatgcctggctccttggctcaacattccttttgcaagatttctccataatgttgtgtgtacatctaggctgtgcacactcactgctgtacagtgttccatgtgtggatataccatgatttacttatcctttcaaccgtggatagacatgtgggtgatttccagttctgagttattattatgaatggtgctgctatggatattctggtacgtgtctttcggtgaacacattgtagccaggttttgacatgctgctttgaagtttagacagttgcaccctgccaggagatttcctttaagacccctgcaccaggccagaaacattcactgcattgcagcaacctgattctgtagttgttgacacaaatccaacacccttctccctaccccagcttgggtaggggttaaaagtagatgaagtagggagggaagctgttttcaagttacaagaaaaagttctttacaactgctggccttgttcatactttattttcctctcactcacttccgtttcttttccaggtaagcctgattgcaagcttcattgtacctgtttctttctgactcagattccagctcagcttacatttttcccactaagtaggcagtgatatttcatcacagcaggtacttacaccttttgttctgatgacttaaagcacaagtaggttttgataagtgcttgcagggtttcattttcaaaagtcctatttctgtgtcatatttgttggctttgagcccagtttctcttgctctgccaacagagcaggttatgcctatttgctcatggaaataacattttcatgagcaaaggctaaccccaaatgctttcctcctaaacgttcttctcatctacaaatccatgtttgaggaactattattttgtcatttcttacaaaaggtttttatttgaaattcagagttgagtaaacccatggaagagactcacatggttgactcactctctgccctctcctgcacatgtgtctcaggatttcttaaacccagccgagcacttccgcaaacctcccagacctgacctcctcctcccttccaagctgctccccttggcttccaaagcagcccccttctcctttcttcctcacacacacactgcaccccactcagctctatccaaccaatgcacggtcctggaaggccctccatacccacttctctcacctctgctcaactgccccctttcgccatagatatattcccagtgtatttttctctcttttggttattaattcttctgaacatgaacttcacatacctatgtatgtatgtatgtatgtgtatgtatatacacatacatatatacatgcagttaatcctcattattcatggatttggtgtttacaagtttgcctacttgccaaaatttatttgttattccaaaatcaatatttacagagcttttgtggtcactcttggacacactcagagctgtgagaaatttgagtctccggaggcacacagtctcaactgaggttaaacaaagtgaccctctgccttcctgcttctgcgcttatactgtaaacaagtgtcctttttgcagtctaatgtcaccttgtttacacatttttgtgcttttcgttggtgattttgcagtttaaaatattccccaagtgggtgctgaagtgctgtccagggttaagcgcaagaaggctgcgatgtgtttagggacaaagtgtgtgtgttagataagctgcatcaagcatgagttacagtgctgttggctgtgatttcaatgttaatgaatcaactatatattacaaaagtgtcttgaaacagaaaaacatatacaatgagattttgtattgattgatgaaaatgtgaacagaggctctcaggaacctactatatttcccctgggagcaaaggttcaggattcactaatgcaatgtttatgaactttctaagatataattactgcaaatcatgagaatcgactgtatatatttcttgtccttctggttaatttgtatttgtttctttatacctttctcactctgttcccaaagaagtggagaggggcagtttcttccaggtataccttacaattctgtcgtctctattgcagcccccagctcagtacaaacaacatagtaggtcctcaaaagattcaaaaggaataaaagacaggagtggagaaaggaaggaacaaaagaaggaacgatggcagaacgaaaagacgcaccatggaagctgagggtgctgcttatctaagcgggcgtggcttcccagaacttctcatctctcactccttaaatgcttcctctttatttcattgaatcattgaactagaataatataatatcagaaatcaagttatattttatgatagatttggctttttctgctgctctttgcaaaatctaacaaaacaaaccttccagtttctttgatttttttttcaaacttttcttctccctctcctcatcctctactccttgatcttcacttggagaaggacaattctagaattcctgaactctaggccaaaaggaagtgggcaatcatggcaagcataaacacatccatggcaagttatcagacaccttttgtgggtactaaacagcagggatgcccacttgtcccttggaagtttgcaaacatactgggaaaatggggactataaaattaaaccaccaaagatcagtgtgggagactgaataattaaagggtatccaggtggaccagtcacaaacgctgtaggagctcaatggagacatcagtgggcatcttcctggaagcagtgaggcttgcatggaaataaaaaacagggggttctaatttttgttattgttcaccaatatcagcaaaaaaggtgggcacaccctcaataaatgtttgcaaattctttacatgtgctaattaatcatatcttaagatgcaaaatacattgagggcaaggtttactcttaacaatggtcaatgtaaatccttactttaaataagcatcttataattatgatttgcatggggggcacattttgtcagatcttatttgtcatcattattttgttttgtttgttaatacactcatcttatcttggagtaggagaattattaggtctgttaatctttcttgttgctcactgttattttgctatggcagctcaagctgagacagaaattggtaccaggatgggtgctgctataacaaatgtataaaaatagcaaagacgtggaatcaacctaggtgcccatcaatgatggattggaaaagaaaatgtggtacatatacacaatggaatactatacagccataaagaagctgtcctttgcagcaacgtggatgcagctagaggccattatcttgagtgaattaacacagaaacaaaattaaatactgcatgttttcacttataagtgggagggagataaacacagggtacacacaggcataaagatagaaacaatagacacaggggactccaaaaggggaaagggaaggagggaggaaagagagttgaaaaactacctattgggtactatgttcatgttcaccatttgggtgacagttcagcagaagcccaaacctcagcattaggcaatatattcatgtaacaaacctgtacatgtacctcctgaatctcaaattaaaataatactactactaataaagaccaaagtatttcagagggaggaagaatttgcatggtgaaatttgcgtagtgaaattggcatccatatggtgcataagggatatctttggatcttctgaattacattatatacattttttaaattaaaataaattctaaaaatgtggaagcagtaatgatgcagggtgatgggtgaaggctaaaagagttttgagatacatgctacaaaaagccaaggttgctgtgaaaaaattgctaaagatgattctggtgaggactcaaaaagagcttctgtcttccttctttgaggatgcgtaaacaatcgtgaacagaatgctagcagaaatataggtggcaaaggctattctgtgaggcctcagatggaaatgaggaacatgttattggacaatggagaaaatcctttttataaagtggcaaataacttactgaattgtttatgttctagtgttttgtggacggtagaactttcaaacaatgaaattggatagttggctgaggccatttctaagcagagtgctgaaagagcagcttggttcctcttgaccactgatagtaaaatttaagaagagagaaatgaattgaagaagaaattgttaatcaaaaaataaccagcacttaaagatttggaaaattcttagcctgtccttactgcaaaaaaaatgagaaaccatgttcagaagagttaggccaagcatggtggctcctgcctataatcccagcactttgggatgccaaagcaggcagattacttgagctcaggagttcaagaccagcctgggcaacatggtgagaccccatctctattttaaaaaataaaaaaagaaaagaaaagaagagattattaagagtgtggctggtcacccatttgataaggagagtagtgtgagtatcaaccatggacctaatcagccatctcaacagaagccagaaatagagttgggattattccaggagaaataatgctttagtcccctgccagttgggactaaaaggaaaagagaaaacaagatggaatgaagtaaggctgtgaatatgcaatccccttcaggaaaagagaggaaggatcccaaaggcaattcagacatcatcagggctgctactcccaccacaggcccagagtggaaaggcccctgggaacaaggctacctccatcttggtttcaaagagtgggactgctactcagcactcatgtgggtgtggccctcacagacagccatgtgggcagtgctacactgagctgaagaagcagggacaccccactgaaagatgggggtgataccttccagtggttctggaagggaggaccaccaccccagtgggcctacagggcagagcattctttgagccaaagaggattgtatttcagttttaaagcctaatgaagtttgacttctagattttggatttcttggtacttgtcacccctttctacctttcaatttctcccttttggaataggaatatctatcctgagcctgttccatcattgtatttcggaagcacataacttgtctggtttcacagattcacagttggaaaggaattttgccttagggtgaattttgagtctcactcatattggatttacatgatatttagatgagactttaaactttagagttgatgctggaatgagttgacttttgagactgttaagatggaatgaatgtattttaaatgcaaggaggatgtgaattttgagagggacaaagggcagaatattatgaactaaacgtttaagtctcccccaaattcatatattgaagccctaacccccaatgtgagggtattacgaggtgaggtctttgggaggtaattaggtttggatgaggtcatgagggtagatcccttgtgatggaatcagtgcccttataagaagaagagagactagagcttcctctctctgccatgtgaagatacagcaagaaggtggccatctgcaagccaggaaaaggggccatggcaaacactgaatctgctcacagtctgaacttgaacttgtcagcctccagaattgtaagaaatgaatatctgttgtttaagccacccagtctataatattttgttttggcagccttagctgacccaggcactcacttatgcttacattctaacttctaaattaaggctgcaatatatgaaacatgatcatagaacttgtaattatctgaggatccagaaaaatcacgagcctgcacaaggtttatccagaggcagaggaaataccagttcactgagaaaaattaaagggacagtagaagaataaaatatagttgtttataattgtatgttacaaattatgtttgtgaagccagttacataaataatcttaaagatttaatagtttctgcctgcatccaaataattgccatgtgcctatgtccatacgcctatgtccatactactttggaaacctctaatgaatgaatgtgtaaagtttggatgggtatttgagagggaaaatacttcttatgaggttgacagttataagcaaagttaggaacaaaaagcaaattcagaaaaaaactcatcttttgttatggtctaaatgtttgtgtcacccccaaaatttatatgttgaaatcctaacccccaagttgatggtattggcagatggggcctttggtgaatggaatttgtgctcttacaaaagggacttcagagagcttgtatgcccttccaccatgtgaagacacaggaagaaggcaccatctatgaaccagaaaatgggccctcaccagacatcacatctgctggcatctttatcaaggacttctcagcctccaaaattgtgagaaataaatttctgttgtgcataagctacccagtctatggtattttgttatagcagcctgaatggactaagacacacttattgaacccccacgtgtttttctgaagaatgaatgcctcacattttacacaagatgtctgtgtgcactggggccgtctagtctaccctggcctggtgatcagggcagggaatcactgaagtttcccattctctaaaagtggaggaaatggcagccatggggaagctgccttctgctaacacaattgagccgtgaaaacaatatacaactatttttgttatattccagtggtcacacagagcaaccccaatacaataggagggcacaccacaaagccatgagtaccaggaggggtgatcactgggagactccttggaagctggctgccactgtgaggcattatctctgtttcacagaggagaaacagaagctccaataaataattgctcaagtcaactcaacttggaacaggcaggtctggggttcaaacccagacaatgagaccccagaacacatccttttagaacactgccctataccctggcctcaccacaggcctttttttctaacttcctctcttcccctcaccgcgcaaaacattgcaaatgagatttttcctttttcttagaccatttcaaaagtcattgttacttaagggtggaggttggaagatttccaaagaataaaatatacagagaatatctaaccaaagttcctaacacatacacaattcagaaaatgtaactcacagacaagggataacaagaccattgacccaatttcagagcttgacgtttacaaaatgaacacaaggcagtgtgggttgtatgcgcgttctgttcagtttctctcctttggggttgtttgggtcagcctgttgtctcatgagactgggtgggctaaattgagcaacattttgctataataagtctgcaagattagaccttaggcaacaaaagccggaaggagaaactacatttcctataaaatgtggaactgtgggataacagtgtaacaacactatgactacaaacagggaaatttatatatgagaaggaactggattgtatgttacctatataaatgatcatgagaaagtcatgttgttcttttgttgtgatcttttaaaccaaatttatagtgcattgaaccaagtaattgtaggccattattttaaagtaggttgtagcacagcatgaattaataatcacaccaattttattttacttcattggatttatttagcaattgtttttagcacttcctatatcccaggccctcttctacgcactttaaatgtattaacacatttcaattaatcctggcaacagcctgagaggtaggtactattactattcccattttacagatggtgaactgaagcatggtgcaattaagtaagcagccaagattcaaccggaattcaaaccaagcaatcaggctccacaacctgcctttttaatctggctctctgccttgtgcaaaaagatggtgagttagtccgttctcgcactgctataaagaaatatctgctgggcgcggtggctcacgcctgtaatcccaacactttgggaggccgaggcgggtggatcatgaggtcaggaattcaaggccagcctggccaagatggtgaaacccgtctctactaaaaatacaaaaattagccaggtgtagtggcaggcatctgtaatcctacctacttgggaggctgaggcagagaattgcttgaacccgggaggcagaggtggcagtgagccgagattgcgccactgcactccagcctgggtgacagagcaagactccatctcaaaaaaaaaaaaaaagaagaagaagccaggcacggtgactcatacctgtaatcccaggactttgggaggccgaggcgtgtggatcacgaggtcaggagttcaagaccagcctggcttatggtgaaaccccatctctactaaaaatacaaaaattagctgggtgtggtggcaggcgcctgtaatcccagctactcgggaggctgaggcagaagaatcacttgaacccaggaggcggaggttgcagtgagccgagatcgcaccactgtactccagtctgggtgacagagcaagactctatctcaaaaaaaaaaaaaaaaaaaaaaaaaaccctgagagtgggtaatttacaaagaaaggagattggccaggcgcggtggctcatgcctgtaattccagcactttgggaggccgaggcgggtggatcacgaggtcaggagatggagaccatcctggctaaaatggtgaaaccccgtctctactaaaaaaaaatacaaaaaattacccgggtgtggtggcggttgcgtgtagtcccagctactccggaggctgaggcaggaaaatggcatgaacccgggaggcggagcttgcagtgagccaagattgcaccactgcagtccggcctgggcgaaagagcggaaatccgtctcaaaaaaaaaaaaaagaaagaaaagaaaagaaaagaaaggaggtttaattggctcatggttctgcaggcttcacaggaatcctggtggcttctgcttctggggaggcctcaggaagcttccaatcacggcggaaggcaaagggggtgcgaggtgtctcacatagtgggagcaagagcaagagagagctagagaggaggtgatgtacacttttaaaaaacctaatctcacaagcactcactcactatcacgggaacagcaccaaaggaacagcaccaaggcgatgatgcgaaaccattcatgagaaatccgcccccatgatccaatcacctccccgccaggccccacctccaacactggggactacaattccacatggatttgatgggaacacacacccaagccatgtctgatggacacatagtttattttcttttgtgactctgcataggccattcttgccactgggaccccttccctcccaatcctcctggctttccctgcctgtcagcaaactcctgctcctttttcaagcatcaactcggatttaccctctgctgtgatgtcttctgtgactcacatgcagatttaggcacctgtttattgtgttctcaatatatcttacccatactatagaaatatttgttgttttttatctacctagtgttaaattaaataagcacgaggccattggccagaggccctctccatattttgagtttctgtggaacaaacagcaacctaatagtatgtaaacaaactgaaacctaatttaggagtatatttttgtaacatatagcctggtttcagccaatcacagagaagcttcagccaataataagcatccaattgatgagaccacgcccaataaggcagatgcctagctgttgccgatcaagtggtttctctacattgcttttgtgttcaccctagaaaagctcattgctcacactgccaagtggagttttctgaacctcttctggttctgagtgctgcctgattcatgaatcattctttgcccaaataaactctgttaaatttaatttgtctaaactgtttcttttaacactagcttctattccgccttctctgacaagcgttcaggaacccaccccacccccaccccgtactttgggtgtagcccatgtgatttaagtctagccaatcagagcactaaggagctacagttcagaggtgatcatgagacccaggttcatcgaactagagtgaatcctgggactgagcatgagcggctgggaagaaacacacaagtttttgttgcaagtctggagctgctagcagacttcacatactgcctgagcatgaagcaaaaataaagagagtgaaaagaatgagagagaatgggaaagagtctgctggtgacattatttgatcctctgaatgatgcctcacttaaattcaagatatattcttggattttgtgcattaacaaattccctttttgagcttaagcctgcttgatttatctatcatttgcaaccaaaggaacattaaccaataaatacatttcactgtatatctgtgtctatatatctatatgtatttcattttaccaaggtgtctccctactaaccataattctttgagggcagtagatgctcaatatttgtcaaatgaattcagctgaagggtgttttgaaggagactgaccttagaggagggacattttaggaaggctaatggacttagtgtgagatgtgatcaagggactcaaccaagttgaagagtaggattgaaagggaagggacaaataccaaagaaagatttaacaaggcagtgatacagagtggggtggagcaatagttagattaaagcctgagtgctaccctgttctgcgtatttgtttcttttggtgtctctttagcagccagcctaaattaaaagtttattgtactggctgattattgcctgtctaaatcacccgtctctgttagtttatcacaagtgaaaaaattaatgatagagaatcagagactcacatataagcaaataagcatgattattataagaaagagcttttattaaacaatactttcaggtcttcataagaataggggtagaatttcagagacccacataactcagtgtgcagtaaatgctgctcctgggcaacttaatggagcataaactgccagcaacggtcccaattgaaatggagactggaaggtgaagttgtccttcctttctgtaaccaccaggcaagaggacacttgtaaggtgtgagtagcagcacccaaaaaccagctgcaggactcagtggaagggaggaataaggtcactcttaaaatcctatcacctcacatagaaaaatagctaagtcctaattaagctcaacatcgccactctcagcttatccctgagacaggtcaggagaagagggaccatttgctttgctctgggattgttgcacttctgcaatctgactttgtaaaaaaaaaaaaaattaatttaaacagttgctaccatatgggatagtgtagctcgatggtttctttctctctctcgtccctctcctgctctgccttctatgtatttaccacccctcttgcagaaatgctctcgtggaatgtggcttttttttttttttttttgagatggactctcactcttgtcactcaggctggagtgcagtggcactatctcggctcactgcaacctccgtctcccgggttcaagcgattctcctgtctcagcctctcaagtaactgggattacaggtgcccaccaccatgcccagctaatttttatatttttagtagagacagggtctcaccatgttggccaggctggtctcgaactcctgacctcacgtgatccacccacctcggccgcttaaagtgctgggattatggatgcgagccaccgtgctcagccggctttccattttttttttttttaagagatgggggtcttactctgttacccaagcagtggcttgatcatagctccctgtagccttgaactcctgggctcaagcaatcctcccacctcagcctccagaatagctgggattacatgtgtaagccactgcactcggctaattctttagtgttttgtagagatgagggtcttgctctgttttggtcttgaattcctagggtctccctatgttgcccaggttggtctcaaatccctgggctcaagtgatcctaccacttcagcctcccaaagtgctgggattacaggtgtaaaccactgtactggccaacttcctgtgttttaaaaatcctcccagttggggccagtgccctaacctaatggatgcacaatgagccagttgaatgtggcctcttttagtcaaaaggaaagattctttttttttttccaagtatttctttatttatattactaggcttaagttacatgaagaaagacaactaagcagttctgcccatttcagaaaaagtttccaatcatcaccaattatgtgacaacaaataactaggaatggtgacagctttgggtcaagaccaacaaggaagaatgggctctggtgctacagttcatttccaacaagaatatggcacaccagccagcacagccatgctaacactgggccttcagtgccaagcacagattcagatctattctctgaagttagcaaatcaagtgaaataactggaattttttttaagtttaaaatgaagcccaagtaagtttaaaaccatactcttgtcatattttcctttcaaaattcacataaaacacacttttcatgccaatagcccagatattttttcttacataacccactatgtagctgcagacagactcttctacctcaagatgtaaacacaggggaaaaaattaatggccatctgtctaatattctctctatacactgctgttggatggaaaatacaaaattttgttttaaaggttccatcttagattctcgcaacctgcaggtcatacatctgactctgatgctaaggtgacagtgaatgtcacctgatgtttgttttcagtaagggggatctgggaaggatgaatttatctctttttcttcaagaattatcagatgatacatgctcctcagagccttcactctcttgaacttcagcactttccaggatcacacagccttccttataacatggctatctccagtggcaaattcataaatccaccccggtttgctattgcaacttttgcagctcacatcttgaaccctgtggctgccagtgagcatgaccagatcttaaagttcactgcacagcaggttaacgaccttgttaaaaggccagggcgcctgtgaactgagtggagatgagttctgggcggttggtcaggatcatgtcagtttgcacaggaacacagacaggtaccaccgatatgatcaaggaaaattctgcccatttttatagctgaagttctaaaatctctgagtggcgatgagatccatggctgccagatctcctcgcctgggatgaaggccccaggattcttgacagttaattaaaaccagaattcatactgaagcaggaaattttccctgacccctcacaggagggggagtgcaggtgagtgggtgcaggaattggggcgagtgctttggggcgctggcaggagcaaaactccatgaggccctgcagcacggtctagtggggtacccatgactcctgaagccccagaaagagtgttacagtgctcttttagcgttgccatctgtggacagcttaagtgttaataactcagtggaaagtcagtgtgacagccttttgcactcgcacccaagttctcgttcgacatctaggaggaatgaggtcacacaacaaattggaggtggtatatgtgggggattttattgccagtgaaagtggctctctgcagaaaggggagctgaaaaagggacagagcaggaaggtaatcttcccctgaagtccagccgtccctgctggactcctctcgaaagctacaacgtcaagccgtccggtgtccttataagaaaacagccttgctggttgcggtggctcacgcctgtaatcccagcactttgggaggcagagcaagagagagctaaggtggaggtgctgcacacttttaagtcaagctgcttctcctctctgccggctgagttctggggttactatagacacaagatgggggcagggcaggctgtgggtggttttggaaaaggcaacattccagcaggaaaacagggatgtaagttttcactttgggccgcggtattacgcttttcaccttgaggccggggccgtcgctggggacccaccctcttctgcccagaatttttctgcatcctgtccctgtcaatacggaataaaatggtacactgccatttcgcgttattcatttttttaaaaatacatatcctttaagctggttatttacctctttttaaaaggaaactgtaaggtcttccagggcagggagtatgtctgtaaagccttctagagctgggctccttgcttcctgatctcactctctcactgtctgtaggctcttgggcaggttatttaatttcttagtgtctcaatttcctcctctataaaacagagataatagtatttagcccagagggttgtggtgaagtgtgaatcatttctccatgtaaaacacataggacaggctgggcatggtggctcacgcctgtaatcccagcactttaggaggcctaggcgggtggatcacctgaggtcaggagttcaagaccagcctgggcaacatggagaaaccccatctctactgaaaatacaaaaattagctgtgcgtgatggcgcacacctgtaatcccagttactcgggagactgaggcaggagaatcacttgaacccgggagcggaggttgcggtgagccgagatcgtgccattgcacttaagcctgggttacaagagcgaaactctgtctcaaaacaaaacacacataggacagagctcagcacagagtagacattaaggattatatcctttgcttggcacaataccttgcacagggcaggcacgcaacagatgtctctggaatgaaggaatgaatgagtgaatgactgggttaagcatgttgccaccaggtggcagaagagcctcactatcaaggcagaacccaaacacgagactcatgagaactccctcctgaagtccagatacacattgaaaaaaaataaaaaaagcactgaaccccatttaggccttgaagtgaagttcctcttctctcttgcccttcctttctctcccatctctgctcactctctgctgtaatgaaccatttctttctttcccacttaatacatattagtcagtttgggctgccacagcaaaatactacagactcagtagtttaaacaacagatatttaatgcatcacagttctggaggttggaagtccatgatcaaagtgccatacgggctggtttctggtgaggcttctcttcctggcttgtagctgtccaccttcccactgttattctcacagggcctcttctctgtgccacacagagagaggaaggagagggagtgggagatggagagatgtcagattcacacaatgaagccctaaccgccattttgactgtatttgcaaatagggtttttttggtttgttttttgagacggagtcttgctctgtcgcccaggctggagtgcagtcgtgcaatctcggctcactgcaacctccgcctcctgggttcacgccattgtcctgtctcagcctcccaagtagctgggactgcaggcacccgccaccacactcagcaaattattttgtaatttagtagagatgggttttcaccatgttggtcaggctggtctcaaactcctgacctcgtgatccgcccgccttggcctcccaaagtgctgggattgcaggtgtgagccactgcaccgacctgcagatagggtttttagggagggagagagagagggagatctggagcgtcttcttataaggacaccagtcctatgggattaggccccacccaagttacctcatttaatcttaattacctccctaaagaccctgtctccaagtacagtcaaaccaggggttagggcttcattgtgtgaatctggaggggacactcttcagtttataacacgtacctttcattttttaattcctaattctcacacttcctaccaatgtggtttttcattcttactctcttgttattcccactcttcccccgacccccatccgccatccctcaatctttatatggcttttctagggctagttgtatttattgtaaactgaaaactccagggggcaccattcatgccatagtcagcataggtttgcatatgtattatgacaatgttgaggctgatggcagcaacatgtcttgaggaaggggagtctttttctcattcacacaaaggtgctggccccttcgttttcctctgttgttttcctgtcctcctccccatcattctctttcttcagcctttctctctctttccttactctcctcttcaggctgaacctgctccatgtccgtaaagagatgatttaattcatcgcaccacacattcatccagtaattttggtgggccaggccctttgtgggtgccagatggttcaccctattcttgcacctttaaaggaatcggtccatttacaccctagaggtcaatacccaatggaatgtgcctccaacatccttggatcattcatggtcttcatttaccttggagcagacattaagactcaagcattggccgggtgcggtggctcacgtctgtaataccagcactttgggaggccgatgtgggtggatcacaaggtcaggagttcaagaccagcctggccaacatggtgaaaccccatctctactaaaaatacaaaaattagctgggcatggtggcacgtgcctgtaatcccagctactcgagaggctgaggcaggagaattgcttgaactgggacctgggaggcggaggtagcagtgagctagatcgcgccattgcactccagcctgggctacagaatgagactctgtctcaaacacaaaaacaaacaaacaaaaaacaacaaaaagacaagactcaagcatggaggagaagagaagagaatataatccaataacataaactaatgtttattgaacacttgtgtgctgcacacagttctcatctctctctatgcatgacatttaatcatcaaaactgccttctcattttgtagatgagaaaactaagctgcagagaacgtggcagagactcctcctggtttcctaacttccattttcttttcctttatttaaataacaggagctcatgagttttggctgggcacatggctgcccaaggagagccgacatttcccagcctcccttgcagtttgatgtggccatataactgcattctagacacctgaggtgtgagtggaaatgatgtctgcaatttcagagttccatccttaaagggaagctgcttgcctctatgtcctcttttccttgtccccaggctgggacagggttaggggagtagtgaggcagctttgacaggagggtgaggacaggaagctggggaagagcagagcaaccactggaaggaagcttcacactcactccccacccactcactactcacccagagcaacttcccatcctgcaaactccaatcacgggaagtatcctatagaggggtatcctttttaaagaaaaaaaacctttgataccatatttttactgtactttttctacgtttgtatatgtttagatatacaaataccattgtgttgcaattacctacagtattcagtacagtaacgtgctgttcaggtttgcagcctagagcaataggctacaccatatagcctaggtgtatagtaggctataccatctaggtttgtgtaagtactgaacactccatgatgtttgcacaacggtcaaattgacaagtgacacatatcttggaacatatccctgttgttaagtgacacttgactgtatttctatttgggggaacagagcattgggaaagaaaacagaaggacccattgccttgaaggaaggatggtagacggaataatgtccaccctggcctcccaaagacgtccaagtcctaattcctggaaatatgagtatgttactttacttggcaaacgggactttgcagatgtgcttcaagtcaggaagttgagatggggagattgtcctggatgatttgggtggaccccatcaaatctcagggggctctctaagggaaagatgaaggtgggagcgtgagagccagataagacactgtgatgatggaagcagaggagagagagaagatgctacactgtgggccttaaagagagaagaaggggccctgatccgaagaatgcagcttctagaagctggaaaaggcaaggaaatggaatctgccctagaacctcactaggaatgaatcgcagctgacaccttgcttttagctaagttaaacccattttggacttctgacctccagacctataaaatactacacttgtgtttttttaagccatcaaatgtgtagtaatttgttagagaagcaataggaaataatagagagtgtgatagggtccctatggggaacgagtggcgcacatataggacataactgaccaaagtttaatgagacactgtgttttacagaggcttggccagggttaaaggcgaacaaacaggatgagaaatcaccaaggcattagcagcagcaacgagccagcacctccctgaggcttgaagggcaacgggaaaggaaaggtgttactagagaccagtgagaggagtcatggcagaggccacccaacagaagtagtggccacaaaggtgggactgtgggttgaacagatccccagaggtgtctgttatgcacagtaagctccaacagtgaaaaatcatttataaagggccgaggacagtggcttgcacctgcaatcccagcactttgggaggtcatggtgggcagattgcttaagcccaggagttccagaccagcctgggcaacatggcaaaacaccatctctactaaaaatttaaaaacttagttaggtgtggtggctggcacctgtagtcgcagctacttgggagggtgaggtaggcggatcacttgaacctgggaggttgaagctgcagtgagctgtaatcatgccactgcactccagcctggatgacagagcaagaccctgtctcaaaaaaaagaaaaaattatcaaggacttttgcctctaataaaatattcacagtggtttccttacttaatttctgaggtcaaaccagaaaatattagcagctgacttaattcaagaaggaggagcttgagtatacgtacttgttggtgtgtcttcaactcttgttctagattttactttgttttaaatatgtaaaaatgcttttagtgattacaacttatgcttcttatttcaacagatattttaaagggaaaaatatataattggatcacaggatataaaaagaaatgcagttatctatatgtgcaaaagcctagctaattgataaaagctataagttgagtcctgccactcaccttggggcaatgattttttatttatttattttattttattattattatttttttagacagagttgcccaggctggagtgcagtggtgcgatctgggctcactgcaacctccacctcccgggttcaagcaattctctgcctcagcctcccaagtagctgggattacagggtgcaccaccacacccagctaatttttgtatttatagtagacatggagtttcaccatcttggccaggatggttccgaactcctgacctcgtgatccaccactcggcctcccaaaatgctgggattacaagcataagccactgcaccacgcccggccaatgacccatttttttcaggcaaagtagcaatgggaaaatataaagtttctctagttttaatatagaagtggttaacctaatcacacaagccatacacagggtcatttgggagaatgtgcaaggaggattgcgtatttttatcttttcatagttttcttcttgataaataagcttctattttcaagccaaatctcatcttgcaatttcctgccaacttcacttctctacaaagtttacctttgcttttcccatctctgccctcaggcatttaacaaacactgtgccttttcatttttccagatttaagtgaaacattttgcagaaatgaggaatgtgataacagcccctgaagccctacctgacagcatgacattaatttgggcctgttttctctcatacttttcaattgctccccaatttatatttaatttgccacaggatataaaaagaaatatttctttaatttatattaaatacatctacattaggagagctagaggttatctaagtgaaactagctcgattatctaaaaaaagtcagaataaaataattataagcaaattggaagaacagccaacgttgttaccaataatttcttagagtttgttcaattattgtttgttatactctgtttccacttctttagccaaaataagctctaagcaaattcaaatctatttgtatagatgaagtctatgaatttaacatgataacttgaaaaaatgtaaaactttggctgggtgtggtggctcacacctgtaatcccagcactgtgggaggctgtggcgggcggatcacctaaggtcgggagctccagaccagcctggccaacattgtgaaaccccatctctactaaaaatacaagcattagcgaggcatggtggtgggcacctgtaatcccagctactcaggaggctgaggcaggagaatcgcttgaacccaggaggcggaggttgcagtgagccaagatcgtaccattgcattccagcctgggcaacaagagcaaaactccgtctcaaaaaaaaaaaaaattaaaacccaaataaattcatgtggatcttacccatatttcccatgatttagataggagttggttttaagtttatttttccactcaatgggggaaaggatttactaggaaaataatgtaaacaatctatttaagaagtcaaatggcttttaagcacttaaaaagctttgatattagcaatttacccataaatattttgttaattacataatttttttctttttaggaaatatttcttcttttcttcttcttttggctaagcctcagcagccaaattttttattttactttattttagtttactttttagagacagggcctccctctgtcacacacgctggagtgcagtggtatgatcatagctcactataaccacaaactcctgggctcaagccatcctccctcctcagcctcccgagtaggtgggactacaggtgtgcaccactacacccagctaatttttgtagtttttgtagagacggggtcttgtcatgttacccaggctggcctcgaactcctgggctcaagcaatcctccttcctcagcctcccaaaatgctgggattataggcgtgagccacagcaccagcctaccaggtatgcttttaatacatatatattgaataaataaacaaattaaagatcatctgacagaaccttcactggataatattattttttcttttcctttttttaaaaaataaggcagggtctcaccgtgttgcccaggcttgtcttgaacttctgggctcaagtgatcctcctgcttcggcctctcaaagtgctgagattacaggactgagccaccacaaccagccttcattggataacatgttatttgacatttcttctatcattgtacattgatgactgttggttgcctgcccagcagccattgcccccattactcctgttagaataaccctgattttgtgtttgtcattttattttattttatagacaaggtctcactccatcacccaggctgcaatgcaatgtagtgatcatagctcactgcaacctggaacgtctgggctcaagtgatcctcccacttcagactcctgagtagctgggactacaggtgtgcaccaccatgccaggctaattattttattttttgcagagatggaatctcacttcatttcccaggctggtcttgaactcctggactcaagccatcctcccgtttcagcccctcaagcactgggattacaggagtgagcccaccacacctggcagcgtccatcttttaaaaacttgattcagggaagggggcatcctattcctgctagagggcaaaatcgtggttgatgtaaggtagtgattttcaactggaggtaattcggcaatattttgcagtttttgttgccacagaggtaagcatattgtgctactggcatctagcgggtagagggcaggggtgcggctaaacattcttcaatgcacaggacagcccccacaacaaagagaaccatccagtccaaaatgtcaatggtgctgaggttgagaaccctgacctaaaccagtcctggtggtctcattccctagctgttagtggtttagatcccatgatgttaagtaattctgaccaatgagacgtgagcagaaattgacaagaggagtatggcagagaatgttaatttctccctacattcactttacctttcttttcagcagaagttacattcagtgttagctaggcgcatgcccaattacagactataattcccagcctccatcgttgcaaggtgtggccaagttttggctaatgggatgtgagaaaaaaataatgagtctaatttctagaccatgtttttaagaaggagaatgcttgttctttactttctctcttaatccctttctgcacactgggtggtactgctaatccagcttcaagcaagcacatgactccagagaatggcagagcaagacagaaagacttacatacattgggactgatacatgaaaagaaaataaattgctttcttctttgaaccatcgtattttttagtttttttgttttagcagtttaagctgtatgtatatgaggtactcctggggaaggttttttcctctgtgatcacacacacacatacacacacacacacacacacacacacggagggaatattcatctaaaggatgttgtaggatttgtgtgagatggctggaaccatggctgctatcttgtgaccatgaggggaggtacctggtggttcaaaactgccctgctaagtgagaacggaataggaaggttgtaaacagcccaaatctttcttaaccttgttaagccattgagttgacgaactttgcatctgtcctgtctcaggacttcttgttaagcaagatggtatatttttcatatcgtttaaatatttggcctttaaattttcagtaatagtccttacagtgatggctttcagacagaaaattaaaaattttaaaaagtgctatcctaactgattctctcaatgtattcaagtgtaaagaaattacatgtctaacctctcatggaattagagggaaaaaatttcatgttattttaagtatgttcagttcttttattaactcatattggtttcccccctactccttacccttgcaaccaagataatttgcatctaagaggttttattctgtttccactgatatgtttagaaattactatatctgaggtgggtatattgggaaaacatacactaccactcctttgcagaaatgagggcttattgcagcagctactcgcccttgcaatgcttcctgcttggaaactcgaaggactacattgagcaggtggaataaagttgaatcgaaggttcaacttacaagcagtcaggaggaggtctgccctgaagcactgtgcagactgggacctgcagcagggctgggagggggagtgtcgaggaaatgccttttgcatgtcaatggagccccgttgctgttctgtgctgcacagccacatgaggtcatcccagattagagggtgcccatgtccaggatcttaaaccaattacttccatctccattgcttcctctaaagcctcactcttagttctacacagtaatactgcctggaaactccccaaggccaccaagctcatactaacaggtttgtgatgtgggcaaactccttacatgatctcaaaatgaaagaagaggctgttcactggaggcaatagcaaatcccctttgttcctctcttggcagatgagggccttcgctctctccctaagggttccgcctgtcaccatctgtgcacccactgtggaagggccagcgctaaggtgaatttccatttacttcctgccagcagatggctcctccttttggtctcatcttgaattgtttgccagaccagccaattagtctcctcacccttcctgaagcgtcccagggaagcaatatcatcaccagcagcctatcattataccacgtcttctaagcaccgtgattctaaatgcctgccgtggaacagaagctcattcgcacatggctctttaacccttccttgaaagacctcaattcaacattctctctctcgctcacacacacacacacacacacacaaatgcacactcacacacagtacctacaacctgatccaagataggaaacaaaatgacagtatgcggcattcaataataaatttaaaaataagacataatttcagacagaatgcagaaggaaaaacacagtaactatattttctgatccccactgaggacacaataaaaacttttttttaggccaggcacggtggctcacgtgtgtaaccccagcactgtgggaggccgaggcgggcggatcacgaggtcaggaggttgagaccatcctgactaacatggtgaaaccctgtctctactaaaaatacaaaaattagcctgacgtagtggcgtgtgcctgtaatcccagctacttgggaggctgaggcaggagaatctcttgaaccagggagttagaggttgaaatgagccgagatcgcaccactgcactccagcctggcgacagagcaagactccatcacaaaaaaaataatgatacaaaaaaattaattaaataaataaaaattaaaaataaaaaaaagtggagggtttttttttttttttttttttgacagagtcttgctctgtcgccaggctggagtgcagtggcgcaatctcggctcactgcaacctccaactctctggttcaagagattctcctgcctcagcctcccgagtagctgggattacaggcacacactaccacgtcctgctaatttttgtatttttagtagagtcgggttttaccatgttggccaggctggtcttgatctcctgacctcgtgatcctcccacctcagcctcctaaagttctgggattacgggaatgagccactgcatccggccaaaactttttttttttttaccttgtggatttgttcatatgaaagaaatcttttaaggatataaaatcaaattgcactgagttacatttaacaaagtatctttatcagaaaagagtatatagaatgacactggcaggattcttcatccccgcaacccaggatgaatgatgactttccaggctaggccaaggagattctcccagcgctattcttagaaacatcaacaaggcccttgtgcacttgttttagggtttttcatcttcagacattcctgcctgatgcctaaagaagacatattattcagggcatcccatttgaatactgtatctgctctgatgcttgagcaaagtgtctgtaaagctagacagaggggacaactgcttccatccatggggcaagggagcaatgatgagatgatggaggttaaagatatttgtgaggcagacacagcatctgtgcagaggggtagaggtatttgtttttccacttttctgctcttgttaccctaacttccttgtgttctgtgattattgcacatgagctggagtagcaggggaggttcagttccttttggtggttgaggtggcaggtaaggaggcatggacacaggacgaaccccacttttgggcaaccgccaccctgaggcaagggtgggaaaagctcactttcccataaataatcactgggctgttgtccttcagtataagtgaatataagcaaagccccaagaacagtgcctggcacataaaatgcagtagctcagggtgggctataaccacttgcatcttctacagcagtgccttgtgccagcatgccctcaataaacatttgttcagtgaaggactgtacagctctgtgccacctggcagctccaactgtcccagtggatcttcctcttcctttgttcttttccttgcaaactttgcaataaagggggcattggcccaacagttaactcccaaatcttgaacaaagaggattacccctgcaactctgttccaaactgggaagcttctggctttgtggtgagctagggactgctgaaaacaactagagattaaaagaagctggaaccagctggagaataaagaaaactgctgcaagcaagtcactgcaggaagtaccagtggtctccaaaaatgcagttgcaccagattttacatacaataaggaggattggtctctctagacaggagagagtcaagtgttcctctgagaaggaaccaactcctgtaaatcaggaaatctcaggctctcactggccaaggggcaatgggacacctccccaaggtgattcatcggctccctctgaacccagaagctcaagcccattgtgctcctttttgtagactcttctcttaccctagtccccaagaatgtgctctgtgagcaggttacacccttcacaagaccctttcatgccctgtgactcccttccccattgtttgcatagtctggcagcttctgccactttccctggtaagccctgccttaaagtgaaccctcttctgtcaatcaccaggg

SEQ ID NO: 2 - DNA, 2060 bp, Homo sapiens
ctctatcgacgagtctggtagctgagcgttgggctgtaggtcgctgtgctgtgtgatcccccagagccatgcccgagatagtggatacctgttcgttggcctctccggcttccgtctgccggaccaagcacctgcacctgcgctgcagcgtcgactttactcgccggacgctgaccgggactgctgctctcacggtccagtctcaggaggacaatctgcgcagcctggttttggatacaaaggaccttacaatagaaaaagtagtgatcaatggacaagaagtcaaatatgctcttggagaaagacaaagttacaagggatcgccaatggaaatctctcttcctatcgctttgagcaaaaatcaagaaattgttatagaaatttcttttgagacctctccaaaatcttctgctctccagtggctcactcctgaacagacttctgggaaggaacacccatatctctttagtcagtgccaggccatccactgcagagcaatccttccttgtcaggacactccttctgtgaaattaacctatactgcagaggtgtctgtccctaaagaactggtggcacttatgagtgctattcgtgatggagaaacacctgacccagaagacccaagcaggaaaatatacaaattcatccaaaaagttccaataccctgctacctgattgctttagttgttggagctttagaaagcaggcaaattggcccaagaactttggtgtggtctgagaaagagcaggtggaaaagtctgcttatgagttttctgagactgaatctatgcttaaaatagcagaagatctgggaggaccgtatgtatggggacagtatgacctattggtcctgccaccatccttcccttatggtggcatggagaatccttgccttacttttgtaactcctactctactggcaggcgacaagtcactctccaatgtcattgcacatgaaatatctcatagctggacagggaatctagtgaccaacaaaacttgggatcacttttggttaaatgagggacatactgtgtacttggaacgccacatttgcggacgattgtttggtgaaaagttcagacattttaatgctctgggaggatggggagaactacagaattcggtaaagacatttggggagacacatcctttcaccaaacttgtggttgatctgacagatatagaccctgatgtagcttattcttcagttccctatgagaagggctttgctttacttttttaccttgaacaactgcttggaggaccagagattttcctaggattcttaaaagcttatgttgagaagttttcctataagagcataactactgatgactggaaggatttcctgtattcctattttaaagataaggttgatgttctcaatcaagttgattggaatgcctggctctactctcctggactgcctcccataaagcccaattatgatatgactctgacaaatgcttgtattgccttaagtcaaagatggattactgccaaagaagatgatttaaattcattcaatgccacagacctgaaggatctctcttctcatcaattgaatgagtttttagcacagacgctccagagggcacctcttccattggggcacataaagcgaatgcaagaggtgtacaacttcaatgccattaacaattctgaaatacgattcagatggctgcggctctgcattcaatccaagtgggaggacgcaattcctttggcgctaaagatggcaactgaacaaggaagaatgaagtttacccggcccttattcaaggatcttgctgcctttgacaaatcccatgatcaagctgtccgaacctaccaagagcacaaagcaagcatgcatcccgtgactgcaatgctggtggggaaagacttaaaagtggattaaagacctgcgtattgatgattttagagatttctcttttttaaatggaattcgtaaagaaatataaaacttcagctcacaattaaaactgtctttttagttttggctttttattgttttgttggtgattttactgaaataaagatgagctacttcttc

SEQ ID NO: 3 - protein, 601 aa, Homo sapiens
MPEIVDTCSLDASVCRTKHLHLRCSVDFTRRTLTGTAALTVQSQEDNLRSLVLDTKDLTIEKVVINGQEVKYALGERQSYKGSPMEISLPIALSKNQEIVIEISFETSPKSSALQWLTPEQTSGKEHPYLFSQCQAIHCRAILPCQDTPSVKLTYTAEVSVPKELVMSAIRDGETPDPEDPSRKIYKFIQKVPIPCYLIALVVGALESRQIGPRTLVWSEKEQVEKSAYEFSETESMLKIAEDLGGPYVWGQYDLLVLPPSFPYGGMENPCLTFVTPTLLAGDKSLSNVIAHEISHSWTGNLVTNKTWDHFWLNEGHTVYLERHICGRLFGEKFRHFNALGGWGELQNSVKTFGETHPFTKLVVDLTDIDPDVAYSSVPYEKGFALLFYLEQLLGGPEIFLGFLKAYVEKFSYKSITTDDWKDFKYFKDKVDVLNQVDWNAWKPGLPPIKPNYDMTLTNACIALSQRWITAKEDDLNSFNATDLKDLSSHQLNEFLAQTLQRAPLPLGHIKRMQEVYNFNAINNSEIRFRWLRLCIQSKWEDAIPLALKMATEQGRMKFWLFKDLAAFDKSHDQAVRTYQEHKASMHPVTAMLVGKDLKVD

SEQ ID NO: 4 - DNA, 964 bp, Homo sapiens
cactccagcctgggcgacagagtgagaccctgtctcaaaacaaaacaaaacaaaaactgctagggagagtgagagccagggaaaagtcaggattccgggaataggcaggaatatgtctcttccatacctgtcccaccttgggtgttcactcctattgtaactttagtcactgcattagcactttgaggggttatttggtcaggacaccgctccccacccccaccccatgccaacaattatactctaagacaccattcctcttacacaatttatttgaccagaggtggacccaacctgggttagagtctcacctctgggaatttggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtcygtcccaggttctgataatctccccatacctagcatccttaaaataatcttccttttccctttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagactgtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaa

SEQ ID NO: 5 - DNA, 979 bp, Homo sapiens
actgctagggagagtgagagccagggaaaagtcaggattccgggaataggcaggaatatgtctcttccatacctgtcccaccttgggtgttcactcctattgtaactttagtcactgcattagcactttgaggggttatttggtcaggacaccgctccccacccccaccccatgccaacaattatactctaagacaccattcctcttacacaatttatttgaccagaggtggacccaacctgggttagagtctcacctctgggaatttggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtccgtcccaggttctgataatctccccatacctagcatccttaaaataatcttccttttccckttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagactgtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccat

SEQ ID NO: 6 - DNA, 991 bp, Homo sapiens
atttatttgaccagaggtggacccaacctgggttagagtctcacctctgggaatttggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtccgtcccaggttctgataatctccccatacctagcatccttaaaataatcttccttttccctttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagacygtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgc

SEQ ID NO: 7 - DNA, 990 bp, Homo sapiens
gaggtggacccaacctgggttagagtctcacctctgggaatttggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtccgtcccaggttctgataatctccccatacctagcatccttaaaataatcttccttttccctttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagactgtgagagggatsggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgcgcccggacttat

SEQ ID NO: 8 - DNA, 990 bp, Homo sapiens
tggaattgtgatagcctccccatgtggtcagagctatttgtaacagtaaagctggagagtggccggcctgtacaacgtggactagagaggcagaggtgagggacaggagcactgacggtgctgcagtcctgggcatcagaccccttctgtccgtcccaggttctgataatctccccatacctagcatccttaaaataatcttccttttccctttttgacttctggtcacttggattgctgttacttgcaatcaaagaattctaacacagctatggttctaattaattctaactaatagagctaatacactaataattctacctagtacagctatgtgtgctgagatgccctggggcactacgttgcattggcaggggtgctttgttatgtttgtcttttatttggttcaagttattttgttgtctttgaacagactgtgagagggatgggaaagactggtgcttggggtggccatctgacccctgatggwcaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgcgcccggacttatgtttaaggttatttaaaaagcaaagcaaaatcctaaccatgt

SEQ ID NO: 9 - DNA, 984 bp, Homo sapiens
tacctagtacagctatgtgtgctgagatgccctggggcactacgttgcattggcaggggtgctttgttatgtttgtcttttatttggttcaagttattttgttgtctttgaacagactgtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacrcccagctaatttttgtatttttagtagagacggggtttcaccatgttggccaggctggtctcaaactcctgacctcaggtgattcacccgccttggcctcccaaagtgctgggattacaggcatgaaccactgcgcccggacttatgtttaaggttatttaaaaagcaaagcaaaatcctaaccatgttgaatttttgaatctgcagcagattcaaattaatgaatttaaatcatatatcaggtaaaatactaccttgacatattttgtgatcatactgagagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagaga

SEQ ID NO: 10 - DNA, 978 bp, Homo sapiens
tttggttcaagttattttgttgtctttgaacagactgtgagagggatgggaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtrattcacccgccttggcctcccaaagtgctgggattacaggcatgaaccactgcgcccggacttatgtttaaggttatttaaaaagcaaagcaaaatcctaaccatgttgaatttttgaatctgcagcagattcaaattaatgaatttaaatcatatatcaggtaaaatactaccttgacatattttgtgatcatactgagagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagagaaatctctaaaatcatcaatacgcaggtctttaatccacttttaagtctttccccaccagcattgcagtcacgggat

SEQ ID NO: 11 - DNA, 978 bp, Homo sapiens
gaaagactggtgcttggggtggccatctgacccctgatggacaggagaccaggacaagcccactggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgarccactgcgcccggacttatgtttaaggttatttaaaaagcaaagcaaaatcctaaccatgttgaatttttgaatctgcagcagattcaaattaatgaatttaaatcatatatcaggtaaaatactaccttgacatattttgtgatcatactgagagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagagaaatctctaaaatcatcaatacgcaggtctttaatccacttttaagtctttccccaccagcattgcagtcacgggatgcatgcttgctttgtgctcttggtaggttcggacagcttgatcatggga

SEQ ID NO: 12 - DNA, 977 bp, Homo sapiens
actggatgagccggaggggtccaggaggagggagttgagagctcctgctagggttgacacattctggtaaggagttcatctgctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgcgcccggacttatgtttaaggttatttaaaaagcaaagcaaaatcctaaccatgttgawtttttgaatctgcagcagattcaaattaatgaatttaaatcatatatcaggtaaaatactaccttgacatattttgtgatcatactgagagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagagaaatctctaaaatcatcaatacgcaggtctttaatccacttttaagtctttccccaccagcattgcagtcacgggatgcatgcttgctttgtgctcttggtaggttcggacagcttgatcatgggatttgtcaaaggcagcaagatccctgccaaaaaagaaaaaattgaaaagaaagaaaggcga

SEQ ID NO: 13 - DNA, 990 bp, Homo sapiens
ctgtccaccaggtaggtggtgtgcaaatacaactaagcattcatgtttaaggtttttttttaattttttatttttcgaggcagagtctccattgcccaggctggagtgcaatggcgccatctcggctcactacaacccctgcctcccagattaaagtgcttatcctccctcagcctcctgagtagctggaattacagtcgtgcctccacgcccagctaatttttgtatttttagtagagacggggtttcaccatgttggccaggctggtctcaaactcctgacctcaggtgattcacccgccttggcctcccaaagtgctgggattacaggcatgaaccactgcgcccggacttatgtttaaggttatttaaaaagcaaagcaaaatcctaaccatgttgaatttttgaatctgcagcagattcaaattaatgaatttaaatcatatatcaggtaaaatactaccttgacatattttgtgatcatactgrgagaaaattaatataaagctaattcaaaattttttaatttgtaaatcaaaagattaaaccttgttaaaatttacaaagaatatgccactataagaagaagtagctcaactttatttcagtaaaatcaccaacaaaacaataaaaagccaaaactaaaaagacagttttaattgtgagctgaagttttatatttctttacgaattccatttaaaaaagagaaatctctaaaatcatcaatacgcaggtctttaatccacttttaagtctttccccaccagcattgcagtcacgggatgcatgcttgctttgtgctcttggtaggttcggacagcttgatcatgggatttgtcaaaggcagcaagatccctgccaaaaaagaaaaaattgaaaagaaagaaaggcgagaaggagacagaggaggagaaagggagggagagaagaaagaaaggagggaaggggttcagaggaaaggaaaaaggaaggagaaagagaataagaa

SEQ ID NO: 14 - DNA, 993 bp, Homo sapiens
caagatccctgccaaaaaagaaaaaattgaaaagaaagaaaggcgagaaggagacagaggaggagaaagggagggagagaagaaagaaaggagggaaggggttcagaggaaaggaaaaaggaaggagaaagagaataagaacacaagtcaatacccaagattaaattaaaggatgtcagcaggggtgacagccagcatcacccaaataaggcaccagtcccagccaatcagatgggtatggtcctgccacagggtcccagagacctccttctgtaccagagactggcctttatactggcagatcagacattttgcagcaagttacagggaagggctagagtggctgggacccgtggctatttaccaagcagcatggaaggattttattatttgaacagagtcctctcatctcctggctaaatatcagccctgtatgtgagagtgagcctcaaagcctttctttttaaaaactgcttttaaaaaaaattttttaatcaagaytttaagagtatgaaaacactaaaatttatatagaatttctgaaaacttcaaataattgagaataaaagtcctgaccacagtgaaataataaatacataataaataatacacgaaataaataataaatacactaaataaaaaggacctaccatacaaaaggtaggattagtcatttttaatgtaactactataaacatcataaaacagaaatacttatttttcccacaaaaggtatactcttatttattttattcattttttttttttgagacagagtctcgcactgtcacccgggctggaggagctggagagcaatggcgcaatctcagctcactgcaacctctgcctcccgggttcaagcgattctcctgcctcagcctcccaagtagctaggattacaggtgcctaccaccacacctggctaattttttgtatttttagtacagacagggtttcactatgttagccaggctggtctcaaactcctgacct

SEQ ID NO: 15 - DNA, 1001 bp, Homo sapiens
caggcgtgagccaccgcgcccggcccaagtatactcttatttaaaaacctatttaaagtatactttactcaattcaaagctagatgggttttaattagggaaagcatataaaatatacttaaaacttaattttgtggtcacatcaaaaaagagataatgacttattttgccaagttttatgatattatatggccatcacttttgatggccaaaactgcaattacttttgcacccacctaaatacttgtgaagtaaatgaaaagcaaacaaaagtaatcatggatatttatggcatgatttttttttccagaatttggacaaaattcataaagaccttgactgagatattcttgtatcttgctgtcaagatacaacttatccccctctcactaagcattcctttattatgtcaagcaacctacccttgacctctatgcaacatttgaacacaaaagagttagctttatctgcttatttctccttacatttaacttcagactytctttcttgtctatacctacccaccaattatcttctagttacctttaaaaatctttgtgtatataaggctatctttgatttatttctattttatcagtatctaactctatttgatccaaaatagtaatccatatataatgcttctaaaaagaggaatgaaattatttcacattttaaatatttataagtgtgaatccctattccaaaattatactgataaactttaacaaattaaaaaatattgtcatatagattacgtttaaatatttgacagttttcttcctgtttcttagatgaattcaaagtacggtctgagtgggttcttacttgaataagggccgggtaaacttcattcttccttgttcagttgccatctttagcgccaaaggaattgcgtcctcccacttggattgaatgcagagccgcagccatctaaaaggaggatttggggggagcatggagtagaaaatgaggaaggggcaggatatgacaggtatatc

SEQ ID NO: 16 - DNA, 1001 bp, Homo sapiens
tgatattatatggccatcacttttgatggccaaaactgcaattacttttgcacccacctaaatacttgtgaagtaaatgaaaagcaaacaaaagtaatcatggatatttatggcatgatttttttttccagaatttggacaaaattcataaagaccttgactgagatattcttgtatcttgctgtcaagatacaacttatccccctctcactaagcattcctttattatgtcaagcaacctacccttgacctctatgcaacatttgaacacaaaagagttagctttatctgcttatttctccttacatttaacttcagactctctttcttgtctatacctacccaccaattatcttctagttacctttaaaaatctttgtgtatataaggctatctttgatttatttctattttatcagtatctaactctatttgatccaaaatagtaatccatatataatgcttctaaaaagaggaatgaaattatttcacattttaaayatttataagtgtgaatccctattccaaaattatactgataaactttaacaaattaaaaaatattgtcatatagattacgtttaaatatttgacagttttcttcctgtttcttagatgaattcaaagtacggtctgagtgggttcttacttgaataagggccgggtaaacttcattcttccttgttcagttgccatctttagcgccaaaggaattgcgtcctcccacttggattgaatgcagagccgcagccatctaaaaggaggatttggggggagcatggagtagaaaatgaggaaggggcaggatatgacaggtatatcttaatattacttctgtagtgatatgaataaccccactatagttatactgtacaccactttatggtatgtcttgattctgagactctcaaatccttatatatacaatttaataattggtgaagagaaagaagaggagctggttcttgaaaaagatcatatatttttaaaggtctggatca

SEQ ID NO: 17 - DNA, 996 bp, Homo sapiens
aaatatttataagtgtgaatccctattccaaaattatactgataaactttaacaaattaaaaaatattgtcatatagattacgtttaaatatttgacagttttcttcctgtttcttagatgaattcaaagtacggtctgagtgggttcttacttgaataagggccgggtaaacttcattcttccttgttcagttgccatctttagcgccaaaggaattgcgtcctcccacttggattgaatgcagagccgcagccatctaaaaggaggatttggggggagcatggagtagaaaatgaggaaggggcaggatatgacaggtatatcttaatattacttctgtagtgatatgaataaccccactatagttatactgtacaccactttatggtatgtcttgattctgagactctcaaatccttatatatacaatttaataattggtgaagagaaagaagaggagctggttcttgaaaaagatcatatatttttaaaggwctggatcaggtaggtgctcacataccttataaatccaatttctgaaggaattaaactttggtttaagcctcacattacaaatttgaattaagaaagatcaggtaggtgctcacataccttatacatgcaatttctgaaggaattaaactttggtttaagcctcacattacaaatttgaattaagaaagattaacatataatagaataaaatatttctaactattcccatttcaaagtagatttagttggttgtggagaaagcctatttaccacggaatccttcattctaattttttttttttcttttaaggcaagagaggtttagagcaaagtctaacaaaaagattaatactaccagattacatattgcaactattccttaaataccactataagtatttatatagaagcagtcagtttgacaaggaattctcaagactcaagtatgtctcatactctgcattccctttctccatctttcaaaggagtttagttttctg

SEQ ID NO: 18 - DNA, 996 bp, Homo sapiens
ggaatccttcattctaattttttttttttcttttaaggcaagagaggtttagagcaaagtctaacaaaaagattaatactaccagattacatattgcaactattccttaaataccactataagtatttatatagaagcagtcagtttgacaaggaattctcaagactcaagtatgtctcatactctgcattccctttctccatctttcaaaggagtttagttttctgctttcttccacagagacaagttaaaatgatgtacctgaatcgtatttcagaattgttaatggcattgaagttgtacacctcttgcattcgctttatgtgccccaatggaagaggtgcctaagagcaaaataaagaagtataccgtatcatttcaacaggattccttggaagaaaggagctggagagaaatgcatagccagattaaaatcctaaatattttataatatagaaataagtcagataaaaataaaagaaacaaattgcacacwaagtaaattctgtgcaaacttattccagatgaggatattctactgggagcacagggataatttactttgtgaagtattcagcattaaatgagaattgctcttcttagactttttagcatgtataaatattatctttcagacttttcctagagtttttctagttattctctataacttatatatcttaaatgcaattccattctccagatgaaatcatagttccttaatttttgcctgattccccctagctttatctttgtatatttcctctgaaatccctgttaaattatctgcatacctacataatagcagttcttaaatgtttgtattatagatctctttgggaatctgatgaataatgtggactctttccctagggggaaaatacacttactacatgaatacaaacttctgtatacaatttcagggggtttataagcatcctatccctaccttaactcaccctaaaagggaggacaagtttgggtgaaggaaagaaa

SEQ ID NO: 19 - DNA, 1001 bp, Homo sapiens
gctttatgtgccccaatggaagaggtgcctaagagcaaaataaagaagtataccgtatcatttcaacaggattccttggaagaaaggagctggagagaaatgcatagccagattaaaatcctaaatattttataatatagaaataagtcagataaaaataaaagaaacaaattgcacactaagtaaattctgtgcaaacttattccagatgaggatattctactgggagcacagggataatttactttgtgaagtattcagcattaaatgagaattgctcttcttagactttttagcatgtataaatattatctttcagacttttcctagagtttttctagttattctctataacttatatatcttaaatgcaattccattctccagatgaaatcatagttccttaatttttgcctgattccccctagctttatctttgtatatttcctctgaaatccctgttaaattatctgcatacctacataatagcagttcttaaakgtttgtattatagatctctttgggaatctgatgaataatgtggactctttccctagggggaaaatacacttactacatgaatacaaacttctgtatacaatttcagggggtttataagcatcctatccctaccttaactcaccctaaaagggaggacaagtttgggtgaaggaaagaaaaaagatgagttcagtttggacaagcagagagtttgtagtgcctgtgagaggcagaggtgcctctaggtagatgataactctcccctccaaccacgacctccttaccttacaggactccacactcactaaccaatctctgctttcatgaactactaatcctgtcgctaataatttagtccattagccccttatggacacatgcaactccaagtctaccctggtagaccaactggttaaggtcatctccaaggctccctgacttgccctaagttttgctatacccattccagaatcaccctaccatgttctctctctctgtgg

SEQ ID NO: 20 - DNA, 996 bp, Homo sapiens
gtattcagcattaaatgagaattgctcttcttagactttttagcatgtataaatattatctttcagacttttcctagagtttttctagttattctctataacttatatatcttaaatgcaattccattctccagatgaaatcatagttccttaatttttgcctgattccccctagctttatctttgtatatttcctctgaaatccctgttaaattatctgcatacctacataatagcagttcttaaatgtttgtattatagatctctttgggaatctgatgaataatgtggactctttccctagggggaaaatacacttactacatgaatacaaacttctgtatacaatttcagggggtttataagcatcctatccctaccttaactcaccctaaaagggaggacaagtttgggtgaaggaaagaaaaaagatgagttcagtttggacaagcagagagtttgtagtgcctgtgagaggcagaggtgcctctaggtagatgrtaactctcccctccaaccacgacctccttaccttacaggactccacactcactaaccaatctctgctttcatgaactactaatcctgtcgctaataatttagtccattagccccttatggacacatgcaactccaagtctaccctggtagaccaactggttaaggtcatctccaaggctccctgacttgccctaagttttgctatacccattccagaatcaccctaccatgttctctctctctgtggccctagaccacccaccagtggtagagcaatttatgaaaccatgatgacccgatgcactaaaaatagattctctctttgatgggtcctttgttgcgtcaaaatcctattcctaatttttgcatcaattccacagaaaattccgctccaaatcttctttcttctcaaggtccttagactgaagacttccctttcatggaagtctttaaaatccagtcattggtttatctcaaaatgcagcaactcctttc

SEQ ID NO: 21 - DNA, 1001 bp, Homo sapiens
tgtgtaaactctttctcacgtcctgttctctcctctccccgcaacttactccctcaagtccggtactcctgccagtctcccaactagtaacttcaccaccatgcaaccttcatggccccagattagttttctacaacccagcatttcatcccgactcttctgctggatttttaaaatcttttcctactgatcagtgtaagatctaaaatttcttagcttagcattgagagtcatcacatctggtcctaccagcttttctagtgttaccttcactgacttccttacccagtgctactgtttactccagcaatgctgcagacgaattccagcccttgctgctccctccaccttcaatttctacctccctgctagccctgggggtgcaaagcaagtctcctccaaaattccctctctgatgcccccagttggaagagtctttcactaattaagtttttccaaatgatacctaaagtatgcctccttttattgctaatgtttttwaaaaaatttttttatgagatggagtttcactctgttgctcaggctggagtacaggggtgtgatctcggctcactgcaacctccgcctcccagtccaagtgattctcctgcctcagcctcctgagtagctgggattacaggcacctgccaccatgcccggctaatttttatatttttagtagagacgagatttcatcatgttggccaggctggtctcgaactcctgacttcaagtgatctgcttgcctcggcctcccaaagtgctgggattacagatgtgagccaccgtgcctggcttattgctaaattttgcatgtgttccccttcctactagattatacgctatttgaagataaggtatatcctttcttacatattttcatatttagcacaatataaaacacagtaagcattcaatgcttttttaaagaaatgaataaattttataaatgattttttccccattagtttccacattaataatcttttgccaagttgggt

SEQ ID NO: 22 - DNA, 998 bp, Homo sapiens
atcttttcctactgatcagtgtaagatctaaaatttcttagcttagcattgagagtcatcacatctggtcctaccagcttttctagtgttaccttcactgacttccttacccagtgctactgtttactccagcaatgctgcagacgaattccagcccttgctgctccctccaccttcaatttctacctccctgctagccctgggggtgcaaagcaagtctcctccaaaattccctctctgatgcccccagttggaagagtctttcactaattaagtttttccaaatgatacctaaagtatgcctccttttattgctaatgtttttaaaaaaatttttttatgagatggagtttcactctgttgctcaggctggagtacaggggtgtgatctcggctcactgcaacctccgcctcccagtccaagtgattctcctgcctcagcctcctgagtagctgggattacaggcacctgccaccatgcccggctaatttttatawttttagtagagacgagatttcatcatgttggccaggctggtctcgaactcctgacttcaagtgatctgcttgcctcggcctcccaaagtgctgggattacagatgtgagccaccgtgcctggcttattgctaaattttgcatgtgttccccttcctactagattatacgctatttgaagataaggtatatcctttcttacatattttcatatttagcacaatataaaacacagtaagcattcaatgcttttttaaagaaatgaataaattttataaatgattttttccccattagtttccacattaataatcttttgccaagttgggtagaacataaatgctgtgcctttctgtccattttaatttctaagattttgagctagtacttaccctctggagcgtctgtgctaaaaactcattcaattgatgagaagagagatccttcaggtctgtggcattgaatgaatttaaatcatcttctttggcctgaaataaatgtt

SEQ ID NO: 23 - DNA, 995 bp, Homo sapiens
accttcaatttctacctccctgctagccctgggggtgcaaagcaagtctcctccaaaattccctctctgatgcccccagttggaagagtctttcactaattaagtttttccaaatgatacctaaagtatgcctccttttattgctaatgtttttaaaaaaatttttttatgagatggagtttcactctgttgctcaggctggagtacaggggtgtgatctcggctcactgcaacctccgcctcccagtccaagtgattctcctgcctcagcctcctgagtagctgggattacaggcacctgccaccatgcccggctaatttttatatttttagtagagacgagatttcatcatgttggccaggctggtctcgaactcctgacttcaagtgatctgcttgcctcggcctcccaaagtgctgggattacagatgtgagccaccgtgcctggcttattgctaaattttgcatgtgttccccttcctactagattatamgctatttgaagataaggtatatcctttcttacatattttcatatttagcacaatataaaacacagtaagcattcaatgcttttttaaagaaatgaataaattttataaatgattttttccccattagtttccacattaataatcttttgccaagttgggtagaacataaatgctgtgcctttctgtccattttaatttctaagattttgagctagtacttaccctctggagcgtctgtgctaaaaactcattcaattgatgagaagagagatccttcaggtctgtggcattgaatgaatttaaatcatcttctttggcctgaaataaatgttacctagttatttttgttcaagtacaatttaataatacttattggtttatctgacataaaagtaaaaattgagaaaaagaaccatatgaatgaacaagattattcaaaataaatttaagcctgagttacttaaataatcctgagattgagttactgtaatttaaatagc

SEQ ID NO: 24 - DNA, 998 bp, Homo sapiens
tgatacctaaagtatgcctccttttattgctaatgtttttaaaaaaatttttttatgagatggagtttcactctgttgctcaggctggagtacaggggtgtgatctcggctcactgcaacctccgcctcccagtccaagtgattctcctgcctcagcctcctgagtagctgggattacaggcacctgccaccatgcccggctaatttttatatttttagtagagacgagatttcatcatgttggccaggctggtctcgaactcctgacttcaagtgatctgcttgcctcggcctcccaaagtgctgggattacagatgtgagccaccgtgcctggcttattgctaaattttgcatgtgttccccttcctactagattatacgctatttgaagataaggtatatcctttcttacatattttcatatttagcacaatataaaacacagtaagcattcaatgcttttttaaagaaatgaataaattttataaatgattttytccccattagtttccacattaataatcttttgccaagttgggtagaacataaatgctgtgcctttctgtccattttaatttctaagattttgagctagtacttaccctctggagcgtctgtgctaaaaactcattcaattgatgagaagagagatccttcaggtctgtggcattgaatgaatttaaatcatcttctttggcctgaaataaatgttacctagttatttttgttcaagtacaatttaataatacttattggtttatctgacataaaagtaaaaattgagaaaaagaaccatatgaatgaacaagattattcaaaataaatttaagcctgagttacttaaataatcctgagattgagttactgtaatttaaatagctgatatgactcctagaatctatattacttaagaaaaagtagattatgggtaggaagagtggaagaaactgttgacattcattgtaccattcgaggtatagaaatttccaaagcaaag

SEQ ID NO: 25 - DNA, 1001 bp, Homo sapiens
aatgaataaattttataaatgattttttccccattagtttccacattaataatcttttgccaagttgggtagaacataaatgctgtgcctttctgtccattttaatttctaagattttgagctagtacttaccctctggagcgtctgtgctaaaaactcattcaattgatgagaagagagatccttcaggtctgtggcattgaatgaatttaaatcatcttctttggcctgaaataaatgttacctagttatttttgttcaagtacaatttaataatacttattggtttatctgacataaaagtaaaaattgagaaaaagaaccatatgaatgaacaagattattcaaaataaatttaagcctgagttacttaaataatcctgagattgagttactgtaatttaaatagctgatatgactcctagaatctatattacttaagaaaaagtagattatgggtaggaagagtggaagaaactgttgacattcattgtaccattygaggtatagaaatttccaaagcaaagaaacatttcaaaatgtatgcatgtcaactaatctatagaccaattcaaaaaggtaaagaatgaaatcgtatatttttaaatattacattaataaattggtaaggccataaactaatgttttcctccatccccacatattctgttttccccacttaatcttagaaaccatctaagaaaaataaaaatgagtctgcacttttcaaatttggatttactctcaaaaatctttgagaagatgattaagcaatattaaataaagcttataaaaataaggatttttaaatcttttagaactacttttataatcttttaaactagggcttttgttactttaaaagaaatatatgcaaatactaaaaaatcaaataggacagaaggaaaaattcttttggatctgctccctgtctccaagtactactcctcagtaactaatattagtagtttctgtatatccttccactaaatttaatgcat

SEQ ID NO: 26 - DNA, 998 bp, Homo sapiens
gattttgagctagtacttaccctctggagcgtctgtgctaaaaactcattcaattgatgagaagagagatccttcaggtctgtggcattgaatgaatttaaatcatcttctttggcctgaaataaatgttacctagttatttttgttcaagtacaatttaataatacttattggtttatctgacataaaagtaaaaattgagaaaaagaaccatatgaatgaacaagattattcaaaataaatttaagcctgagttacttaaataatcctgagattgagttactgtaatttaaatagctgatatgactcctagaatctatattacttaagaaaaagtagattatgggtaggaagagtggaagaaactgttgacattcattgtaccattcgaggtatagaaatttccaaagcaaagaaacatttcaaaatgtatgcatgtcaactaatctatagaccaattcaaaaaggtaaagaatgaaatcgtatatttttaaatawtacattaataaattggtaaggccataaactaatgttttcctccatccccacatattctgttttccccacttaatcttagaaaccatctaagaaaaataaaaatgagtctgcacttttcaaatttggatttactctcaaaaatctttgagaagatgattaagcaatattaaataaagcttataaaaataaggatttttaaatcttttagaactacttttataatcttttaaactagggcttttgttactttaaaagaaatatatgcaaatactaaaaaatcaaataggacagaaggaaaaattcttttggatctgctccctgtctccaagtactactcctcagtaactaatattagtagtttctgtatatccttccactaaatttaatgcataggtatatacccttttaaataaatattttgcatcttccccctcttcagaactctctttaatagcaatacttcttttccctttacaacttatccttaatatgagaactta

SEQ ID NO: 27 - DNA, 1001 bp, Homo sapiens
aaataatcctgagattgagttactgtaatttaaatagctgatatgactcctagaatctatattacttaagaaaaagtagattatgggtaggaagagtggaagaaactgttgacattcattgtaccattcgaggtatagaaatttccaaagcaaagaaacatttcaaaatgtatgcatgtcaactaatctatagaccaattcaaaaaggtaaagaatgaaatcgtatatttttaaatattacattaataaattggtaaggccataaactaatgttttcctccatccccacatattctgttttccccacttaatcttagaaaccatctaagaaaaataaaaatgagtctgcacttttcaaatttggatttactctcaaaaatctttgagaagatgattaagcaatattaaataaagcttataaaaataaggatttttaaatcttttagaactacttttataatcttttaaactagggcttttgttactttaaaagaaatataygcaaatactaaaaaatcaaataggacagaaggaaaaattcttttggatctgctccctgtctccaagtactactcctcagtaactaatattagtagtttctgtatatccttccactaaatttaatgcataggtatatacccttttaaataaatattttgcatcttccccctcttcagaactctctttaatagcaatacttcttttccctttacaacttatccttaatatgagaacttacagctccagctcattttctgtgcaaaaacctgcaaatctaaactatatattaattaaggatatatttatgtggtaaaaacataaaaagcaagagaatgataaaccaaaattcaggacaatggtaacctggatgggtcagcaaggagggtggagaggggcataagatggggagggatgctacagaggtaccgctaagattttacttcttatgctagtggtgggtcacacaattgttttatacaccatatgaatatgttataaat

SEQ ID NO: 28 - DNA, 1001 bp, Homo sapiens
aaccatctaagaaaaataaaaatgagtctgcacttttcaaatttggatttactctcaaaaatctttgagaagatgattaagcaatattaaataaagcttataaaaataaggatttttaaatcttttagaactacttttataatcttttaaactagggcttttgttactttaaaagaaatatatgcaaatactaaaaaatcaaataggacagaaggaaaaattcttttggatctgctccctgtctccaagtactactcctcagtaactaatattagtagtttctgtatatccttccactaaatttaatgcataggtatatacccttttaaataaatattttgcatcttccccctcttcagaactctctttaatagcaatacttcttttccctttacaacttatccttaatatgagaacttacagctccagctcattttctgtgcaaaaacctgcaaatctaaactatatattaattaaggatatatttatgtggtaaaaacmtaaaaagcaagagaatgataaaccaaaattcaggacaatggtaacctggatgggtcagcaaggagggtggagaggggcataagatggggagggatgctacagaggtaccgctaagattttacttcttatgctagtggtgggtcacacaattgttttatacaccatatgaatatgttataaatattcttttgcatttatttactatttaagacaaatcattgagaaataaaatacataaggaaaagagtgcattagtgaatacagtgtcctgaatctgttcctaacaatgcctgtttctactaatattgaagagttgatcattatccaccttaactgctgggcccaaaggaatatttgagcagaaattagtagcagttttaactagcaccaaataagctggaatacatttttcaaactaaaacagagaattttaatacactcacactgttaaaaaatcctgtttcccatagaaatctcttatacttttcttcatgacaagt

SEQ ID NO: 29 - DNA, 996 bp, Homo sapiens
aataaggatttttaaatcttttagaactacttttataatcttttaaactagggcttttgttactttaaaagaaatatatgcaaatactaaaaaatcaaataggacagaaggaaaaattcttttggatctgctccctgtctccaagtactactcctcagtaactaatattagtagtttctgtatatccttccactaaatttaatgcataggtatatacccttttaaataaatattttgcatcttccccctcttcagaactctctttaatagcaatacttcttttccctttacaacttatccttaatatgagaacttacagctccagctcattttctgtgcaaaaacctgcaaatctaaactatatattaattaaggatatatttatgtggtaaaaacataaaaagcaagagaatgataaaccaaaattcaggacaatggtaacctggatgggtcagcaaggagggtggagaggggcataagatggggagggatgctacaraggtaccgctaagattttacttcttatgctagtggtgggtcacacaattgttttatacaccatatgaatatgttataaatattcttttgcatttatttactatttaagacaaatcattgagaaataaaatacataaggaaaagagtgcattagtgaatacagtgtcctgaatctgttcctaacaatgcctgtttctactaatattgaagagttgatcattatccaccttaactgctgggcccaaaggaatatttgagcagaaattagtagcagttttaactagcaccaaataagctggaatacatttttcaaactaaaacagagaattttaatacactcacactgttaaaaaatcctgtttcccatagaaatctcttatacttttcttcatgacaagtttgtcaactacacaaaacaggttttaaaaggcaatagctgaactgattgcacagctggaggccattatcctaagtgaattaacacaggaacagaaaacc

SEQ ID NO: 30 - DNA, 977 bp, Homo sapiens
ttatttttcattaaagaattatcaagggctcatccttactttggcttcagtaaagggttctattttagtacatatatgaagaagctcctctttaagaagcttcatagaaagtgaacaaagagcaaaagtgcttcgattctttgcaccactaatagtcagcagctggtcacccaagatcattttagatttacctggtatgtgaaattgccatattggaagcagtatcttataaatgatttaaaaggaaaagaagaaaggtaagatgcaaatatttttgcatactttttttttttaagagttaagaagcaagaaaaatcaggattaatgccttcaacatcaatttttccccccataaaacttaattttctaggctgggcacagtggctcatgcctgatgcctgtaattccagcactttgggaggctaaggtgggaggatcactggagaccaggagtttgagaccagcctgtacaacacagaccctgtttgtayaaaaagttttaaattagccaggcatggaggcacatgcctgtagtcccagttactcgggaggctgaggtgggacaactgactgagcccaggaggttgaggctgcaatgagccatgatcacgccactgtagtccagcctgggcaacagagcaagaccctgtctcaaacccttaattttctatattgagagtagatataatatcaccttagataaacctgactttcaaatagcctttccaaatataactgtttgtgatttaaagtaccctccctgcttcatgagtaaagacatatttgcacaattcaaaaaggaatcaaaaatcacacattattacttacagtaatccatctttgacttaaggcaatacaagcatttgtcagagtcatatcataactgcaaagataaagattacattgtttaaaaatgcacgtgcttttgcagaaatgcagttttaaagctacagtacatacttaaatttcaaagtccc

SEQ ID NO: 31 - DNA, 974 bp, Homo sapiens
tatcttataaatgatttaaaaggaaaagaagaaaggtaagatgcaaatatttttgcatactttttttttttaagagttaagaagcaagaaaaatcaggattaatgccttcaacatcaatttttccccccataaaacttaattttctaggctgggcacagtggctcatgcctgatgcctgtaattccagcactttgggaggctaaggtgggaggatcactggagaccaggagtttgagaccagcctgtacaacacagaccctgtttgtataaaaagttttaaattagccaggcatggaggcacatgcctgtagtcccagttactcgggaggctgaggtgggacaactgactgagcccaggaggttgaggctgcaatgagccatgatcacgccactgtagtccagcctgggcaacagagcaagaccctgtctcaaacccttaattttctatattgagagtagatataatatcaccttagataaayctgactttcaaatagcctttccaaatataactgtttgtgatttaaagtaccctccctgcttcatgagtaaagacatatttgcacaattcaaaaaggaatcaaaaatcacacattattacttacagtaatccatctttgacttaaggcaatacaagcatttgtcagagtcatatcataactgcaaagataaagattacattgtttaaaaatgcacgtgcttttgcagaaatgcagttttaaagctacagtacatacttaaatttcaaagtcccttttaaataaggaaaacaaactccaaagtgaggaaaataggaaatattttacctaacttacatactactggcatcatccaagaactcacaaacccaaatggataccacattaatgaaacacccatctatcttttagaaagaatgccaaagcacctcagcaaaagactgtcatgtgctcgagtagtatatgctaaagtagttggaatcagttgagcatat

SEQ ID NO: 32 - DNA, 984 bp, Homo sapiens
ttttctatattgagagtagatataatatcaccttagataaacctgactttcaaatagcctttccaaatataactgtttgtgatttaaagtaccctccctgcttcatgagtaaagacatatttgcacaattcaaaaaggaatcaaaaatcacacattattacttacagtaatccatctttgacttaaggcaatacaagcatttgtcagagtcatatcataactgcaaagataaagattacattgtttaaaaatgcacgtgcttttgcagaaatgcagttttaaagctacagtacatacttaaatttcaaagtcccttttaaataaggaaaacaaactccaaagtgaggaaaataggaaatattttacctaacttacatactactggcatcatccaagaactcacaaacccaaatggataccacattaatgaaacacccatctatcttttagaaagaatgccaaagcacctcagcaaaagactgtcatgtgctcvagtagtatatgctaaagtagttggaatcagttgagcatatttagtacatggcaggaacagttctaggcactcaagacaacaagatgaacaacatcaagtccttgctgtcatggattttacttggttgttccaaacatctaatcatctaacaaacctgcaagcacctgctacataattggcaccgttctagatgctagacccttgagagagcccgataccattgcctgatgatttcattcctttttagaagaaaatgaaattaacacatggtaattgttaagcaaattataccaatatttgtgtgttctcaacttagaaatcatattttgcaacaatgggaaagaacatgtagtgtgtgcaaaattcttgcaaaacatccctctttctccgtaaatcatgcttgcttgtactgaaatgcttgtattagggaacagagaggcacctgccccttagagcctaaatgaagtaagttttgattagaagttaccact

SEQ ID NO: 33 - DNA, 988 bp, Homo sapiens
acttacagtaatccatctttgacttaaggcaatacaagcatttgtcagagtcatatcataactgcaaagataaagattacattgtttaaaaatgcacgtgcttttgcagaaatgcagttttaaagctacagtacatacttaaatttcaaagtcccttttaaataaggaaaacaaactccaaagtgaggaaaataggaaatattttacctaacttacatactactggcatcatccaagaactcacaaacccaaatggataccacattaatgaaacacccatctatcttttagaaagaatgccaaagcacctcagcaaaagactgtcatgtgctcgagtagtatatgctaaagtagttggaatcagttgagcatatttagtacatggcaggaacagttctaggcactcaagacaacaagatgaacaacatcaagtccttgctgtcatggattttacttggttgttccaaacatctaatcatctaacaaayctgcaagcacctgctacataattggcaccgttctagatgctagacccttgagagagcccgataccattgcctgatgatttcattcctttttagaagaaaatgaaattaacacatggtaattgttaagcaaattataccaatatttgtgtgttctcaacttagaaatcatattttgcaacaatgggaaagaacatgtagtgtgtgcaaaattcttgcaaaacatccctctttctccgtaaatcatgcttgcttgtactgaaatgcttgtattagggaacagagaggcacctgccccttagagcctaaatgaagtaagttttgattagaagttaccactgaatctcccttaaagagagttgtgactgggactccgtttgttccctaggggagacaataaaaaggtcaacacagctcccacctcgaagcagctgccagtttattacatgaagtgtcaggctgtggactgcaggcatgccattttgtcttcaagaacaggtggg

SEQ ID NO: 34 - DNA, 992 bp, Homo sapiens
tggataccacattaatgaaacacccatctatcttttagaaagaatgccaaagcacctcagcaaaagactgtcatgtgctcgagtagtatatgctaaagtagttggaatcagttgagcatatttagtacatggcaggaacagttctaggcactcaagacaacaagatgaacaacatcaagtccttgctgtcatggattttacttggttgttccaaacatctaatcatctaacaaacctgcaagcacctgctacataattggcaccgttctagatgctagacccttgagagagcccgataccattgcctgatgatttcattcctttttagaagaaaatgaaattaacacatggtaattgttaagcaaattataccaatatttgtgtgttctcaacttagaaatcatattttgcaacaatgggaaagaacatgtagtgtgtgcaaaattcttgcaaaacatccctctttctccgtaaatcatgcttgcttgtacygaaatgcttgtattagggaacagagaggcacctgccccttagagcctaaatgaagtaagttttgattagaagttaccactgaatctcccttaaagagagttgtgactgggactccgtttgttccctaggggagacaataaaaaggtcaacacagctcccacctcgaagcagctgccagtttattacatgaagtgtcaggctgtggactgcaggcatgccattttgtcttcaagaacaggtgggatcagaggtccttgactgatcagaatacactgctttcaaccaaaacattattagcattgatttcttaaaaaataatagcaaagtagaaaacctttagctggtctgtttcttcgtgtcctgaaacttccttattagtgtaattaaaagtactaagttaagaattagcctgggaaaggaccctacttatggcaaagtcttcagaaaagtaaagagcaaaaccagatatgtgccttgttctcatggtgctgacagtatag

SEQ ID NO: 35 - DNA, 988 bp, Homo sapiens
gagcccgataccattgcctgatgatttcattcctttttagaagaaaatgaaattaacacatggtaattgttaagcaaattataccaatatttgtgtgttctcaacttagaaatcatattttgcaacaatgggaaagaacatgtagtgtgtgcaaaattcttgcaaaacatccctctttctccgtaaatcatgcttgcttgtactgaaatgcttgtattagggaacagagaggcacctgccccttagagcctaaatgaagtaagttttgattagaagttaccactgaatctcccttaaagagagttgtgactgggactccgtttgttccctaggggagacaataaaaaggtcaacacagctcccacctcgaagcagctgccagtttattacatgaagtgtcaggctgtggactgcaggcatgccattttgtcttcaagaacaggtgggatcagaggtccttgactgatcagaatacactgctttcaacyaaaacattattagcattgatttcttaaaaaataatagcaaagtagaaaacctttagctggtctgtttcttcgtgtcctgaaacttccttattagtgtaattaaaagtactaagttaagaattagcctgggaaaggaccctacttatggcaaagtcttcagaaaagtaaagagcaaaaccagatatgtgccttgttctcatggtgctgacagtatagcgaagaggaaatactttaatcatacgaataaataaatgtaaagttagaactgtgcaactgctacgaagagaggatatagcactaaaaagccctagaatgggagatttgacctggccagggatgtcaagaaatgcttccaagaggaagtggttcttgagctgagattggaattaactgggcaaagggctccgggtagagaaaacagcatgctcaggtactatgttggaggacatatggggagttcgagaaactccaaaactgccagtgtgactgaagcaaaggga

SEQ ID NO: 36 - DNA, 985 bp, Homo sapiens
tctcaacttagaaatcatattttgcaacaatgggaaagaacatgtagtgtgtgcaaaattcttgcaaaacatccctctttctccgtaaatcatgcttgcttgtactgaaatgcttgtattagggaacagagaggcacctgccccttagagcctaaatgaagtaagttttgattagaagttaccactgaatctcccttaaagagagttgtgactgggactccgtttgttccctaggggagacaataaaaaggtcaacacagctcccacctcgaagcagctgccagtttattacatgaagtgtcaggctgtggactgcaggcatgccattttgtcttcaagaacaggtgggatcagaggtccttgactgatcagaatacactgctttcaaccaaaacattattagcattgatttcttaaaaaataatagcaaagtagaaaacctttagctggtctgtttcttcgtgtcctgaaacttccttattagwgtaattaaaagtactaagttaagaattagcctgggaaaggaccctacttatggcaaagtcttcagaaaagtaaagagcaaaaccagatatgtgccttgttctcatggtgctgacagtatagcgaagaggaaatactttaatcatacgaataaataaatgtaaagttagaactgtgcaactgctacgaagagaggatatagcactaaaaagccctagaatgggagatttgacctggccagggatgtcaagaaatgcttccaagaggaagtggttcttgagctgagattggaattaactgggcaaagggctccgggtagagaaaacagcatgctcaggtactatgttggaggacatatggggagttcgagaaactccaaaactgccagtgtgactgaagcaaagggagctagagtgttaggagcttataatccccactaaaggattttgtcttagcccaagagcaaagagataccagtggagactgctaagcaggaggacaa

SEQ ID NO: 37 - DNA, 978 bp, Homo sapiens
catgaagtgtcaggctgtggactgcaggcatgccattttgtcttcaagaacaggtgggatcagaggtccttgactgatcagaatacactgctttcaaccaaaacattattagcattgatttcttaaaaaataatagcaaagtagaaaacctttagctggtctgtttcttcgtgtcctgaaacttccttattagtgtaattaaaagtactaagttaagaattagcctgggaaaggaccctacttatggcaaagtcttcagaaaagtaaagagcaaaaccagatatgtgccttgttctcatggtgctgacagtatagcgaagaggaaatactttaatcatacgaataaataaatgtaaagttagaactgtgcaactgctacgaagagaggatatagcactaaaaagccctagaatgggagatttgacctggccagggatgtcaagaaatgcttccaagaggaagtggttcttgagctgagawgaattaactgggcaaagggctccgggtagagaaaacagcatgctcaggtactatgttggaggacatatggggagttcgagaaactccaaaactgccagtgtgactgaagcaaagggagctagagtgttaggagcttataatccccactaaaggattttgtcttagcccaagagcaaagagataccagtggagactgctaagcaggaggacaacatgacacatttgtgcttttaaaggtttactctagctttagtgtggagagtggctgggagaagtcagaacagatacaagtgcacagtttgggtgccagaacagtcttccaggatgtgaagatgtgatactgaacttggacagtggtagtagaaatggagagatgtggatagactcagatatttaaatacatatacaaatgatgagagcattataaaaagaggatcgtggaagccaagattctgtgctgcaatggatcaaagtattttctgtggtttgagattttct

SEQ ID NO: 38 - DNA, 979 bp, Homo sapiens
ggattttgtcttagcccaagagcaaagagataccagtggagactgctaagcaggaggacaacatgacacatttgtgcttttaaaggtttactctagctttagtgtggagagtggctgggagaagtcagaacagatacaagtgcacagtttgggtgccagaacagtcttccaggatgtgaagatgtgatactgaacttggacagtggtagtagaaatggagagatgtggatagactcagatatttaaatacatatacaaatgatgagagcattataaaaagaggatcgtggaagccaagattctgtgctgcaatggatcaaagtattttctgtggtttgagattttctaagatactctctctttacagaattcccgggcacacgaatgattccagggttcctccagcactttggtattacttgaaagcaatcttaagggatctagaatgaaccaacgcccaaaaaggatcccttagcagyggtgatatcaaagaaacacttttgaagaactaattttccacccagatttccccaattttaaaagcaatgggcaaagccttctccactcctaaacttcctggaactgtcttttggctatatcaggcccctgaagttagagtctttgaaagactccaaactccaaattctatgcttttattctcaggctcctcataattctacagcacaccagactgctgaccactctccgtaccacttttaaattatttcttcccacagctttcttaacaatgaacctttgaaatctttttagttttccatttattttgctacctttcctctgtcctagctctaaaatgaagatcctctaaggttctacagtttacttcttgtattctcctttgtaagtcatctccaagacgatgtccaaatccatcaccattaaaattaatagtttcctcacccacaacacttaatattttaaaaaaaatacttttcattgtattataattacttgatac

SEQ ID NO: 39 - DNA, 985 bp, Homo sapiens
tcttccaggatgtgaagatgtgatactgaacttggacagtggtagtagaaatggagagatgtggatagactcagatatttaaatacatatacaaatgatgagagcattataaaaagaggatcgtggaagccaagattctgtgctgcaatggatcaaagtattttctgtggtttgagattttctaagatactctctctttacagaattcccgggcacacgaatgattccagggttcctccagcactttggtattacttgaaagcaatcttaagggatctagaatgaaccaacgcccaaaaaggatcccttagcagcggtgatatcaaagaaacacttttgaagaactaattttccacccagatttccccaattttaaaagcaatgggcaaagccttctccactcctaaacttcctggaactgtcttttggctatatcaggcccctgaagttagagtctttgaaagactccaaactccaaattctaygcttttattctcaggctcctcataattctacagcacaccagactgctgaccactctccgtaccacttttaaattatttcttcccacagctttcttaacaatgaacctttgaaatctttttagttttccatttattttgctacctttcctctgtcctagctctaaaatgaagatcctctaaggttctacagtttacttcttgtattctcctttgtaagtcatctccaagacgatgtccaaatccatcaccattaaaattaatagtttcctcacccacaacacttaatattttaaaaaaaatacttttcattgtattataattacttgatacatacatatttgctctgtgagttccttattcatcatattagtgcctgacaataaatgtgtgctggattgagctgaatctttattacatctctgctcagtcatttttaatttcttcttttctcaccacagccaatcagttgccaatagattctagcccccaaacgtctcttc

SEQ ID NO: 40 - DNA, 998 bp, Homo sapiens
aaaagcaatgggcaaagccttctccactcctaaacttcctggaactgtcttttggctatatcaggcccctgaagttagagtctttgaaagactccaaactccaaattctatgcttttattctcaggctcctcataattctacagcacaccagactgctgaccactctccgtaccacttttaaattatttcttcccacagctttcttaacaatgaacctttgaaatctttttagttttccatttattttgctacctttcctctgtcctagctctaaaatgaagatcctctaaggttctacagtttacttcttgtattctcctttgtaagtcatctccaagacgatgtccaaatccatcaccattaaaattaatagtttcctcacccacaacacttaatattttaaaaaaaatacttttcattgtattataattacttgatacatacatatttgctctgtgagttccttattcatcatattagtgcctgacaataaatgtgtsctggattgagctgaatctttattacatctctgctcagtcatttttaatttcttcttttctcaccacagccaatcagttgccaatagattctagcccccaaacgtctcttctctcagttactcctttcttttccactgcctttgtatgacttcaggtcctcataatctctagcaaggctgttgtaaaaattaacgagataatgtatggcacttcttaatgaagtgctaggaaaaaaatctaaagtattatttttgctgataccttttttagacgttaaaagggtttactgatgatttgtgccacctgtttccaacacaaaattcgaaacattctatcgtaatcacccctccctacctgagctcctgtttcccaccacagcctatgataaccaggactgcccagttagtggggcgctctgaccacatttgttccatactcagaactcccagtaacttctcaaccaaacacttctcggcctggctgtttaaagtgcttta

SEQ ID NO: 41 - DNA, 1001 bp, Homo sapiens
tctctcctgctgctccctgaacatcaattaaactggcctgtttagtgtaagagaagctggtaggcaattttggtgatccaaaagaaaggcaacaagagaacatgccatggaacatgccatggtcagtgtcctcacacaactcgtgaaagaccagggttcaggtccgattgaaggagggggttcagtataaaaagcagtatattgaggccgggcacggtggctcacgcctgtaatcccaacactctgggagacaaaggcaggtggattgtttgagctcaggagttcgagaccagcctgggcaatatggtgaaaccctgcctctagcaaaagtacaaaaacagccgggtgtggtagtgcgcatctgtggtcccagctacttgtaaggctgaggtaggaggatcacttgagcctggaaggcagagggtgcagtgagctaagatcacatcactgcacgccaggctgagccacagagtgagaccctgtttctaaaaaaaaagaagraagaaagcagtatattggaggcaataagactgccagggtttgaatctcaacttttactactcactagctgtgcaacctagggcaagacactttacctagctaaacctaacttacctccttgggaaatggggataataacttataacagtgttgtaattaacataatacttataaaatatttttattgcagaagtttgaaggaagatacaatagcttattgtctaaatccctcaccatccttgtgcagaaaggaggcactcaattacttgaagtgaaaaaccatatttgtaaactgcagaaattattcttttggcctcagggttaaggccaaaacacctaagaactctgctttcatcatttactagtaacagtttcaggaaggcatactattctttcagatattttgaggctctctaggagttaggagaatgagaaggaaagcattagcaggcaagtacttacttgggctttatgggaggcagtccaggagagtagagcca

SEQ ID NO: 42 - DNA, 1001 bp, Homo sapiens
ttagtgtaagagaagctggtaggcaattttggtgatccaaaagaaaggcaacaagagaacatgccatggaacatgccatggtcagtgtcctcacacaactcgtgaaagaccagggttcaggtccgattgaaggagggggttcagtataaaaagcagtatattgaggccgggcacggtggctcacgcctgtaatcccaacactctgggagacaaaggcaggtggattgtttgagctcaggagttcgagaccagcctgggcaatatggtgaaaccctgcctctagcaaaagtacaaaaacagccgggtgtggtagtgcgcatctgtggtcccagctacttgtaaggctgaggtaggaggatcacttgagcctggaaggcagagggtgcagtgagctaagatcacatcactgcacgccaggctgagccacagagtgagaccctgtttctaaaaaaaaagaaggaagaaagcagtatattggaggcaataagactgccagggttwgaatctcaacttttactactcactagctgtgcaacctagggcaagacactttacctagctaaacctaacttacctccttgggaaatggggataataacttataacagtgttgtaattaacataatacttataaaatatttttattgcagaagtttgaaggaagatacaatagcttattgtctaaatccctcaccatccttgtgcagaaaggaggcactcaattacttgaagtgaaaaaccatatttgtaaactgcagaaattattcttttggcctcagggttaaggccaaaacacctaagaactctgctttcatcatttactagtaacagtttcaggaaggcatactattctttcagatattttgaggctctctaggagttaggagaatgagaaggaaagcattagcaggcaagtacttacttgggctttatgggaggcagtccaggagagtagagccaggcattccaatcaacttgattgagaacatcaacctatgaat

SEQ ID NO: 43 - DNA, 974 bp, Homo sapiens
gagacaaaggcaggtggattgtttgagctcaggagttcgagaccagcctgggcaatatggtgaaaccctgcctctagcaaaagtacaaaaacagccgggtgtggtagtgcgcatctgtggtcccagctacttgtaaggctgaggtaggaggatcacttgagcctggaaggcagagggtgcagtgagctaagatcacatcactgcacgccaggctgagccacagagtgagaccctgtttctaaaaaaaaagaaggaagaaagcagtatattggaggcaataagactgccagggtttgaatctcaacttttactactcactagctgtgcaacctagggcaagacactttacctagctaaacctaacttacctccttgggaaatggggataataacttataacagtgttgtaattaacataatacttataaaatatttttattgcagaagtttgaaggaagatacaatagcttattktctaaatccctcaccatccttgtgcagaaaggaggcactcaattacttgaagtgaaaaaccatatttgtaaactgcagaaattattcttttggcctcagggttaaggccaaaacacctaagaactctgctttcatcatttactagtaacagtttcaggaaggcatactattctttcagatattttgaggctctctaggagttaggagaatgagaaggaaagcattagcaggcaagtacttacttgggctttatgggaggcagtccaggagagtagagccaggcattccaatcaacttgattgagaacatcaacctatgaatagtaagaattcacagtttacaatagaatgccctttcctgtcaaaaaaaaatttaaacttgtaagtccttagatatataattttgtctaatctgctatatcaagataatttctaaatcttttttaaaaattaatattttaaattgatagatcataattgtgtatacttatgtgacacaat

SEQ ID NO: 44 - DNA, 990 bp, Homo sapiens
acctagctaaacctaacttacctccttgggaaatggggataataacttataacagtgttgtaattaacataatacttataaaatatttttattgcagaagtttgaaggaagatacaatagcttattgtctaaatccctcaccatccttgtgcagaaaggaggcactcaattacttgaagtgaaaaaccatatttgtaaactgcagaaattattcttttggcctcagggttaaggccaaaacacctaagaactctgctttcatcatttactagtaacagtttcaggaaggcatactattctttcagatattttgaggctctctaggagttaggagaatgagaaggaaagcattagcaggcaagtacttacttgggctttatgggaggcagtccaggagagtagagccaggcattccaatcaacttgattgagaacatcaacctatgaatagtaagaattcacagtttacaatagaatgccctttcctgtcraaaaaaaatttaaacttgtaagtccttagatatataattttgtctaatctgctatatcaagataatttctaaatcttttttaaaaattaatattttaaattgatagatcataattgtgtatacttatgtgacacaatgcgatgttttgatatatgtactcaatgtggactaagtcaagctaatatatccattacctcatctaactctatcttctaaaatttatattcatcaccatactattgatgacttctctgaaataggaaaattctacaggtagttcatgtggttaagatcacatttaaaatagaaaaaatatgcaatgagaggttgagtcctaaagttctgaaccaatactactattagataatacaagttaacctaatcagtcaataaatagagatatatcgagcatgaaaaatagaaaaggtttttaaatccaaccttatctttaaaataggaatacaggaaatccttccagtcatcagtagttatgctcttat

SEQ ID NO: 45 - DNA, 999 bp, Homo sapiens
aattgtgtatacttatgtgacacaatgcgatgttttgatatatgtactcaatgtggactaagtcaagctaatatatccattacctcatctaactctatcttctaaaatttatattcatcaccatactattgatgacttctctgaaataggaaaattctacaggtagttcatgtggttaagatcacatttaaaatagaaaaaatatgcaatgagaggttgagtcctaaagttctgaaccaatactactattagataatacaagttaacctaatcagtcaataaatagagatatatcgagcatgaaaaatagaaaaggtttttaaatccaaccttatctttaaaataggaatacaggaaatccttccagtcatcagtagttatgctcttataggaaaacttctcaacataagcttttaagaatcctaggaaaatctctaagagtaaaaaagaaaagaaatcaattcatagaaaggtaattatttgacattttgtgtgcgtrtttggcattgtactattaaccacagagaacagagaacattcagagaatagggaaatctacgaggactttcagagtgaaagaatgttcaaaaaaggaggtgggacttaagttgggccttgaagaatatatgtaattcagtggaagggagaagagaaattctaattataggtaaggggataacacatgaagacacagaaaaggaatgcataacccaagttctaaaagcaataaccttcacatgactagaaaggagaaaaataagactggacaggcagaatggatccaggtgacagacagccttccaagtcaatcaaccaaggagaacacctcaatgtccatcagtgggggatgggtacataactcagcatagctttatcatgaactagtatgatggcattaaaaagtatgaaacagatttatatgtactgacacagaagggtgtatgtgaaatatcgagcaaaacaaaacacaaatgcagagccaatatatagcatgaccca

SEQ ID NO: 46 - DNA, 992 bp, Homo sapiens
taactctatcttctaaaatttatattcatcaccatactattgatgacttctctgaaataggaaaattctacaggtagttcatgtggttaagatcacatttaaaatagaaaaaatatgcaatgagaggttgagtcctaaagttctgaaccaatactactattagataatacaagttaacctaatcagtcaataaatagagatatatcgagcatgaaaaatagaaaaggtttttaaatccaaccttatctttaaaataggaatacaggaaatccttccagtcatcagtagttatgctcttataggaaaacttctcaacataagcttttaagaatcctaggaaaatctctaagagtaaaaaagaaaagaaatcaattcatagaaaggtaattatttgacattttgtgtgcgtgtttggcattgtactattaaccacagagaacagagaacattcagagaatagggaaatctacgaggactttcagagtgaaagawtgttcaaaaaaggaggtgggacttaagttgggccttgaagaatatatgtaattcagtggaagggagaagagaaattctaattataggtaaggggataacacatgaagacacagaaaaggaatgcataacccaagttctaaaagcaataaccttcacatgactagaaaggagaaaaataagactggacaggcagaatggatccaggtgacagacagccttccaagtcaatcaaccaaggagaacacctcaatgtccatcagtgggggatgggtacataactcagcatagctttatcatgaactagtatgatggcattaaaaagtatgaaacagatttatatgtactgacacagaagggtgtatgtgaaatatcgagcaaaacaaaacacaaatgcagagccaatatatagcatgacccattttttgtaattaaaataattacatgtatttatttgtctgcttgttaatttacacctagaaaatgatctggagccatttaca

SEQ ID NO: 47 - DNA, 988 bp, Homo sapiens
ccatactattgatgacttctctgaaataggaaaattctacaggtagttcatgtggttaagatcacatttaaaatagaaaaaatatgcaatgagaggttgagtcctaaagttctgaaccaatactactattagataatacaagttaacctaatcagtcaataaatagagatatatcgagcatgaaaaatagaaaaggtttttaaatccaaccttatctttaaaataggaatacaggaaatccttccagtcatcagtagttatgctcttataggaaaacttctcaacataagcttttaagaatcctaggaaaatctctaagagtaaaaaagaaaagaaatcaattcatagaaaggtaattatttgacattttgtgtgcgtgtttggcattgtactattaaccacagagaacagagaacattcagagaatagggaaatctacgaggactttcagagtgaaagaatgttcaaaaaaggaggtgggacttaarttgggccttgaagaatatatgtaattcagtggaagggagaagagaaattctaattataggtaaggggataacacatgaagacacagaaaaggaatgcataacccaagttctaaaagcaataaccttcacatgactagaaaggagaaaaataagactggacaggcagaatggatccaggtgacagacagccttccaagtcaatcaaccaaggagaacacctcaatgtccatcagtgggggatgggtacataactcagcatagctttatcatgaactagtatgatggcattaaaaagtatgaaacagatttatatgtactgacacagaagggtgtatgtgaaatatcgagcaaaacaaaacacaaatgcagagccaatatatagcatgacccattttttgtaattaaaataattacatgtatttatttgtctgcttgttaatttacacctagaaaatgatctggagccatttacaccaaactgctaacagtggttacccctg

SEQ ID NO: 48 - DNA, 975 bp, Homo sapiens
gtctatatctgtcagatcaaccacaagtttggtgaaaggatgtgtctccccaaatgtctttacctgcaagacatgaaataacatggagaaacatatagaaagactgctatcaccacgcaaataagctaataaggaggtattacttcactcagtggtgtaactttaggggaatctaaaacttggagactggaacactaggatatgttggcataaacttctggaagtctattaatagaatgcttacttaagtaatattctctgttgtttcttgctcaataatacaggctttattcttataaaaagactagaaaaatgatttaatgcctggtcagcaaatttggctttcaggagacaacacttaaaaatgacataccaaataagatgcaaacatagtaaacagctatattaatagcaaagacccagtgaggtcccacagctccctatttagaccaggtcatcaaaactaccttacatagaacagtgaacrgtgtggatcaacacagtgttataccagcattgacttcactttccacacttgtaaaaatgactttttggttgctacacagtaaagacgcttttataaaaactcagtttttaacacctatacaactttggatgaaggtttttaaaactttgactcctttaccgaattctgtagttctccccatcctcccagagcattaaaatgtctgaacttttcaccaaacaatcgtccgcaaatgtggcgttccaagtacacagtatgtccctcatttaacctgaaaaaaaaatattttaataaaaacacggacacagctgagaagaaaagacatttcaatcaagatattttctttttggcttttctacagaggaaagcagttgtaaggcatgaccactacagtctaagccgactctggctcccaggcagtcaatccagagcaatgggaagcccagcccagcagatggcagcagggaaagttaagccctgcttctgct

SEQ ID NO: 49 - DNA, 977 bp, Homo sapiens
tatcaccacgcaaataagctaataaggaggtattacttcactcagtggtgtaactttaggggaatctaaaacttggagactggaacactaggatatgttggcataaacttctggaagtctattaatagaatgcttacttaagtaatattctctgttgtttcttgctcaataatacaggctttattcttataaaaagactagaaaaatgatttaatgcctggtcagcaaatttggctttcaggagacaacacttaaaaatgacataccaaataagatgcaaacatagtaaacagctatattaatagcaaagacccagtgaggtcccacagctccctatttagaccaggtcatcaaaactaccttacatagaacagtgaacagtgtggatcaacacagtgttataccagcattgacttcactttccacacttgtaaaaatgactttttggttgctacacagtaaagacgcttttataaaaactcagtttttaayacctatacaactttggatgaaggtttttaaaactttgactcctttaccgaattctgtagttctccccatcctcccagagcattaaaatgtctgaacttttcaccaaacaatcgtccgcaaatgtggcgttccaagtacacagtatgtccctcatttaacctgaaaaaaaaatattttaataaaaacacggacacagctgagaagaaaagacatttcaatcaagatattttctttttggcttttctacagaggaaagcagttgtaaggcatgaccactacagtctaagccgactctggctcccaggcagtcaatccagagcaatgggaagcccagcccagcagatggcagcagggaaagttaagccctgcttctgctcttgcatgtccctatgttaaaagtgggagtatatcaggaattaaacttaacacctagactgaacctaacactcctaacgctgtaataagtgttacagaatttttaagaa

SEQ ID NO: 50 - DNA, 982 bp, Homo sapiens
aacttacattggagagtgacttgtcgcctgcctacaaaaaaagaaaattaccttagtattttagtaatcgaattacagactatctaaaagactgcctacctaaatacttagcatactggcactggtgatccactgtatttctactacagcacttcaaagaaggtaaaagagaccttaattgaaaaaacaaaaaaaatacagaactaaaaattagcatcattctttcttgccctaattctagggaatttttgcaatacaatgaaagccagtctatttgtgtctaacttccatgaaacatttcttctacttccatttttatatctgctcttatttacccatcactttctttctctcctattacccaaaatatatttaataaaactttagagtgtcctatgtgtcctgtgctgtattttatttattttattgctaatccatcactcattttggttctaagaagaatttaaagtagctcacaggcatattaaacatagcagcgtwctatggccctaatcctttcctgtacatggtgtactgattttttttttaattgtacctacacaccaagtgtaattggtatagtctgattgtctggatacataatttatcaatgaattgttgttacacagcacccccatgcccaactccccaaataccgtgaacataatattctccttctccaaatggcctgattattttcctttcaaaaacaagatggaggcctggtggggtggttcatgcctgtaatcccagcactttgggaggccaaggcaggtggatcacgaggtcagaggatcgagactaccctggccaatatagtgaaaccccatctctactaaaattacaaaaattagctgggcatggtggtgtgcacctatagtcccagctactcaagaggctgaggcaggagaatcgcttgaacccgggggcagaggttgcagtgagctgagattgtgccactgcactccaacctgggcaacag

SEQ ID NO: 51 - DNA, 978 bp, Homo sapiens
gtttattgcctcttgtaagacctcttgagggtctcatttcatccctcaatttcacaactatagaaacccagtcacaactcataagaaactattttttttttttttttttttttttgagacggagtctcgctctgtcacccaggctggagtgcagtggcacgatcttggctcactgcaagctccacctcccaggttcacaccattctcctgcctcagcctccctagtagctgggactacaggtgcccgccaccacgcccagctaattttttcttttttttgtatttttagtagagacagggtttcactgtgttagccaggatggtctcaattccctgacctcatgatccgcctgcctcggccacccaaattgctgggattacaggcgtgagccaccacgcccagcgttttttttttttttttttttaaatatacagggtctcattctgttgcccaggctgagtacagaggggccatcacagctcactgcagcctccrcctcctgggctcaagcaatactcccacctcagccttctgagtagctgggactacaggcatacactaccatgcccgattaattttttatttttttgtagagacatgatctcacttatgttgcccggcctggtcttgaactcctgggctcaagcgatcctcccactttggcctcccaaagtgacgggattacaggcatgagccacagagcccagcctgtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggact

SEQ ID NO: 52 - DNA, 971 bp, Homo sapiens
ctccctagtagctgggactacaggtgcccgccaccacgcccagctaattttttcttttttttgtatttttagtagagacagggtttcactgtgttagccaggatggtctcaattccctgacctcatgatccgcctgcctcggccacccaaattgctgggattacaggcgtgagccaccacgcccagcgttttttttttttttttttttaaatatacagggtctcattctgttgcccaggctgagtacagaggggccatcacagctcactgcagcctccacctcctgggctcaagcaatactcccacctcagccttctgagtagctgggactacaggcatacactaccatgcccgattaattttttatttttttgtagagacatgatctcacttatgttgcccggcctggtcttgaactcctgggctcaagcgatcctcccactttggcctcccaaagtgacgggattacaggcatgagccacagagcycagcctgtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggactgggcggtgggggaaagaggagctattgtttaattggtgcagagtttcagttctgcaaaatgaaaaatttctgaagatctgtttcacaacaatgtggatatacttaacactactgaaccgcacacttaaaaacagttaagtgtgcttaaaactaagaatgaacaaaaaattaagaaggaagggcactttatttgtaaaatattgataaaat

SEQ ID NO: 53 - DNA, 974 bp, Homo sapiens
agtagctgggactacaggtgcccgccaccacgcccagctaattttttcttttttttgtatttttagtagagacagggtttcactgtgttagccaggatggtctcaattccctgacctcatgatccgcctgcctcggccacccaaattgctgggattacaggcgtgagccaccacgcccagcgttttttttttttttttttttaaatatacagggtctcattctgttgcccaggctgagtacagaggggccatcacagctcactgcagcctccacctcctgggctcaagcaatactcccacctcagccttctgagtagctgggactacaggcatacactaccatgcccgattaattttttatttttttgtagagacatgatctcacttatgttgcccggcctggtcttgaactcctgggctcaagcgatcctcccactttggcctcccaaagtgacgggattacaggcatgagccacagagcccagcctrtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggactgggcggtgggggaaagaggagctattgtttaattggtgcagagtttcagttctgcaaaatgaaaaatttctgaagatctgtttcacaacaatgtggatatacttaacactactgaaccgcacacttaaaaacagttaagtgtgcttaaaactaagaatgaacaaaaaattaagaaggaagggcactttatttgtaaaatattgataaaatatcttacat

SEQ ID NO: 54 - DNA, 983 bp, Homo sapiens
atttttttgtagagacatgatctcacttatgttgcccggcctggtcttgaactcctgggctcaagcgatcctcccactttggcctcccaaagtgacgggattacaggcatgagccacagagcccagcctgtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggactgggcggtgggggaaagaggagctattgtttaattggtgcagagtttcagttctgcaaaatgaaaaatttctgaagatctgtttcacracaatgtggatatacttaacactactgaaccgcacacttaaaaacagttaagtgtgcttaaaactaagaatgaacaaaaaattaagaaggaagggcactttatttgtaaaatattgataaaatatcttacatttctgtaatatttgtaggcttccaagttctttaatatattttatctcatttgtttcacataaccaccctatgaggtagaaagtcagacattataatttcaaggataaggaaacagagattgagagtgacttgttcaagcttacatgagaatccagatctctaaaggtaagagcatgctcattttacaatacttgaaaaaataaggggtaactggtcaagatttttaaatgtaaaattaatttgttgcctacattttagatttgaatttttctagagctgtcagcttgatatcttgagaaatatgcaaatgattgaccaattaaccttgagagaagttcaagatgcctaagttttgatctttccacaaa

SEQ ID NO: 55 - DNA, 980 bp, Homo sapiens
acagagcccagcctgtaagactattctagaacaggaatgggtataaactttgtcatgcacttaaaggttgaatactcttatataagaagaaacaaatagaaaatgaaggaaatcctgtcagatgctataacgtggataaaccttaagggcattatgacaccttgaatgaaataagccagacacaaagagataaaatcatactgtatgattctacttatgtgaggtatctaaagtaatcaaattcataggaacagaaaatagaatgggtgttaccaaggactgggcggtgggggaaagaggagctattgtttaattggtgcagagtttcagttctgcaaaatgaaaaatttctgaagatctgtttcacaacaatgtggatatacttaacactactgaaccgcacacttaaaaacagttaagtgtgcttaaaactaagaatgaacaaaaaattaagaaggaagggcactttatttgtaaaataytgataaaatatcttacatttctgtaatatttgtaggcttccaagttctttaatatattttatctcatttgtttcacataaccaccctatgaggtagaaagtcagacattataatttcaaggataaggaaacagagattgagagtgacttgttcaagcttacatgagaatccagatctctaaaggtaagagcatgctcattttacaatacttgaaaaaataaggggtaactggtcaagatttttaaatgtaaaattaatttgttgcctacattttagatttgaatttttctagagctgtcagcttgatatcttgagaaatatgcaaatgattgaccaattaaccttgagagaagttcaagatgcctaagttttgatctttccacaaacctgaaaatttttccaaaagctcacctgctttctaaagctccaacaactaaagcaatcaggtagcagggtattggaacctaaagagggcaaacaaacgcacaccacgtgctt

SEQ ID NO: 56 - DNA, 992 bp, Homo sapiens
gtaggcttccaagttctttaatatattttatctcatttgtttcacataaccaccctatgaggtagaaagtcagacattataatttcaaggataaggaaacagagattgagagtgacttgttcaagcttacatgagaatccagatctctaaaggtaagagcatgctcattttacaatacttgaaaaaataaggggtaactggtcaagatttttaaatgtaaaattaatttgttgcctacattttagatttgaatttttctagagctgtcagcttgatatcttgagaaatatgcaaatgattgaccaattaaccttgagagaagttcaagatgcctaagttttgatctttccacaaacctgaaaatttttccaaaagctcacctgctttctaaagctccaacaactaaagcaatcaggtagcagggtattggaacctaaagagggcaaacaaacgcacaccacgtgcttgcattagtgttcacaaatgttacacartaagacaattcatatttaaaagtaagtaaattccctttcaaatctcctaatattagtagggataactttgcttttatacttctcaaatagttctcatctttaacatatagcttaaaattgtgatataaaacattgttcaaaacatctatttgccttttattctgctaggaacaaaagcttctcacacatgaaaaacaagatcacacatactattaaaaggtgcattttgagcatttctcaaaaagtaacctacaggaagcgcatttcccatatgtttgcctttttcctcctgacttttaaaaggttttggttttctttttttattcctttatgtttcaaagcactattggcatgttgtagaggcacacagagttacccggcaataagtagatgccaaagttatgggagcttggaaccacagaagctgcagtggaagtcaaattatccattgtgaggtcaattaagaaaacacacacacacacacacacacacacacac

SEQ ID NO: 57 - DNA, 999 bp, Homo sapiens
accgccaatgaaaacaaaaatctagaccctaggatcttactttttggatgaatttgtatattttcctgcttgggtcttctgggtcaggtgtttctccatcacgaatagcactcataagtgccaccagttctttagggacagacacctaatcaaggagaaaaatcatttctagtcataaataaaagcttctatgtgtcttaaaccatatatgtaaaataaccttttcttcccattcttgactatctaataaacagactatgaacacaaaaagtatatacatatacaaaaagtatatatatacacacacatatatatgaacacaacgtgtatagatgtgtatatatatgcacacatatatatgtgtatatatataaaacacatatacaaaaagtatatatatacacatatacatatcagttttgtaaataaaattagcaatatgggaaactggcttctttaaaagtgaatgtgaaatttctatccattcacccatgcacaytaagagcagagttttggtagaaactggattaaaatcccagctctgccacctaataactaactgcacaaacttgggcaaataaatataacccccgagcctcagtttccccatcaagtaagtgtaaaacttcaaaggcttgctgcaaggaataaataatataagtgaagagcccagcaccatccctggcaatggcagccaccatccctgctcccgctacactcacaaaacagattcaaaaggacgttatatactcactgtaggacagaatggttttgaacaattttgttttgaaaacacacacttggagttacaaatagaggaacattttaaaagtagtaactgtgaaaaactaaaatttattgctaaaaactgtcaaataattttctctggaaatccatacggaaaagacccttatgcggcaaaccatatagtcatttaactgtgtatcctagctccatgattctgaaagtttgatttctgatgaatgccagaataaagga

SEQ ID NO: 58 - DNA, 981 bp, Homo sapiens
tatatatatgcacacatatatatgtgtatatatataaaacacatatacaaaaagtatatatatacacatatacatatcagttttgtaaataaaattagcaatatgggaaactggcttctttaaaagtgaatgtgaaatttctatccattcacccatgcacattaagagcagagttttggtagaaactggattaaaatcccagctctgccacctaataactaactgcacaaacttgggcaaataaatataacccccgagcctcagtttccccatcaagtaagtgtaaaacttcaaaggcttgctgcaaggaataaataatataagtgaagagcccagcaccatccctggcaatggcagccaccatccctgctcccgctacactcacaaaacagattcaaaaggacgttatatactcactgtaggacagaatggttttgaacaattttgttttgaaaacacacacttggagttacaaatagaggaacaytttaaaagtagtaactgtgaaaaactaaaatttattgctaaaaactgtcaaataattttctctggaaatccatacggaaaagacccttatgcggcaaaccatatagtcatttaactgtgtatcctagctccatgattctgaaagtttgatttctgatgaatgccagaataaaggactcccccaagtattaatgatcaaacaagaatatattccagtaggggctagactttcatgttcttcttgcatggctcaggacccaaagctgtgactgaggcaggcacagaattagaagttcctgaaccagtgctacaacaattgtagattctaaagcacaaaactattcaggaaataattcggttcagccacctcccttcatttaggtggtgatacgttatatatatgtgccagctgaggttgcgaggtcataaaacttgttcaagtgtcacatcattatttatttatttttttagaaatggggtctcgctatgtcgccc

SEQ ID NO: 59 - DNA, 988 bp, Homo sapiens
cattttaaaagtagtaactgtgaaaaactaaaatttattgctaaaaactgtcaaataattttctctggaaatccatacggaaaagacccttatgcggcaaaccatatagtcatttaactgtgtatcctagctccatgattctgaaagtttgatttctgatgaatgccagaataaaggactcccccaagtattaatgatcaaacaagaatatattccagtaggggctagactttcatgttcttcttgcatggctcaggacccaaagctgtgactgaggcaggcacagaattagaagttcctgaaccagtgctacaacaattgtagattctaaagcacaaaactattcaggaaataattcggttcagccacctcccttcatttaggtggtgatacgttatatatatgtgccagctgaggttgcgaggtcataaaacttgttcaagtgtcacatcattatttatttatttttttagaaatggggtctcgctatgtcrcccaggctggccttgaacttctgagttcaagtgatcttcccacctcagcctcccaagtagttgggacttcacgcagttattaagtggtggagaagagccagagccctgggattctttgcctccaagtataatatatcactgcactatcctagatgtaatttggttgtgggatgatttgggaagcaagaaggccccataaatatgggttggtcctcattctatttgcttggtctaagtaggtctagcctccgggatagtgattatttagtaattacagtccgccttttccaaaaaggattagcagtacctaccaagggaataagttggaattgcatacagacaagtctggaatatatgcccactaggcttatatggctacagaatgcatttatagaaacttaaatcatgcaaatgtcaatttttaaaagttaagtaaaaattgttcctaagttcttatttctagatccaggattctgaatttcttctttttgttt

SEQ ID NO: 60 - DNA, 997 bp, Homo sapiens
tatgggttggtcctcattctatttgcttggtctaagtaggtctagcctccgggatagtgattatttagtaattacagtccgccttttccaaaaaggattagcagtacctaccaagggaataagttggaattgcatacagacaagtctggaatatatgcccactaggcttatatggctacagaatgcatttatagaaacttaaatcatgcaaatgtcaatttttaaaagttaagtaaaaattgttcctaagttcttatttctagatccaggattctgaatttcttctttttgtttgtttgtttttttgttttttgggttttttttttgagacggagtctggctctgtcgcccaggctggagtgcagtggtgccatctcagctcactccaagctctgcctcctgggttcatgccattctcctgcctcagcctgccgagtagctgggactacaggtgcccgccaccatgcccggctaattttttgtgttttttttagtacagayggggtttcaccatgttagccaggatggtctcgatctcctgacctcgtgatccacccatctcggcctcccaaagtgctgggattacaggtgtgagccaccacacctagccctgaattcctttttaaaagtcagattggtttccatttcctttttttcacagttaaaatgtttaaaactgcctttaaagtagagattcagaatgagtgccacagcctctttgtttacatatttcaggtagaatttcattaagaaaaataattctagctctaggaattcaattatcatctctgcttatcatttataccatatttactgatatgcatcatttaattgagttaataattcgtaatatttacctctgcagtataggttaatttcacagaaggagtgtcctgacaaggaaggattgctctgcagtggatggcctgaaaaagggagaaacaagaagaaatagctatttatctttcgcataagtcattaagaaatcattaaaaat

SEQ ID NO: 61 - DNA, 961 bp, Homo sapiens
aatctatggttaaccctcacatttcagttgaagcatggagaaactcttaagcagtgtttcctactctatggtctgggtgacagtagtgcccagtgagaagcttttagaaacctgagaaaaaagggctctgtagcaaaacagacctgagaagtatggcatactgcaccactgtcttgcagagccactagaatattagccgcctgaaggctctgaacagacctacaataaagaaacctgtttgatttcttacatttatgttaacacaaaacccatttctctctggtttaacacctaatgggatgtcagtattctaatgaacacagcctgagaaatgttgctgtaatcctgacacttcaatcttgcagcaaaccttgtaagtaaaacaaagaagcaaagaagggagaaagaacagtctctttcaataccatctagacatattcattcatatcatatgcaaagtgtttctgtactgccacaccaatcgtyattaacattggttccatccagtatgaccacaggccaggtgccgtggctcactcctgcaatcccagcactttgggaggctcagatgagaggattgcttgagctctagaatttgagaccagcctgggcaacatagtgagaccttacctctacacaaaaaaaattagctgggcatggtggtgcacacctgtagtcccagctactcaggaggctgaggtaaaaggatcgcttgagcccaggagttctaggctgcagtgacccaagttcgcaccattgcactacagcctgggcaacacagcaagaccctgtctccaaaaaaaaaaaaaaaagagcacctacaatcttatacccggtctgtttacaaataagtctgtctactgctggtgaacaatgaaatgaaaacccagcctcattgagacagtctactaaactcaaaggaattctgatattaacacccttctctgaagctattacaaat

SEQ ID NO: 62 - DNA, 965 bp, Homo sapiens
tgcaccactgtcttgcagagccactagaatattagccgcctgaaggctctgaacagacctacaataaagaaacctgtttgatttcttacatttatgttaacacaaaacccatttctctctggtttaacacctaatgggatgtcagtattctaatgaacacagcctgagaaatgttgctgtaatcctgacacttcaatcttgcagcaaaccttgtaagtaaaacaaagaagcaaagaagggagaaagaacagtctctttcaataccatctagacatattcattcatatcatatgcaaagtgtttctgtactgccacaccaatcgttattaacattggttccatccagtatgaccacaggccaggtgccgtggctcactcctgcaatcccagcactttgggaggctcagatgagaggattgcttgagctctagaatttgagaccagcctgggcaacatagtgagaccttacctctacacaaaaaaarttagctgggcatggtggtgcacacctgtagtcccagctactcaggaggctgaggtaaaaggatcgcttgagcccaggagttctaggctgcagtgacccaagttcgcaccattgcactacagcctgggcaacacagcaagaccctgtctccaaaaaaaaaaaaaaaagagcacctacaatcttatacccggtctgtttacaaataagtctgtctactgctggtgaacaatgaaatgaaaacccagcctcattgagacagtctactaaactcaaaggaattctgatattaacacccttctctgaagctattacaaatcctaaacatacttcattccaccacaagctttcttaaaacccccaaactccaggtcttttcatttcagttctagaaaattctccaaagatataggctcccaaatgacctctagatggattaagtaggactagcagagccacctggttctctctcccaaaatagatt

SEQ ID NO: 63 - DNA, 981 bp, Homo sapiens
tgggcatggtggtgcacacctgtagtcccagctactcaggaggctgaggtaaaaggatcgcttgagcccaggagttctaggctgcagtgacccaagttcgcaccattgcactacagcctgggcaacacagcaagaccctgtctccaaaaaaaaaaaaaaaagagcacctacaatcttatacccggtctgtttacaaataagtctgtctactgctggtgaacaatgaaatgaaaacccagcctcattgagacagtctactaaactcaaaggaattctgatattaacacccttctctgaagctattacaaatcctaaacatacttcattccaccacaagctttcttaaaacccccaaactccaggtcttttcatttcagttctagaaaattctccaaagatataggctcccaaatgacctctagatggattaagtaggactagcagagccacctggttctctctcccaaaatagatttccaaraccatgcctctatagttccttaatggtttctagttaggtgacatggcaacaccaaaggggtttttaaatgtatttcattggataaggccaaacccaggcaaatatgcatacagaacaaccgtaagcaaattcatcaaacaaaatcatgtctacatgattcctatcacctcaatcatttattaatttagctgaaatctgtttcccatattcccaccattgctgccaataagaaatggaataatatattcaaaattaacattttcatgactcataaatcttgcatttcttgccaactttggttaatagacattctattaagacatactgcctgaaaatcagatatttatgagatacagattgtgcaatttgtacactcttgcgtagaacatttcatctcttctagattattaaactgagggtttcttagattaaaaagatgtttcaagtggccatagaaagtaaacaggtctgattcatatgctaattccttttttaaatgg

SEQ ID NO: 64 - DNA, 981 bp, Homo sapiens
acccaagttcgcaccattgcactacagcctgggcaacacagcaagaccctgtctccaaaaaaaaaaaaaaaagagcacctacaatcttatacccggtctgtttacaaataagtctgtctactgctggtgaacaatgaaatgaaaacccagcctcattgagacagtctactaaactcaaaggaattctgatattaacacccttctctgaagctattacaaatcctaaacatacttcattccaccacaagctttcttaaaacccccaaactccaggtcttttcatttcagttctagaaaattctccaaagatataggctcccaaatgacctctagatggattaagtaggactagcagagccacctggttctctctcccaaaatagatttccaagaccatgcctctatagttccttaatggtttctagttaggtgacatggcaacaccaaaggggtttttaaatgtatttcattggataaggccaaayccaggcaaatatgcatacagaacaaccgtaagcaaattcatcaaacaaaatcatgtctacatgattcctatcacctcaatcatttattaatttagctgaaatctgtttcccatattcccaccattgctgccaataagaaatggaataatatattcaaaattaacattttcatgactcataaatcttgcatttcttgccaactttggttaatagacattctattaagacatactgcctgaaaatcagatatttatgagatacagattgtgcaatttgtacactcttgcgtagaacatttcatctcttctagattattaaactgagggtttcttagattaaaaagatgtttcaagtggccatagaaagtaaacaggtctgattcatatgctaattccttttttaaatggacttgtattgaaatttgaacctaacacacaggaatattgggagggatgaaacatgtaaagaatctagcacaatgcctggaaatagagca

SEQ ID NO: 65 - DNA, 994 bp, Homo sapiens
aaactcaaaggaattctgatattaacacccttctctgaagctattacaaatcctaaacatacttcattccaccacaagctttcttaaaacccccaaactccaggtcttttcatttcagttctagaaaattctccaaagatataggctcccaaatgacctctagatggattaagtaggactagcagagccacctggttctctctcccaaaatagatttccaagaccatgcctctatagttccttaatggtttctagttaggtgacatggcaacaccaaaggggtttttaaatgtatttcattggataaggccaaacccaggcaaatatgcatacagaacaaccgtaagcaaattcatcaaacaaaatcatgtctacatgattcctatcacctcaatcatttattaatttagctgaaatctgtttcccatattcccaccattgctgccaataagaaatggaataatatattcaaaattaacattttcatgactcayaaatcttgcatttcttgccaactttggttaatagacattctattaagacatactgcctgaaaatcagatatttatgagatacagattgtgcaatttgtacactcttgcgtagaacatttcatctcttctagattattaaactgagggtttcttagattaaaaagatgtttcaagtggccatagaaagtaaacaggtctgattcatatgctaattccttttttaaatggacttgtattgaaatttgaacctaacacacaggaatattgggagggatgaaacatgtaaagaatctagcacaatgcctggaaatagagcaaacgtttaatgaagtcagttcccttaattgtaaattatttgattactatgaaaagtaggtattttttctttcagaagacagtttgaaatgtattatccttgtgacaggttatctctaattgtatggctctttacccttagttttaaaacagaaaacaaaagtagtttaagtcatgcaatttta

SEQ ID NO: 66 - DNA, 999 bp, Homo sapiens
ttgggagggatgaaacatgtaaagaatctagcacaatgcctggaaatagagcaaacgtttaatgaagtcagttcccttaattgtaaattatttgattactatgaaaagtaggtattttttctttcagaagacagtttgaaatgtattatccttgtgacaggttatctctaattgtatggctctttacccttagttttaaaacagaaaacaaaagtagtttaagtcatgcaattttaaaggtacagttaatatattgatataatacatacttttgtaaatgtgtaagaaaaatatggaaaagctacattccaaactcaatggtggttacctctgggcaatggtgtctggaaaaggtttggaaattaaatctttcactttccatttctttactattagcatttttcataaccagtacatattatttattaatttttcctttcattttatgactatttactgagtacctactctctgctaagttctaagtcaggcctagagagyccaatctaggtggacatatttccaaactgaaagaagcttcttatttaaagtaaggcatgagtgtattaatagtgaaagataaaatgaaaatatataattcatcttatatgtttctataagatcaattaatacattttattaggtaaaacctacataatccataaaaccactgttcattttgcttcattcaaccataggtgctgaaattttctgcatcagaaatcattctggaatcctttttacctggcactgactaaagagatatgggtgttccttcccagaagtctgttcaggagtgagccactggagagcagaagattttggagaggtctcaaaagaaatttctataacaatttcttgatttctgtatgaaacacataaatatattagtagagtatgattccatctagtgaaaatttaaactcataatacatacactgaataatataaataacatagtatgcattctcatcactgattggcagtaagctctaggta

SEQ ID NO: 67 - DNA, 1001 bp, Homo sapiens
aagctacattccaaactcaatggtggttacctctgggcaatggtgtctggaaaaggtttggaaattaaatctttcactttccatttctttactattagcatttttcataaccagtacatattatttattaatttttcctttcattttatgactatttactgagtacctactctctgctaagttctaagtcaggcctagagagtccaatctaggtggacatatttccaaactgaaagaagcttcttatttaaagtaaggcatgagtgtattaatagtgaaagataaaatgaaaatatataattcatcttatatgtttctataagatcaattaatacattttattaggtaaaacctacataatccataaaaccactgttcattttgcttcattcaaccataggtgctgaaattttctgcatcagaaatcattctggaatcctttttacctggcactgactaaagagatatgggtgttccttcccagaagtctgttcaggagtragccactggagagcagaagattttggagaggtctcaaaagaaatttctataacaatttcttgatttctgtatgaaacacataaatatattagtagagtatgattccatctagtgaaaatttaaactcataatacatacactgaataatataaataacatagtatgcattctcatcactgattggcagtaagctctaggtatgccacatcctcagtgggtaagtctcctctcagttttcctacctaattgccagcctgtgggtccttttacctctcccatgctaactgctagcgaaggcttaatggcaactaacagtggttgactaccccgttgtgtgtcacgtactttgcatctgtgatatcatttaatattttattagagtgaaaaagtaaaagaaatcatttttggggcttcaactaccacagcagcaggtgccacagcatgacacagagcagtgctagtctgcaaactgttaccggcccaggacaagacaagaccag

SEQ ID NO: 68 - DNA, 1001 bp, Homo sapiens
tatataattcatcttatatgtttctataagatcaattaatacattttattaggtaaaacctacataatccataaaaccactgttcattttgcttcattcaaccataggtgctgaaattttctgcatcagaaatcattctggaatcctttttacctggcactgactaaagagatatgggtgttccttcccagaagtctgttcaggagtgagccactggagagcagaagattttggagaggtctcaaaagaaatttctataacaatttcttgatttctgtatgaaacacataaatatattagtagagtatgattccatctagtgaaaatttaaactcataatacatacactgaataatataaataacatagtatgcattctcatcactgattggcagtaagctctaggtatgccacatcctcagtgggtaagtctcctctcagttttcctacctaattgccagcctgtgggtccttttacctctcccatgctaactgctagcraaggcttaatggcaactaacagtggttgactaccccgttgtgtgtcacgtactttgcatctgtgatatcatttaatattttattagagtgaaaaagtaaaagaaatcatttttggggcttcaactaccacagcagcaggtgccacagcatgacacagagcagtgctagtctgcaaactgttaccggcccaggacaagacaagaccagaagttgagagtcagcattgcaaaacttttagagtcatttttgtctgttgaatctaataataaaaaatgtgtgcttgtatttcatctcttcttcctcatatttcatttttattgcattgtacaaaagtatcagtctatgacagattgaagaggatagaaattggtcctttaccccagagagtttgagaagcactgataataaggaaacagcagaggtttagagaccagcagccctgctggtgttcgaatcctgactctatcacttactggtactgtaaacttggggaaattatt

SEQ ID NO: 69 - DNA, 983 bp, Homo sapiens
gcattgtacaaaagtatcagtctatgacagattgaagaggatagaaattggtcctttaccccagagagtttgagaagcactgataataaggaaacagcagaggtttagagaccagcagccctgctggtgttcgaatcctgactctatcacttactggtactgtaaacttggggaaattatttgacctccctatgccacagtttccttgtagaatggggtgaataccatctacctcacaagctagacttaagtgtttcccttctcttaaagggaaagagaaggcatgaaaacactggcctctgaacaactggggtagatcacccttgttctaggccaatagttttcaccctctttcccctcaagaggtggcatatactcccagtgtgacaattctggttgccactttcttgaataagttatttctctaaggttccctttcctcatctttaagtgtagattataccagcagggttactgtaaggattagayacaagaatgcatttaaagcacttatcccaagattgctgcactgtaacagttctatctttggcattatcattgtcccattaataaatgcagctggcctctggggcaagggcaaggagggtgcaacttgtaaagctgcccaggttatcttgaaatgccttcttatgatggcatgcccccaccatcactctagatattagtaaaaggatgaatcgtttagaaactaacagttcccaaagtccttgttgtattatatacaaacaacatttttagtatcttaagtatatataattttaactgctgtatcaactttaatctgaacagaagatcaggataagtagtgtaccaatcattacatatttacaaactaaaatttaaaaagaaaaaatatttaaattagttaagaatatgtttccccattatttagctgtaaaagagaaagatcataacattcatacttgctcaaagcgataggaagagagatttccattggcgat

SEQ ID NO: 70 - DNA, 991 bp, Homo sapiens
ggaaacagcagaggtttagagaccagcagccctgctggtgttcgaatcctgactctatcacttactggtactgtaaacttggggaaattatttgacctccctatgccacagtttccttgtagaatggggtgaataccatctacctcacaagctagacttaagtgtttcccttctcttaaagggaaagagaaggcatgaaaacactggcctctgaacaactggggtagatcacccttgttctaggccaatagttttcaccctctttcccctcaagaggtggcatatactcccagtgtgacaattctggttgccactttcttgaataagttatttctctaaggttccctttcctcatctttaagtgtagattataccagcagggttactgtaaggattagatacaagaatgcatttaaagcacttatcccaagattgctgcactgtaacagttctatctttggcattatcattgtcccattaataaatgcagctrgcctctggggcaagggcaaggagggtgcaacttgtaaagctgcccaggttatcttgaaatgccttcttatgatggcatgcccccaccatcactctagatattagtaaaaggatgaatcgtttagaaactaacagttcccaaagtccttgttgtattatatacaaacaacatttttagtatcttaagtatatataattttaactgctgtatcaactttaatctgaacagaagatcaggataagtagtgtaccaatcattacatatttacaaactaaaatttaaaaagaaaaaatatttaaattagttaagaatatgtttccccattatttagctgtaaaagagaaagatcataacattcatacttgctcaaagcgataggaagagagatttccattggcgatcccttgtaactttgtctttctccaagagcatatttgacttcttgtccattgatcactactttttctattgtaaggtcctttgtatccaaaacctaaa

SEQ ID NO: 71 - DNA, 999 bp, Homo sapiens
tcctttgtatccaaaacctaaaattaatatttttaaatagtaagaaaatagtttcatttaccagaaaaaactcatattagatataggctacaacaactagttgcttatggagagtaaaatacagagtgaaattagaagaattgaagagtcaaaagctagtctaggtctcattttttgggactctaagcatcttgaaaatttttgggttctaagatttgcatatatattgttaaataaccctaggacagtcacacaaattttgggctttaagtaaaagtcaaatctaaatcaaaatatgtttgcttctgactcctaaaattttctctattatgaaaaactttatctataacttaagtttctttcactctggctcctcaatacattacacaatatatttcctcctagaactcatgtactttcaaacttcatgttcgttaagcaaatcagcaaactgtatatcactgtggttgtatatctagaaaaagcccaacctggtatggyaactcagaccaaatgattctgcagaggattgggaggccatatctacttgccatggccaattaaggacaactgctttgggcatgaaggagtgacatcaagtgtcagagtattttctatccccaaaatcctgagccctacaaatcatactctttaattatctctcaactaatctcttgtcctagaatcttgaaccttcctatgccacaagactgtttcctaacaacataaacaaaattctacttgatggatctacccactaaatattctagttttcctccttccttccttaaactccaagggagtttttgactgctatgactactacttctacttcttcattaatcatcctccctttccccttcttccatctggcttcttgctattgaaagggcagcccccaccccgatcaacaaagtcttttctgtccaataaccttgacctctgtctactcacagcccttatggactatgtcatctggttaaaaccccttccttcact

SEQ ID NO: 72 - DNA, 1000 bp, Homo sapiens
tgtcctagaatcttgaaccttcctatgccacaagactgtttcctaacaacataaacaaaattctacttgatggatctacccactaaatattctagttttcctccttccttccttaaactccaagggagtttttgactgctatgactactacttctacttcttcattaatcatcctccctttccccttcttccatctggcttcttgctattgaaagggcagcccccaccccgatcaacaaagtcttttctgtccaataaccttgacctctgtctactcacagcccttatggactatgtcatctggttaaaaccccttccttcacttctttgcctgtacgcatacatcataaatggttctctatttgtctaatgtttttttcctttcccctcctttattccaattcaaaaatatggatatgtcccaatgttccagccccggtcctttgattttcttgccatatccttcactccctagctcttactcatgcccacatcttcaaytagtatctctgtgaagatgcctgccattctagttctacagttgtattccctccccaggacctcagtcgaatcgcctgctcaacatttccatgggacatagcaccacacattgaataggcttctaaaaattccaaaaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggcagaactgctttctaccagatctccctacctctggcattatttttttccttttctgaaatctgacctggctacatgtgaggccaagaaccagccatttcccagctgcccctgggtactttcttttgggggtacctcatttgttatccttactctaaattagtagaagatacggtt

SEQ ID NO: 73 - DNA, 1001 bp, Homo sapiens
actgctatgactactacttctacttcttcattaatcatcctccctttccccttcttccatctggcttcttgctattgaaagggcagcccccaccccgatcaacaaagtcttttctgtccaataaccttgacctctgtctactcacagcccttatggactatgtcatctggttaaaaccccttccttcacttctttgcctgtacgcatacatcataaatggttctctatttgtctaatgtttttttcctttcccctcctttattccaattcaaaaatatggatatgtcccaatgttccagccccggtcctttgattttcttgccatatccttcactccctagctcttactcatgcccacatcttcaattagtatctctgtgaagatgcctgccattctagttctacagttgtattccctccccaggacctcagtcgaatcgcctgctcaacatttccatgggacatagcaccacacattgaataggcttctaaaaattccaraaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggcagaactgctttctaccagatctccctacctctggcattatttttttccttttctgaaatctgacctggctacatgtgaggccaagaaccagccatttcccagctgcccctgggtactttcttttgggggtacctcatttgttatccttactctaaattagtagaagatacggtttatatcttatttaaaataatagggttactccttcatattctagtacctctctagtctcttcatagtctagtacctagttctgaatagctattcagaatagctaacttgttttaaaaacttgatttgagtatcttg

SEQ ID NO: 74 - DNA, 1001 bp, Homo sapiens
ctttgcctgtacgcatacatcataaatggttctctatttgtctaatgtttttttcctttcccctcctttattccaattcaaaaatatggatatgtcccaatgttccagccccggtcctttgattttcttgccatatccttcactccctagctcttactcatgcccacatcttcaattagtatctctgtgaagatgcctgccattctagttctacagttgtattccctccccaggacctcagtcgaatcgcctgctcaacatttccatgggacatagcaccacacattgaataggcttctaaaaattccaaaaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggyagaactgctttctaccagatctccctacctctggcattatttttttccttttctgaaatctgacctggctacatgtgaggccaagaaccagccatttcccagctgcccctgggtactttcttttgggggtacctcatttgttatccttactctaaattagtagaagatacggtttatatcttatttaaaataatagggttactccttcatattctagtacctctctagtctcttcatagtctagtacctagttctgaatagctattcagaatagctaacttgttttaaaaacttgatttgagtatcttgtgtttataacacatgcttatatagatgaattaactgggtcatttcccagtggaacatattctgttttctatattggctaaactttccaaatctgttcagaatcagaagtgtcatagtgacaactattttttgtgaaacgttttgatatcccctgtgtctgttatagctcttggccctaccctttcctataa

SEQ ID NO: 75 - DNA, 1001 bp, Homo sapiens
cccaatgttccagccccggtcctttgattttcttgccatatccttcactccctagctcttactcatgcccacatcttcaattagtatctctgtgaagatgcctgccattctagttctacagttgtattccctccccaggacctcagtcgaatcgcctgctcaacatttccatgggacatagcaccacacattgaataggcttctaaaaattccaaaaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggcagaactgctttctaccagatctccctacctctggcattatttttttccttttctgaaatctgacctggctacatgtgaggccaagaaccagccayttcccagctgcccctgggtactttcttttgggggtacctcatttgttatccttactctaaattagtagaagatacggtttatatcttatttaaaataatagggttactccttcatattctagtacctctctagtctcttcatagtctagtacctagttctgaatagctattcagaatagctaacttgttttaaaaacttgatttgagtatcttgtgtttataacacatgcttatatagatgaattaactgggtcatttcccagtggaacatattctgttttctatattggctaaactttccaaatctgttcagaatcagaagtgtcatagtgacaactattttttgtgaaacgttttgatatcccctgtgtctgttatagctcttggccctaccctttcctataatacttactgtactgcattataatgatttctttttccattagactaagggttctaaaacagagaatgttacttaggtctgtattcccagggtttag

SEQ ID NO: 76 - DNA, 1001 bp, Homo sapiens
gtattccctccccaggacctcagtcgaatcgcctgctcaacatttccatgggacatagcaccacacattgaataggcttctaaaaattccaaaaatgatttttatactccctgaatcagatttctccccagatttcttgattctgttaaaagaactcttccagttacctaaggtttgatcccatttcccaaccccacacagccacttaaaagttgttctttcacaatgtcttcatacttttcctttctttccactactaacccaggtcaggccctggactggcagaactgctttctaccagatctccctacctctggcattatttttttccttttctgaaatctgacctggctacatgtgaggccaagaaccagccatttcccagctgcccctgggtactttcttttgggggtacctcatttgttatccttactctaaattagtagaagatacggtttatatcttatttaaaataatagggttactccttcatattctagyacctctctagtctcttcatagtctagtacctagttctgaatagctattcagaatagctaacttgttttaaaaacttgatttgagtatcttgtgtttataacacatgcttatatagatgaattaactgggtcatttcccagtggaacatattctgttttctatattggctaaactttccaaatctgttcagaatcagaagtgtcatagtgacaactattttttgtgaaacgttttgatatcccctgtgtctgttatagctcttggccctaccctttcctataatacttactgtactgcattataatgatttctttttccattagactaagggttctaaaacagagaatgttacttaggtctgtattcccagggtttagcactctgcctcaaaaacactaggtgtcaattaatgcatgaagcaggtcctagaccaagagaaaacaaaaaatgcaatgtttaagctgtattatctcaagtcctaagtctcaactatcatttgc

SEQ ID NO: 77 - DNA, 999 bp, Homo sapiens
accctttcctataatacttactgtactgcattataatgatttctttttccattagactaagggttctaaaacagagaatgttacttaggtctgtattcccagggtttagcactctgcctcaaaaacactaggtgtcaattaatgcatgaagcaggtcctagaccaagagaaaacaaaaaatgcaatgtttaagctgtattatctcaagtcctaagtctcaactatcatttgcaaactactttttaaaattccccttcaaatttcagcgatgttatttttaaaaaatagtcaaaaactgtaataagaaagaaaaataaagaaaactggattgttgacaagttggatttagtactttttaagaaacgtgttaagcatcaacagctctactaattataggatataatttatatgtttcacagtatcctctttgaacaataccctccatccccctaaaaagcagttgtacttctcagtagctggtcagttgacatggaatagrtatctgattccttttttgcacaggctggtaggaagctccatgtcaaccctgtggcccacttcttttaaagtatagagggctttatgccatgggttttgtttctcctatccctattctctcttcctgcaaattatttaattatttttaatcttatactatatatgttgcttcaagcagtctcagtcctttctagaacaaagcagagtttttttaaaaaaagctttatgcctcattatgatgtctaaatttacattttctacttgctatgtgcagggatatgatgaaaaaaaataggtttatgtgtgaaacacaaagctaaaactaaaaaaccaccttgatttgattcccagttgagacatttacttagtgaaaacaagatggtttgcagtcagaattacctattgttaactgctggcttctgccttggccatggcactaaaacctcttgagccactaaccaaaagaacacctaaacatttctgaaggtttcagtgaaaaga

SEQ ID NO: 78 - DNA, 996 bp, Homo sapiens
gttctaaaacagagaatgttacttaggtctgtattcccagggtttagcactctgcctcaaaaacactaggtgtcaattaatgcatgaagcaggtcctagaccaagagaaaacaaaaaatgcaatgtttaagctgtattatctcaagtcctaagtctcaactatcatttgcaaactactttttaaaattccccttcaaatttcagcgatgttatttttaaaaaatagtcaaaaactgtaataagaaagaaaaataaagaaaactggattgttgacaagttggatttagtactttttaagaaacgtgttaagcatcaacagctctactaattataggatataatttatatgtttcacagtatcctctttgaacaataccctccatccccctaaaaagcagttgtacttctcagtagctggtcagttgacatggaataggtatctgattccttttttgcacaggctggtaggaagctccatgtcaaccctgtggcccayttcttttaaagtatagagggctttatgccatgggttttgtttctcctatccctattctctcttcctgcaaattatttaattatttttaatcttatactatatatgttgcttcaagcagtctcagtcctttctagaacaaagcagagtttttttaaaaaaagctttatgcctcattatgatgtctaaatttacattttctacttgctatgtgcagggatatgatgaaaaaaaataggtttatgtgtgaaacacaaagctaaaactaaaaaaccaccttgatttgattcccagttgagacatttacttagtgaaaacaagatggtttgcagtcagaattacctattgttaactgctggcttctgccttggccatggcactaaaacctcttgagccactaaccaaaagaacacctaaacatttctgaaggtttcagtgaaaagaaacaaatgtatgaaagttatcataaatttggaggatcaaacttcagtgtaaataaccca

SEQ ID NO: 79 - DNA, 993 bp, Homo sapiens
ttaaagtatagagggctttatgccatgggttttgtttctcctatccctattctctcttcctgcaaattatttaattatttttaatcttatactatatatgttgcttcaagcagtctcagtcctttctagaacaaagcagagtttttttaaaaaaagctttatgcctcattatgatgtctaaatttacattttctacttgctatgtgcagggatatgatgaaaaaaaataggtttatgtgtgaaacacaaagctaaaactaaaaaaccaccttgatttgattcccagttgagacatttacttagtgaaaacaagatggtttgcagtcagaattacctattgttaactgctggcttctgccttggccatggcactaaaacctcttgagccactaaccaaaagaacacctaaacatttctgaaggtttcagtgaaaagaaacaaatgtatgaaagttatcataaatttggaggatcaaacttcagtgtaaataacccaaaactkgaaaagaattttagaaagcttagaatttgtccgattaagtctccttcagcattcctcaacatcacaaactctaagaacggagaggaaaagaagacatgacgtctctcctgattccgcactggcactgggtcttcccatctcacctctgaaatacagctggcactattatcaatgtagcccatgttaagcttaggcactgttttctaattgaaatcatccattaatcaaacttttgaatgtcctctacatgccagacatagactatactaggaagctgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataat

SEQ ID NO: 80 - DNA, 977 bp, Homo sapiens
agacatttacttagtgaaaacaagatggtttgcagtcagaattacctattgttaactgctggcttctgccttggccatggcactaaaacctcttgagccactaaccaaaagaacacctaaacatttctgaaggtttcagtgaaaagaaacaaatgtatgaaagttatcataaatttggaggatcaaacttcagtgtaaataacccaaaacttgaaaagaattttagaaagcttagaatttgtccgattaagtctccttcagcattcctcaacatcacaaactctaagaacggagaggaaaagaagacatgacgtctctcctgattccgcactggcactgggtcttcccatctcacctctgaaatacagctggcactattatcaatgtagcccatgttaagcttaggcactgttttctaattgaaatcatccattaatcaaacttttgaatgtcctctacatgccagacatagactatactaggaagytgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataattccaggctctaatttgctaaacagacatgccaaccagaaatcactattttaaaatcttacttttctctagatttggggaatgtaaaaacaatgagcagatttttagattgggacattcttttcaaaatttaaacatcctgactcttgcttacttatagaacagagataaagtttttattctacaaaagtgatgagaacacatggatacacagtggggaacacacactggggcttactggagggtggagggtaggagaagggaaaggatcagga

SEQ ID NO: 81 - DNA, 982 bp, Homo sapiens
agaaagcttagaatttgtccgattaagtctccttcagcattcctcaacatcacaaactctaagaacggagaggaaaagaagacatgacgtctctcctgattccgcactggcactgggtcttcccatctcacctctgaaatacagctggcactattatcaatgtagcccatgttaagcttaggcactgttttctaattgaaatcatccattaatcaaacttttgaatgtcctctacatgccagacatagactatactaggaagctgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataattccaggctctaayttgctaaacagacatgccaaccagaaatcactattttaaaatcttacttttctctagatttggggaatgtaaaaacaatgagcagatttttagattgggacattcttttcaaaatttaaacatcctgactcttgcttacttatagaacagagataaagtttttattctacaaaagtgatgagaacacatggatacacagtggggaacacacactggggcttactggagggtggagggtaggagaagggaaaggatcaggaaaagtaactaatgggtactaggcttaatacctgggtgacaaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcata

SEQ ID NO: 82 - DNA, 991 bp, Homo sapiens
aatcaaacttttgaatgtcctctacatgccagacatagactatactaggaagctgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataattccaggctctaatttgctaaacagacatgccaaccagaaatcactattttaaaatcttacttttctctagatttggggaatgtaaaaacaatgagcagatttttagattgggacattcttttcaaaatttaaacatcctgactcttgcttacttatagaacagagataaagtttttattctacaaaagtgatgagaacacatggatacacagtggggaacacacaytggggcttactggagggtggagggtaggagaagggaaaggatcaggaaaagtaactaatgggtactaggcttaatacctgggtgacaaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggt

SEQ ID NO: 83 - DNA, 993 bp, Homo sapiens
catagactatactaggaagctgagatacaaagagttatgaaacacagtctctacattcaagagtccacaatctagtggaggaaagaaacaagttaactttaaataaatactaattaactaattaataaggataagctcctggtctaaggcttttgtcataaataagcaaacaattataaacatgttattttgtaccataaattgccttccttgtataacatgtaacattattataattccaggctctaatttgctaaacagacatgccaaccagaaatcactattttaaaatcttacttttctctagatttggggaatgtaaaaacaatgagcagatttttagattgggacattcttttcaaaatttaaacatcctgactcttgcttacttatagaacagagataaagtttttattctacaaaagtgatgagaacacatggatacacagtggggaacacacactggggcttactggagggtggagggtaggaraagggaaaggatcaggaaaagtaactaatgggtactaggcttaatacctgggtgacaaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtg

SEQ ID NO: 84 - DNA, 1000 bp, Homo sapiens
ctgggtgacaaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtgtcctgaattgcttaatttttaaatctctagtacttttgaatgtaggaygtataaactcatgttcaaatatggcagtctcacagtgtggtttttctttttttattattatactttaagttctggggtacatgtgcagaacgtgcaggtttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttc

SEQ ID NO: 85 - DNA, 1000 bp, Homo sapiens
aaaataatctgtacaacaaaccctcatgacacaagtttacctatgtaacaaacctgcacatttgaagtacacctgaacttcaaataataaattttttaagtttttattttacaaaacaaaggtaagtgtgaggtcacattaagcagcaaaaagctataaaaattttcattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtgtcctgaattgcttaatttttaaatctctagtacttttgaatgtaggacgtataaachcatgttcaaatatggcagtctcacagtgtggtttttctttttttattattatactttaagttctggggtacatgtgcagaacgtgcaggtttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatc

SEQ ID NO: 86 - DNA, 1001 bp, Homo sapiens
cattcttttacttttatcagcatagtttataatttaatttttttaaataaaggtgaagaacaagaactttccagttaactaagagctttgagtgggtttggggcttagtcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtgtcctgaattgcttaatttttaaatctctagtacttttgaatgtaggacgtataaactcatgttcaaatatggcagtctcacagtgtggtttttctttttttattattatactttaagttctggggtacatgtgcagaacgtgcaggtttgttacataagtatacacatgccatggtggtttgctgcacccatcaacccgtcagctacattaggtatttctccwaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatcagtttagcaaaatgttgagggatgaagagtaagaaagtaagtacttattagttcatattaatgaaatcaaaattcagatccttcctacacaagtaggaaaaagaggcctgaaagccaccaattcttatctgcccgatctgatctgattgcttattgatgtgctttag

SEQ ID NO: 87 - DNA, 997 bp, Homo sapiens
tcaaggttttattatatcttaaaccaattggaatatttcttctgaaatatatgttgcagctaaagattcaaggaagaatttgctgttcatatattagaaaaacctctttaaatttcttccactagcgacctcggttttggtttgcaattattcacatctgaacacaagtgtcctgaattgcttaatttttaaatctctagtacttttgaatgtaggacgtataaactcatgttcaaatatggcagtctcacagtgtggtttttctttttttattattatactttaagttctggggtacatgtgcagaacgtgcaggtttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagamcatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatcagtttagcaaaatgttgagggatgaagagtaagaaagtaagtacttattagttcatattaatgaaatcaaaattcagatccttcctacacaagtaggaaaaagaggcctgaaagccaccaattcttatctgcccgatctgatctgattgcttattgatgtgctttagtagatttcaccatgctacactgtgtaaaatacacatgtagcatcctgccctggtgaagaagccgaatttggctgtcttttcatgaccctcttatttttaaaatg

SEQ ID NO: 88 - DNA, 992 bp, Homo sapiens
gaacgtgcaggtttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatcagtttagcaaaatgttgagggatgaagagtaagaaagtaagtacttattagttcatattaatgaaatcaaaatkcagatccttcctacacaagtaggaaaaagaggcctgaaagccaccaattcttatctgcccgatctgatctgattgcttattgatgtgctttagtagatttcaccatgctacactgtgtaaaatacacatgtagcatcctgccctggtgaagaagccgaatttggctgtcttttcatgaccctcttatttttaaaatgatcttctatgaaattcttcaggtgaaaggtaccttcagatgaaaggtataaaccaaatactattgggcaatttgagcaagaacattaaatataggttatgatacagataaaatcattgaataatattccatgaatctacaacctttcttcattccaatggttatagagtttgtagaagtatgtgttttctaagtgaaataactacttggctccttggaaccaactattaaaaaagcgtattgaatcatccttagaaaatttgaacgtcccatccgttcttaaattattagaagaaagttg

SEQ ID NO: 89 - DNA, 992 bp, Homo sapiens
ttgttacataagtatacacatgccatggtggtttgctgcacccatcaacccgtcagctacattaggtatttctcctaatgctatccctcccctaggcccctacccccaacaggccctggtgtgtgatgttcccctccctgtgtccatgtgttctcattgttcaactctcacttatgagtgagaacatgcggtgtttagttttgaaactgcattgaaataggacttcagccctgcccaggcaaagttgctactgcaattaaagatagcatggtacttcaagaagaccaaagtgcgatctgcaaggaaatagatgccttcctgcttataatatcttaattttctttcttatggtacttttgttgattacctatcagtacatagaggaatcgacctatttttcaaatcaatcagtttagcaaaatgttgagggatgaagagtaagaaagtaagtacttattagttcatattaatgaaatcaaaattcagatccttcctayacaagtaggaaaaagaggcctgaaagccaccaattcttatctgcccgatctgatctgattgcttattgatgtgctttagtagatttcaccatgctacactgtgtaaaatacacatgtagcatcctgccctggtgaagaagccgaatttggctgtcttttcatgaccctcttatttttaaaatgatcttctatgaaattcttcaggtgaaaggtaccttcagatgaaaggtataaaccaaatactattgggcaatttgagcaagaacattaaatataggttatgatacagataaaatcattgaataatattccatgaatctacaacctttcttcattccaatggttatagagtttgtagaagtatgtgttttctaagtgaaataactacttggctccttggaaccaactattaaaaaagcgtattgaatcatccttagaaaatttgaacgtcccatccgttcttaaattattagaagaaagttgataagattaaaa

SEQ ID NO: 90 - DNA, 1001 bp, Homo sapiens
ttggctccttggaaccaactattaaaaaagcgtattgaatcatccttagaaaatttgaacgtcccatccgttcttaaattattagaagaaagttgataagattaaaaagtagaaaggaccctgaagagagagagctgcgcctagagttagcaagcagggactgttagtttcaaagtagggcggaaagaagaggcctgcccggccggggctggaaatcctaagaggcttgagaacgactagcagggagatccagggaactaggagggagacggatgggtggtgccctgcagacctgtggattgaaataagtgttcccgggaggcaaccgtgggatcagggatcgacaggacatgggatctgagacttgggtgagattgttgactgaggaaggtgcccagggggctgggaaaagtctggggcctgaagaagggggttctgggccgcaggccgaagcaatggggaggccatggagtaattagagccaggaactaaaattatggkggctactgcaaagatgacacctaagggctgggtgagttgagaggagtggacgaggcgctggatgtgcccagggacctcggagagaggatccaggcgaggggcggaggagacatacgtataagtgggggctgagggaagggatgcagaggcgtaagcggggttgagaaggggtgctgtgagagatctgggggctgaagtgcacaacatgagttggatggaggctacagaagagcagacggggacgtggggctaggcaggggccgcggcggggtgagccggagatccgggagcccgcaaggactagggtcgaggggcagggagcccgggagaggcgggcactgggcaggcgccccactgtaccaggctgcgcagattgtcctcctgagactggaccgtgagagcagcagtcccggtcagcgtccggcgagtaaagtcgacgctgcagcgcaggtgcaggtgcttggtccggcagacggaagccggagaggccaacgaacagg

SEQ ID NO: 91 - DNA, 909 bp, Homo sapiens
agtaaagatttcagaggtgtgagggatagttgatgggtttagcatgctggtatggttcaattctctatcaaaagtgacgaaatttagctccagcaacaacaacaaaaaactgctatatttctggatatccttgtgttggcccctgcaagccaaaggaaaacaaaataaaaccaaaaaatcccaaactatgaaatctaataccttacacatgcataggtcctaattcatagggtgtaagaatttgtcatcaacatttgcattttcggatttttttggcaaatgtcctgttgcccaggctggatacagtggcatgatcatgggtaactgcacattcaacctcctggactcaagcgattctcgtgcctcagcctccaagtagctgggactacaggcgcccrccaccacgcctggctaatttttatatttttttagagatggggttttgtcatgttgcccaagctggtctcaaacttctgagctcaagggatccacctgccttggccttccaaagtgctgggattacaggtacgagccaccacacagagccgcaaacattttttgaggtcaccaaatctagggtgacaaatacaatagataacatagaattcatttagtcaaataatacacagtcaaatcatcttatttatctagtatggagaaaggatagtttgttttaataagaacgtcattatcatcatcttctattattgattaccaggaacccacagagtttatgccacttgtgtttaaataaaaatatccacacacaaccacaaataaattcctccattaatatattcatcaaaaaataaattacagtaggaattgttttctgagataccactcaccccaaatatagaatgtacaaaatttgcaatttacaagcaattggagtattattgatatcca

SEQ ID NO: 92 - DNA, 847 bp, Homo sapiens
ctcgatgaagaaggaaaaccaaggaagtccgtgtcttggatgacaagtgacatctggaaaaataaaggagcagtgtggtcagggagcctgatgaaattctgactatggatgactcactgttttgtgtaaaaagggggaagagaatttattctaaaaatttgttcatatctacataaaatacttctggagggatgctcaagaaactcatggtattgtttgcctgtgtggacagagaaggaaggccaaagaacagaggtgaaaagtagatatttcaactgaataatcttgtaagccttttgaattttaatgtgaatatatttcccagtcaaaaggttatttattgatatgaaaaaaaataaaggtcactggaatcccaaaccacaaacaaaaacagcccttgctgacttcctgtggacttcatagtgtctaccactggccccrcggggctctgcagcttccacttgagtggctcgatacaccctgcgtcagccatgctgaaccaaggtgttcaagctctctgcactctctggcccttccttgagcctgcatgcccttcccactcccactcttcccgcaaccttggcagggctctcctcctccccttcaggactctgccccccaccaccctccagtctgggctagagtctagtagaatctcccttgctaagagaacaaggtgcatgtgacacccttctcttcctcccttcagtgtgtgagcaaatagaagaaatgattttagccacatttttaatgttcaccttacaacatagttgaggcaatcctgaccagtttctccatcttctgtgaaatttcttcttccttgtgcagccatgcgcatgaattctat

SEQ ID NO: 93 - skipped

SEQ ID NO: 94 - DNA, 20 bp, Homo sapiens
ggaaggaggacacttctgga

SEQ ID NO: 95 - DNA, 21 bp, Homo sapiens
gctgtgaatggctaaacttgg

SEQ ID NO: 96 - DNA, 20 bp, Homo sapiens
cacagaagctgcagtggaag

SEQ ID NO: 97 - DNA, 21 bp, Homo sapiens
caaatggaggagtcaagacca

SEQ ID NO: 98 - DNA, 26 bp, Homo sapiens
gcagtttaagctgtatgtatatgagg

SEQ ID NO: 99 - DNA, 714 bp, Homo sapiens
atttgcattttcggatttttttggcaaatgtcctgttgcccaggctggatacagtggcatgatcatgggtaactgcacattcaacctcctggactcaagcgattctcgtgcctcagcctccaagtagctgggactacaggcgcccgccaccacgcctggctaatttttatatttttttagagatggggttttgtcatgttgcccaagctggtctcaaacttctgagctcaagggatccacctgccttggccttccaaagtgctgggattacaggtacgagccaccacacagagccgcaaacattttttgaggtcaccaaatctagggtgacaaatacaatagatamcatagaattcatttagtcaaataatacacagtcaaatcatcttatttatctagtatggagaaaggatagtttgttttaataagaacgtcattatcatcatcttctattattgattaccaggaacccacagagtttatgccacttgtgtttaaataaaaatatccacacacaaccacaaataaattcctccattaatatattcatcaaaaaataaattacagtaggaattgttttctgagataccactcaccccaaatatagaatgtacaaaatttgcaatttacaagcaattggagtattattgatatccaatggggaattgagaatgcttcaaaaaatgaggctttccactgcatctataaaagaag

SEQ ID NO: 100 - DNA, 708 bp, Homo sapiens
tttgtttaagacagtgttatcttgggttttctgtccttcacagggaactcaatctttactaagactcctggtctcagttgggtgagtttatcagttttgccccagatacttgcccttatctgttggttttccaccacattatcgtggacagatctttcttccttcttgcttgtgttatctgctagagcattctttctaatgtaatcatctcactcccctgcttaaaatccttcaaggtcttactaacattgccagttgatattatctgccttttttgatttaaggcccattttcaaatactagaatttttggcatacaatccaagggattaaaagatgaaygtaagctttttttttaaagaaagctttggcaaattttttttaaataaccagttattcacagtatattataatattatatttgtatgcttttatgattttttaaatctgaaattatattaaaatgaaagatgagtctcatttcttgtataagttcacttttttgttgttgttgttttggcatttgatgtttgtaagagttgagaaccctaattttctgagaaatgacatggaagactgcagcagtacctctggactccacagttgggtgctcttcgagaccatgttgccatttaaacagaatggtttcctccctttgctctgcctgctgatgtggtctagctagctcctgattaaactctgcctcttg

SEQ ID NO: 101 - DNA, 718 bp, Homo sapiens
tctaggctgtgcacactcactgctgtacagtgttccatgtgtggatataccatgatttacttatcctttcaaccgtggatagacatgtgggtgatttccagttctgagttattattatgaatggtgctgctatggatattctggtacgtgtctttcggtgaacacattgtagccaggttttgacatgctgctttgaagtttagacagttgcaccctgccaggagatttcctttaagacccctgcaccaggccagaaacattcactgcattgcagcaacctgattctgtagttgttgacacaaatccaacacccttctccctaccccagcttgggtaggggttaaaagtagatgaartagggagggaagctgttttcaagttacaagaaaaagttctttacaactgctggccttgttcatactttattttcctctcactcacttccgtttcttttccaggtaagcctgattgcaagcttcattgtacctgtttctttctgactcagattccagctcagcttacatttttcccactaagtaggcagtgatatttcatcacagcaggtacttacaccttttgttctgatgacttaaagcacaagtaggttttgataagtgcttgcagggtttcattttcaaaagtcctatttctgtgtcatatttgttggctttgagcccagtttctcttgctctgccaacagagcaggttatgcctattt

SEQ ID NO: 102 - DNA, 701 bp, Homo sapiens
ttttttcaaacttttcttctccctctcctcatcctctactccttgatcttcacttggagaaggacaattctagaattcctgaactctaggccaaaaggaagtgggcaatcatggcaagcataaacacatccatggcaagttatcagacaccttttgtgggtactaaacagcagggatgcccacttgtcccttggaagtttgcaaacatactgggaaaatggggactataaaattaaaccaccaaagatcagtgtgggagactgaataattaaagggtatccaggtggaccagtcacaaacgctgtaggagctcaatggagacatcagtgggcaycttcctggaagcagtgaggcttgcatggaaataaaaaacagggggttctaatttttgttattgttcaccaatatcagcaaaaaaggtgggcacaccctcaataaatgtttgcaaattctttacatgtgctaattaatcatatcttaagatgcaaaatacattgagggcaaggtttactcttaacaatggtcaatgtaaatccttactttaaataagcatcttataattatgatttgcatggggggcacattttgtcagatcttatttgtcatcattattttgttttgtttgttaatacactcatcttatcttggagtaggagaattattaggtctgttaatctttcttgttgctcactgttattttg

SEQ ID NO: 103 - DNA, 716 bp, Homo sapiens
tatgaaccagaaaatgggccctcaccagacatcacatctgctggcatctttatcaaggacttctcagcctccaaaattgtgagaaataaatttctgttgtgcataagctacccagtctatggtattttgttatagcagcctgaatggactaagacacacttattgaacccccacgtgtttttctgaagaatgaatgcctcacattttacacaagatgtctgtgtgcactggggccgtctagtctaccctggcctggtgatcagggcagggaatcaytgaagtttcccattctctaaaagtggaggaaatggcagccatggggaagctgccttctgctaacacaattgagccgtgaaaacaatatacaactatttttgttatattccagtggtcacacagagcaaccccaatacaataggagggcacaccacaaagccatgagtaccaggaggggtgatcactgggagactccttggaagctggctgccactgtgaggcattatctctgtttcacagaggagaaacagaagctccaataaataattgctcaagtcaactcaacttggaacaggcaggtctggggttcaaacccagacaatgagaccccagaacacatccttttagaacactgccctataccctggcctcaccacaggcctttttttctaacttcctctcttcccctcaccgcgcaaaacattgcaaatgagattttt

SEQ ID NO: 104 - DNA, 886 bp, Homo sapiens
gagggcacaccacaaagccatgagtaccaggaggggtgatcactgggagactccttggaagctggctgccactgtgaggcattatctctgtttcacagaggagaaacagaagctccaataaataattgctcaagtcaactcaacttggaacaggcaggtctggggttcaaacccagacaatgagaccccagaacacatccttttagaacactgccctataccctggcctcaccacaggcctttttttctaacttcctctcttcccctcaccgcgcaaaacattgcaaatgagatttttcctttttcttagaccatttcaaaagtcattgttacttaagggtggaggttggaagatttccaaagaataaaatatacagagaatatctaaccaaagttcctaacacatacacaattcagaaaatgtaactcacagacaagggataacaagaccattgacccartttcagagcttgacgtttacaaaatgaacacaaggcagtgtgggttgtatgcgcgttctgttcagtttctctcctttggggttgtttgggtcagcctgttgtctcatgagactgggtgggctaaattgagcaacattttgctataataagtctgcaagattagaccttaggcaacaaaagccggaaggagaaactacatttcctataaaatgtggaactgtgggataacagtgtaacaacactatgactacaaacagggaaatttatatatgagaaggaactggattgtatgttacctatataaatgatcatgagaaagtcatgttgttcttttgttgtgatcttttaaaccaaatttatagtgcattgaaccaagtaattgtaggccattattttaaagtaggttgtagcacagcatgaattaa

SEQ ID NO: 105 - DNA, 836 bp, Homo sapiens
gaggaggtgatgtacacttttaaaaaacctaatctcacaagcactcactcactatcacgggaacagcaccaaaggaacagcaccaaggcgatgatgcgaaaccattcatgagaaatccgcccccatgatccaatcacctccccgccaggccccacctccaacactggggactacaattccacatggatttgatgggaacacacacccaagccatgtctgatggacacatagtttattttcttttgtgactctgcataggccattcttgccactgggaccccttccctcccaatcctcctggctttccctgcctgtcagcaaactcctgctcctttttcaagcatcaactcggatttaccctctgctgtgatgtcttctgtgactcacatgcagatttaggcacctgtttattrtgttctcaatatatcttacccatactatagaaatatttgttgttttttatctacctagtgttaaattaaataagcacgaggccattggccagaggccctctccatattttgagtttctgtggaacaaacagcaacctaatagtatgtaaacaaactgaaacctaatttaggagtatatttttgtaacatatagcctggtttcagccaatcacagagaagcttcagccaataataagcatccaattgatgagaccacgcccaataaggcagatgcctagctgttgccgatcaagtggtttctctacattgcttttgtgttcaccctagaaaagctcattgctcacactgccaagtggagttttctgaacctcttctggttctgagtgctgcctgattcatgaatcattctttgcccaaataaac

SEQ ID NO: 106 - DNA, 704 bp, Homo sapiens
gtttctctacattgcttttgtgttcaccctagaaaagctcattgctcacactgccaagtggagttttctgaacctcttctggttctgagtgctgcctgattcatgaatcattctttgcccaaataaactctgttaaatttaatttgtctaaactgtttcttttaacactagcttctattccgccttctctgacaagcgttcaggaacccaccccacccccaccccgtactttgggtgtagcccatgtgatttaagtctagccaatcagagcactaaggagctacagttcagaggtgatcatgagacccaggttcatcgaactagagtgaatcctgggactsagcatgagcggctgggaagaaacacacaagtttttgttgcaagtctggagctgctagcagacttcacatactgcctgagcatgaagcaaaaataaagagagtgaaaagaatgagagagaatgggaaagagtctgctggtgacattatttgatcctctgaatgatgcctcacttaaattcaagatatattcttggattttgtgcattaacaaattccctttttgagcttaagcctgcttgatttatctatcatttgcaaccaaaggaacattaaccaataaatacatttcactgtatatctgtgtctatatatctatatgtatttcattttaccaaggtgtctccctactaaccataattcttt

SEQ ID NO: 107 - DNA, 758 bp, Homo sapiens
aactagagtgaatcctgggactgagcatgagcggctgggaagaaacacacaagtttttgttgcaagtctggagctgctagcagacttcacatactgcctgagcatgaagcaaaaataaagagagtgaaaagaatgagagagaatgggaaagagtctgctggtgacattatttgatcctctgaatgatgcctcacttaaattcaagatatattcttggattttgtgcattaacaaattccctttttgagcttaagcctgcttgatttatctatcatttgcaaccaaaggaacattaaccaataaatacatttcactgtatatctgtgtctatatatctrtatgtatttcattttaccaaggtgtctccctactaaccataattctttgagggcagtagatgctcaatatttgtcaaatgaattcagctgaagggtgttttgaaggagactgaccttagaggagggacattttaggaaggctaatggacttagtgtgagatgtgatcaagggactcaaccaagttgaagagtaggattgaaagggaagggacaaataccaaagaaagatttaacaaggcagtgatacagagtggggtggagcaatagttagattaaagcctgagtgctaccctgttctgcgtatttgtttcttttggtgtctctttagcagccagcctaaattaaaagtttattgtactggctgattattgcctgtctaaatcacccgtctctgttagtttatcacaagtgaaaaaatta

SEQ ID NO: 108 - DNA, 758 bp, Homo sapiens
tagagtgaatcctgggactgagcatgagcggctgggaagaaacacacaagtttttgttgcaagtctggagctgctagcagacttcacatactgcctgagcatgaagcaaaaataaagagagtgaaaagaatgagagagaatgggaaagagtctgctggtgacattatttgatcctctgaatgatgcctcacttaaattcaagatatattcttggattttgtgcattaacaaattccctttttgagcttaagcctgcttgatttatctatcatttgcaaccaaaggaacattaaccaataaatacatttcactgtatatctgtgtctatatatctataygtatttcattttaccaaggtgtctccctactaaccataattctttgagggcagtagatgctcaatatttgtcaaatgaattcagctgaagggtgttttgaaggagactgaccttagaggagggacattttaggaaggctaatggacttagtgtgagatgtgatcaagggactcaaccaagttgaagagtaggattgaaagggaagggacaaataccaaagaaagatttaacaaggcagtgatacagagtggggtggagcaatagttagattaaagcctgagtgctaccctgttctgcgtatttgtttcttttggtgtctctttagcagccagcctaaattaaaagtttattgtactggctgattattgcctgtctaaatcacccgtctctgttagtttatcacaagtgaaaaaattaatg

SEQ ID NO: 109 - DNA, 759 bp, Homo sapiens
ggctgggaagaaacacacaagtttttgttgcaagtctggagctgctagcagacttcacatactgcctgagcatgaagcaaaaataaagagagtgaaaagaatgagagagaatgggaaagagtctgctggtgacattatttgatcctctgaatgatgcctcacttaaattcaagatatattcttggattttgtgcattaacaaattccctttttgagcttaagcctgcttgatttatctatcatttgcaaccaaaggaacattaaccaataaatacatttcactgtatatctgtgtctatatatctatatgtatttcattttaccaaggtgtctccctamtaaccataattctttgagggcagtagatgctcaatatttgtcaaatgaattcagctgaagggtgttttgaaggagactgaccttagaggagggacattttaggaaggctaatggacttagtgtgagatgtgatcaagggactcaaccaagttgaagagtaggattgaaagggaagggacaaataccaaagaaagatttaacaaggcagtgatacagagtggggtggagcaatagttagattaaagcctgagtgctaccctgttctgcgtatttgtttcttttggtgtctctttagcagccagcctaaattaaaagtttattgtactggctgattattgcctgtctaaatcacccgtctctgttagtttatcacaagtgaaaaaattaatgatagagaatcagagactcacatataagcaa

SEQ ID NO: 110 - DNA, 760 bp, Homo sapiens
accaataaatacatttcactgtatatctgtgtctatatatctatatgtatttcattttaccaaggtgtctccctactaaccataattctttgagggcagtagatgctcaatatttgtcaaatgaattcagctgaagggtgttttgaaggagactgaccttagaggagggacattttaggaaggctaatggacttagtgtgagatgtgatcaagggactcaaccaagttgaagagtaggattgaaagggaagggacaaataccaaagaaagatttaacaaggcagtgatacagagtggggtggagcaatagttagattaaagcctgagtgctaccctgttctgcgtatttgtttcttttggtgtctctttagcagccagcctaaattaaaagtttattgtrctggctgattattgcctgtctaaatcacccgtctctgttagtttatcacaagtgaaaaaattaatgatagagaatcagagactcacatataagcaaataagcatgattattataagaaagagcttttattaaacaatactttcaggtcttcataagaataggggtagaatttcagagacccacataactcagtgtgcagtaaatgctgctcctgggcaacttaatggagcataaactgccagcaacggtcccaattgaaatggagactggaaggtgaagttgtccttcctttctgtaaccaccaggcaagaggacacttgtaaggtgtgagtagcagcacccaaaaaccagctgcaggac

SEQ ID NO: 111 - DNA, 759 bp, Homo sapiens
ggggtggagcaatagttagattaaagcctgagtgctaccctgttctgcgtatttgtttcttttggtgtctctttagcagccagcctaaattaaaagtttattgtactggctgattattgcctgtctaaatcacccgtctctgttagtttatcacaagtgaaaaaattaatgatagagaatcagagactcacatataagcaaataagcatgattattataagaaagagcttttattaaacaatactttcaggtcttcataagaataggggtagaatttcagagacccacataactcagtgtgcagtaaatgctgctcctgggcaacttaatggagcataaactgccagcaacggtcccaattgaaatggagactggaaggtgaagttgtccttcctttcygtaaccaccaggcaagaggacacttgtaaggtgtgagtagcagcacccaaaaaccagctgcaggactcagtggaagggaggaataaggtcactcttaaaatcctatcacctcacatagaaaaatagctaagtcctaattaagctcaacatcgccactctcagcttatccctgagacaggtcaggagaagagggaccatttgctttgctctgggattgttgcacttctgcaatctgactttgtaaaaaaaaaaaaaattaatttaaacagttgctaccatatgggatagtgtagctcgatggtttctttctctctctcgtccctctcctgctctgccttctatgtatttaccacccctctt

SEQ ID NO: 112 - DNA, 644 bp, Homo sapiens
tggtgaagtgtgaatcatttctccatgtaaaacacataggacaggctgggcatggtggctcacgcctgtaatcccagcactttaggaggcctaggcgggtggatcacctgaggtcaggagttcaagaccagcctgggcaacatggagaaaccccatctctactgaaaatacaaaaattagctgtgcgtgatggcgcacacctgtaatcccagttactcgggagactgaggcaggagaatcacttgaacccgggagcggaggttgcggtgagccgagatcgtgccattgcacttaagcctgggttacaagagcgaaactctgtctcaaaacaaaacacacataggayagagctcagcacagagtagacattaaggattatatcctttgcttggcacaataccttgcacagggcaggcacgcaacagatgtctctggaatgaaggaatgaatgagtgaatgactgggttaagcatgttgccaccaggtggcagaagagcctcactatcaaggcagaacccaaacacgagactcatgagaactccctcctgaagtccagatacacattgaaaaaaaataaaaaaagcactgaaccccatttaggccttgaagtgaagttcctcttctctcttgcccttcctttctct

SEQ ID NO: 113 - DNA, 767 bp, Homo sapiens
aagaccagcctgggcaacatggagaaaccccatctctactgaaaatacaaaaattagctgtgcgtgatggcgcacacctgtaatcccagttactcgggagactgaggcaggagaatcacttgaacccgggagcggaggttgcggtgagccgagatcgtgccattgcacttaagcctgggttacaagagcgaaactctgtctcaaaacaaaacacacataggacagagctcagcacagagtagacattaaggattatatcctttgcttggcacaataccttgcacagggcaggcacgcaacagatgtctctggaatgaaggaatgaatgagtgaatgactgggttaagcatgttgccaccaggtggcagaagagcctcactatcaaggcagaacccaaacacgagaytcatgagaactccctcctgaagtccagatacacattgaaaaaaaataaaaaaagcactgaaccccatttaggccttgaagtgaagttcctcttctctcttgcccttcctttctctcccatctctgctcactctctgctgtaatgaaccatttctttctttcccacttaatacatattagtcagtttgggctgccacagcaaaatactacagactcagtagtttaaacaacagatatttaatgcatcacagttctggaggttggaagtccatgatcaaagtgccatacgggctggtttctggtgaggcttctcttcctggcttgtagctgtccaccttcccactgttattctcacagggcct

SEQ ID NO: 114 - DNA, 720 bp, Homo sapiens
gatccccagaggtgtctgttatgcacagtaagctccaacagtgaaaaatcatttataaagggccgaggacagtggcttgcacctgcaatcccagcactttgggaggtcatggtgggcagattgcttaagcccaggagttccagaccagcctgggcaacatggcaaaacaccatctctactaaaaatttaaaaacttagttaggtgtggtggctggcacctgtagtcgcagctacttgggagggtgaggtaggcggatcacttgaacctgggaggttgaagctgcagtgagctgtaatcatgccactgcactccagcctggatgacagagcaagaccctgtctcaaaaaaaaraaaaaattatcaaggacttttgcctctaataaaatattcacagtggtttccttacttaatttctgaggtcaaaccagaaaatattagcagctgacttaattcaagaaggaggagcttgagtatacgtacttgttggtgtgtcttcaactcttgttctagattttactttgttttaaatatgtaaaaatgcttttagtgattacaacttatgcttcttatttcaacagatattttaaagggaaaaatatataattggatcacaggatataaaaagaaatgcagttatctatatgtgcaaaagcctagctaattgataaaagctataagttgagtcctgccactcaccttggggcaatgattttttattt

SEQ ID NO: 115 - DNA, 720 bp, Homo sapiens
atcatttataaagggccgaggacagtggcttgcacctgcaatcccagcactttgggaggtcatggtgggcagattgcttaagcccaggagttccagaccagcctgggcaacatggcaaaacaccatctctactaaaaatttaaaaacttagttaggtgtggtggctggcacctgtagtcgcagctacttgggagggtgaggtaggcggatcacttgaacctgggaggttgaagctgcagtgagctgtaatcatgccactgcactccagcctggatgacagagcaagaccctgtctcaaaaaaaagaaaaaattatcaaggacttttgcctctaataaaatattcacagtggyttccttacttaatttctgaggtcaaaccagaaaatattagcagctgacttaattcaagaaggaggagcttgagtatacgtacttgttggtgtgtcttcaactcttgttctagattttactttgttttaaatatgtaaaaatgcttttagtgattacaacttatgcttcttatttcaacagatattttaaagggaaaaatatataattggatcacaggatataaaaagaaatgcagttatctatatgtgcaaaagcctagctaattgataaaagctataagttgagtcctgccactcaccttggggcaatgattttttatttatttattttattttattattattatttttttagacagagttgcccag

SEQ ID NO: 116 - DNA, 762 bp, Homo sapiens
tttgtatttatagtagacatggagtttcaccatcttggccaggatggttccgaactcctgacctcgtgatccaccactcggcctcccaaaatgctgggattacaagcataagccactgcaccacgcccggccaatgacccatttttttcaggcaaagtagcaatgggaaaatataaagtttctctagttttaatatagaagtggttaacctaatcacacaagccatacacagggtcatttgggagaatgtgcaaggaggattgcgtatttttatcttttcatagttttcttcttgataaataagcttctattttcaagccaaatctcatcttgcaatttcctgccaacttcacttctctacaaagtttacctttgcttttcccatctctgccctsaggcatttaacaaacactgtgccttttcatttttccagatttaagtgaaacattttgcagaaatgaggaatgtgataacagcccctgaagccctacctgacagcatgacattaatttgggcctgttttctctcatacttttcaattgctccccaatttatatttaatttgccacaggatataaaaagaaatatttctttaatttatattaaatacatctacattaggagagctagaggttatctaagtgaaactagctcgattatctaaaaaaagtcagaataaaataattataagcaaattggaagaacagccaacgttgttaccaataatttcttagagtttgttcaattattgtttgttatact

SEQ ID NO: 117 - DNA, 818 bp, Homo sapiens
ttatcttttcatagttttcttcttgataaataagcttctattttcaagccaaatctcatcttgcaatttcctgccaacttcacttctctacaaagtttacctttgcttttcccatctctgccctcaggcatttaacaaacactgtgccttttcatttttccagatttaagtgaaacattttgcagaaatgaggaatgtgataacagcccctgaagccctacctgacagcatgacattaatttgggcctgttttctctcatacttttcaattgctccccaatttatatttaatttgccacaggatataaaaagaaatatttctttaatttatattaaatacatctacattaggagagctagaggttatctaagtgaaactagctcgattatctaaaaaaagtcagaataaaataattataagcaaattggaagaacagccaacgttgttaccaataatttyttagagtttgttcaattattgtttgttatactctgtttccacttctttagccaaaataagctctaagcaaattcaaatctatttgtatagatgaagtctatgaatttaacatgataacttgaaaaaatgtaaaactttggctgggtgtggtggctcacacctgtaatcccagcactgtgggaggctgtggcgggcggatcacctaaggtcgggagctccagaccagcctggccaacattgtgaaaccccatctctactaaaaatacaagcattagcgaggcatggtggtgggcacctgtaatcccagctactcaggaggctgaggcaggagaatcgcttgaacccaggaggcggaggt

SEQ ID NO: 118 - DNA, 813 bp, Homo sapiens
tgcagaaatgaggaatgtgataacagcccctgaagccctacctgacagcatgacattaatttgggcctgttttctctcatacttttcaattgctccccaatttatatttaatttgccacaggatataaaaagaaatatttctttaatttatattaaatacatctacattaggagagctagaggttatctaagtgaaactagctcgattatctaaaaaaagtcagaataaaataattataagcaaattggaagaacagccaacgttgttaccaataatttcttagagtttgttcaattattgtttgttatactctgtttccacttctttagccaaaataagctctaagcaaattcaaatctatttgtatagatgaagtctatgaatttaacatgataacytgaaaaaatgtaaaactttggctgggtgtggtggctcacacctgtaatcccagcactgtgggaggctgtggcgggcggatcacctaaggtcgggagctccagaccagcctggccaacattgtgaaaccccatctctactaaaaatacaagcattagcgaggcatggtggtgggcacctgtaatcccagctactcaggaggctgaggcaggagaatcgcttgaacccaggaggcggaggttgcagtgagccaagatcgtaccattgcattccagcctgggcaacaagagcaaaactccgtctcaaaaaaaaaaaaaattaaaacccaaataaattcatgtggatcttacccatatttcccatgatttagataggagttggttttaagtttatttttccactcaatgggggaaagg

SEQ ID NO: 119 - DNA, 756 bp, Homo sapiens
catctctactaaaaatacaagcattagcgaggcatggtggtgggcacctgtaatcccagctactcaggaggctgaggcaggagaatcgcttgaacccaggaggcggaggttgcagtgagccaagatcgtaccattgcattccagcctgggcaacaagagcaaaactccgtctcaaaaaaaaaaaaaattaaaacccaaataaattcatgtggatcttacccatatttcccatgatttagataggagttggttttaagtttatttttccactcaatgggggaaaggatttactaggaaaataatgtaaacaatctatttaagaagtcaaatggcttttaagcacttaaaaagctttgatattagcaatttacccataaatattttgttaattacawaatttttttctttttaggaaatatttcttcttttcttcttcttttggctaagcctcagcagccaaattttttattttactttattttagtttactttttagagacagggcctccctctgtcacacacgctggagtgcagtggtatgatcatagctcactataaccacaaactcctgggctcaagccatcctccctcctcagcctcccgagtaggtgggactacaggtgtgcaccactacacccagctaatttttgtagtttttgtagagacggggtcttgtcatgttacccaggctggcctcgaactcctgggctcaagcaatcctccttcctcagcctcccaaaatgctgggattatagg

SEQ ID NO: 120 - DNA, 641 bp, Homo sapiens
tgtgagatggctggaaccatggctgctatcttgtgaccatgaggggaggtacctggtggttcaaaactgccctgctaagtgagaacggaataggaaggttgtaaacagcccaaatctttcttaaccttgttaagccattgagttgacgaactttgcatctgtcctgtctcaggacttcttgttaagcaagatggtatatttttcatatcgtttaaatatttggcctttaaattttcagtaatagtccttacagtgatggctttcagacagaaaattaaaaattttaaaaagtgctatcctaactgattctctcaatgtattcaagtgtaaagaaattwcatgtctaacctctcatggaattagagggaaaaaatttcatgttattttaagtatgttcagttcttttattaactcatattggtttcccccctactccttacccttgcaaccaagataatttgcatctaagaggttttattctgtttccactgatatgtttagaaattactatatctgaggtgggtatattgggaaaacatacactaccactcctttgcagaaatgagggcttattgcagcagctactcgcccttgcaatgcttcctgcttggaaactcgaaggactacattgagcaggtgga

SEQ ID NO: 121 - DNA, 619 bp, Homo sapiens
agagtttcccattcaacagattcttctatggaacacaaatttgcagtgcccattgaagaaacagaagttgctttcaaacagatgtgttggtctctgtttttagtttcaggctataaaccttttgagggcaggtactaaccaccaggttagtacagttatggtgctttagaatctaatctcaagagaaaacatcatttcaaggttcatgttttttcagcctccaaattgggtgtacatgatccacctttaaggctttttgttttttgcttttgtgcccctttatatctcttgcaggaagacttgtctctttccctccaccrcacatttgtacacagactgccacctccacgttaaaaaagaaggcaggaaggggttgtacttgaagtgaccagcaaacattatcttcaagccttaacctcttttgaaagatggtctttgccaataggggagagacagtttctggaggaaacttccatggtgaatacccagccaaagtaagctttttaaaactgctcctgacccagaaggcacatttcaatataggctgactaaatggagaccctctttcaggccctagactacttggccattggcatccatgaacttgctgcaatcagtg

SEQ ID NO: 122 - DNA, 623 bp, Homo sapiens
aaaataattaaaaaattatcctggcacagtagcatgtgcctgtagtcccagctactcaggaggctgaggatcacttgagcccaggagttcaaggctgcagtgagctaggattgcaccactgtgctcgctctagcctgggttacagagacaggtgtctaaaaaataaaaagaaaaatagaaaagccctctaagaagcttccgtctccgctgctccacttcctacctctcgagttccttgtgaccctcctgtatgctctcctagcaaatgattgttttccactgcaccccacccacttcccacatcctcaagcactgaatgtastattactcagattgccctgagcttgcctgtcttcattttgcctactcctagaccgaccccaacacccagaacagaatccagcctctagctgatacttgaatctgtgaaattgacgtagtaaatgggaccagctctgtccttctcttaccttaacttccccttccttctttcctagagagaccttaacttaatgactctctacttcttttctttcaagggaagattgttctgcccatcgccccctcgggattctgtctccatctagtagagggaattttataatcccctcttcattggtgct

SEQ ID NO: 123 - DNA, 548 bp, Homo sapiens
gagaccccatctctattttaaaaaataaaaaaagaaaagaaaagaagagattattaagagtgtggctggtcacccatttgataaggagagtagtgtgagtatcaaccatggacctaatcagccatctcaacagaagccagaaatagagttgggattattccaggagaaataatgctttagtcccctgccagttgggactaaaaggaaaagagaaaacaagatggaatgaagtaaggctgtgaatatgcaatmcccttcaggaaaagagaggaaggatcccaaaggcaattcagacatcatcagggctgctactcccaccacaggcccagagtggaaaggcccctgggaacaaggctacctccatcttggtttcaaagagtgggactgctactcagcactcatgtgggtgtggccctcacagacagccatgtgggcagtgctacactgagctgaagaagcagggacaccccactgaaagatgggggtgataccttccagtggttctggaagggaggaccaccaccccagtgggcctacagggcagagca

SEQ ID NO: 124 - DNA, 20 bp, Artificial sequence
  Synthetic primer
ggaaggaggacacttctgga

SEQ ID NO: 125 - DNA, 21 bp, Artificial sequence
  Synthetic primer
gctgtgaatggctaaacttgg

SEQ ID NO: 126 - DNA, 20 bp, Artificial sequence
  Synthetic primer
cacagaagctgcagtggaag

SEQ ID NO: 127 - DNA, 21 bp, Artificial sequence
  Synthetic primer
caaatggaggagtcaagacca

SEQ ID NO: 128 - DNA, 26 bp, Artificial sequence
  Synthetic primer
gcagtttaagctgtatgtatatgagg

SEQ ID NO: 129 - DNA, 21 bp, Artificial sequence
  Synthetic primer
tgaaagccatcactgtaagga